(19) United States
(12) Patent Application Publication
(10) Pub. No.: US 2026/0098277 A1
(43) Pub. Date: Apr. 9, 2026

(54) BEST1 VECTORS AND USES THEREOF

(71) Applicant: ALDEVRON, LLC, Fargo, ND (US)

(72) Inventors: Ricardo SALADANA-MEYER, Stoneham, MA (US); Jin HUH, Chapel Hill, NC (US); Jodi KENNEDY, Dedham, MA (US); Rosario FERNANDEZ GODINO, Cambridge, MA (US)

(21) Appl. No.: 19/113,376

(22) PCT Filed: Sep. 18, 2023
(86) PCT No.: PCT/US2023/074441

§ 371 Date: Mar. 19, 2025

Related U.S. Application Data
(60) Provisional application No. 63/408,356, filed on Sep. 20, 2022.

(51) Int. Cl. C12N 15/85

(52) U.S. Cl. CPC C12N 15/85; C12N 2800/107

(57) ABSTRACT

Provided herein are expression constructs, nucleic acid vectors, pharmaceutical compositions, and methods for improved expression of functional BEST1 in subjects having mutations in BEST1 and associated retinal dystrophies. Improved expression constructs include BEST 1-targeted short hairpin RNAs (shRNAs), BEST1 coding sequences, promoters driving expression of shRNAs and coding sequences, and/or additional regulatory elements to improve BEST1 expression. Expression constructs, nucleic acid vectors and pharmaceutical compositions thereof, and methods of use thereof, can provide effective treatments for BEST1-associated disorders, such as autosomal dominant bestrophinopathies.

### CROSS-REFERENCE TO RELATED APPLICATIONS

This application claims the benefit of U.S. Provisional Patent Application No. 63/408,356, filed Sep. 20, 2022, the entire contents of which are hereby incorporated by reference in their entirety.

### DESCRIPTION OF THE TEXT FILE SUBMITTED ELECTRONICALLY

This application contains a Sequence Listing in XML format submitted electronically herewith via Patent Center. The contents of the XML copy, created on Sep. 14, 2023, is named “IGT-006PC2_135234-5006.xml” and is 141,885 bytes in size. The Sequence Listing is incorporated herein by reference in its entirety.

### BACKGROUND

Visual impairment and blindness constitute a major global health concern, impacting millions of patients suffering from a wide variety of ocular pathologies. Bestrophin 1 (BEST1)-associated retinal dystrophies (e.g., bestrophinopathies) are chronic and progressive disorders of visual function, which are caused by various autosomal dominant and autosomal recessive forms of BEST1 mutations, which can impact Ca²⁺ signaling and homeostasis in retinal cells. Due in part to complex biological mechanisms and restricted access to the retina, safe and effective treatments for many BEST1-associated retinal dystrophies are lacking. Thus, there is a need in the field for therapeutic vectors providing functional BEST1 expression in the eye.

### SUMMARY

Provided herein are nucleic acid vectors, which can be delivered as nonviral compositions for modulating expression of bestrophin 1 (BEST1) in target cells (e.g., ocular cells, e.g., retinal pigment epithelial (RPE) cells) having aberrant BEST1 expression (e.g., caused by a mutation in native BEST1, e.g., an autosomal dominant bestrophinopathy).

In one aspect, the invention includes a DNA vector comprising: (a) a BEST1-encoding sequence, wherein the BEST1-encoding sequence is a DNA sequence encoding a bestrophin 1 (BEST1) RNA transcript; and (b) a short hairpin RNA (shRNA)-encoding sequence, wherein the shRNA-encoding sequence is a DNA sequence encoding an shRNA, wherein the shRNA is not capable of targeting the BEST1 RNA transcript. In some embodiments, the shRNA-encoding sequence comprises SEQ ID NO: 1 and/or SEQ ID NO: 3, and may comprise SEQ ID NO: 1 and SEQ ID NO: 3 connected by a loop-encoding sequence, e.g., wherein the loop-encoding sequence comprises SEQ ID NO: 2. In some embodiments, the shRNA-encoding sequence comprises SEQ ID NO: 4.

In some embodiments, the BEST1 RNA transcript is altered from a native BEST1 RNA sequence (e.g., from the native BEST1 RNA transcript). In some embodiments, the BEST1 RNA transcript does not comprise SEQ ID NO: 7. In some embodiments, the BEST1 RNA transcript comprises a stretch of 21 consecutive bases that has between 70% and 90% complementarity to the shRNA (e.g. lacks 100% complementarity by 1, 2, 3, 4, 5, or 6 mismatched bases). In some embodiments, the BEST1-encoding sequence comprises SEQ ID NO: 6.

In some embodiments, the DNA vector further comprises a first promoter operably linked to the shRNA-encoding sequence. In some embodiments, the first promoter comprises an RNA polymerase III promoter, e.g., a type III RNA polymerase III promoter, e.g., a U6 promoter.

In some embodiments, the DNA vector further comprises a second promoter operably linked to the BEST1-encoding sequence. In some embodiments, the second promoter comprises a native BEST1 promoter, a native MYO7A promoter, a native ABCA4 promoter, a retroviral Rous sarcoma virus (RSV) LTR promoter, an SV40 promoter, a dihydrofolate reductase promoter, a β-actin promoter, a phosphoglycerol kinase (PGK) promoter, a cytomegalovirus (CMV) enhancer/beta-actin (CAG) promoter, an elongation factor 1 alpha (EF1A) promoter, an interphotoreceptor retinoid-binding protein (IBRP) promoter, a rhodopsin kinase (RK) promoter, or a functional variant thereof.

In some embodiments, the DNA vector further comprises a regulatory element operably linked to the BEST1-encoding sequence. In some embodiments, the regulatory element comprises a sequence derived from intron 6 of ABCA4. In some embodiments, the regulatory element is derived from the 5′ half of ABCA4 intron 6. In some embodiments, the sequence derived from ABCA4 intron 6 comprises at least 90% identity to (e.g., at least 95%, at least 96%, at least 97%, at least 98%, at least 99%, or 100% identity to) at least 500 consecutive nucleotides within ABCA4 intron 6 (e.g., SEQ ID NO: 12), e.g., at least 200, at least 300, at least 400, at least 500, at least 600, at least 700, at least 800, at least 900, at least 1000, at least 1100, at least 1200, at least 1300, at least 1400, at least 1500, or at least 1600 consecutive nucleotides within a native human ABCA4 intron 6 (e.g., SEQ ID NO: 12). In some embodiments, the at least 500 consecutive nucleotides include any of nucleotides 3,158-4,822 of ABCA4 intron 6. In some embodiments, the regulatory element comprises the nucleic acid sequence of SEQ ID NO: 8 or a functional variant thereof (e.g., a functional variant having at least 90% sequence identity to SEQ ID NO: 8). In some embodiments, the regulatory element is downstream of the sequence encoding BEST1.

In some embodiments, the regulatory element comprises a scaffold/matrix attachment region (S/MAR) sequence. In some embodiments, the S/MAR sequence comprises an interferon-beta S/MAR sequence or a functional variant thereof. In some embodiments, the S/MAR sequence comprises the nucleic acid sequence of SEQ ID NO: 9 or 10, or a functional variant thereof.

In some embodiments, the DNA vector further comprises a chicken β-globin insulator (cHS4) comprising the nucleotide sequence of SEQ ID NO: 11 or a functional variant thereof.

In some embodiments, the DNA vector is a nonviral vector. In some embodiments, the DNA vector is a synthetic circular DNA vector.

In some embodiments, the DNA vector comprises multiple shRNA-encoding sequences encoding multiple copies of the same shRNA or encoding multiple different shRNAs. In some embodiments, the DNA vector comprises 2, 3, 4, or more copies of the same shRNA-encoding sequence (e.g., SEQ ID NO: 4). In some embodiments, the DNA vector comprises 2, 3, 4, or more different shRNA-encoding sequences. In some embodiments, each of the different shRNA-encoding sequences encodes an shRNA that cannot target the BEST1 RNA transcript. In some embodiments, each of the different shRNA-encoding sequences encodes an shRNA that can target a native BEST1 RNA.

Embodiments are also provided in which an shRNA-encoding sequence is not included in the DNA vector. In any of the embodiments described above, the shRNA-encoding sequence and any or all regulatory or other sequences operably linked thereto may be omitted from the DNA vector to make a DNA vector that includes the BEST1-encoding sequence and any or all sequences operably linked thereto.

In another aspect, provided herein is a DNA vector comprising: (a) a BEST1-encoding sequence; and (b) a regulatory element operably linked to the BEST1-encoding sequence, wherein the regulatory element comprises a sequence derived from intron 6 of ABCA4. In some embodiments, the sequence derived from ABCA4 intron 6 comprises SEQ ID NO: 8 or a functional variant thereof. In some embodiments, the DNA vector further comprises a promoter operably linked to the BEST1-encoding sequence. In some embodiments, the promoter comprises a native BEST1 promoter, a native MYO7A promoter, a retroviral Rous sarcoma virus (RSV) LTR promoter, an SV40 promoter, a dihydrofolate reductase promoter, a β-actin promoter, a phosphoglycerol kinase (PGK) promoter, a cytomegalovirus (CMV) enhancer/beta-actin (CAG) promoter, an elongation factor 1 alpha (EF1A) promoter, an interphotoreceptor retinoid-binding protein (IBRP) promoter, a rhodopsin kinase (RK) promoter, or a functional variant thereof. In some embodiments, the DNA vector further comprises a scaffold/matrix attachment region (S/MAR) sequence operably linked to the BEST1-encoding sequence. In some embodiments, the S/MAR sequence comprises an interferon-beta S/MAR sequence or a functional variant thereof. In some embodiments, the S/MAR sequence comprises the nucleic acid sequence of SEQ ID NO: 9 or 10, or a functional variant thereof. In some embodiments, the DNA vector further comprises a chicken β-globin insulator (cHS4) comprising the nucleotide sequence of SEQ ID NO: 11 or a functional variant thereof.

In another aspect, provided herein is a DNA vector comprising: (a) a BEST1-encoding sequence; and (b) a promoter operably linked to the BEST1-encoding sequence, wherein the promoter comprises a native MYO7A promoter or a functional variant thereof or a native ABCA4 promoter or functional variant thereof. In some embodiments, the native MYO7A promoter or functional variant thereof comprises SEQ ID NO: 21, 22, or 23, or a functional variant thereof. In some embodiments, the native ABCA4 promoter or functional variant thereof comprises SEQ ID NO: 24, 25, 26, or 27, or a functional variant thereof. In some embodiments, the DNA vector further comprises a regulatory element operably linked to the BEST1-encoding sequence. In some embodiments, the regulatory element comprises a sequence derived from intron 6 of ABCA4. In some embodiments, the sequence derived from ABCA4 intron 6 comprises SEQ ID NO: 8 or a functional variant thereof. In some embodiments, the regulatory element comprises a scaffold/matrix attachment region (S/MAR) sequence. In some embodiments, the S/MAR sequence comprises an interferon-beta S/MAR sequence or a functional variant thereof. In some embodiments, the S/MAR sequence comprises the nucleic acid sequence of SEQ ID NO: 9 or 10, or a functional variant thereof. In some embodiments, the DNA vector further comprises a chicken β-globin insulator (cHS4) comprising the nucleotide sequence of SEQ ID NO: 11 or a functional variant thereof.

In another aspect, provided herein a DNA vector comprising: (a) a BEST1-encoding sequence; and (b) a promoter operably linked to the BEST1-encoding sequence, wherein the promoter comprises a modified promoter derived from a native BEST1 promoter, or a functional variant thereof, wherein the promoter comprises SEQ ID NO: 17, 18, 19, 20, or 40, or a functional variant thereof. In some embodiments, the DNA vector further comprises a regulatory element operably linked to the BEST1-encoding sequence. In some embodiments, the regulatory element comprises a sequence derived from intron 6 of ABCA4. In some embodiments, the sequence derived from ABCA4 intron 6 comprises SEQ ID NO: 8 or a functional variant thereof. In some embodiments, the regulatory element comprises a scaffold/matrix attachment region (S/MAR) sequence. In some embodiments, the S/MAR sequence comprises an interferon-beta S/MAR sequence or a functional variant thereof. In some embodiments, the S/MAR sequence comprises the nucleic acid sequence of SEQ ID NO: 9 or 10, or a functional variant thereof. In some embodiments, the DNA vector further comprises a chicken β-globin insulator (cHS4) comprising the nucleotide sequence of SEQ ID NO: 11 or a functional variant thereof.

In another aspect, provided herein is a pharmaceutical composition comprising a therapeutically effective amount of the DNA vector of any one of the previous embodiments and a pharmaceutically acceptable carrier. In some embodiments, the DNA vector is a nonviral vector and is naked. In other embodiments, the DNA vector is a nonviral vector and is formulated as a liposomal or nanoparticulate formulation. In some embodiments, the pharmaceutical composition is formulated for ocular administration (e.g., subretinal or intravitreal injection).

In another aspect, the invention provides a method of expressing functional BEST1 in a target retinal cell of a subject (e.g., an RPE cell), the method comprising administering to the subject the nucleic acid vector or the pharmaceutical composition of any one of the previous aspects. In some embodiments, the subject has an ocular disorder. In some embodiments, the ocular disorder is autosomal recessive bestrophinopathy, Best vitelliform macular dystrophy, autosomal dominant vitreoretinochoroidopathy, autosomal dominant microcornea, rod-cone dystrophy, early-onset cataract posterior staphyloma syndrome, or retinitis pigmentosa.

In another aspect, the invention provides a method of treating an ocular disorder in a subject, the method comprising administering to the subject a therapeutically effective amount of the DNA vector or the pharmaceutical composition of any one of the previous aspects. In some embodiments, the ocular disorder is autosomal recessive bestrophinopathy, Best vitelliform macular dystrophy, autosomal dominant vitreoretinochoroidopathy, autosomal dominant microcornea, rod-cone dystrophy, early-onset cataract posterior staphyloma syndrome, or retinitis pigmentosa.

In some embodiments of any of the previous aspects involving methods of delivering, expressing, or treating described herein, the administering comprises in vivo electroporation. In some embodiments, the in vivo electroporation comprises: (a) contacting an electrode (e.g., a needle electrode, e.g., a monopolar needle electrode) to an interior region of an eye of the subject (e.g., the retina, the subretinal space, or the vitreous humor), wherein an extracellular space in the retina of the eye comprises the DNA vector of any of the previous aspects; and (b) while the electrode is contacting the interior region of the eye (e.g., the retina, the subretinal space, or the vitreous humor), transmitting one or more pulses of electrical energy through the electrode at conditions suitable for electrotransfer of the DNA vector into a retinal cell (e.g., an RPE cell). In some embodiments, the administering comprises subretinal injection or intravitreal injection.

In another aspect, the invention provides a method of expressing functional BEST1 in a target retinal cell (e.g., an RPE cell) of a subject, the method comprising: (a) contacting an electrode (e.g., a needle electrode, e.g., a monopolar needle electrode) to an interior region of an eye of the subject (e.g., a retina, a subretinal space, or a vitreous humor), wherein an extracellular space in the retina of the eye comprises a nonviral vector comprising: (i) a BEST1-encoding sequence which is a DNA sequence encoding a BEST1 RNA transcript (e.g., any of the BEST1-encoding sequences disclosed herein); and (ii) an shRNA-encoding sequence which is a DNA sequence encoding an shRNA that is capable of targeting the BEST1 RNA endogenous to the subject and is not capable of targeting the BEST1 RNA transcript (e.g., any of the shRNA-encoding sequences disclosed herein); and (b) while the electrode is contacting the interior region of the eye, transmitting one or more pulses of electrical energy through the electrode at conditions suitable for electrotransfer of the DNA vector into the target retinal cell (e.g., the RPE cell). In some embodiments, the shRNA-encoding sequence comprises SEQ ID NO: 1. In some embodiments, the shRNA-encoding sequence comprises SEQ ID NO: 3. In some embodiments, the shRNA-encoding sequence comprises a loop-encoding sequence connecting SEQ ID NO: 1 to SEQ ID NO: 3. In some embodiments, the loop-encoding sequence comprises SEQ ID NO: 2. In some embodiments, the shRNA-encoding sequence comprises SEQ ID NO: 4. In some embodiments, the BEST1 RNA transcript is altered from a native BEST1 sequence (e.g., from the native BEST1 RNA transcript). In some embodiments, the BEST1 RNA transcript does not comprise SEQ ID NO: 7. In some embodiments, the BEST1 RNA transcript comprises a stretch of 21 consecutive bases that has between 70% and 90% complementarity to the shRNA (e.g. lacks 100% complementarity by 1, 2, 3, 4, 5, or 6 mismatched bases). In some embodiments, the DNA sequence encoding the BEST1 RNA transcript comprises SEQ ID NO: 6.

In some embodiments, the DNA vector further comprises a first promoter operably linked to the shRNA-encoding sequence. In some embodiments, the first promoter comprises an RNA polymerase III promoter, e.g., a type III RNA polymerase III promoter, e.g., a U6 promoter. In some embodiments, the DNA vector further comprises a second promoter operably linked to BEST1-encoding sequence. In some embodiments, the second promoter comprises a native BEST1 promoter, a native MYO7A promoter, a native ABCA4 promoter, a retroviral Rous sarcoma virus (RSV) LTR promoter, an SV40 promoter, a dihydrofolate reductase promoter, a β-actin promoter, a phosphoglycerol kinase (PGK) promoter, a cytomegalovirus (CMV) enhancer/beta-actin (CAG) promoter, an elongation factor 1 alpha (EF1A) promoter, an interphotoreceptor retinoid-binding protein (IBRP) promoter, a rhodopsin kinase (RK) promoter, or a functional variant thereof.

In some embodiments, the DNA vector further comprises a regulatory element operably linked to the DNA sequence encoding the BEST1 RNA transcript. In some embodiments, the regulatory element comprises a sequence derived from intron 6 of ABCA4. In some embodiments, the regulatory element is derived from the 5′ half of ABCA4 intron 6. In some embodiments, the sequence derived from ABCA4 intron 6 comprises at least 90% identity to (e.g., at least 95%, at least 96%, at least 97%, at least 98%, at least 99%, or 100% identity to) at least 500 consecutive nucleotides within ABCA4 intron 6 (e.g., SEQ ID NO: 12), e.g., at least 200, at least 300, at least 400, at least 500, at least 600, at least 700, at least 800, at least 900, at least 1000, at least 1100, at least 1200, at least 1300, at least 1400, at least 1500, or at least 1600 consecutive nucleotides within a native human ABCA4 intron 6 (e.g., SEQ ID NO: 12). In some embodiments, the at least 500 consecutive nucleotides include any of nucleotides 3,158-4,822 of ABCA4 intron 6. In some embodiments, the sequence derived from ABCA4 intron 6 comprises SEQ ID NO: 8 or a functional variant thereof.

In some embodiments, the regulatory element comprises a scaffold/matrix attachment region (S/MAR) sequence. In some embodiments, the S/MAR sequence comprises an interferon-beta S/MAR sequence or a functional variant thereof. In some embodiments, the S/MAR sequence comprises the nucleic acid sequence of SEQ ID NO: 9 or 10, or a functional variant thereof. In some embodiments, the DNA vector further comprises a chicken β-globin insulator (cHS4) comprising the nucleotide sequence of SEQ ID NO: 11 or a functional variant thereof.

In some embodiments of any of the preceding aspects, the method includes delivering a nonviral vector (e.g., a naked nucleic acid vector (e.g., a naked circular DNA vector (e.g., a synthetic and/or supercoiled circular DNA vector))) to the extracellular space of the retina, e.g., by subretinal injection or by intravitreal injection. In some embodiments, the interior region of the eye contacting the electrode comprises the vitreous humor. In some embodiments, the electrode is within 10 mm of the retina upon transmission of the one or more pulses of electrical energy. In some embodiments, the interior region of the eye contacting the electrode comprises the retina. In some embodiments, the interior region of the eye contacting the electrode comprises the subretinal space. In some embodiments, the conditions suitable for electrotransfer of the nonviral vector into the target retinal cell comprise a field strength at the target retinal cell from 10 V/cm to 1,500 V/cm. In some embodiments, 1 to 12 pulses of electrical energy are transmitted. In some embodiments, the total number of pulses of electrical energy are transmitted within 1-20 seconds. In some embodiments, the pulses of electrical energy are square waveforms. In some embodiments, the pulses of electrical energy have an amplitude from 5 V to 250 V. In some embodiments, each of the pulses of electrical energy is from 10 to 200 milliseconds in duration. In some embodiments, the target retinal cell is a retinal epithelial (RPE) cell. In other embodiments, the target retinal cell is a photoreceptor. In some embodiments, the nonviral vector is a circular DNA vector.

### INCORPORATION BY REFERENCE

Each patent, publication, and non-patent literature cited in the application is hereby incorporated by reference in its entirety as if each was incorporated by reference individually. To the extent publications and patents or patent applications incorporated by reference conflict with the disclosure contained in the specification, the specification is intended to supersede and/or take precedence over any such conflicting material.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 is a bar graph showing knockdown of endogenous BEST1 mRNA by various short hairpin RNA (shRNA) sequences (sh1-sh4).

FIG. 2 shows representative human therapeutic C³DNA expression construct designs.

FIG. 3 shows representative human therapeutic C³DNA expression constructs with native promoter elements relevant for retinal pigment epithelial (RPE) cell expression. Promoter elements were identified by mapping ABCA4 chromatin regulatory elements using ChIP-Seq for H3K27ac and ATAC-Seq in the indicated cell types (fetal retinal pigment epithelium cells (Fetal RPE); induced pluripotent stem cell retinal pigment epithelium cells (iPSC RPE)).

FIG. 4 shows the transfection efficiency of C³DNA vectors containing various regulatory elements as assessed by relative DNA copy number compared to a genomic locus region.

FIG. 5 shows ocular transgene mRNA expression normalized to DNA copy number, resulting from various regulatory elements. Constructs with the most effective regulatory elements denoted with an *.

FIG. 6 shows representative human ocular therapeutic C³DNA expression construct designs with native MYO7A promoter elements identified by mapping MYO7A chromatin regulatory elements using ChIP-Seq for H3K27ac and ATAC-Seq in the indicated cell types (fetal retinal pigment epithelium cells (Fetal RPE); induced pluripotent stem cell retinal pigment epithelium cells (iPSC RPE)).

FIG. 7 shows the results of transfection experiments in iRPE cells, using vectors containing the regulatory elements shown in FIG. 6.

FIG. 8 shows the relative GFP and mRNA expression of transgene in the RPE/choroid and neural retina (NR) layers of a pig eye, following subretinal delivery by in vivo electrotransfer of C³DNA having full-length or truncated S/MAR sequences.

FIG. 9 shows the effects of including interferon-β scaffold matrix attachment region (S/MAR) sequences in expression constructs after 19 days as assessed by fluorescence activated cell sorting (FACS), and qPCR.

### DETAILED DESCRIPTION

The present invention provides constructs for improved expression of bestrophin 1 (BEST1) transgenes (e.g., for expression in the eye, e.g., in retinal pigment epithelial (RPE) cells), nucleic acid vectors thereof, pharmaceutical compositions thereof, and methods of use thereof (e.g., methods of treatment). The invention is based, in part, on the discovery that co-expression of an exogenous BEST1 RNA transcript and a short hairpin RNA (shRNA) that binds to a natively expressed BEST1 (e.g., an shRNA having SEQ ID NO: 1) can improve BEST1 expression in target cells having aberrant native BEST1 expression (e.g., autosomal dominant bestrophinopathies). Moreover, nucleic acid vectors that provide such co-expression can be delivered to target cells (e.g., RPE cells) can be delivered in the form of non-viral vectors (e.g., naked nucleic acid vectors) using electrotransfer. Such nucleic acid vectors, pharmaceutical compositions thereof, and methods of use thereof can provide effective, durable treatments for BEST1-associated ocular diseases.

### I. Definitions

Unless defined otherwise, technical and scientific terms used herein have the same meaning as commonly understood by one of ordinary skill in the art to which this invention belongs and by reference to published texts, which provide one skilled in the art with a general guide to many of the terms used in the present application. In the event of any conflicting definitions between those set forth herein and those of a referenced publication, the definition provided herein shall control.

As used herein, the term “expression construct” refers to a nucleic acid sequence (e.g., DNA sequence) that is expressed by a cell upon delivery to the cell, e.g., by a nucleic acid vector containing the expression construct. An expression construct may include a sequence of interest (e.g., one or more transgenes, e.g., therapeutic transgenes) and regulatory elements operably linked thereto, which can enhance expression and/or persistence of the DNA vector in a target cell.

As used herein, the terms “vector” and “nucleic acid vector” are used interchangeably and refer to a nucleic acid molecule capable of delivering a therapeutic sequence to which is it linked into a target cell in which the therapeutic sequence can then be transcribed, replicated, processed, and/or expressed in the target cell. After a target cell or host cell processes the therapeutic sequence of the vector, the therapeutic sequence is not considered a vector. One type of vector is a “plasmid”, which refers to a circular double stranded DNA loop containing a bacterial backbone into which additional DNA segments may be ligated. Another type of vector is a phage vector. Another type of vector is a viral vector (e.g., adeno-associated viral (AAV) vector), wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “recombinant vectors” or “expression vectors”). Any of the nucleic acid vectors described herein may be referred to as “isolated nucleic acid vectors.”

As used herein, the term “circular DNA vector” refers to a DNA vector in a circular form. Such circular form is typically capable of being amplified into concatemers by rolling circle amplification. A linear double-stranded nucleic acid having conjoined strands at its termini (e.g., covalently conjugated backbones, e.g., by hairpin loops or other structures) is not a circular vector, as used herein. The term “circular DNA vector” is used interchangeably herein with the terms “covalently closed and circular DNA vector” and “C³DNA.” A skilled artisan will understand that such circular vectors include vectors that are covalently closed with supercoiling and complex DNA topology, as is described herein. In particular embodiments, a circular DNA vector is supercoiled (e.g., monomeric supercoiled). In other embodiments, a circular DNA vector is relaxed circular or relaxed open circular (covalently closed without supercoiling). In certain instances, a circular DNA vector lacks a bacterial origin of replication. In some embodiments, a circular DNA vector comprises a bacterial origin of replication.

The term “synthetic,” as used herein, describes a vector (e.g., circular DNA vector) that was produced in a cell-free process in which bacterial cells are absent from their production from templates. Exemplary cell-free processes for producing synthetic circular DNA vectors are provided in Example 4 and in International Patent Publication WO 2019/178500, which is incorporated herein by reference in its entirety.

As used herein, the term “protein” refers to a plurality of amino acids attached to one another through peptide bonds (i.e., as a primary structure), including multimeric (e.g., dimeric, trimeric, etc.) proteins that are non-covalently associated (e.g., proteins having quaternary structure). Thus, the term “protein” encompasses peptides (e.g., polypeptides), native proteins, recombinant proteins, and fragments thereof. In some embodiments, a protein has a primary structure and no secondary, tertiary, or quaternary structure in physiological conditions. In some embodiments, a protein has a primary and secondary structure and no tertiary or quaternary structure in physiological conditions. In particular embodiments, a protein has a primary structure, a secondary structure, and a tertiary structure, but no quaternary structure in physiological conditions (e.g., a monomeric protein having one or more folded alpha-helices and/or beta sheets). In some embodiments, any of the proteins described herein have a length of at least 25 amino acids (e.g., 50 to 1,000 amino acids).

As used herein, the term “therapeutic protein” refers to a protein that can treat a disease or disorder in a subject. In some embodiments, a therapeutic protein is a therapeutic replacement protein administered to replace a defective (e.g., mutated) protein in a subject. In some embodiments, a therapeutic protein is the same or functionally similar to a native protein that is not defective in a subject.

As used herein, the term “therapeutic replacement protein” refers to a protein that is structurally similar to (e.g., structurally identical to) a protein that is endogenously expressed by a normal (e.g., healthy) individual. A therapeutic replacement protein can be administered to an individual that suffers from a disorder associated with a dysfunction of (or lack of) the protein to be replaced. In some embodiments, the therapeutic replacement protein corrects a defect in a protein resulting from a mutation (e.g., a point mutation, an insertion mutation, a deletion mutation, or a splice variant mutation) in the gene encoding the protein. Therapeutic replacement proteins do not include non-endogenous proteins, such as proteins associated with a pathogen (e.g., as part of a vaccine). Therapeutic replacement proteins may include enzymes, growth factors, hormones, interleukins, interferons, cytokines, anti-apoptosis factors, anti-diabetic factors, coagulation factors, anti-tumor factors, liver-secreted proteins, or neuroprotective factors. In some instances, the therapeutic replacement protein is monogenic.

As used herein, the term “backbone sequence” refers to a portion of plasmid DNA outside the therapeutic sequence that includes one or more bacterial origins of replication or fragments thereof, one or more drug resistance genes or fragments thereof, one or more recombination sites, or any combination thereof. In some embodiments, the backbone sequence includes one or more bacterial origins of replication. Backbone sequences include truncated plasmid backbones of 20 base pairs or more (e.g., 31-40, e.g., 38 base pairs), which may include, e.g., a functional origin of replication.

As used herein, the term “recombination site” refers to a nucleic acid sequence that is a product of site-specific recombination, which includes a first sequence that corresponds to a portion of a first recombinase attachment site and a second sequence that corresponds to a portion of a second recombinase attachment site. One example of a hybrid recombination site is attR, which is a product of site-specific recombination and includes a first sequence that corresponds to a portion of attP and a second sequence that corresponds to a portion of attB. Alternatively, recombination sites can be generated from Cre/Lox recombination. Thus, a vector generated from Cre/Lox recombination (e.g., a vector including a LoxP site) includes a recombination site, as used herein. Other site-specific recombination events that generate recombination sites involve, e.g., lambda integrase, FLP recombinase, and Kw recombinase. Nucleic acid sequences that result from non-site-specific recombination events (e.g., ITR-mediated intermolecular recombination) are not recombination sites, as defined herein.

As used herein, the term “flank,” “flanking,” and “flanked” refer to a pair of regions or points on a nucleic acid molecule (e.g., a plasmid DNA vector) that are outside a reference region of the nucleic acid molecule. In some embodiments, a pair of regions or points flanking a reference region on a nucleic acid are adjacent to (i.e., abut) the reference region (i.e., there are no intervening bases between the reference point and the flanking point). In other embodiments, a pair of regions or points on a nucleic acid molecule that flank a reference region are separated from the reference region by one or more intervening bases (e.g., up to 1,000 intervening bases).

As used herein, the term “operably linked” refers to an arrangement of elements, wherein the components so described are configured so as to perform their usual function. A nucleic acid is “operably linked” when it is placed into a functional relationship with another nucleic acid sequence. For example, a promoter is operably linked to one or more heterologous genes if it affects the transcription of the one or more heterologous genes. Further, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence, and the promoter sequence can still be considered “operably linked” to the coding sequence.

As used herein, the terms “scaffold/matrix attachment region” and “S/MAR” each refers to a DNA sequence of at least 200 nucleotides which mediates attachment of the DNA to a nuclear matrix of a eukaryotic cell, wherein the DNA sequence has at least three sequence motifs ATTA per 100 nucleotides over a stretch of at most 200 nucleotides. Exemplary S/MAR sequences are described in Liebich et al., Nucleic Acids Res. 2002, 30:312-374 and in International Patent Publication No. WO 2019/060253, the S/MAR descriptions of each of which are incorporated herein by reference.

The term “bestrophin 1 (BEST)” refers to any native BEST1 (also known as ARB, BMB, BEST, RP50, VMD2, TU15B, or Best1V1Delta2) from any vertebrate source, including mammals such as primates (e.g., human and cynomolgus monkeys) and rodents (e.g., mice and rats), unless otherwise indicated, as well as functional variants (e.g., natural or synthetic variants), e.g., mutants, muteins, analogs, subunits, receptor complexes, isotypes, splice variants, and fragments thereof. Functional variants can be determined on the basis of known BEST1 signaling (e.g., Ca²⁺ signaling in RPE cells). BEST1 encompasses full-length, unprocessed BEST1, as well as any form of BEST1 that results from native processing in the cell. An exemplary human BEST1 sequence is provided as National Center for Biotechnology Information (NCBI) Gene ID: 7439. In some instances, the BEST1 is encoded by a sequence having at least 95% sequence identity to any one of SEQ ID NO: 5 (e.g., at least 96%, at least 97%, at least 98%, at least 99% or 100% sequence identity to SEQ ID NO: 5). In some instances, the BEST1 is encoded by a sequence having at least 95% sequence identity to any one of SEQ ID NOs: 13-16 (e.g., at least 96%, at least 97%, at least 98%, at least 99% or 100% sequence identity to any one of SEQ ID NOs 13-16).

As used herein, the term “ABCA4 intron 6” refers to a native nucleic acid sequence beginning from the nucleotide directly 3′ (i.e., downstream) to the 3′ end of ABCA4 exon 6 and ending on the nucleotide directly 5′ (i.e., upstream) to the 5′ end of ABCA4 exon 7. An exemplary sequence of a native human ABCA4 intron 6 is given by SEQ ID NO: 12. As used herein, nucleotide numbering of human ABCA4 intron 6 begins at the first position of intron 6 according to NG 009073; i.e., nucleotide 1 of ABCA4 intron 6 corresponds to chromosome 1, strand (−), position 94,564,349 according to GRCh37/hg19. For example, nucleotide 3,158 of ABCA4 intron 6 corresponds to GRCh37/hg19 position 94,561,192 of chromosome 1, strand (−).

The terms “regulatory element” and “control element” are used interchangeably herein and each refer to a non-coding nucleic acid region, such as a promoter, enhancer, and silencer, which function to affect gene expression (e.g., level of expression and/or persistence of expression). In some embodiments, a regulatory element is not transcribed into mRNA. In other embodiments, a regulatory element is transcribed into mRNA but not translated into protein. Suitable regulatory elements are described in International Publication No. WO 2021/055760, which is incorporated herein by reference in its entirety.

A regulatory element is “derived” from a reference sequence (e.g., a native intron) when it contains a functional sequence, or functional variant of a sequence, contained within the reference sequence (e.g., a functional sequence, or functional variant of a sequence, having at least 20, at least 30, at least 40, at least 50, at least 100, at least 150, at least 200, at least 300, at least 400, or at least 500 nucleotide bases having at least 90%, 95%, 96%, 97%, 98%, 99%, or 100% sequence identity with the reference sequence). A regulatory element derived from a reference sequence need not have the same level of function or type of function as the reference sequence; the functional sequence of the regulatory element must confer a detectable function (e.g., improve the level and/or persistence of expression, compared to an expression construct lacking the functional sequence of the regulatory element).

The term “promoter” refers to a regulatory element that regulates transcription of a gene (e.g., an shRNA encoding sequence or a BEST1-encoding sequence) operably linked thereto and includes (a) one or more sequence sufficient to direct transcription and/or (b) recognition sites for RNA polymerase and other transcription factors required for efficient transcription. In some embodiments, the promoter is operably linked 5′ to the gene (e.g., operably linked upstream of the gene). Some promoters can direct cell-specific expression.

As used herein, the term “naked” refers to a nucleic acid molecule (e.g., a circular DNA vector) that is not encapsulated in a lipid envelope (e.g., a liposome) or a polymer matrix and is not physically associated with (e.g., covalently or non-covalently bound to) a solid structure (e.g., a particulate structure) upon administration to the individual. In some instances of the present invention, a pharmaceutical composition includes a naked circular DNA vector.

As used herein, the term “isolated” means artificially produced and not integrated into a native host genome. For example, isolated nucleic acid vectors include nucleic acid vectors that are naked, as well as those that are encapsulated in a lipid envelope (e.g., a liposome) or a polymer matrix. In some embodiments, the term “isolated” refers to a DNA vector that is: (i) synthetic, e.g., amplified in vitro (e.g., in a cell-free environment), for example, by rolling-circle amplification or polymerase chain reaction (PCR); (ii) recombinantly produced by molecular cloning; (iii) purified, as by restriction endonuclease cleavage and gel electrophoretic fractionation, or column chromatography; or (iv) synthesized by, for example, chemical synthesis. An isolated nucleic acid vector is one which is readily manipulable by recombinant DNA techniques well-known in the art. Thus, a nucleotide sequence contained in a vector in which 5′ and 3′ restriction sites are known or for which PCR primer sequences have been disclosed is considered isolated, but a nucleic acid sequence existing in its native state in its natural host is not. An isolated nucleic acid vector may be substantially purified, but need not be.

As used herein, the terms “individual” and “subject” are used interchangeably and include any mammal in need of treatment or prophylaxis, e.g., by a nucleic acid vector, or pharmaceutical composition thereof, described herein. In some embodiments, the individual or subject is a human. In other embodiments, the individual or subject is a non-human mammal (e.g., a non-human primate (e.g., a monkey), a mouse, a pig, a rabbit, a cat, or a dog). The individual or subject may be male or female.

As used herein, an “effective amount” or “effective dose” of a DNA vector, or pharmaceutical composition thereof, refers to an amount sufficient to achieve a desired biological, pharmacological, or therapeutic effect, e.g., when administered to the individual according to a selected administration form, route, and/or schedule. As will be appreciated by those of ordinary skill in this art, the absolute amount of a particular composition that is effective can vary depending on such factors as the desired biological or pharmacological endpoint, the agent to be delivered, the target tissue, etc. Those of ordinary skill in the art will further understand that an “effective amount” can be contacted with cells or administered to a subject in a single dose or through use of multiple doses. An effective amount of a composition to treat a disease may slow or stop disease progression or increase partial or complete response, relative to a reference population, e.g., an untreated or placebo population, or a population receiving the standard of care treatment.

As used herein, “treatment” (and grammatical variations thereof such as “treat” or “treating”) refers to clinical intervention in an attempt to alter the natural course of the individual being treated, which can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and improved prognosis. In some embodiments, nucleic acid vectors of the invention are used to delay development of a disease or to slow the progression of a disease (e.g., retinal degeneration).

As used herein, a “target cell” refers to a cell that expresses a therapeutic protein encoded by a therapeutic gene. In some embodiments, a target cell is a retinal cell (e.g., a RPF cell or a photoreceptor). For example, in particular embodiments, a target cell is a retinal pigment epithelial (RPE) cell.

As used herein, “delivering,” “to deliver,” and grammatical variations thereof, means causing an agent (e.g., a DNA vector) to access a target cell. The agent can be delivered by administration of the agent to an individual having the target cell (e.g., systemically or locally administering the agent) such that the agent gains access to the organ or tissue in which the target cell resides (e.g., retina). Additionally, or alternatively, the agent can be delivered by applying a stimulus to a tissue or organ harboring the agent, wherein the stimulus causes the agent to enter the target cell. Thus, in some instances, an agent is delivered to a target cell by transmitting an electric field into a tissue harboring the agent at conditions suitable for electrotransfer of the agent into a target cell within the tissue.

As used herein, “electrotransfer” refers to movement of a molecule (e.g., a nucleic acid vector, e.g., a naked nucleic acid vector) across a membrane of a target cell (e.g., from outside to inside the target cell, e.g., a retinal cell) that is caused by transmission of an electric field (e.g., a pulsed electric field) to the microenvironment in which the cell resides (e.g., retina). Electrotransfer may occur as a result of electrophoresis, i.e., movement of a molecule (e.g., a nucleic acid, e.g., a naked nucleic acid) along an electric field, based on a charge of the molecule. Electrophoresis can induce electrotransfer, for example, by moving a molecule (e.g., a nucleic acid, e.g., a naked nucleic acid) into proximity of a cell membrane to allow a biotransport process (e.g., endocytosis including pinocytosis or phagocytosis) or passive transport (e.g., diffusion or lipid partitioning) to carry the molecule into the cell. Additionally, or alternatively, electrotransfer may occur as a result of electroporation, e.g., generation of pores in the target cell caused by transmission of an electric field (e.g., a pulsed electric field), wherein the size, shape, and duration of the pores are suitable to accommodate movement of a molecule (e.g., a nucleic acid vector, e.g., a naked nucleic acid vector) from outside the target cell to inside the target cell. Thus, in some instances, electrotransfer occurs as a result of a combination of electrophoresis and electroporation.

As used herein, “administering” is meant a method of giving a dosage of an agent (e.g., a DNA vector) of the invention or a composition thereof (e.g., a pharmaceutical composition, e.g., a pharmaceutical composition including a DNA vector) to an individual. The compositions utilized in the methods described herein can be administered intraocularly, for example, subretinally, intravitreally, or suprachoroidally.

The terms “level of expression” or “expression level” are used interchangeably and generally refer to the amount of a polynucleotide or an amino acid product or protein in a biological sample (e.g., retina). “Expression” generally refers to the process by which gene-encoded information is converted into the structures present and operating in the cell. Therefore, according to the invention, “expression” may refer to transcription into a polynucleotide, translation into a protein, or post-translational modification of the protein. Fragments of the transcribed polynucleotide, the translated protein, or the post-translationally modified protein shall also be regarded as expressed whether they originate from a transcript generated by alternative splicing or a degraded transcript, or from a post-translational processing of the protein, e.g., by proteolysis. “Expressed genes” include those that are transcribed into a polynucleotide as mRNA and then translated into a protein, and also those that are transcribed into RNA but not translated into a protein (for example, transfer and ribosomal RNAs).

As used herein, the term “expression persistence” refers to the duration of time during which a therapeutic sequence, or a functional portion thereof (e.g., one or more coding sequences of a therapeutic DNA vector), is expressible in the cell in which it was transfected (“intra-cellular persistence”) or any progeny of the cell in which it was transfected (“trans-generational persistence”). A therapeutic sequence, or functional portion thereof, may be expressible if it is not silenced, e.g., by DNA methylation and/or histone methylation and compaction. Expression persistence can be assessed by detecting or quantifying (i) mRNA transcribed from the therapeutic sequence in the target cell or progeny thereof (e.g., through qPCR, RNA-seq, or any other suitable method) and (ii) protein translated from the therapeutic sequence in the target cell or progeny thereof (e.g., through Western blot, ELISA, or any other suitable method). In some instances, expression persistence is assessed by detecting or quantifying therapeutic DNA in the target cell or progeny thereof (e.g., the presence of therapeutic circular DNA vector in the target cell, e.g., through episomal DNA copy number analysis) in conjunction with either or both of (i) mRNA transcribed from the therapeutic sequence in the target cell or progeny thereof and (ii) protein translated from the therapeutic sequence in the target cell or progeny thereof. Expression persistence of a therapeutic sequence, or a functional portion thereof, can be quantified relative to a reference vector, such as a control vector produced in bacteria (e.g., a circular vector produced in bacteria or having one or more bacterial signatures not present in the vector of the invention (e.g., a plasmid)), using any gene expression characterization method known in the art. Expression persistence can be quantified at any given time point following administration of the vector. For example, in some embodiments, expression of a DNA vector of the invention persists for at least two weeks after administration if it is detectable in the target cell, or progeny thereof, two weeks after administration of the DNA vector. In some embodiments, expression of a DNA vector “persists” in a target cell if it is detectable in the target cell at one week, two weeks, three weeks, four weeks, six weeks, two months, three months, four months, five months, six months, seven months, eight months, nine months, ten months, eleven months, one year, or longer after administration. In some embodiments, expression of a DNA vector is said to persist for a given period after administration if any detectable fraction of the original expression level remains (e.g., at least 1%, at least 5%, at least 10%, at least 20%, at least 30%, at least 40%, at least 50%, at least 70%, or at least 100% of the original expression level) after the given period of time (e.g., one week, two weeks, three weeks, four weeks, six weeks, two months, three months, four months, five months, six months, seven months, eight months, nine months, ten months, eleven months, one year, or longer after administration).

As used herein, “intra-cellular persistence” refers to the duration of time during which a therapeutic sequence, or a functional portion thereof (e.g., one or more coding sequences of a therapeutic DNA vector), is expressible in the cell in which it was transfected (e.g., a target cell, such as a post-mitotic cell or a quiescent cell). Intra-cellular persistence can be assessed by detecting or quantifying (i) mRNA transcribed from the therapeutic sequence in the target cell and (ii) protein translated from the therapeutic sequence in the target cell. In some instances, intra-cellular persistence is assessed by detecting or quantifying therapeutic DNA in the target cell (e.g., the presence of DNA vector in the target cell) in conjunction with either or both of (i) mRNA transcribed from the therapeutic sequence in the target cell and (ii) protein translated from the therapeutic sequence in the target cell. In some embodiments, a DNA vector of the invention exhibits improved intra-cellular persistence relative to a reference vector (e.g., a DNA vector containing a regulatory element exhibits improved intra-cellular persistence relative to a reference vector that does not contain the regulatory element; or a synthetic circular DNA vector exhibits improved intra-cellular persistence relative to a reference vector that is not synthetic, e.g., a plasmid DNA vector).

As used herein, “trans-generational persistence” refers to the duration of time during which a therapeutic sequence, or a functional portion thereof (e.g., one or more coding sequences of a therapeutic DNA vector), is expressible in progeny of the cell in which the gene was transfected (e.g., progeny of the target cell, such as first-generation, second-generation, third-generation, or fourth-generation descendants of the cell in which the gene was transfected, e.g., through a therapeutic circular DNA vector). Trans-generational persistence accounts for any dilution of a gene over cell divisions and may therefore be useful in measuring persistence of a vector in a dividing tissue over time. In some embodiments, the therapeutic circular DNA vector of the invention exhibits improved trans-generational persistence relative to a reference vector (e.g., a plasmid DNA vector). Trans-generational persistence can be assessed by detecting or quantifying (i) mRNA transcribed from the therapeutic sequence in progeny of the target cell and (ii) protein translated from the therapeutic sequence in progeny of the target cell. In some instances, intra-cellular persistence is assessed by detecting or quantifying therapeutic DNA in progeny of the target cell (e.g., the presence of therapeutic circular DNA vector in progeny of the target cell) in conjunction with either or both of (i) mRNA transcribed from the therapeutic sequence in progeny of the target cell and (ii) protein translated from the therapeutic sequence in progeny of the target cell. In some embodiments, the DNA vector of the invention exhibits improved trans-generational persistence relative to a reference vector (e.g., a DNA vector containing a regulatory element exhibits improved trans-generational persistence relative to a reference vector that does not contain the regulatory element; or a synthetic circular DNA vector exhibits improved trans-generational persistence relative to a reference vector that is not synthetic, e.g., a plasmid DNA vector).

As used herein, a “functional variant” of a nucleic acid sequence differs in at least one nucleic acid residue from the reference nucleic acid sequence, such as a naturally occurring nucleic acid sequence, wherein relevant functional activity of the variant is at least 90% of the level of relevant functional activity of the reference nucleic acid sequence (e.g., substantially similar to the relevant function of the reference nucleic acid sequence). In this context, the difference in at least one nucleic acid residue may consist, for example, in a mutation of an nucleic acid residue to another nucleic acid, a deletion or an insertion. A variant may encode a homolog, isoform, or transcript variant of a therapeutic protein or a fragment thereof encoded by the reference nucleic acid sequence, wherein the homolog, isoform or transcript variant is characterized by a degree of identity or homology, respectively, as defined herein.

In some instances, a functional variant of a polynucleotide or polypeptide includes at least one nucleic acid substitution (e.g., 1-100 nucleic acid or amino acid substitutions, 1-50 nucleic acid or amino acid substitutions, 1-20 nucleic acid or amino acid substitutions, 1-10 nucleic acid or amino acid substitutions, e.g., 1 nucleic acid or amino acid substitution, 2 nucleic acid or amino acid substitutions, 3 nucleic acid or amino acid substitutions, 4 nucleic acid or amino acid substitutions, 5 nucleic acid or amino acid substitutions, 6 nucleic acid or amino acid substitutions, 7 nucleic acid or amino acid substitutions, 8 nucleic acid or amino acid substitutions, 9 nucleic acid or amino acid substitutions, or 10 nucleic acid or amino acid substitutions). Nucleic acid substitutions that result in the expressed polypeptide having an exchanged in amino acids from the same class are referred to herein as conservative substitutions. In particular, these are amino acids having aliphatic side chains, positively or negatively charged side chains, aromatic groups in the side chains or amino acids, the side chains of which can form hydrogen bridges, e.g., side chains which have a hydroxyl function. By conservative substitution, e.g., an amino acid having a polar side chain may be replaced by another amino acid having a corresponding polar side chain, or, for example, an amino acid characterized by a hydrophobic side chain may be substituted by another amino acid having a corresponding hydrophobic side chain (e.g., serine (threonine) by threonine (serine) or leucine (isoleucine) by isoleucine (leucine)).

In order to determine the percentage to which two sequences (e.g., nucleic acid sequences, e.g., DNA or amino acid sequences) are identical, the sequences can be aligned in order to be subsequently compared to one another. For this purpose, gaps can be inserted into the sequence of the first sequence and the component at the corresponding position of the second sequence can be compared. If a position in the first sequence is occupied by the same component as is the case at a corresponding position in the second sequence, the two sequences are identical at this position. The percentage, to which two sequences are identical, is a function of the number of identical positions divided by the total number of positions. The percentage to which two sequences are identical can be determined using a mathematical algorithm. A preferred, but not limiting, example of a mathematical algorithm, which can be used is the algorithm of Karlin et al. (1993), PNAS USA, 90:5873-5877 or Altschul et al. (1997), Nucleic Acids Res., 25:3389-3402. Such an algorithm can be integrated, for example, in the BLAST program. Sequences which are identical to the sequences of the present invention to a certain extent can be identified by this program.

As used herein, “complementarity,” and grammatical variations thereof, refers to the percentage of nucleotide bases of a given sequence that pairs through hydrogen bonding with a reference sequence. In the absence of a given percentage of complementarity, the terms “complement” and “complementary” refer to 100% complementarity.

As used herein, a given sequence (e.g., a BEST1-targeted shRNA) is “100% complementary to,” or has “100% complementarity” with a reference sequence (e.g., a BEST1 RNA transcript) if each of the nucleotide bases of the given sequence pairs through hydrogen bonding with the reference sequence, thereby hybridizing to form a double stranded sequence (e.g., through Watson-Crick base-pairing, e.g., each A pairs with a T or U, and each C pairs with a G). For instance, a binding domain that is in an antisense orientation to a binding site is complementary to the binding site. RNA pairing includes G pairing with U; therefore, an RNA binding domain having G-U pairing with its binding site can be 100% complementary with the binding site. Accordingly, a binding domain that is exactly the reverse complement of its binding domain (i.e., A's of the binding domain are paired with U's of the binding site) can be modified to replace any one or more of the A's with G's without substantially affecting binding.

As used herein, a given sequence (e.g., a BEST1-targeted shRNA) is “at least X % complementary to,” or has “X % complementarity” with a reference sequence (e.g., a BEST1 RNA transcript) if X % of the nucleotide bases of the given sequence pairs through hydrogen bonding with the reference sequence, e.g., hybridizing to form a double stranded sequence (e.g., through Watson-Crick base-pairing, e.g., A pairs with T or U, and C pairs with G). For instance, a binding domain sequence having a length of 150 bases is at least 90% complementary to a binding site having a length of 150 bases if at least 135 of its 150 residues pair through hydrogen bonding with the binding site through Watson-Crick base pairing, leaving 15 or fewer mismatched nucleotides.

The term “pharmaceutically acceptable” means safe for administration to a mammal, such as a human. In some embodiments, a pharmaceutically acceptable composition is approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which a vector or composition of the invention is administered. Examples of suitable pharmaceutical carriers are described in “Remington's Pharmaceutical Sciences,” Mack Publishing Co., Easton, PA., 23^{rd} edition, 2020.

The terms “a” and “an” mean “one or more of.” For example, “a gene” is understood to represent one or more such genes. As such, the terms “a” and “an,” “one or more of a (or an),” and “at least one of a (or an)” are used interchangeably herein.

As used herein, the term “about” refers to a value within +10% variability from the reference value, unless otherwise specified.

The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively unless the context specifically refers to a disjunctive use.

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the present disclosure, subject to any specifically excluded limit in the stated range. Where the stated range includes one or both of the limits, ranges excluding either or both of those included limits are also included in the present disclosure, unless the context clearly dictates otherwise.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Certain specific details of this description are set forth in order to provide a thorough understanding of various embodiments. However, one skilled in the art will understand that the present disclosure may be practiced without these details. In other instances, well-known techniques or methods have not been shown or described in detail to avoid unnecessarily obscuring descriptions of the embodiments. Further, headings provided herein are for convenience only and do not limit the scope or meaning of the claimed disclosure.

Although methods and materials similar or equivalent to those described herein can be used in the practice or testing of the present disclosure, suitable methods, and materials are described below.

For any conflict in definitions between various sources or references, the definition provided herein shall control.

### II. Expression Constructs

Embodiments disclosed herein include expression constructs that provide for expression of a transgene, such as a therapeutic sequence, by a nucleic acid vector (e.g., a nonviral DNA vector (e.g., a naked DNA vector), a circular DNA vector (e.g., a supercoiled circular DNA vector), and/or a synthetic DNA vector (e.g., a synthetic circular DNA vector)). Various elements can be included in such expression constructs, such as therapeutic genes, promoters, and regulatory elements that can enhance expression and/or persistence of the DNA vector in a target cell (e.g., a retinal cell, e.g., an RPE cell). Nucleic acid vectors of the invention can include any of the expression constructs described herein, or combination thereof.

### A. BEST1-Encoding Sequences, RNA Transcripts, and Proteins

Some embodiments of expression constructs disclosed herein include one or more coding sequences for BEST1. In some instances, such BEST1-encoding sequences are DNA sequences that encode a BEST1 RNA transcript. In some embodiments, the BEST1-encoding sequence expresses functional BEST1 to treat or prevent a bestrophinopathy, e.g., a bestrophinopathy associated with a BEST1 dominant mutation or a BEST1 recessive mutation, e.g., an autosomal recessive bestrophinopathy, Best's vitelliform macular dystrophy, BEST1 adult-onset vitelliform macular dystrophy, or autosomal dominant vitreoretinochoroidopathy.

In some embodiments, the BEST1-encoding sequence is a cDNA of BEST1, e.g., a cDNA of BEST1 containing one or more silent mutations (e.g., codon-optimization mutations).

In some instances, the BEST1-encoding sequence encodes a BEST1 RNA transcript that is altered from a native BEST1 sequence (e.g., a native human BEST1 sequence). Alternations can be silent mutations or codon-optimizations that are translated in the same BEST1 amino acid sequence. Such alterations can be made to prevent the shRNA from targeting (i.e., binding to and knocking down (e.g., detectably reducing, reducing by 10-50%, reducing by 50-100%, or substantially blocking, relative to a reference vector not including the shRNA) translation of the BEST1 RNA transcript).

In some instances, the BEST1 RNA transcript encoded by the BEST1-encoding sequence comprises a stretch of 21 consecutive bases that has between 70% and 90% complementarity to the shRNA encoded by the shRNA-encoding sequence (e.g. lacks 100% complementarity by 1, 2, 3, 4, 5, or 6 mismatched bases).

In some embodiments, the BEST1-encoding sequence is a codon-optimized BEST1-encoding sequence. In some embodiments, the BEST1-encoding sequence encodes a functional variant of BEST1. In some embodiments, the BEST1-encoding sequence is or comprises SEQ ID NO: 6. In some embodiments, the BEST1-encoding sequence includes a sequence having at least 75, 80, 85, 90, 95, 98, 99, 99.5, or 99.9% sequence identity or 100% sequence identity to SEQ ID NO: 6. In any of the embodiments disclosed herein, the BEST1-encoding sequence, regulatory elements, promoters, enhancers, and other expression construct components may be human sequences. In some embodiments, the BEST1-encoding sequence encodes a polypeptide that has at least 90% sequence identity, or at least 95% sequence identity, or at least 97%, or at least 98% or at least 99% (or 100%) sequence identity to the polypeptide encoded by SEQ ID NO: 6.

In some embodiments, the genes and/or coding sequences included in expression constructs and nucleic acid vectors described herein (e.g., nonviral DNA vectors, e.g., naked DNA vectors or synthetic circular DNA vectors) are greater than 4.5 Kb in length (e.g., one or more coding sequences, together or each alone, are from 4.5 Kb to 25 Kb, from 4.6 Kb to 24 Kb, from 4.7 Kb to 23 Kb, from 4.8 Kb to 22 Kb, from 4.9 Kb to 21 Kb, from 5.0 Kb to 20 Kb, from 5.5 Kb to 18 Kb, from 6.0 Kb to 17 Kb, from 6.5 Kb to 16 Kb, from 7.0 Kb to 15 Kb, from 7.5 Kb to 14 Kb, from 8.0 Kb to 13 Kb, from 8.5 Kb to 12.5 Kb, from 9.0 Kb to 12.0 Kb, from 9.5 Kb to 11.5 Kb, or from 10.0 Kb to 11.0 Kb in length, e.g., from 4.5 Kb to 8 Kb, from 8 Kb to 10 Kb, from 10 Kb to 15 Kb, from 15 Kb to 20 Kb in length, or greater, e.g., from 4.5 Kb to 5.0 Kb, from 5.0 Kb to 5.5 Kb, from 5.5 Kb to 6.0 Kb, from 6.0 Kb to 6.5 Kb, from 6.5 Kb to 7.0 Kb, from 7.0 Kb to 7.5 Kb, from 7.5 Kb to 8.0 Kb, from 8.0 Kb to 8.5 Kb, from 8.5 Kb to 9.0 Kb, from 9.0 Kb to 9.5 Kb, from 9.5 Kb to 10 Kb, from 10 Kb to 10.5 Kb, from 10.5 Kb to 11 Kb, from 11 Kb to 11.5 Kb, from 11.5 Kb to 12 Kb, from 12 Kb to 12.5 Kb, from 12.5 Kb to 13 Kb, from 13 Kb to 13.5 Kb, from 13.5 Kb to 14 Kb, from 14 Kb to 14.5 Kb, from 14.5 Kb to 15 Kb, from 15 Kb to 15.5 Kb, from 15.5 Kb to 16 Kb, from 16 Kb to 16.5 Kb, from 16.5 Kb to 17 Kb, from 17 Kb to 17.5 Kb, from 17.5 Kb to 18 Kb, from 18 Kb to 18.5 Kb, from 18.5 Kb to 19 Kb, from 19 Kb to 19.5 Kb, from 19.5 Kb to 20 Kb, from 20 Kb to 21 Kb, from 21 Kb to 22 Kb, from 22 Kb to 23 Kb, from 23 Kb to 24 Kb, from 24 Kb to 25 Kb in length, or greater, e.g., about 4.5 Kb, about 5.0 Kb, about 5.5 Kb, about 6.0 Kb, about 6.5 Kb, about 7.0 Kb, about 7.5 Kb, about 8.0 Kb, about 8.5 Kb, about 9.0 Kb, about 9.5 Kb, about 10 Kb, about 11 Kb, about 12 Kb, about 13 Kb, about 14 Kb, about 15 Kb, about 16 Kb, about 17 Kb, about 18 Kb, about 19 Kb, about 20 Kb in length, or greater).

### B. Short Hairpin RNAs and Encoding Sequences

Nucleic acid vectors of the invention include expression constructs that encode short hairpin RNAs (shRNAs). Such shRNAs can bind to endogenous BEST1 RNA transcripts (e.g., mutated BEST1 RNA in patients having BEST1-associated retinal dystrophies (e.g., bestrophinopathies)). Thus, without wishing to be bound by theory, the invention provides BEST1 vectors that can treat dominant bestrophinopathies by knocking down endogenous BEST1 expression with shRNA. As noted in the aforementioned section, such shRNA sequences can be non-complementary (e.g., less than 100% complementary) to a BEST1 RNA transcript encoded by the same vector. In some instances, the portion of shRNA sequences that are non-complementary to the BEST1 RNA transcript are 100% complementary to a corresponding portion of functional endogenous BEST1 (e.g., NM_004183.3 (SEQ ID NO: 5)) or to a corresponding portion of endogenous BEST1 having impaired functionality. In some embodiments, the shRNA sequence encoded in a vector has a target sequence of 15-30 bases (e.g., 20-25 bases, e.g., 21 bases) that has one, two, three, four, five, six, or more bases that are mismatched (imparting less than 100% complementarity) to a BEST1 RNA transcript.

In some instances, an expression construct includes a DNA sequence encoding an shRNA comprising SEQ ID NO: 1 (GCCTACGACTGGATTAGTATC). Additionally, or alternatively, the shRNA-encoding sequence includes SEQ ID NO: 3 (GATACTAATCCAGTCGTAGGC). In some instances, the shRNA-encoding sequence includes SEQ ID NO: 1 and SEQ ID NO: 3, wherein SEQ ID NO: 1 and SEQ ID NO: 3 are connected by a loop-encoding sequence (e.g., a loop-encoding sequence comprising SEQ ID NO: 2 (CTCGAG)). In some instances, the shRNA-encoding sequence includes SEQ ID NO: 4, or a functional variant thereof (e.g., a functional variant of SEQ ID NO: 4 having at least 90%, at least 91%, at least 92%, at least 93%, at least 94%, at least 95%, at least 96%, at least 97%, at least 98%, at least 99%, or 100% sequence identity to SEQ ID NO: 4).

In some embodiments, an expression construct may include multiple shRNA-encoding sequences. The multiple shRNA-encoding sequences may be multiple copies of the same shRNA-encoding sequence. For example, embodiments may include 2, 3, 4, 5, 6, 7, 8, 9, 10, or more copies of the same shRNA-encoding sequence (e.g., SEQ ID NO: 4). The multiple shRNA-encoding sequences may also encode different shRNAs. For example, embodiments may include 2, 3, 4, 5, 6, 7, 8, 9, 10, or more different shRNA-encoding sequences. In some embodiments, all of the different shRNA-encoding sequences encode shRNAs that are capable of targeting endogenous BEST1 RNA but are not capable of targeting a BEST1 RNA transcript encoded on the DNA vector. Some embodiments may include multiple copies of multiple different shRNA-encoding sequences. For example, a DNA vector may include 2, 3, 4, or more copies of a first shRNA-encoding sequence, 2, 3, 4, or more copies of a second shRNA-encoding sequence, 2, 3, 4, or more copies of a third shRNA-encoding sequence. In some embodiments, all of the different shRNA-encoding sequences encode shRNAs that are capable of targeting endogenous BEST1 RNA but are not capable of targeting a BEST1 RNA transcript encoded on the DNA vector.

In some embodiments, expression constructs and DNA vectors provided herein do not include any shRNA-encoding sequences.

### C. Promoters

Expression constructs disclosed herein can include one or more promoters. In some embodiments, the one or more promoters includes a native sequence derived from the endogenous promoter of a BEST1 coding sequence. In some embodiments, a promoter includes a native sequence of the same gene to which it is operably linked. For example, a BEST1 coding sequence can be operably linked to, and be under the control of, a sequence derived from the native BEST1 genetic locus, such as a sequence upstream of the BEST1 transcription start site. In some embodiments, the promoter sequence and coding sequence are derived from native sequences of the same species. For example, an expression construct may include an BEST1 native promoter sequence from the human genome and the BEST1 coding sequence from the human genome or a functional variant thereof or a BEST1 native promoter sequence from the human genome and the BEST1 coding sequence from the human genome or a functional variant thereof.

In some instances, a promoter driving expression of a BEST1-encoding sequence in the nucleic acid vectors described herein comprises a nucleic acid sequence having at least 80% identity to any one of SEQ ID NOs: 17-20 (e.g., at least 85%, at least 90%, at least 91%, at least 92%, at least 93%, at least 94%, at least 95%, at least 96%, at least 97%, at least 98%, at least 99%, or 100% identity to any one of SEQ ID NOs: 17-20). In some instances, the promoter includes a nucleic acid sequence having at least 80% identity (e.g., at least 85%, at least 90%, at least 91%, at least 92%, at least 93%, at least 94%, at least 95%, at least 96%, at least 97%, at least 98%, at least 99%, or 100% identity to) at least 50, 60, 70, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, or 2400 nucleotides of any one of SEQ ID NOs: 17-20. In some instances, a promoter driving expression of a BEST1-encoding sequence in the nucleic acid vectors described herein comprises any one of SEQ ID NOs: 17-20 or at least 50, 60, 70, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, or 2400 consecutive nucleotides of any one of SEQ ID NOs: 17-20.

In some instances, a promoter driving expression of a BEST1-encoding sequence in the nucleic acid vectors described herein comprises a nucleic acid sequence having at least 80% identity to SEQ ID NO: 40 (e.g., at least 85%, at least 90%, at least 91%, at least 92%, at least 93%, at least 94%, at least 95%, at least 96%, at least 97%, at least 98%, at least 99%, or 100% identity to SEQ ID NO: 40). In some instances, the promoter includes a nucleic acid sequence having at least 80% identity (e.g., at least 85%, at least 90%, at least 91%, at least 92%, at least 93%, at least 94%, at least 95%, at least 96%, at least 97%, at least 98%, at least 99%, or 100% identity to) at least 50, 60, 70, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, or 950 nucleotides of SEQ ID NO: 40. In some instances, a promoter driving expression of a BEST1-encoding sequence in the nucleic acid vectors described herein comprises SEQ ID NO: 40 or at least 50, 60, 70, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, or 950 consecutive nucleotides of SEQ ID NO: 40.

In some instances, a promoter driving expression of a BEST1-encoding sequence can be a known promoter referred to as a VMD2 promoter, e.g., any of the VMD2 promoters described in International Patent Publication No. WO 2019/195727.

In some instances, a promoter driving expression of a BEST-encoding sequence is a native promoter of another ocular gene or gene endogenously expressed in RPE cells. In some instances, the promoter driving expression of a BEST coding sequence is a native MYO7A promoter, such as any of the native MYO7A promoters described in the Examples below. In other instances, the promoter driving expression of a BEST1 coding sequence is a native ABCA4 promoter.

In some embodiments, the expression construct includes one or more of the following constructs that include sequences derived from native promoter sequences: MYO7A Promoter HS1/2_Intron1 (SEQ ID NO: 21), MYO7A Promoter HS1-3 (SEQ ID NO: 22), MYO7A Promoter Min (SEQ ID NO: 23), ABCA4 Promoter Exon_Intron1_Short (SEQ ID NO: 24), ABCA4 Promoter Exon_Intron1_large (SEQ ID NO: 25), or ABCA4 Promoter_Large (SEQ ID NO: 26), ABCA4 Promoter Short (SEQ ID NO: 27), or functional variants thereof. In some embodiments, the expression construct includes a sequence having at least 75, 80, 85, 90, 95, 98, 99, 99.5 or 99.9% sequence identity or having 100% sequence identity, to any of SEQ ID NOs: 21-27, or a sequence identity between any two of these values. In some embodiments, the expression construct includes a sequence having at least 75, 80, 85, 90, 95, 98, 99, 99.5 or 99.9% sequence identity to 50, 60, 70, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, or 4000 nucleotides of any of SEQ ID NOs: 21-27.

Expression of shRNA-encoding sequences can be driven by a separate promoter than the promoter driving expression of BEST1. Promoters suitable for driving expression of shRNA are known in the art. In some embodiments, the promoter operably linked to (and driving expression of) the shRNA-encoding sequence (e.g., a sequence comprising SEQ ID NO: 1 and/or SEQ ID NO: 3, e.g., a sequence comprising SEQ ID NOs: 1-3; e.g., a sequence comprising or consisting of SEQ ID NO: 4) is an RNA polymerase III promoter, e.g., a type III RNA polymerase III promoter, e.g., a U6 promoter. In some instances, a U6 promoter is operably linked to the shRNA-encoding sequence (e.g., a sequence comprising SEQ ID NO: 1 and/or SEQ ID NO: 3, e.g., a sequence comprising SEQ ID NOs: 1-3; e.g., a sequence comprising or consisting of SEQ ID NO: 4).

In addition to the sequence sufficient to direct transcription, a promoter sequence of the invention can also include sequences of other regulatory elements that are involved in modulating transcription (e.g., enhancers, Kozak sequences, and introns). For example, in some embodiments disclosed herein, the expression construct includes sequences derived from a BEST1 native promoter. In some embodiments, regulatory elements, such as promoters, introns, insulators, enhancers, or other elements, are derived from native sequences of the same species as the gene to which they are operably linked in expression constructs.

In some embodiments, promoters included in expression constructs disclosed herein are tissue-specific promoters in that, in normal operation, they drive expression only when present in certain tissue types, such as, for example, ocular tissue. In some embodiments, a promoter used in an expression construct is not tissue-specific but is capable of driving expression in any tissue type. In some embodiments, the promoter is an inducible promoter. In some embodiments, the promoter is a constitutive promoter. In some embodiments, the construct described herein comprises a cytomegalovirus (CMV) enhancer/beta-actin (CAG) promoter (e.g., SEQ ID NO: 28 or a functional variant thereof), elongation factor 1 alpha (EF1A) promoter (e.g., SEQ ID NO: 29 or a functional variant thereof), interphotoreceptor retinoid-binding protein (IBRP) promoter, rhodopsin kinase (RK) promoter (e.g., G protein-coupled receptor kinase 1 (GRK1) promoter), SV40 promoter, dihydrofolate reductase promoter, β-actin promoter, phosphoglycerol kinase (PGK) promoter, or functional variants thereof.

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources. Many other systems have been described and can be readily selected by one of skill in the art. Examples of inducible promoters regulated by exogenously supplied promoters include zinc-inducible sheep metallothionine (MT) promoters, dexamethasone-inducible mouse mammary tumor virus promoters, T7 polymerase promoter systems, ecdysone insect promoters, tetracycline-repressible systems, tetracycline-inducible systems, RU486-inducible systems, and rapamycin-inducible systems. Still other types of inducible promoters which may be useful in this context are those which are regulated by a specific physiological state, e.g., temperature, acute phase, a particular differentiation state of the cell, or in replicating cells only.

### D. Other Regulatory Elements

In addition to promoters, expression constructs and nucleic acid vectors described herein can include other regulatory elements operably linked to the shRNA sequence and/or the BEST1 coding sequence, which can include, for example, appropriate transcription initiation, termination, and enhancer sequences; efficient RNA processing signals such as splicing and polyadenylation (polyA) signals; sequences that stabilize cytoplasmic mRNA; sequences that enhance translation efficiency (i.e., Kozak consensus sequence); and sequences that enhance stability of the encoded product.

For expression constructs that include genes encoding a protein, a polyadenylation (poly-A, or pA) sequence can be inserted following the gene (e.g., operably linked 3′ to the gene, e.g., directly linked 3′ to the gene).

The precise nature of the regulatory sequences needed for gene expression in host cells may vary between species, tissues or cell types, but shall in general include, as necessary, 5′ non-transcribed and 5′ non-translated sequences involved with the initiation of transcription and translation respectively, such as a TATA box, capping sequence, CAAT sequence, enhancer elements, and the like. Such 5′ non-transcribed regulatory sequences may include a promoter region that includes a promoter sequence for transcriptional control of the operably joined gene. Regulatory sequences may also include enhancer sequences or upstream activator sequences. The vectors of the disclosure may optionally include 5′ leader or signal sequences.

In some embodiments, expression constructs disclosed herein include scaffold-matrix attachment regions (S/MARs). Without being bound by theory, it is believed that S/MAR elements can help establish long-term gene expression from a DNA vector through the interaction of the S/MAR element with the nuclear matrix. Known S/MAR constructs include the human IFN-γ S/MAR (SEQ ID NO: 9) and the human APOB S/MAR (NCBI Gene ID 106632268). Other known S/MAR elements can be included in expression constructs disclosed herein, as can functional variants thereof. In some embodiments, a variant (SEQ ID NO: 10) of the IFN-γ S/MAR comprising tandem repeats of a functional portion of the IFN-γ S/MAR is included in expression constructs provided herein. In some embodiments, the expression construct includes a sequence having at least 75, 80, 85, 90, 95, 98, 99, 99.5 or 99.9% sequence identity or having 100% sequence identity to SEQ ID NO: 9 or 10. S/MAR elements can be operably linked either 5′ or 3′ to a coding sequence of an expression construct.

In some embodiments, expression constructs disclosed herein include chromatin insulator elements. In some embodiments, the one or more chromatin insulator elements may include one or more chicken hypersensitive site-4 elements (cHS4; SEQ ID NO: 11), which is a chromatin insulator from the chicken β-globin locus control region. In some embodiments, the expression construct includes a sequence having at least 75, 80, 85, 90, 95, 98, 99, 99.5 or 99.9% sequence identity or having 100% sequence identity to SEQ ID NO: 11.

In some embodiments, expression constructs disclosed herein include a regulatory element derived from (e.g., containing a portion of, or a variant thereof) a native sequence of ABCA4 intron 6. As described in the Examples provided herein, such regulatory elements, e.g., SEQ ID NO: 8, can enhance persistence and expression levels of genes operably linked thereto. Thus, some embodiments of the invention feature a regulatory element derived from a native sequence of ABCA4 intron 6, e.g., a sequence in the 5′ half of ABCA4 intron 6 (i.e., a sequence that is upstream from the midpoint between the 5′ and 3′ end of ABCA4 intron 6) or a sequence in the 5′ third of ABCA4 intron 6 (i.e., a sequence that is within the 5′-most 33.3% of ABCA4 intron 6).

In some instances, a regulatory element derived from ABCA4 intron 6 has one or more (one, two, three, four, five, or more) sequences that are identical to at least 100 consecutive nucleotides within a native human ABCA4 intron 6 (e.g., SEQ ID NO: 12), e.g., at least 200, at least 300, at least 400, at least 500, at least 600, at least 700, at least 800, at least 900, at least 1000, at least 1100, at least 1200, at least 1300, at least 1400, at least 1500, or at least 1600 consecutive nucleotides within a native human ABCA4 intron 6 (e.g., SEQ ID NO: 12), e.g., from 100-5000, from 500 to 2500, from 1000 to 2000, or from 1500 to 1700 consecutive nucleotides within a native human ABCA4 intron 6 (e.g., SEQ ID NO: 12). In some instances, sequences shared between the regulatory element and ABCA4 intron 6 include nucleotides wholly or partially within nucleotides 3158-4822 of ABCA4 intron 6 (numbering starting from the 5′ end of ABCA4 intron 6), e.g., SEQ ID NO: 8.

In some instances, a regulatory element derived from ABCA4 intron 6 has one or more (one, two, three, four, five, or more) sequences that are identical to at least 100 consecutive nucleotides within the 5′ half of a native human ABCA4 intron 6 (e.g., SEQ ID NO: 12), e.g., at least 200, at least 300, at least 400, at least 500, at least 600, at least 700, at least 800, at least 900, at least 1000, at least 1100, at least 1200, at least 1300, at least 1400, at least 1500, or at least 1600 consecutive nucleotides within the 5′ half of a native human ABCA4 intron 6 (e.g., SEQ ID NO: 12), e.g., from 100-5000, from 500 to 2500, from 1000 to 2000, or from 1500 to 1700 consecutive nucleotides within the 5′ half of a native human ABCA4 intron 6 (e.g., SEQ ID NO: 12). In some instances, sequences shared between the regulatory element and ABCA4 intron 6 include nucleotides wholly or partially within nucleotides 3158-4822 of ABCA4 (numbering starting from the 5′ end of ABCA4 intron 6), e.g., SEQ ID NO: 8.

In some instances, a regulatory element derived from ABCA4 intron 6 has one or more (one, two, three, four, five, or more) sequences that are identical to at least 100 consecutive nucleotides within the 5′ third of a native human ABCA4 intron 6 (e.g., SEQ ID NO: 12), e.g., at least 200, at least 300, at least 400, at least 500, at least 600, at least 700, at least 800, at least 900, at least 1000, at least 1100, at least 1200, at least 1300, at least 1400, at least 1500, or at least 1600 consecutive nucleotides within the 5′ third of a native human ABCA4 intron 6 (e.g., SEQ ID NO: 12), e.g., from 100-5000, from 500 to 2500, from 1000 to 2000, or from 1500 to 1700 consecutive nucleotides within the 5′ third of a native human ABCA4 intron 6 (e.g., SEQ ID NO: 12). In some instances, sequences shared between the regulatory element and ABCA4 intron 6 include nucleotides wholly or partially within nucleotides 3158-4822 of ABCA4 (numbering starting from the 5′ end of ABCA4 intron 6), e.g., SEQ ID NO: 8.

In some instances, a regulatory element is a functional variant of any of the aforementioned ABCA4 intron 6-derived regulatory elements. For example, in some instances, a regulatory element derived from ABCA4 intron 6 has one or more (one, two, three, four, five, or more) sequences that are at least 90% identical (e.g., at least 95% identical, at least 96% identical, at least 97% identical, at least 98% identical, or at least 99% identical) to at least 100 consecutive nucleotides within a native human ABCA4 intron 6 (e.g., SEQ ID NO: 12), e.g., at least 200, at least 300, at least 400, at least 500, at least 600, at least 700, at least 800, at least 900, at least 1000, at least 1100, at least 1200, at least 1300, at least 1400, at least 1500, or at least 1600 consecutive nucleotides within a native human ABCA4 intron 6 (e.g., SEQ ID NO: 12), e.g., from 100-5000, from 500 to 2500, from 1000 to 2000, or from 1500 to 1700 consecutive nucleotides within a native human ABCA4 intron 6 (e.g., SEQ ID NO: 12). In some instances, sequences shared between the regulatory element and ABCA4 intron 6 include nucleotides wholly or partially within nucleotides 3158-4822 of ABCA4 (numbering starting from the 5′ end of ABCA4 intron 6), e.g., SEQ ID NO: 8.

In some instances, a regulatory element derived from ABCA4 intron 6 has one or more (one, two, three, four, five, or more) sequences that are at least 90% identical (e.g., at least 95% identical, at least 96% identical, at least 97% identical, at least 98% identical, or at least 99% identical) to at least 100 consecutive nucleotides within the 5′ half of a native human ABCA4 intron 6 (e.g., SEQ ID NO: 12), e.g., at least 200, at least 300, at least 400, at least 500, at least 600, at least 700, at least 800, at least 900, at least 1000, at least 1100, at least 1200, at least 1300, at least 1400, at least 1500, or at least 1600 consecutive nucleotides within the 5′ half of a native human ABCA4 intron 6 (e.g., SEQ ID NO: 12), e.g., from 100-5000, from 500 to 2500, from 1000 to 2000, or from 1500 to 1700 consecutive nucleotides within the 5′ half of a native human ABCA4 intron 6 (e.g., SEQ ID NO: 12). In some instances, sequences shared between the regulatory element and ABCA4 intron 6 include nucleotides wholly or partially within nucleotides 3158-4822 of ABCA4 (numbering starting from the 5′ end of ABCA4 intron 6), e.g., SEQ ID NO: 8.

In some instances, a regulatory element derived from ABCA4 intron 6 has one or more (one, two, three, four, five, or more) sequences that are at least 90% identical (e.g., at least 95% identical, at least 96% identical, at least 97% identical, at least 98% identical, or at least 99% identical) to at least 100 consecutive nucleotides within the 5′ third of a native human ABCA4 intron 6 (e.g., SEQ ID NO: 12), e.g., at least 200, at least 300, at least 400, at least 500, at least 600, at least 700, at least 800, at least 900, at least 1000, at least 1100, at least 1200, at least 1300, at least 1400, at least 1500, or at least 1600 consecutive nucleotides within the 5′ third of a native human ABCA4 intron 6 (e.g., SEQ ID NO: 12), e.g., from 100-5000, from 500 to 2500, from 1000 to 2000, or from 1500 to 1700 consecutive nucleotides within the 5′ third of a native human ABCA4 intron 6 (e.g., SEQ ID NO: 12). In some instances, sequences shared between the regulatory element and ABCA4 intron 6 include nucleotides wholly or partially within nucleotides 3158-4822 of ABCA4 intron 6 (numbering starting from the 5′ end of ABCA4 intron 6), e.g., SEQ ID NO: 8.

In some instances, a regulatory element derived from ABCA4 intron 6 has one or more (one, two, three, four, five, or more) sequences that are identical to at least 100 consecutive nucleotides of nucleotides 3158-4822 of ABCA4 intron 6, e.g., at least 200, at least 300, at least 400, at least 500, at least 600, at least 700, at least 800, at least 900, at least 1000, at least 1100, at least 1200, at least 1300, at least 1400, at least 1500, or at least 1600 consecutive nucleotides of nucleotides 3158-4822 of ABCA4 intron 6.

In some instances, a regulatory element derived from ABCA4 intron 6 has one or more (one, two, three, four, five, or more) sequences that are at least 90% identical (e.g., at least 95% identical, at least 96% identical, at least 97% identical, at least 98% identical, or at least 99% identical) to at least 100 consecutive nucleotides of nucleotides 3158-4822 of ABCA4 intron 6, e.g., at least 200, at least 300, at least 400, at least 500, at least 600, at least 700, at least 800, at least 900, at least 1000, at least 1100, at least 1200, at least 1300, at least 1400, at least 1500, or at least 1600 consecutive nucleotides of nucleotides 3158-4822 of ABCA4 intron 6.

In some embodiments, the expression construct includes a sequence having at least 75, 80, 85, 90, 95, 98, 99, 99.5 or 99.9% sequence identity or having 100% sequence identity to SEQ ID NO: 8.

In some embodiments, the expression construct includes a sequence having at least 75, 80, 85, 90, 95, 98, 99, 99.5 or 99.9% sequence identity or having 100% sequence identity to nucleotides 3158-4822 of ABCA4 intron 6.

In some of any of the aforementioned instances, the regulatory element derived from ABCA4 intron 6 has been mutated in one or more positions (e.g., one, two, three, or more positions), relative to the native ABCA4 intron 6 sequence, to remove a recognition site of a restriction enzyme, e.g., a type IIs restriction enzyme (e.g., BsaI), which can improve manufacturing efficiency by streamlining cell-free production of synthetic circular DNA vectors using the methods described in the Examples herein (e.g., by consolidating steps by using a type IIs restriction enzyme). For instance, nucleotide 3530 of native human ABCA4 intron 6 (SEQ ID NO: 12), which is a G, can be deleted to remove a BsaI recognition site in a regulatory element derived from ABCA4 intron 6, thereby facilitating an improved, BsaI-based manufacturing process. For example, in some embodiments, a nucleotide sequence from nucleotides 3158-4822 of native ABCA4 intron 6 is modified to delete of G3530, thereby producing the ABCA4 intron 6-derived regulatory element of SEQ ID NO: 8.

Promoters, coding sequences, shRNA sequences, and other elements can be included in expression constructs disclosed herein in any suitable order that provides for effective expression and/or persistence of functional BEST1. In some embodiments, an expression construct includes, in a 5′ to 3′ direction, a first promoter (e.g., an RNA polymerase III promoter, e.g., a U6 promoter), an shRNA-encoding sequence (e.g., a sequence comprising SEQ ID NO: 1, a sequence comprising SEQ ID NO: 1 and SEQ ID NO: 3, e.g., a sequence comprising SEQ ID NOs: 1-3; e.g., a sequence comprising or consisting of SEQ ID NO: 4) operably linked to the first promoter, a second promoter (e.g., an ocular promoter, e.g., a native BEST1 promoter), and a BEST1 encoding sequence operably linked to the second promoter. In some instances, a regulatory element is operably linked to (e.g., upstream or downstream of) the BEST1 encoding sequence. Sequence elements disclosed herein can be arranged in other suitable combinations and orders.

### III. Nucleic Acid Vectors

Provided herein are nucleic acid vectors that include any of the expression constructs described herein, or components (e.g., regulatory elements) or combinations thereof. The nucleic acid vectors can be produced according to methods for production of plasmid DNA vectors, nanoplasmid vectors (as described in, e.g., WO 2008/153733 and WO 2014/035457), minicircle DNA vectors (as described in, e.g., U.S. Pat. Nos. 8,828,726 and 9,233,174), mini-intronic plasmids (described in, e.g., Lu et al., Mol. Ther. 2013, 21:954 and U.S. Pat. No. 9,347,073), synthetic circular DNA vectors as described herein and in WO 2019/178500, closed-ended DNA vectors (as described, e.g., in U.S. 2020/0283794 and 2021/0071197), doggybone DNA vectors (as described, e.g., in U.S. 2015/0329902 and U.S. Pat. No. 9,499,847), or ministring DNA vectors (as described, e.g., in U.S. Pat. Nos. 9,290,778 and USRE48908E1). In particular embodiments, any of the nucleic acid vectors described herein comprise a therapeutic sequence (e.g., a BEST1 encoding sequence). In some instances, a nucleic acid vector of the invention is a nonviral DNA vector (e.g., a naked DNA vector), a circular DNA vector (e.g., a supercoiled circular DNA vector), and/or a synthetic DNA vector (e.g., a synthetic circular DNA vector)), which comprises: a BEST1-encoding sequence which is a DNA sequence encoding a BEST1 RNA transcript and an shRNA-encoding sequence which is a DNA sequence encoding an shRNA comprising SEQ ID NO: 1 and/or SEQ ID NO: 3 (e.g., a sequence comprising SEQ ID NOs: 1-3; e.g., a sequence comprising or consisting of SEQ ID NO: 4), wherein the shRNA is not capable of targeting the BEST1 RNA transcript.

In some instances, the nucleic acid vectors are circular DNA vectors that persist intracellularly (e.g., in dividing or in quiescent cells, such as post-mitotic cells) as episomes, e.g., in a manner similar to AAV vectors. In any of the embodiments described herein, a circular DNA vector may be a non-integrating vector. Circular DNA vectors provided herein can be naked DNA vectors, devoid of components inherent to viral vectors (e.g., viral proteins) and substantial components of bacterial plasmid DNA, such as immunogenic components (e.g., immunogenic bacterial signatures (such as CpG islands or CpG motifs) or components additionally, or otherwise associated with reduced persistence (e.g., CpG islands or CpG motifs). Circular DNA vectors feature one or more therapeutic sequences and may lack plasmid backbone elements, such as (i) a bacterial origin of replication and/or (ii) a drug resistance gene and/or (iii) a recombination site. Synthetic circular DNA vectors lacking an origin or replication can be synthesized through various means known in the art and described herein. Synthesis methods may involve use of phage polymerase, such as Phi29 polymerase, as a replication tool using, e.g., rolling circle amplification. Particular methods of cell-free synthesis of synthetic circular DNA vectors are further described in International Patent Publication No. WO 2019/178500, which is hereby incorporated by reference.

In other embodiments, therapeutic circular DNA vectors described herein can be non-synthetic vectors (e.g., containing bacterial backbone sequences such as origin of replication and/or recombination).

Such nucleic acid vectors can be in vivo-produced, and may lack a selectable marker (e.g., drug resistance gene) and optionally a recombination site, e.g., by using engineered bacterial cells to produce circular DNA vectors from a parental plasmid. Bacterial cells (e.g., E. coli) can be engineered to contain a Rep gene encoding a bacterial replication protein, which is optionally integrated into the bacterial genome. The engineered cells can be transfected with a parental plasmid having a vector sequence and a backbone sequence. The vector sequence includes an ori sequence corresponding to the Rep gene and does not include a selectable marker. The backbone sequence includes a selectable marker and does not include the ori sequence included in the vector sequence. The parental plasmid may also have restriction enzyme recognition sequences, or site-specific recombination sequences, or transposase recognition sequences flanking the vector sequence arranged so that the plasmid backbone sequence can be separated from the vector sequence inside the cell by restriction digestion, site-specific recombination, or transposase action. In the case of restriction digestion, the circular DNA vector is then formed by self-ligation of the vector sequence. In the case of site-specific recombination or transposase action, the circular DNA vector is formed as recombination or transposase action is completed. Expression of the rep protein after separation of the vector sequence and formation of the circular DNA vector can maintain the circular DNA vector at a high copy number, despite the circular DNA vector lacking a selectable marker. In contrast, maintenance of the plasmid backbone sequence in the engineered bacterial cell after separation can be avoided by changing the culture conditions to remove selective pressure for the selectable marker. Culturing of a population of bacterial cells with a high copy number of circular DNA vector under conditions in which the parental plasmid is not maintained can efficiently produce a high yield of highly pure circular DNA vector. Such methods are described in WO 2023/122625 and 63/509,458 (filed Jun. 21, 2023), which are hereby incorporated by reference in their entireties.

One benefit of using a transposase-based system is the ability to further reduce the backbone size within the circular DNA vector. For instance, use of a site-specific recombinase results in a recombination site (e.g., an attachment site) within the vector, near or adjacent to the replication origin. In contrast, use of a transposase allows the replication origin to directly connect the 5′ end of the therapeutic sequence to 3′ end of the therapeutic sequence without intervening sequences. In some instances, use of a transposase allows for a “scarless” backbone by positioning the resulting sequence of the transposition (the transposase overhang) within the therapeutic sequence without modifying the function of the therapeutic sequence. As an example, piggybac transposase produces a four-bp transposase overhang of TTAA. By positioning the plasmid backbone within the sequence of interest at a TTAA site, one can design the system such that, upon transposase-mediated excision of the plasmid backbone from the sequence of interest, the original sequence of interest is restored, leaving only the original TTAA sequence as the transposase scar. This leaves the backbone within the circular DNA vector free of a transposase scar. Thus, the plasmid backbone sequences in the vector can consist entirely of replication origin.

Additionally, or alternatively, the transposase scar may be positioned within the vector backbone (e.g., within the sequence containing the replication origin). For instance, if the parental plasmid contains inverted repeats (left-end) and (right-end) flanking the backbone, and or transposase overhang sequences flanking the therapeutic sequence, the transposase scar will be positioned between the 3′ and 5′ ends of the sequence of interest (e.g., next to the origin of replication).

In some embodiments, the engineered bacterial cells for producing the circular DNA vector of this disclosure include a Rep gene encoding a bacterial replication protein directing replication from ColE2-P9 origin, and which may be integrated into the bacterial genome. Alternatively, the Rep gene is included on an extrachromosomal DNA molecule such as, for example, a plasmid or a bacterial artificial chromosome (“BAC”). The engineered bacterial cells further comprise a parental plasmid comprising a vector sequence and a backbone sequence. The vector sequence includes a replication origin (ori) sequence corresponding to the Rep gene and does not include a selectable marker. The backbone sequence includes a gene encoding a selectable marker and does not include the ori sequence included in the vector sequence. The parental plasmid also has enzyme recognition sequences (e.g., restriction enzyme recognition sequences, site-specific recombination sequences, or transposase recognition sequences) flanking the vector sequence arranged so that the plasmid backbone sequence can be separated from the vector sequence inside the cell by restriction digestion, transposition, or site-specific recombination.

In some embodiments, a short origin of replication is used in the circular DNA vector to minimize bacterial sequences, such as a ColE2-P9 replication origin, or a functional variant thereof. In such embodiments, the Rep gene encodes a ColE2-P9 replication protein. In some exemplary embodiments, the Rep gene encodes a ColE2-P9 replication protein that has the amino acid sequence set forth in SEQ ID NO: 41 (or a functional variant thereof, for example, having at least 80%, or at least 90%, or at least 95%, or at least 97%, or at least 98%, or at least 99% sequence identity thereto). Other suitable replication proteins include replication proteins encoded by naturally-occurring plasmids, including, for example, those that are related to ColE2-P9, such as ColE3-CA38.

In some exemplary embodiments, the ori (e.g., one strand) comprises or consists of a nucleotide sequence as set forth in SEQ ID NO: 42. In some embodiments, the ori sequence is a functional fragment of the ColE2-P9 ori sequence that has the DNA sequence (on one strand) set forth in SEQ ID NO: 42. The 40 base pair functional fragment set forth in SEQ ID NO: 42 is capable of supporting vector replication in a cell expressing the ColE2-P9 replication protein. In some embodiments, the ori is ColE2-P9 origin and is no more than about 40 nucleotides in length, or no more than 38 nucleotides in length, no more than 37 nucleotides in length, or no more than 36 nucleotides in length, or no more than 34 nucleotides in length, or no more than 30 nucleotides in length. In various embodiments, the ColE2-P9 origin is from 20 to 40 nucleotides in length, or from 30 to 40 nucleotides in length, or from 34 to 40 nucleotides in length, thereby minimizing bacterial-derived sequences in the circular vector. In some embodiments, the ori sequence is a naturally occurring ori sequence.

In some instances, the ori sequence is a functional variant of a naturally occurring ori, such as, for example, an ori sequence that has been modified to be shorter than a corresponding naturally occurring ori sequence, while still retaining the ability to support replication initiation. Such functional variants of the ColE2-P9 replication origin include SEQ ID NOs: 43-51. Such sequences are shown herein as a single strand for convenience, although it is recognized that the origin will be present in the vector as double-stranded DNA. In some embodiments, the functional variant has 1, 2, 3, 4, or 5 nucleotide substitutions with respect to a origin sequence of SEQ ID NOS: 43-51. With respect to SEQ ID NO: 51, each X is selected from A, T, C, or G. In some embodiments: X1 is A, T, or C; X2 is A, T, or C; X3 is A, T, or G; X4 is A, T, or C; X5 is A, T, or G; X6 is C; X7 is A.

In some instances, circular DNA vectors provided herein are naked DNA vectors and are devoid of components inherent to viral vectors (e.g., viral proteins) and substantial components of bacterial plasmid DNA, such as immunogenic components (e.g., immunogenic bacterial signatures (e.g., CpG motifs)) or components additionally or otherwise associated with reduced persistence (e.g., CpG islands). For example, in some embodiments, the circular DNA vector contains DNA in which at least 50% (e.g., at least 60%, at least 70%, at least 80%, at least 90%, at least 95%, at least 97%, at least 99%, or essentially all) of the DNA lacks one or more elements of bacterial plasmid DNA, such as immunogenic components (e.g., immunogenic bacterial signatures (e.g., CpG motifs)) or components additionally or otherwise associated with reduced persistence (e.g., CpG islands). In some embodiments, at least 50% (e.g., at least 60%, at least 70%, at least 80%, at least 90%, at least 95%, at least 97%, at least 99%, or essentially all) of the DNA lacks CpG methylation. In some embodiments, the circular DNA vector contains DNA in which at least 50% (e.g., at least 60%, at least 70%, at least 80%, at least 90%, at least 95%, at least 97%, at least 99%, or essentially all) of the DNA lacks bacterial methylation signatures, such as Dam methylation and Dcm methylation. For examples, in some embodiments, the circular DNA vector contains DNA in which at least 50% (e.g., at least 60%, at least 70%, at least 80%, at least 90%, at least 95%, at least 97%, at least 99%, or essentially all) of the GATC sequences are unmethylated (e.g., by Dam methylase). Additionally, or alternatively, the circular DNA vector contains DNA in which at least 50% (e.g., at least 60%, at least 70%, at least 80%, at least 90%, at least 95%, at least 97%, at least 99%, or essentially all) of the CCAGG sequences and/or CCTGG sequences are unmethylated (e.g., by Dcm methylase.

In some embodiments, the circular DNA vector is persistent in vivo (e.g., the therapeutic circular DNA vector exhibits improved expression persistence (e.g., intra-cellular persistence and/or trans-generational persistence) and/or therapeutic persistence relative to a reference vector, e.g., a circular DNA vector having one or more bacterial signatures not present in the vector of the disclosure).

In some embodiments, expression of a circular DNA vector persists for at least two weeks, at least three weeks, at least four weeks, at least six weeks, at least two months, at least three months, at least four months, at least five months, at least six months, at least seven months, at least eight months, at least nine months, at least ten months, at least eleven months, at least one year, or longer after administration. In some embodiments, the circular DNA vector persists for at least about six months or at least one year, or at least 18 months, or at least two years in ocular cells (such as photoreceptor cells and/or RPE cells). In some embodiments, the expression level of the circular DNA vector does not decrease by more than 90%, or by more than 50%, or by more than 25%, or by more than 10% in the 1 week or more, e.g., 2 weeks, 3 weeks, 5 weeks, 7 weeks, 9 weeks or more, 13 weeks or more, or 18 weeks or more following transfection from levels observed within the first 1, 2, or 3 days. In some embodiments, administration of the nucleic acid vector of this disclosure (e.g., to retinal cells) is no more than 4 times per year, or no more than 2 times per year, or no more than once per year, or even less frequently (e.g., once every two years).

In embodiments, the circular DNA vector is monomeric. In some embodiments, the circular DNA vector is supercoiled, e.g., following treatment with a topoisomerase (e.g., gyrase).

In some embodiments involving circular DNA vectors (e.g., circular DNA vectors), the 3′ end of the therapeutic sequence is connected to the 5′ end of the therapeutic sequence in a therapeutic circular DNA vector (e.g., circular DNA vector) by a non-bacterial sequence of no more than 30 bp (e.g., from 3 bp to 24 bp, from 4 bp to 18 bp, from 5 bp to 12 bp, or from 6 bp to 10 bp; e.g., from 3 bp to 5 bp, from 4 bp to 6 bp, from 8 bp to 12 bp, from 12 bp to 18 bp, from 18 bp to 24 bp, or from 24 bp to 30 bp; e.g., 3 bp, 4 bp, 5 bp, 6 bp, 7 bp, or 8 bp). For example, the 3′ end of the therapeutic sequence may be connected to the 5′ end of the therapeutic sequence by a non-bacterial sequence corresponding to sticky end or overhang of the type IIs restriction enzyme cut site (e.g., TTTT or AAAA).

Generally, a circular DNA vector is capable of having a higher ratio of therapeutic sequence to non-therapeutic sequence (e.g., sequence connecting the 3′ end of the therapeutic sequence to the 5′ end of the therapeutic sequence), relative to a circular DNA vector made in vivo, such as a plasmid, which contains a bacterial backbone limiting the ratio. In some instances, the ratio of therapeutic sequence (e.g., from the 5′ end of any functional upstream elements to the 3′ end of any downstream functional elements, such as an S/MAR or polyA tail) to non-therapeutic sequence (e.g., a restriction site overhang, an origin of replication, and/or an inert artifact of cloning) in a circular DNA vector (e.g., circular DNA vector) of the invention is at least 10 (e.g., a therapeutic sequence is about 5000 bp and the non-therapeutic sequence is less than about 500 bp). For example, in some instances, the ratio of therapeutic sequence (e.g., from the 5′ end of any functional upstream elements to the 3′ end of any downstream functional elements, such as an S/MAR or polyA tail) to non-therapeutic sequence (e.g., a restriction site overhang, an origin of replication, and/or an inert artifact of cloning) in a circular DNA vector (e.g., circular DNA vector) of the invention is at least 50 (e.g., a therapeutic sequence is about 5000 bp and the non-therapeutic sequence is less than about 100 bp). In some instances, the ratio of therapeutic sequence (e.g., from the 5′ end of any functional upstream elements to the 3′ end of any downstream functional elements, such as an S/MAR or polyA tail) to non-therapeutic sequence (e.g., a restriction site overhang, an origin of replication, and/or an inert artifact of cloning) in a circular DNA vector (e.g., circular DNA vector) of the invention is at least 100 (e.g., a therapeutic sequence is about 8000 bp and the non-therapeutic sequence is less than about 80 bp). In some instances, the ratio of therapeutic sequence (e.g., from the 5′ end of any functional upstream elements to the 3′ end of any downstream functional elements, such as an S/MAR or polyA tail) to non-therapeutic sequence (e.g., a restriction site overhang, an origin of replication, and/or an inert artifact of cloning) in a circular DNA vector (e.g., circular DNA vector) of the invention is at least 500 (e.g., a therapeutic sequence is about 8000 bp and the non-therapeutic sequence is less than about 16 bp). In some instances, the ratio of therapeutic sequence (e.g., from the 5′ end of any functional upstream elements to the 3′ end of any downstream functional elements, such as an S/MAR or poly A tail) to non-therapeutic sequence (e.g., a restriction site overhang, an origin of replication, and/or an inert artifact of cloning) in a circular DNA vector (e.g., circular DNA vector) of the invention is at least 1,000 (e.g., a therapeutic sequence is about 8000 bp and the non-therapeutic sequence is less than about 8 bp). In some instances, the ratio of therapeutic sequence (e.g., from the 5′ end of any functional upstream elements to the 3′ end of any downstream functional elements, such as an S/MAR or polyA tail) to non-therapeutic sequence (e.g., a restriction site overhang, an origin of replication, and/or an inert artifact of cloning) in a circular DNA vector (e.g., circular DNA vector) of the invention is about 2,000 (e.g., a therapeutic sequence is about 8000 bp and the non-therapeutic sequence is about 4 bp, e.g., corresponding to a restriction site overhang, e.g., a type IIs restriction site overhang).

In some embodiments, the therapeutic sequence includes a reporter sequence. Such reporter sequences can be useful in verifying therapeutic gene sequence expression, for example, in specific cells and tissues. Reporter sequences that may be provided in a transgene include, without limitation, DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements which drive their expression, the reporter sequences provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA), and immunohistochemistry. For example, where the marker sequence is the LacZ gene, the presence of the vector carrying the signal is detected by assays for β-galactosidase activity. Where the transgene is green fluorescent protein or luciferase, the vector carrying the signal may be measured visually by color or light production in a luminometer. In some embodiments, the therapeutic sequence lacks a reporter sequence.

Expression constructs described herein can be assembled into viral vectors, such as vectors consisting of, or derived from, adeno-associated virus (AAV), adenovirus, Retroviridae family virus, parvovirus, coronavirus, rhabdovirus, paramyxovirus, picornavirus, alphavirus, herpes virus, or poxvirus.

In some instances, the nucleic acid vector is a non-viral DNA vector (e.g., the DNA vector is not encapsulated within a viral capsid). Additionally, or alternatively, in some embodiments, the nucleic acid vector is not encapsulated in an envelope (e.g., a lipid envelope) or a matrix (e.g., a polymer matrix) and is not physically associated with (e.g., covalently or non-covalently bound to) a solid structure (e.g., a particulate structure) prior to and upon administration to the individual. In some embodiments, the nucleic acid vector is untethered to any adjacent nucleic acid vectors such that, in a solution of nucleic acid vectors, each nucleic acid vector is free to diffuse independently of adjacent nucleic acid vectors. In some embodiments, the nucleic acid vector is associated with another agent in liquid solution, such as a charge-altering molecule or a stabilizing molecule.

The nucleic acid vector may be a naked DNA vector, i.e., not complexed with another agent (e.g., encapsulated within, conjugated to, or non-covalently bound to another agent). Naked DNA vectors may be co-formulated (e.g., in solution) with agents that are not complexed with the naked DNA vector, such as buffering agents and/or agents that are generally recognized as safe (GRAS) by the U.S. Food and Drug Administration.

### IV. Pharmaceutical Compositions

Nucleic acid vectors, such as any of the DNA vectors (e.g., nonviral DNA vectors (e.g., naked DNA vectors), circular DNA vectors (e.g., supercoiled circular DNA vectors), and/or synthetic DNA vectors (e.g., synthetic circular DNA vectors)) described herein can be included in pharmaceutical compositions, e.g., formulated for administration to a subject, e.g., for treatment of a disease or disorder (e.g., an ocular disease or disorder).

In some instances, the pharmaceutical composition includes a therapeutically effective amount of any of the nucleic acid vectors described herein. In some embodiments, the pharmaceutical composition contains at least 1.0 mg nucleic acid vector in a pharmaceutically acceptable carrier (e.g., from 1.0 mg to 10 g, from 1.0 mg to 5.0 g, from 1.0 mg to 1.0 g, from 1.0 mg to 500 mg, from 1.0 mg to 200 mg, from 1.0 mg to 100 mg, from 1.0 mg to 50 mg, from 1.0 mg to 25 mg, from 1.0 mg to 20 mg, from 1.0 mg to 15 mg, from 1.0 mg to 10 mg, from 1.0 mg to 5.0 mg, from 2.0 mg to 10 g, from 2.0 mg to 5.0 g, from 2.0 mg to 1.0 g, from 2.0 mg to 500 mg, from 2.0 mg to 200 mg, from 2.0 mg to 100 mg, from 2.0 mg to 50 mg, from 2.0 mg to 25 mg, from 2.0 mg to 20 mg, from 2.0 mg to 15 mg, from 2.0 mg to 10 mg, from 2.0 mg to 5.0 mg, from 5.0 mg to 10 g, from 5.0 mg to 5.0 g, from 5.0 mg to 1.0 g, from 5.0 mg to 500 mg, from 5.0 mg to 200 mg, from 5.0 mg to 100 mg, from 5.0 mg to 50 mg, from 5.0 mg to 25 mg, from 5.0 mg to 20 mg, from 5.0 mg to 15 mg, from 5.0 mg to 10 mg, from 10 mg to 10 g, from 10 mg to 5.0 g, from 10 mg to 1.0 g, from 10 mg to 500 mg, from 10 mg to 200 mg, from 10 mg to 100 mg, from 10 mg to 50 mg, from 10 mg to 25 mg, from 10 mg to 20 mg, or from 10 mg to 15 mg). In some embodiments, a pharmaceutical composition contains at least 2.0 mg nucleic acid vector in a pharmaceutically acceptable carrier. In some embodiments, a pharmaceutical composition produced by any of the methods described herein contains at least 5.0 mg circular DNA vector in a pharmaceutically acceptable carrier. In some embodiments, a pharmaceutical composition produced by any of the methods described herein contains at least 10.0 mg circular DNA vector in a pharmaceutically acceptable carrier.

In some embodiments, a pharmaceutical composition contains therapeutic circular DNA vector that is at least 60% supercoiled monomer, at least 70% supercoiled monomer, at least 80% supercoiled monomer, or at least 90% supercoiled monomer (e.g., 60% to 80% supercoiled monomer, 60% to 90% supercoiled monomer, 60% to 95% supercoiled monomer, 60% to 99% supercoiled monomer, 60% to 99.5% supercoiled monomer, 60% to 99.9% supercoiled monomer, 65% to 80% supercoiled monomer, 65% to 90% supercoiled monomer, 65% to 95% supercoiled monomer, 65% to 99% supercoiled monomer, 65% to 99.5% supercoiled monomer, 65% to 99.9% supercoiled monomer, 70% to 80% supercoiled monomer, 70% to 90% supercoiled monomer, 70% to 95% supercoiled monomer, 70% to 99% supercoiled monomer, 70% to 99.5% supercoiled monomer, 70% to 99.9% supercoiled monomer, 75% to 80% supercoiled monomer, 75% to 90% supercoiled monomer, 75% to 95% supercoiled monomer, 75% to 99% supercoiled monomer, 75% to 99.5% supercoiled monomer, 75% to 99.9% supercoiled monomer, 80% to 85% supercoiled monomer, 80% to 90% supercoiled monomer, 80% to 95% supercoiled monomer, 80% to 99% supercoiled monomer, 80% to 99.5% supercoiled monomer, 80% to 99.9% supercoiled monomer, 85% to 90% supercoiled monomer, 85% to 95% supercoiled monomer, 85% to 99% supercoiled monomer, 85% to 99.5% supercoiled monomer, 85% to 99.9% supercoiled monomer, 90% to 95% supercoiled monomer, 90% to 99% supercoiled monomer, 90% to 99.5% supercoiled monomer, 90% to 99.9% supercoiled monomer, 95% to 99% supercoiled monomer, 95% to 99.5% supercoiled monomer, 95% to 99.9% supercoiled monomer, 98% to 99% supercoiled monomer, 98% to 99.5% supercoiled monomer, or 98% to 99.9% supercoiled monomer; e.g., about 60% supercoiled monomer, about 65% supercoiled monomer, about 70% supercoiled monomer, about 75% supercoiled monomer, about 80% supercoiled monomer, about 85% supercoiled monomer, about 90% supercoiled monomer, about 95% supercoiled monomer, about 96% supercoiled monomer, about 97% supercoiled monomer, about 98% supercoiled monomer, about 99% supercoiled monomer, or about 99.9% supercoiled monomer). In any of these instances, supercoiled monomer is calculated using densitometry analysis of gel electrophoresis.

In other embodiments, a pharmaceutical composition contains circular DNA vector that is not supercoiled (i.e., relaxed circular DNA), e.g., at least 50%, at least 60%, at least 70%, at least 80%, at least 90%, at least 95%, or 100% of the circular DNA vector in the pharmaceutical composition is not supercoiled.

In some embodiments, percent supercoiled monomer is determined by agarose gel electrophoresis or capillary electrophoresis. Additionally, or alternatively, percent supercoiled monomer is determined by anion exchange-HPLC.

In some embodiments, the pharmaceutical composition is substantially devoid of impurities. For instance, in some embodiments, the pharmaceutical composition contains <1.0% protein content by mass (e.g., <0.9%, <0.8%, <0.7%, <0.6%, <0.5%, <0.4%, <0.3%, <0.2%, <0.1%, <0.05%, or <0.01% protein content by mass). In some instances, protein content is determined by bicinchoninic acid assay. Additionally or alternatively, protein content is determined by ELISA.

In some instances, the pharmaceutical composition contains <1.0% RNA content by mass (e.g., <0.9%, <0.8%, <0.7%, <0.6%, <0.5%, <0.4%, <0.3%, <0.2%, <0.1%, <0.05%, or <0.01% RNA content by mass). In some embodiments, the RNA content is determined by agarose gel electrophoresis. In some embodiments, the RNA content is determined by quantitative PCR. In some embodiments, the RNA content is determined by fluorescence assay (e.g., Ribogreen).

In some embodiments, the pharmaceutical composition contains <1.0% gDNA content by mass (e.g., <0.9%, <0.8%, <0.7%, <0.6%, <0.5%, <0.4%, <0.3%, <0.2%, <0.1%, <0.05%, or <0.01% gDNA content by mass). In some embodiments, the gDNA content is determined by agarose gel electrophoresis or capillary electrophoresis. In some embodiments, the gDNA content is determined by quantitative PCR. In some embodiments, the gDNA content is determined by Southern blot.

In some embodiments, the pharmaceutical composition contains <40 EU/mg endotoxin. In some embodiments, the pharmaceutical composition contains <20 EU/mg endotoxin. In some embodiments, the pharmaceutical composition contains <10 EU/mg endotoxin. In some embodiments, the pharmaceutical composition contains <5 EU/mg endotoxin (e.g., <4 EU/mg endotoxin, <3 EU/mg endotoxin, <2 EU/mg endotoxin, <1 EU/mg endotoxin, <0.5 EU/mg endotoxin), e.g., as measured by Limulus Amebocyte Lysate (LAL) assay.

Pharmaceutical compositions provided herein may include one or more pharmaceutically acceptable carriers, such as excipients and/or stabilizers that are nontoxic to the individual being treated (e.g., human patient) at the dosages and concentrations employed. In some embodiments, the pharmaceutically acceptable carrier is an aqueous pH buffered solution. Examples of pharmaceutically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as tween, polyethylene glycol (PEG), and pluronics.

If the pharmaceutical composition is provided in liquid form, the pharmaceutically acceptable carrier may be water (e.g., pyrogen-free water), isotonic saline, or a buffered aqueous solution, e.g., a phosphate buffered solution or a citrate buffered solution. Injection of the pharmaceutical composition may be carried out in water or a buffer, such as an aqueous buffer, e.g., containing a sodium salt (e.g., at least 50 mM of a sodium salt), a calcium salt (e.g., at least 0.01 mM of a calcium salt), or a potassium salt (e.g., at least 3 mM of a potassium salt). According to a particular embodiment, the sodium, calcium, or potassium salt may occur in the form of their halogenides, e.g., chlorides, iodides, or bromides, in the form of their hydroxides, carbonates, hydrogen carbonates, or sulfates, etc. Without being limited thereto, examples of sodium salts include NaCl, NaI, NaBr, Na₂CO₂, NaHCO₂, and Na₂SO₄. Examples of potassium salts include, e.g., KCl, KI, KBr, K₂CO₂, KHCO₂, and K₂SO₄. Examples of calcium salts include, e.g., CaCl₂), CaI₂, CaBr₂, CaCO₂, CaSO₄, and Ca(OH)₂. Additionally, organic anions of the aforementioned cations may be contained in the buffer. According to a particular embodiment, the buffer suitable for injection purposes as defined above, may contain salts selected from sodium chloride (NaCl), calcium chloride (CaCl₂)) or potassium chloride (KCl), wherein further anions may be present. CaCl₂) can also be replaced by another salt, such as KCl. In some embodiments, salts in the injection buffer are present in a concentration of at least 50 mM sodium chloride (NaCl), at least 3 mM potassium chloride (KCl), and at least 0.01 mM calcium chloride (CaCl₂)). The injection buffer may be hypertonic, isotonic, or hypotonic with reference to the specific reference medium, i.e., the buffer may have a higher, identical or lower salt content with reference to the specific reference medium, wherein preferably such concentrations of the afore mentioned salts may be used, which do not lead to damage of cells due to osmosis or other concentration effects. Reference media can be liquids such as blood, lymph, cytosolic liquids, other body liquids, or common buffers. Such common buffers or liquids are known to a skilled person. Ringer-Lactate solution is particularly preferred as a liquid basis.

One or more compatible solid or liquid fillers, diluents, or encapsulating compounds may be suitable for administration to a person. The constituents of the pharmaceutical composition according to the invention are capable of being mixed with the nucleic acid vector according to the invention as defined herein, in such a manner that no interaction occurs, which would substantially reduce the pharmaceutical effectiveness of the (pharmaceutical) composition according to the invention under typical use conditions. Pharmaceutically acceptable carriers, fillers and diluents can have sufficiently high purity and sufficiently low toxicity to make them suitable for administration to an individual being treated. Some examples of compounds which can be used as pharmaceutically acceptable carriers, fillers, or constituents thereof are sugars, such as lactose, glucose, trehalose, and sucrose; starches, such as corn starch or potato starch; dextrose; cellulose and its derivatives, such as sodium carboxymethylcellulose, ethylcellulose, cellulose acetate; powdered tragacanth; malt; gelatin; tallow; solid glidants, such as stearic acid, magnesium stearate; calcium sulfate; vegetable oils, such as groundnut oil, cottonseed oil, sesame oil, olive oil, corn oil and oil from theobroma; polyols, such as polypropylene glycol, glycerol, sorbitol, mannitol, and polyethylene glycol; or alginic acid.

The choice of a pharmaceutically acceptable carrier can be determined, according to the manner in which the pharmaceutical composition is administered.

Suitable unit dose forms for injection include sterile solutions of water, physiological saline, and mixtures thereof. The pH of such solutions may be adjusted to about 7.4. Suitable carriers for injection include hydrogels, devices for controlled or delayed release, polylactic acid, and collagen matrices. Suitable pharmaceutically acceptable carriers for topical application include those which are suitable for use in lotions, creams, gels and the like. If the pharmaceutical composition is to be administered perorally, tablets, capsules and the like are the preferred unit dose form.

Further additives which may be included in the pharmaceutical composition are emulsifiers, such as tween; wetting agents, such as sodium lauryl sulfate; coloring agents; pharmaceutical carriers; stabilizers; antioxidants; and preservatives.

The pharmaceutical composition according to the present invention may be provided in liquid or in dry (e.g., lyophilized) form. In a particular embodiment, the nucleic acid vector of the pharmaceutical composition is provided in lyophilized form. Lyophilized compositions including nucleic acid vector of the invention may be reconstituted in a suitable buffer, advantageously based on an aqueous carrier, prior to administration, e.g., Ringer-Lactate solution, Ringer solution, or a phosphate buffer solution.

In certain embodiments of the invention, any of the therapeutic circular DNA vectors of the invention can be complexed with one or more cationic or polycationic compounds, e.g., cationic or polycationic polymers, cationic or polycationic peptides or proteins, e.g., protamine, cationic or polycationic polysaccharides, and/or cationic or polycationic lipids.

According to a particular embodiment, a non-viral nucleic acid vector of the invention (e.g., a naked DNA vector, a circular DNA vector may be complexed with lipids to form one or more liposomes, lipoplexes, or lipid nanoparticles. Therefore, in one embodiment, the pharmaceutical composition comprises liposomes, lipoplexes, and/or lipid nanoparticles comprising any of the non-viral nucleic acid vectors of the invention.

Lipid-based formulations can be effective delivery systems for nucleic acid vectors due to their biocompatibility and their ease of large-scale production. Cationic lipids have been widely studied as synthetic materials for delivery of nucleic acids. After mixing together, nucleic acids are condensed by cationic lipids to form lipid/nucleic acid complexes known as lipoplexes. These lipid complexes are able to protect genetic material from the action of nucleases and deliver it into cells by interacting with the negatively charged cell membrane. Lipoplexes can be prepared by directly mixing positively charged lipids at physiological pH with negatively charged nucleic acids.

Conventional liposomes include of a lipid bilayer that can be composed of cationic, anionic, or neutral phospholipids and cholesterol, which encloses an aqueous core. Both the lipid bilayer and the aqueous space can incorporate hydrophobic or hydrophilic compounds, respectively. Liposome characteristics and behavior in vivo can be modified by addition of a hydrophilic polymer coating, e.g., polyethylene glycol (PEG), to the liposome surface to confer steric stabilization. Furthermore, liposomes can be used for specific targeting by attaching ligands (e.g., antibodies, peptides, and carbohydrates) to its surface or to the terminal end of the attached PEG chains.

Liposomes are colloidal lipid-based and surfactant-based delivery systems composed of a phospholipid bilayer surrounding an aqueous compartment. They may present as spherical vesicles and can range in size from 20 nm to a few microns. Cationic lipid-based liposomes are able to complex with negatively charged nucleic acids via electrostatic interactions, resulting in complexes that offer biocompatibility, low toxicity, and the possibility of the large-scale production required for in vivo clinical applications. Liposomes can fuse with the plasma membrane for uptake; once inside the cell, the liposomes are processed via the endocytic pathway and the genetic material is then released from the endosome/carrier into the cytoplasm.

Cationic liposomes can serve as delivery systems for therapeutic circular DNA vectors. Cationic lipids, such as MAP, (1,2-dioleoyl-3-trimethylammonium-propane) and DOTMA (N-[1-(2,3-diolcoyloxy)propyl]-N,N, N-trimethyl-ammonium methyl sulfate) can form complexes or lipoplexes with negatively charged nucleic acids to form nanoparticles by electrostatic interaction, providing high in vitro transfection efficiency. Furthermore, neutral lipid-based nanoliposomes for nucleic acid vector delivery as e.g., neutral 1,2-dioleoyl-sn-glycero-3-phosphatidylcholine (DOPC)-based nanoliposomes are available.

Thus, in one embodiment of the invention, the therapeutic circular DNA vector of the invention is complexed with cationic lipids and/or neutral lipids and thereby forms liposomes, lipid nanoparticles, lipoplexes or neutral lipid-based nanoliposomes in the present pharmaceutical compositions.

In a particular embodiment, a pharmaceutical composition comprises the nucleic acid vector (e.g., nonviral DNA vector (e.g., naked DNA vector), circular DNA vector (e.g., supercoiled circular DNA vector), and/or synthetic DNA vector (e.g., synthetic circular DNA vector)) of the invention that is formulated together with a cationic or polycationic compound and/or with a polymeric carrier. Accordingly, in a further embodiment of the invention, the nucleic acid vector is associated with or complexed with a cationic or polycationic compound or a polymeric carrier, optionally in a weight ratio selected from a range of about 5:1 (w/w) to about 0.25:1 (w/w), e.g., from about 5:1 (w/w) to about 0.5:1 (w/w), e.g., from about 4:1 (w/w) to about 1:1 (w/w) or of about 3:1 (w/w) to about 1:1 (w/w), e.g., from about 3:1 (w/w) to about 2:1 (w/w) of nucleic acid vector to cationic or polycationic compound and/or with a polymeric carrier; or optionally in a nitrogen/phosphate (N/P) ratio of nucleic acid vector to cationic or polycationic compound and/or polymeric carrier in the range of about 0.1-10, e.g., in a range of about 0.3-4 or 0.3-1, e.g., in a range of about 0.5-1 or 0.7-1, e.g., in a range of about 0.3-0.9 or 0.5-0.9. For example, the N/P ratio of the therapeutic circular DNA vector to the one or more polycations is in the range of about 0.1 to 10, including a range of about 0.3 to 4, of about 0.5 to 2, of about 0.7 to 2 and of about 0.7 to 1.5.

Pharmaceutical compositions may also involve association of the nucleic acid vectors described herein with a vehicle, transfection or complexation agent for increasing the transfection efficiency and/or the expression of the therapeutic gene according to the invention.

In some instances, the nucleic acid vector is complexed with one or more polycations, preferably with protamine or oligofectamine. Further cationic or polycationic compounds, which can be used as transfection or complexation agent may include cationic polysaccharides, for example chitosan, polybrene, cationic polymers, e.g., polyethyleneimine (PEI), cationic lipids, e.g., DOTMA: [1-(2,3-sioleyloxy)propyl)]-N,N,N-trimethylammonium chloride, DMRIE, di-C14-amidine, DOTIM, SAINT, DC-Chol, BGTC, CTAP, DOPE, LEAP, DOPE: Dioleyl phosphatidylethanol-amine, DOSPA, DODAB, DOIC, DMEPC, DOGS: Dioctadecylamidoglicylspermin, DIMRI: Dimyristo-oxypropyl dimethyl hydroxyethyl ammonium bromide, MAP: dioleoyloxy-3-(trimethylammonio)propane, DC-6-14:0,0-ditetradecanoyl-N-(α-trimethylammonioacetyl)diethanolamine chloride, CLIP1: rac-[(2,3-dioctadecyloxypropyl)(2-hydroxyethyl)]-dimethylammonium chloride, CLIP6: rac-[2 (2,3-dihexadecyloxypropyl-oxymethyloxy)ethyl]trimethylammonium, CLIP9: rac-[2 (2,3-dihexadecyloxypropyl-oxysuccinyloxy)ethyl]-trimethylammonium, oligofectamine, or cationic or polycationic polymers, e.g., modified polyaminoacids, such as β-aminoacid-polymers or reversed polyamides, etc., modified polyethylenes, such as PVP (poly(N-ethyl-4-vinylpyridinium bromide)), etc., modified acrylates, such as pDMAEMA (poly(dimethylaminoethyl methylacrylate)), etc., modified amidoamines such as pAMAM (poly(amidoamine)), etc., polybetaaminoester (PBAE) or modified PBAE (e.g., diamine end modified 1,4 butanediol diacrylate-co-5-amino-1-pentanol polymers, or polymers described in U.S. Pat. No. 8,557,231; PEGylated PBAE, such as those described in U.S. Patent Application No. 2018/0112038; any suitable polymer disclosed in Green et al., Acc. Chem. Res. 2008, 41 (6): 749-759, such as diamine end modified 1,4 butanediol diacrylate-co-5-amino-1-pentanol polymers; any suitable modified PBAE as described in International Patent Publication No. WO 2020/077159 or WO 2019/070727; PBAE 457 as disclosed in Shen et al., Sci. Adv. 2020, 6: caba1606, etc.), dendrimers, such as polypropylamine dendrimers or pAMAM based dendrimers, etc., polyimine(s), such as PEI: poly(ethyleneimine), poly(propyleneimine), etc., polyallylamine, sugar backbone based polymers, such as cyclodextrin based polymers, dextran based polymers, chitosan, etc., silan backbone based polymers, such as PMOXA-PDMS copolymers, etc., block polymers consisting of a combination of one or more cationic blocks (e.g., selected from a cationic polymer as mentioned above) and of one or more hydrophilic or hydrophobic blocks (e.g., polyethyleneglycol); etc.

In some instances, the pharmaceutical composition contains a nucleic acid vector encapsulated in a nanoparticle or microparticle, e.g., a biodegradable nanoparticle or microparticle (e.g., a cationic biodegradable polymeric nanoparticle or microparticle, such as PBAE or a modified PBAE (such as a polymer of formula (I) of International Patent Publication No. WO 2019/070727, or PBAE 457 as disclosed in Shen et al., Sci. Adv. 2020, 6: caba1606), a PEG-PBAE (or modified PBAE) copolymer), or a pH-sensitive nanoparticle or microparticle (e.g., a nanoparticle having a polymer of formula (I) of U.S. Pat. No. 10,792,374 (ECO)).

According to a particular embodiment, the pharmaceutical composition includes the nucleic acid vector encapsulated within or attached to a polymeric carrier (e.g., any of the aforementioned polymers described herein). A polymeric carrier used according to the invention may be a polymeric carrier formed by disulfide-crosslinked cationic components. The disulfide-crosslinked cationic components may be the same or different from each other. The polymeric carrier can also contain further components. It is also particularly preferred that the polymeric carrier used according to the present invention comprises mixtures of cationic peptides, proteins or polymers and optionally further components as defined herein, which are crosslinked by disulfide bonds as described herein. In this context, the disclosure of WO 2012/013326 is incorporated herein by reference. In this context, the cationic components that form basis for the polymeric carrier by disulfide-crosslinkage are typically selected from any suitable cationic or polycationic peptide, protein or polymer suitable for this purpose, particular any cationic or polycationic peptide, protein or polymer capable of complexing the nucleic acid vector as defined herein or a further nucleic acid comprised in the composition, and thereby preferably condensing the nucleic acid vector. The cationic or polycationic peptide, protein or polymer, may be a linear molecule; however, branched cationic or polycationic peptides, proteins or polymers may also be used

Every disulfide-crosslinking cationic or polycationic protein, peptide or polymer of the polymeric carrier, which may be used to complex the nucleic acid vector according to the invention included as part of the pharmaceutical composition of the invention may contain at least one SH moiety (e.g., at least one cysteine residue or any further chemical group exhibiting an SH moiety) capable of forming a disulfide linkage upon condensation with at least one further cationic or polycationic protein, peptide or polymer as cationic component of the polymeric carrier as mentioned herein.

Such polymeric carriers used to complex the nucleic acid vector may be formed by disulfide-crosslinked cationic (or polycationic) components. In particular, such cationic or polycationic peptides or proteins or polymers of the polymeric carrier, which comprise or are additionally modified to comprise at least one SH moiety, can be selected from proteins, peptides, and polymers as a complexation agent.

In other embodiments, the nucleic acid vector may be administered naked in a suitable buffer without being associated with any further vehicle, transfection, or complexation agent.

### V. Methods of Use

### A. Methods of Expression and Treatment

Provided herein are methods of inducing expression (e.g., persistent expression) of functional BEST1 in a target cell in a subject in need thereof (e.g., as part of a gene therapy regimen) by administering to the subject any of the nucleic acid vectors described herein, or pharmaceutical compositions thereof. Thus, some embodiments of the present methods include administering to a subject a DNA vector having (a) a BEST1-encoding sequence which is a DNA sequence encoding a BEST1 RNA transcript and (b) an shRNA-encoding sequence which is a DNA sequence encoding a shRNA comprising SEQ ID NO: 1 and/or SEQ ID NO: 3 (e.g., a sequence comprising SEQ ID NOs: 1-3; e.g., a sequence comprising or consisting of SEQ ID NO: 4), wherein the shRNA is not capable of targeting the BEST1 RNA transcript, or a pharmaceutical composition thereof. Some embodiments include administering to a subject any of the DNA vectors described herein that include a BEST1-encoding sequence but do not include an shRNA-encoding sequence.

Target cells (e.g., ocular cells, such as RPE cells) or tissues (e.g., retina) of a subject can be characterized by examining a nucleic acid sequence (e.g., an RNA sequence, e.g., an mRNA sequence) of the host cell, such as by Southern Blotting or PCR analysis, to detect or quantify the presence (e.g., persistence) of the therapeutic sequence or transgene delivered. Alternatively, expression of the therapeutic sequence or transgene in the subject can be characterized (e.g., quantitatively or qualitatively) by monitoring the progress of a disease being treated by delivery of the therapeutic sequence (e.g., associated with a defect or mutation targeted by the therapeutic sequence). In some embodiments, transcription or expression (e.g., persistent transcription or persistent expression) of the therapeutic sequence or transgene is confirmed by observing a decline in one or more symptoms associated with the disease.

Accordingly, the invention provides methods of treating a BEST1-associated disease (e.g., a bestrophinopathy) in a subject by administering to the subject any of the therapeutic nucleic acid vectors (e.g., therapeutic nonviral DNA vectors (e.g., therapeutic naked DNA vectors), therapeutic circular DNA vectors (e.g., therapeutic supercoiled circular DNA vectors), and/or therapeutic synthetic DNA vectors (e.g., therapeutic synthetic circular DNA vectors)), or pharmaceutical compositions thereof, described herein. Any of the therapeutic vectors, or pharmaceutical compositions thereof, described herein can be administered (e.g., by ocular administration) to a subject in a dosage from 1 μg to 10 mg of DNA (e.g., from 5 μg to 5.0 mg, from 10 μg to 2.0 mg, or from 100 μg to 1.0 mg of DNA, e.g., from 10 μg to 20 μg, from 20 μg to 30 μg, from 30 μg to 40 μg, from 40 μg to 50 μg, from 50 μg to 75 μg, from 75 μg to 100 μg, from 100 μg to 200 μg, from 200 μg to 300 μg, from 300 μg to 400 μg, from 400 μg to 500 μg, from 500 μg to 1.0 mg, from 1.0 mg to 5.0 mg, or from 5.0 mg to 10 mg of DNA, e.g., about 10 μg, about 20 μg, about 30 μg, about 40 μg, about 50 μg, about 60 μg, about 70 μg, about 80 μg, about 90 μg, about 100 μg, about 150 μg, about 200 μg, about 250 μg, about 300 μg, about 350 μg, about 400 μg, about 450 μg, about 500 μg, about 600 μg, about 700 μg, about 750 μg, about 1.0 mg, about 2.0 mg, about 2.5 mg, about 5.0 mg, about 7.5 mg, or about 10 mg of DNA).

In some embodiments, administration of a circular DNA vector of the invention, or a pharmaceutical composition thereof, is less likely to induce an immune response in a subject compared with administration of other gene therapy vectors (e.g., plasmid DNA vectors and/or viral vectors).

In some instances, the therapeutic circular DNA vectors, and pharmaceutical compositions thereof, provided herein are amenable to repeat dosing due to their ability to transfect target cells without triggering an immune response or inducing a reduced immune response relative to a reference vector, such as a plasmid DNA vector or an AAV vector, as discussed above. Thus, the invention provides methods of repeatedly administering the therapeutic circular DNA vectors and pharmaceutical compositions described herein. Any of the aforementioned dosing quantities may be repeated at a suitable frequency and duration. In some embodiments, the subject receives a dose about twice per day, about once per day, about five times per week, about four times per week, about three times per week, about twice per week, about once per week, about twice per month, about once per month, about once every six weeks, about once every two months, about once every three months, about once every four months, twice per year, once yearly, or less frequently. In some embodiments, the number and frequency of doses corresponds with the rate of turnover of the target cell. It will be understood that in long-lived post-mitotic target cells transfected using the vectors described herein, a single dose of vector may be sufficient to maintain expression of the heterologous gene within the target cell for a substantial period of time. Thus, in other embodiments, a therapeutic circular DNA vector provided herein may be administered to a subject in a single dose. The number of occasions in which a therapeutic circular DNA vector is delivered to the subject can be that which is required to maintain a clinical (e.g., therapeutic) benefit.

Methods of the invention include administration of a nucleic acid vector, or pharmaceutical composition thereof, through any suitable route. The nucleic acid vector, or pharmaceutical composition thereof, can be administered systemically or locally, e.g., ocularly (e.g., subretinally, intravitreally, suprachoroidally, by eye drop, intraocularly, intraorbitally), peri-ocularly (e.g., into the ciliary muscle or another peri-ocular tissue), intravenously, intramuscularly, intravitreally (e.g., by intravitreal injection), intradermally, intrahepatically, intracerebrally, intramuscularly, percutaneously, intraarterially, intraperitoneally, intralesionally, intracranially, intraarticularly, intraprostatically, intrapleurally, intratracheally, intrathecally, intranasally, intravaginally, intrarectally, intratumorally, subcutaneously, subconjunctivally, intravesicularly, mucosally, intrapericardially, intraumbilically, orally, topically, transdermally, by inhalation, by aerosolization, by injection (e.g., by jet injection), by electroporation, by implantation, by infusion (e.g., by continuous infusion), by localized perfusion bathing target cells directly, by catheter, by lavage, in creams, or in lipid compositions.

Any suitable means of ocular administration known in the art or described herein may be used as part of the methods provided herein. Methods of delivering a nucleic acid vector, or pharmaceutical composition thereof, to a target retinal cell (e.g., RPE cell) include administering the nucleic acid vector, or composition thereof, to the eye by intraocular injection (e.g., injection to the posterior of the eye or the anterior of the eye by, e.g., subretinal injection, suprachoroidal injection, intravitreal injection, periocular injection, sub-tenton injection, posterior juxtascleral injection, intracameral injection, subconjunctival injection, or retrobulbar injection) or intraocular implant. In some embodiments of any of the methods described herein, the administration of the DNA vector is via an intraocular implant (e.g., a controlled release or depot implant, an intravitreal implant, a subconjunctival implant, or an episcleral implant). In other embodiments, the administration of the DNA vector is not via an intraocular implant (e.g., a controlled release or depot implant, an intravitreal implant, a subconjunctival implant, or an episcleral implant). In some embodiments of any of the methods described herein, the administration of the DNA vector is via iontophoresis (e.g., the method includes administration of the nucleic acid vector to the intraocular space by iontophoresis and subsequent delivery to the retina by transmitting a current through an electrode contacting an interior region of the eye).

In some instances, administration of the nucleic acid vector, or pharmaceutical composition thereof, is non-surgical. For example, in some embodiments, administration of the nucleic acid vector, or pharmaceutical composition thereof, does not utilize general anesthesia and/or does not involve retrobulbar anesthesia (i.e., retrobulbar block)). Additionally, or alternatively, administration of the nucleic acid vector, or pharmaceutical composition thereof, does not involve injection using a needle larger than 28 gauge. Additionally, or alternatively, administration of the nucleic acid vector, or pharmaceutical composition thereof, does not involve use of a guidance mechanism that is typically required for ocular drug delivery via shunt or cannula.

In some instances, administration of the nucleic acid vector, or pharmaceutical composition thereof, is by injection (e.g., microneedle injection) into an outer tissue of the eye, e.g., the suprachoroidal space, sclera, cornea, corneal stroma, conjunctiva, subconjunctival space, or subretinal space. Alternatively, administration of the DNA vector is by injection (e.g., microneedle injection) into a site proximal to the outer tissue, such as the trabecular meshwork, ciliary body, aqueous humor or vitreous humor.

Microneedles for injecting a nucleic acid vector, or pharmaceutical composition thereof, to an eye include hollow microneedles, which may include an elongated housing for holding the proximal end of the microneedle. Microneedles may further include a means for conducting a drug formulation therethrough. For example, the means may be a flexible or rigid conduit in fluid connection with the base or proximal end of the microneedle. The means may also include a pump or other devices for creating a pressure gradient for inducing fluid flow through the device. The conduit may in operable connection with a source of the drug formulation. The source may be any suitable container. In one embodiment, the source may be in the form of a conventional syringe. The source may be a disposable unit, dose container. In one embodiment, the microneedle has an effective length of about 50 μm to about 2000 μm. In another particular embodiment, the microneedle has an effective length of from about 150 μm to about 1500 μm, from about 300 μm to about 1250 μm, from about 500 μm to about 1250 μm, from about 500 μm to about 1500 μm, from about 600 μm to about 1000 μm, or from about 700 μm to about 1000 μm. In one embodiment, the effective length of the microneedle is about 600 μm, about 700 μm, about 800 μm or about 1000 μm. In various embodiments, the proximal portion of the microneedle has a maximum width or cross-sectional dimension of from about 50 μm to 600 μm, from about 50 μm to about 400 μm, from about 50 μm to about 500 μm, from about 100 μm to about 400 μm, from about 200 μm to about 600 μm, or from about 100 μm to about 250 μm, with an aperture diameter of about 5 μm to about 400 μm. In a particular embodiment, the proximal portion of the microneedle has a maximum width or cross-sectional dimension of about 600 μm. In various embodiments, the microneedle has a bevel height from 50 μm to 500 μm, 100 μm to 500 μm, 100 μm to 400 μm, 200 μm to 400 μm, or 300 μm to 500 μm.

In particular instances, administration of the nucleic acid vector, or pharmaceutical composition thereof, is by suprachoroidal injection, which can be accomplished in a minimally invasive, non-surgical manner. For instance, suprachoroidal injection can provide nucleic acid delivery over a larger tissue area and to less accessible target tissues in a single administration as compared to other types of administration (e.g., subretinal injection). Without wishing to be bound by theory, upon entering the suprachoroidal space, a pharmaceutical composition can flow circumferentially toward the retinochoroidal tissue, macula, and optic nerve in the posterior segment of the eye. In addition, a portion of the infused pharmaceutical composition may remain in the suprachoroidal space as a depot, or remain in tissue overlying the suprachoroidal space, for example the sclera, near the microneedle insertion site, serving as additional depot of the pharmaceutical composition that can subsequently diffuse into the suprachoroidal space and into other adjacent posterior tissues.

Suprachoroidal injection can be performed using any suitable method known in the art or described herein. For example, in some instances, the nucleic acid vector is suprachoroidally administered through a microneedle (e.g., a hollow microneedle). In some instances, the nucleic acid vector is suprachoroidally administered through a microneedle array. Exemplary microneedles suitable for use in suprachoroidal administration of nucleic acid vectors described herein are described, e.g., in U.S. Patent Application No. 2017/0273827, which is incorporated herein by reference.

Suprachoroidal injection can be performed using methods known in the art. For example, a microneedle tip can be placed into the eye so that the drug formulation flows into the suprachoroidal space and to the posterior ocular tissues surrounding the suprachoroidal space. In one embodiment, insertion of the microneedle is in the sclera of the eye. In one embodiment, drug flow into the suprachoroidal space is achieved without contacting underlying tissues with the microneedle, such as choroid and retina tissues. In some embodiments, the one or more microneedles are inserted perpendicularly, or at an angle from 80° to 100°, into the eye, e.g., into the sclera, reaching the suprachoroidal space in a short penetration distance. Exemplary methods suitable for use in suprachoroidal administration of nucleic acid vectors described herein are described, e.g., in International Patent Publication No. WO 2014/074823, which is incorporated herein by reference.

In some instances, the present methods of delivering a nucleic acid vector, or pharmaceutical composition thereof, involve administration intravitreally. Intravitreal administration can be conducted using any suitable method known in the art or described herein. For instance, contemplated herein are intravitreal injection methods involving the InVitria Injection Assistant (FCI Ophthalmics, Pembroke, MA), Rapid Access Vitreal Injection (RAVI) Gude (Katalyst Surgical, Chesterfield, MO), Doi-Umeatsu Intravitreal Injection Guide (Duckworth & Kent Ltd., England), Malosa Intravitreal Injection Guide (Beaver-Visitec International, Waltham, MA), or automated injection guides.

Any suitable dose of nucleic acid vector, or pharmaceutical composition thereof, may be administered. For instance, in embodiments involving subretinal administration of nucleic acid vector (e.g., naked DNA vector), each eye may be injected with one or more blebs (e.g., two blebs per eye) each having a volume from 20-500 μL (e.g., from 50-250 μL; e.g., 50-100 μL, 100-150 μL, 150-200 μL, or 200-250 μL; e.g., about 50 μL, about 75 μL, about 100 μL, about 150 μL, or about 200 μL), e.g., one bleb, two blebs, three blebs, four blebs, or more, per eye. In embodiments involving subretinal administration of naked nucleic acid vector, the injection volume (e.g., pharmaceutical composition) contains the nucleic acid vector at a concentration from 0.5 mg/mL to 5 mg/mL (e.g., from 1.0 mg/mL to 2.5 mg/mL; e.g., from 0.5 mg/mL to 1.0 mg/mL, from 1.0 mg/mL to 1.5 mg/mL, from 1.5 mg/mL to 2.0 mg/mL, from 2.0 mg/mL to 2.5 mg/mL, from 2.5 mg/mL to 3.0 mg/mL, from 3.0 mg/mL to 4.0 mg/mL, or from 4.0 mg/mL to 5.0 mg/mL; e.g., about 0.5 mg/mL, about 1.0 mg/mL, about 1.5 mg/mL, about 2.0 mg/mL, about 2.5 mg/mL, about 3.0 mg/mL, about 4.0 mg/mL, or about 5.0 mg/mL. In particular instances (e.g., wherein naked nucleic acid vector is administered subretinally), the injection volume (e.g., pharmaceutical composition) contains the nucleic acid vector at a concentration of 1.5 mg/mL. In some embodiments involving subretinal administration, naked nucleic acid vector is administered to each eye in an amount from 20 μg to 2.0 mg (e.g., from 100 μg to 1.0 mg, or from 200 μg to 500 μg; e.g., from 20 μg to 50 μg, from 50 μg to 100 μg, from 100 μg to 150 μg, from 150 μg to 200 μg, from 200 μg to 250 μg, from 250 μg to 300 μg, from 300 μg to 350 μg, from 350 μg to 400 μg, from 400 μg to 500 μg, from 500 μg to 750 μg, from 750 μg to 1.0 mg, from 1.0 mg to 1.5 mg, or from 1.5 mg to 2.0 mg; e.g., about 20 μg, about 25 μg, about 30 μg, about 40 μg, about 50 μg, about 60 μg, about 70 μg, about 75 μg, about 80 μg, about 90 μg about 100 μg, about 125 μg, about 150 μg, about 175 μg, about 200 μg, about 225 μg, about 250 μg, about 275 μg, about 300 μg, about 350 μg, about 400 μg, about 500 μg, about 600 μg, about 700 μg, about 800 μg, about 900 μg, about 1.0 mg, about 1.1. mg, about 1.2 mg, about 1.3 mg, about 1.4 mg, about 1.5 mg, about 1.6 mg, about 1.7 mg, about 1.8 mg, about 1.9 mg, or about 2.0 mg). In some embodiments involving subretinal administration, naked nucleic acid vector is administered to each eye in an amount from 10⁸ to 10¹⁵ vector copies (e.g., DNA vector molecules, e.g., circular DNA vector molecules) (e.g., from 10⁸ to 10⁹, from 10⁹ to 10¹⁰, from 10¹⁰ to 10¹¹, from 10¹¹ to 10¹², from 10¹² to 10¹³, from 10¹³ to 10¹⁴, or from 10¹⁴ to 10¹⁵ vector copies; e.g., about 1×10¹¹ vector copies, about 5×10¹¹ vector copies, about 1×10¹² vector copies, about 5×10¹² vector copies, about 1×10¹³ vector copies, about 2.5×10¹³ vector copies, or about 5×10¹³ vector copies). In particular embodiments, naked nucleic acid vector is administered subretinally (e.g., in two 75 μL-blebs per eye) at a total dose per eye of about 2.5×10¹³ vector copies. In other embodiments, naked nucleic acid vector is administered subretinally (e.g., in two 75 μL-blebs per eye) at a total dose per eye of about 5×10¹² vector copies. In other embodiments, naked nucleic acid vector is administered subretinally (e.g., in two 75 μL-blebs per eye) at a total dose per eye of about 5×10¹¹ vector copies.

Nucleic acid vectors described herein can be delivered into cells via in vivo electrotransfer (e.g., in vivo electroporation), e.g., by transmitting electrical energy into the tissue in which the target ocular cell resides. Such methods involve electrotransfer of the nucleic acid vector from the extracellular space in the posterior of the eye (e.g., the suprachoroidal space, choroid, retina, or vitreous) into the target ocular cell (e.g., retinal cell). For example, in some instances in which an individual is being treated for a retinal disease or disorder, the method involves transmitting electrical energy into the retina to cause electrotransfer of the nucleic acid vector from the extracellular space of the retina into one or more retinal cell types (e.g., a photoreceptor and/or a RPE cell).

In some instances, an electrode is positioned within the interior of the individual's eye, and an electric field is transmitted through the electrode into a target ocular tissue (e.g., retina at conditions suitable for electrotransfer of the therapeutic agent (e.g., nucleic acid vector) into the target cell (e.g., target retinal cell). An electric field (e.g., a pulsed electric field (PEF)) transmitted into a target ocular tissue can promote transfer of a nucleic acid vector into a target ocular cell. Such electrotransfer can occur through any one of several mechanisms (and combinations thereof), including electrophoresis, electrokinetically driven drug uptake, and/or electroporation. Transmission of electric fields involve conditions suitable for such mechanisms. Suitable means of generating electric fields for electrotransfer of nucleic acids in mammalian tissue are known in the art, and any suitable means known in the art or described herein can be adapted for use as part of the present invention.

Various means of generating and transmitting an electric field into a tissue are contemplated herein as part of the present methods. Devices and systems having electrodes suitable for transmitting electric fields in mammalian tissues are commercially available and can be useful in the methods disclosed herein. In some instances, the electric field is transmitted through an electrode comprising a needle (e.g., a needle positioned within the vitreous humor or in the subretinal space). Suitable needle electrodes include CLINIPORATOR® electrodes marketed by IGEA® and needle electrodes marketed by AMBU®. Electrodes (e.g., needle electrodes) can be made from any suitable conductive material, such as metal or metal alloy, such as platinum, stainless steel, nickel, titanium, and combinations thereof, such as platinum/iridium alloy or nitinol.

In some embodiments, the electrode used as part of methods described herein is a substantially planar electrode, such as any of the substantially planar electrodes described in International Patent Application No. PCT/US2022/021209, the disclosure of which are hereby incorporated by reference in its entirety. In some embodiments, the electrode used as part of methods described herein is a substantially planar electrode as described in International Patent Application No. PCT/US2022/021209, the disclosure of which are hereby incorporated by reference in its entirety. Such substantially planar electrodes are composed of a shape memory material (e.g., a shape memory alloy) that allows the structure of an elongate conductor (e.g., a wire electrode) to relax into a preformed, substantially planar electrode when unsheathed. The substantially planar electrode is approximately perpendicular to the longitudinal axis of the sheath and/or the proximal portion of the wire (e.g., the region that does not include the substantially planar electrode).

Electrodes (e.g., a substantially planar electrodes or a non-substantially planar electrodes (e.g., substantially axial wire electrodes)) for use in the present methods may be monopolar. In some embodiments involving electrotransfer using a monopolar electrode, a ground electrode is attached to the individual (e.g., attached to the skin of an individual) at a point other than the eye. In some embodiments, the ground electrode is a pad contacting the skin of the buttocks, leg, torso, neck (e.g., the posterior of the neck), or head (e.g., the posterior of the head) of the individual. In some embodiments, the monopolar electrode transmits electrical energy upon becoming positively charged. In some embodiments, the monopolar electrode transmits electrical energy upon becoming negatively charged.

Alternatively, electrodes may be bipolar (e.g., a substantially planar electrodes or a non-substantially planar electrodes may be bipolar (e.g., substantially axial wire electrodes may be bipolar)). In a bipolar embodiment, an auxiliary electrode may be in electrical communication with the primary electrode (e.g., substantially planar electrode or a non-substantially planar electrode (e.g., substantially axial wire electrode)). The auxiliary electrode may be proximal to the primary electrode (i.e., closer to the operator), e.g., part of, or connected to, a sheath housing a primary wire electrode. In some embodiments involving electrotransfer using a bipolar electrode, electrical energy (e.g., current) is transmitted upon application of a positive voltage to the primary electrode and a negative voltage to the auxiliary electrode. In some embodiments involving electrotransfer using a bipolar electrode, electrical energy (e.g., current) is transmitted upon application of a negative voltage to the primary electrode and a positive voltage to the auxiliary electrode.

In some instances, methods of the invention involve contacting an electrode (e.g., a substantially planar electrode or a non-substantially planar electrode (e.g., a substantially axial wire electrode)) to an interior region of the eye such that electrical energy transmitted from the electrode is sufficient to cause electrotransfer at the target tissue (e.g., the retina, e.g., the macula). Thus, methods of the invention may include positioning the electrode into electrical communication with the target tissue (e.g., retina, e.g., the macula). In particular instances, the interior region of the eye contacting the electrode includes the vitreous humor. For example, the electrode may be positioned wholly or partially within the vitreous humor upon transmission of the electric field. In instances in which the electrode is positioned within the vitreous humor (e.g., wholly within the vitreous humor), the electrode may be positioned in electrical communication with the interface of the vitreous humor with the retina (e.g., an interface at the macula).

In any of the aforementioned embodiments, the proximity of the electrode (e.g., a substantially planar electrode or the tip of a needle electrode) to the target cell results in a voltage (e.g., potential) at the target cell that is within 20% of the amplitude of the waveform of the applied energy (e.g., within 19%, within 18%, within 17%, within 16%, within 15%, within 14%, within 13%, within 12%, within 11%, within 10%, within 9%, within 8%, within 7% within 6%, within 5%, within 4%, within 3%, within 2%, or within 1% of the amplitude of the waveform of the applied energy).

It will be appreciated that a variety of suitable conditions, electrical parameters, and algorithms thereof may be used for electrotransfer.

The voltage source may be configured to generate an electric field strength, e.g., at a target cell, from about 10 V/cm to about 1,500 V/cm (e.g., from about 10 V/cm to about 100 V/cm, e.g., about 10 V/cm, 20 V/cm, 30 V/cm, 40 V/cm, 50 V/cm, 60 V/cm, 70 V/cm, 80 V/cm, 90 V/cm, or 100 V/cm, e.g., from about 100 V/cm to about 1,000 V/cm, e.g., about 200 V/cm, 300 V/cm, 400 V/cm, 500 V/cm, 600 V/cm, 700 V/cm, 800 V/cm, 900 V/cm, or 1,000 V/cm, e.g., from about 1,000 V/cm to about 1,500 V/cm, e.g., about 1,110 V/cm, 1,200 V/cm, 1,300 V/cm, 1,400 V/cm, or 1,500 V/cm). In some embodiments, the voltage source is be configured to generate an electric field strength, e.g., at a target cell, from about 10 V/cm to about 1,000 V/cm (e.g., from about 10 V/cm to 500 V/cm or from about 500 V/cm to about 1,000 V/cm). In some embodiments, the field strength is from 50 V/cm to 300 V/cm. In some embodiments, the field strength is about 100 V/cm at the target cell.

In some embodiments, the voltage (e.g., potential) at the target cell is from 5 V to 100 V (e.g., from 10 V to 80V, from 15 V to 70 V, from 20 V to 60 V, or from 30 V to 50 V; e.g., about 10 V, about 15 V, about 20 V, about 25 V, about 30 V, about 35 V, about 40 V, about 45 V, about 50 V, about 55 V, about 60 V, about 65 V, about 70 V). In some embodiments, the voltage (e.g., potential) at the target cell is from 20 V to 60 V. In some embodiments, the voltage (e.g., potential) at the target cell is from 30 V to 50 V, e.g., about 35 V to 45 V. In any of the aforementioned embodiments, close proximity of the electrode to the target cell results in a voltage (e.g., potential) at the target cell that is within 20% of the amplitude of the waveform of the applied energy (e.g., within 19%, within 18%, within 17%, within 16%, within 15%, within 14%, within 13%, within 12%, within 11%, within 10%, within 9%, within 8%, within 7% within 6%, within 5%, within 4%, within 3%, within 2%, or within 1% of the amplitude of the waveform of the applied energy). For instance, a 40 V amplitude pulse from a monopolar intravitreal electrode positioned near the retina may result in a voltage (e.g., potential) of 35 V at a target retinal cell. It will be understood that waveform amplitudes required to achieve a given voltage at a target cell will depend on the electrode configuration (e.g., monopolar vs bipolar), electrode shape, distance between electrode and the target cell, and material properties (e.g., conductivity) of the tissue (e.g., vitreous and retina).

In some embodiments, the current resulting from the pulsed electric field is from 10 μA to 1 A (e.g., from 10 μA to 500 mA, from 10 μA to 200 mA, from 10 μA to 100 mA, from 10 μA to 50 mA, or from 10 μA to 25 mA; e.g., from 50 μA to 500 mA, from 100 μA to 200 mA, or from 1 mA to 100 mA; e.g., from 10 μA to 20 μA, from 20 μA to 30 μA, from 30 μA to 50 μA, from 50 μA to 100μ, from 100 μA to 150μ, from 150 μA to 200μ, from 200 μA to 300 μA, from 300 μA to 400μ, from 400 μA to 500μ, from 500 μA to 600μ, from 600 μA to 800 μA, from 800 μA to 1 mA, from 1 mA to 10 mA, from 10 mA to 20 mA, from 20 mA to 30 mA, from 30 mA to 40 mA, from 40 mA to 50 mA, from 50 mA to 60 mA, from 60 mA to 70 mA, from 70 mA to 80 mA, from 80 mA to 90 mA, from 90 mA to 100 mA, from 100 mA to 200 mA, from 200 mA to 300 mA, from 300 mA to 500 mA, or from 500 mA to 1 A; e.g., about 1 mA, about 5 mA, about 10 mA, about 15 mA, about 20 mA, about 25 mA, about 30 mA, about 35 mA, about 40 mA, about 45 mA, about 50 mA, about 60 mA, about 70 mA, about 80 mA, about 90 mA, or about 100 mA).

In some embodiments, the pulses of electrical energy have an amplitude of about 20 V. In some embodiments in which the pulses of electrical energy have an amplitude of about 20 V, the current is between 5 mA and 50 mA (e.g., from 10 mA to 40 mA, e.g., from 5 mA to 10 mA, from 10 mA to 15 mA, from 15 mA to 20 mA, from 20 mA to 30 mA, or from 40 mA to 50 mA). In some embodiments, the pulses of electrical energy have an amplitude of about 40 V. In some embodiments in which the pulses of electrical energy have an amplitude of about 40 V, the current is between 10 mA and 100 mA (e.g., from 20 mA to 80 mA, or from 30 mA to 70 mA, e.g., from 10 mA to 20 mA, from 20 mA to 30 mA, from 30 mA to 40 mA, from 40 mA to 50 mA, from 50 mA to 60 mA, from 60 mA to 70 mA, from 70 mA to 80 mA, from 80 mA to 90 mA, or from 90 mA to 100 mA).

In some embodiments, the electrode is positioned within about 10 mm (e.g., within 9 mm, 8 mm, 7 mm, 6 mm, 5 mm, 4.5 mm, 4 mm, 3.5 mm, 3 mm, 2.5 mm, 2 mm, 1.5 mm, 1 mm, or 0.5 mm, 0.45 mm, 0.40 mm, 0.35 mm, 0.30 mm, 0.25 mm, 0.20 mm, 0.15 mm, or 0.10 mm) of the retinal interface. The electrode may be from 0.1 to about 0.5 mm (e.g., about 0.15 mm, 0.2 mm, 0.25 mm, 0.30 mm, 0.35 mm, 0 40 mm, 0.45 mm, or 0.5 mm), or from about 0.5 mm to 5 mm (e.g., about 0.5 mm, 1 mm, 1.5 mm, 2 mm, 2.5 mm, 3 mm, 3.5 mm, 4 mm, 4.5 mm, or 5 mm) from the retinal interface upon transmission of the one or more pulses. In some embodiments, the electrode (e.g., substantially planar electrode) is within about 1 mm from the retinal interface upon transmission of the one or more pulses.

The target cell (e.g., the target retinal cell, which may be a retinal cell (e.g., RPE cell), e.g., in the macula) may be within about 5 mm (e.g., 4.5 mm, 4 mm, 3.5 mm, 3 mm, 2.5 mm, 2 mm, 1.5 mm, 1 mm, or 0.5 mm) from the retinal interface (e.g., at the macula). For example, the target cell may be from about 0.01 mm to about 1 mm (e.g., from about 0.01 mm to about 0.1 mm, e.g., about 0.02 mm, 0.03 mm, 0.04 mm, 0.05 mm, 0.06 mm, 0.07 mm, 0.08 mm, 0.09 mm, or 0.1 mm, e.g., from about 0.1 mm to about 1 mm, e.g., about 0.2 mm, 0.3 mm, 0.4 mm, 0.5 mm, 0.6 mm, 0.7 mm, 0.8 mm, 0.9 mm, or 1 mm) from the retinal interface.

It will be appreciated that a variety of suitable electrical parameters and algorithms thereof may be used. The voltage source may be configured to generate an electric field strength, e.g., at a target cell (e.g., a retinal cell), from about 10 V/cm to about 1,500 V/cm (e.g., from about 10 V/cm to about 100 V/cm, e.g., about 10 V/cm, 20 V/cm, 30 V/cm, 40 V/cm, 50 V/cm, 60 V/cm, 70 V/cm, 80 V/cm, 90 V/cm, or 100 V/cm, e.g., from about 100 V/cm to about 1,000 V/cm, e.g., about 200 V/cm, 300 V/cm, 400 V/cm, 500 V/cm, 600 V/cm, 700 V/cm, 800 V/cm, 900 V/cm, or 1,000 V/cm, e.g., from about 1,000 V/cm to about 1,500 V/cm, e.g., about 1,110 V/cm, 1,200 V/cm, 1,300 V/cm, 1,400 V/cm, or 1,500 V/cm). In some embodiments, the voltage source is be configured to generate an electric field strength, e.g., at a target cell (e.g., a retinal cell), from about 10 V/cm to about 1,000 V/cm (e.g., from about 10 V/cm to 500 V/cm or from about 500 V/cm to about 1,000 V/cm). In some embodiments, the field strength is from 50 V/cm to 300 V/cm. In some embodiments, the field strength is about 100 V/cm at the target cell (e.g., the target retinal cell).

In some embodiments, the total number of pulses of electrical energy are delivered within 1-60 seconds (e.g., within 1-5 seconds, 5-10 seconds, 10-15 seconds, 15-20 seconds, 20-30 seconds, 30-40 seconds, 40-50 seconds, or 50-60 seconds). In some embodiments, the total number of pulses of electrical energy are delivered within 1-20 seconds. For example, the total number of pulses of electrical energy may be delivered within 1-5 seconds, 5-10 seconds, 10-15 seconds, or 15-20 seconds, e.g., 1, 2, 3, 4, 5, 6, 7, 8, 9, 10, 11, 12, 13, 14, 15, 16, 17, 18, 19, or 20 seconds. The pulses of electrical energy may be, e.g., square waveforms. The pulses of electrical energy may have an amplitude from 5 V to 500 V. For example, the pulses of electrical energy may have an amplitude of about 5 V, 10 V, 15 V, 20 V, 25 V, 30 V, 35V, 40 V, 45 V, 50 V, 60 V, 70 V, 80 V, 90 V, 100 V, 125 V, 150 V, 175 V, 200 V, 225V, 250 V, 275 V, 300 V, 325 V, 350 V, 375 V, 400 V, 425 V, 450 V, 475 V, or 500 V. In some embodiments, the pulses of electrical energy have an amplitude of about 5-250 V (e.g., about 20 V). Any of the aforementioned voltages can be the tops of square-waveforms, peaks in sinusoidal waveforms, peaks in sawtooth waveforms, root mean square (RMS) voltages of sinusoidal waveforms, or RMS voltages of sawtooth waveforms.

In some embodiments, about 1-12 pulses (e.g., 1, 2, 3, 4, 5, 6, 7, 8, 9, 10, 11, or 12 pulses) of electrical energy are transmitted during use. In some embodiments, about 4-12 pulses of electrical energy are transmitted during use.

In some embodiments, each of the pulses is from about 0.01 ms to about 200 ms in duration, from about 0.1 ms to about 200 ms in duration, or from about 1 ms to about 200 ms in duration (e.g., 0.10 ms to about 200 ms in duration. For example, each of the pulses may be about 10 ms, 20 ms, 30 ms, 40 ms, 50 ms, 60 ms, 70 ms, 80 ms, 90 ms, 100 ms, 110 ms, 120 ms, 130 ms, 140 ms, 150 ms, 160 ms, 170 ms, 180 ms, 190 ms, or 200 ms). In some embodiments, each of the pulses is about 20 ms in duration. In some embodiments, each of the pulses is about 50 ms in duration. In some embodiments, each of the pulses is from about 0.01 ms to about 1 ms (e.g., from 0.01 ms to 0.05 ms, from 0.05 ms to 0.1 ms, from 0.1 ms to 0.25 ms, from 0.25 ms to 0.5 ms, from 0.5 ms to 0.75 ms, or from 0.75 ms to 1.0 ms; e.g., about 0.01 ms, about 0.05 ms, about 0.1 ms, about 0.2 ms, about 0.3 ms, about 0.4 ms, about 0.5 ms, about 0.6 ms, about 0.7 ms, about 0.8 ms, about 0.9 ms, or about 1.0 ms) in duration.

In some embodiments, each of the pulses of electrical energy is from about 10 ms to about 200 ms. For example, each of the pulses of electrical energy may be about 10 ms, 20 ms, 30 ms, 40 ms, 50 ms, 60 ms, 70 ms, 80 ms, 90 ms, 100 ms, 110 ms, 120 ms, 130 ms, 140 ms, 150 ms, 160 ms, 170 ms, 180 ms, 190 ms, or 200 ms. In some embodiments, each of the pulses of electrical energy is from about 50 ms. In some embodiments, each of the pulses of electrical energy is less than 10 ms. For example, each of the pulses of electrical energy may be from about 10 us to about 10 ms, e.g., from about 10 us to about 100 μs, e.g., about 20 μs, 30 μs, 40 μs, 50 μs, 60 μs, 70 μs, 80 μs, 90 μs, or 100 μs, e.g., from about 100 μs to about 1 ms, e.g., about 200 μs, 300 μs, 400 μs, 500 μs, 600 μs, 700 μs, 800 μs, 900 μs, or 1 ms, e.g., from about 1 ms to about 10 ms, e.g., about 2 ms, 3 ms, 4 ms, 5 ms, 6 ms, 7 ms, 8 ms, 9 ms, or 10 ms.

In some embodiments, the total number of pulses of electrical energy are transmitted within 1-20 seconds (e.g., within 6-12 seconds, e.g., within 1-3 seconds, within 3-6 seconds, within 6-10 seconds, within 10-15 seconds, or within 15-20 seconds, e.g., within one second, within two seconds, within three seconds, within four seconds, within five seconds, within six seconds, within seven seconds, within eight seconds, within nine seconds, within ten seconds, within 11 seconds, within 12 seconds, within 13 seconds, within 14 seconds, within 15 seconds, within 16 seconds, within 17 seconds, within 18 seconds, within 19 seconds, within 20 seconds). The pulses of electrical energy may have an amplitude from 5 V to 1,500 V. For example, the pulses of electrical energy may have an amplitude from about 5 V to 500 V, from about 500 V to about 1,000 V, or from about 1,000 V to about 1,500 V. In some embodiments, the pulses of electrical energy have an amplitude from 5 V to 500 V. For example, the pulses of electrical energy may have an amplitude of about 5 V, 10 V, 15 V, 20 V, 25 V, 30 V, 40 V, 50 V, 100 V, 125 V, 150 V, 175 V, 200 V, 225 V, 250 V, 275 V, 300 V, 325 V, 350 V, 375 V, 400 V, 425 V, 450 V, 475 V, or 500 V. In some embodiments, the pulses of electrical energy have an amplitude of from about 5 V to about 250 V.

An electric field suitable for electrotransfer can be transmitted to a target ocular cell at or near the time of administration of the nucleic acid vector or pharmaceutical composition thereof (e.g., as part of the same procedure). For example, the present invention includes methods in which an electric field is transmitted within one hour of administration of the nucleic acid vector or pharmaceutical composition thereof (e.g., within 55 minutes, within 50 minutes, within 45 minutes, within 40 minutes, within 35 minutes, within 30 minutes, within 25 minutes, within 20 minutes, within 15 minutes, within 10 minutes, within 5 minutes, within 4 minutes, within 3 minutes, within 2 minutes, within 90 seconds, within 60 seconds, within 45 seconds, with 30 seconds, within 20 seconds, within 15 seconds, within 10 seconds, within 9 seconds, within 8 seconds, within 7 seconds, within 6 seconds, within 5 seconds, within 4 seconds, within 3 seconds, within 2 seconds, or within 1 second) of administration of the nucleic acid vector or pharmaceutical composition thereof (e.g., simultaneously with administration of the nucleic acid vector or pharmaceutical composition thereof or after administration but within any of the aforementioned durations). In some embodiments, an electric field is transmitted within 24 hours of administration of the nucleic acid vector or pharmaceutical composition thereof (e.g., within 22 hours, within 20 hours, within 18 hours, within 16 hours, within 14 hours, within 12 hours, within 10 hours, within 8 hours, within 6 hours, within 5 hours, within 4 hours, within 3 hours, within 2 hours, within 90 minutes, within 60 minutes, within 45 minutes, within 30 minutes, within 20 minutes, within 15 minutes, within 10 minutes, within 8 minutes, within 6 minutes, within 5 minutes, within 4 minutes, within 3 minutes, or within 2 minutes) of administration of the nucleic acid vector or pharmaceutical composition thereof. In some embodiments, an electric field is transmitted within 7 days of administration of the nucleic acid vector or pharmaceutical composition thereof (e.g., within 6 days, within 5 days, within 4 days, within 3 days, or within 2 days) of administration of the nucleic acid vector or pharmaceutical composition thereof.

An electric field suitable for electrotransfer can be transmitted at or near the site of administration (e.g., injection) of the nucleic acid vector, or pharmaceutical composition thereof. For instance, in some embodiments, the nucleic acid vector or pharmaceutical composition thereof is administered intravitreally, and the electrode is positioned at or near the site of intravitreal administration for transmission of the electric field (e.g., within 10 mm, within 8 mm, within 6 mm, within 5 mm, within 4 mm, within 3 mm, within 2 mm, or within 1 mm of the site of intravitreal administration). In other embodiments, the nucleic acid vector or pharmaceutical composition thereof is administered (e.g., injected) subretinally, and the electrode is positioned at or near the site of subretinal administration for transmission of the electric field (e.g., within 10 mm, within 8 mm, within 6 mm, within 5 mm, within 4 mm, within 3 mm, within 2 mm, or within 1 mm of the site of subretinal administration). In other embodiments, the nucleic acid vector or pharmaceutical composition thereof is administered suprachoroidally, and the electrode is positioned at or near the site of suprachoroidal administration for transmission of the electric field (e.g., within 10 mm, within 8 mm, within 6 mm, within 5 mm, within 4 mm, within 3 mm, within 2 mm, or within 1 mm of the site of suprachoroidal administration).

In some instances, the nucleic acid vector or pharmaceutical composition thereof is administered at a location that is exposed to the electric field (or will be exposed to the electric field, in the event of subsequent electric field transmission). In some embodiments, the nucleic acid vector or pharmaceutical composition thereof is delivered at a location that is exposed to (or will be exposed to) a voltage that is 1% or more of the maximum tissue voltage (e.g., at least 5% of the maximum tissue voltage, at least 10% of the maximum tissue voltage, at least 20% of the maximum tissue voltage, at least 30% of the maximum tissue voltage, at least 40% of the maximum tissue voltage, at least 50% of the maximum tissue voltage, at least 60% of the maximum tissue voltage, at least 70% of the maximum tissue voltage, at least 80% of the maximum tissue voltage, or at least 90% of the maximum tissue voltage, e.g., from 1% to 10% of the maximum tissue voltage, from 10% to 20% of the maximum tissue voltage, from 20% to 30% of the maximum tissue voltage, from 30% to 40% of the maximum tissue voltage, from 40% to 50% of the maximum tissue voltage, from 50% to 60% of the maximum tissue voltage, from 60% to 70% of the maximum tissue voltage, from 70% to 80% of the maximum tissue voltage, from 80% to 90% of the maximum tissue voltage, from 90% to 95% of the maximum tissue voltage, or from 95% to 100% of the maximum tissue voltage).

Alternatively, the site of administration can be in a region of tissue away from the electric field. For example, administration of the nucleic acid vector or pharmaceutical composition thereof may be systemic (e.g., intravenous), while the electric field is transmitted in the eye (e.g., in the vitreous humor or in the subretinal space).

In any of the methods described herein involving electrotransfer (e.g., by PEF), a paralytic may be administered according to standard procedures, which can help reduce the risk and/or severity of muscle contractions upon transmission of electrical energy.

Additionally, or alternatively, nucleic acid vectors or pharmaceutical compositions thereof can be administered to host cells ex vivo, such as by cells explanted from an individual patient, followed by reimplantation of the host cells into a patient, e.g., after selection for cells which have incorporated the vector. Thus, in some aspects, the disclosure provides transfected host cells and methods of administration thereof for treating a disease.

Additionally or alternatively, the present invention includes methods of treating a subject having a disease or disorder by administering to the subject the nucleic acid vector, or pharmaceutical composition thereof, of the invention. Assessment of the efficiency of transfection of any of the nucleic acid vectors described herein can be performed using any method known in the art or described herein. Isolating a transfected cell can also be performed in accordance with standard techniques. For example, a cell comprising a therapeutic gene can express a visible marker, such as a fluorescent protein (e.g., GFP) or other reporter protein, encoded by the sequence of the heterologous gene that aids in the identification and isolation of a cell or cells comprising the heterologous gene.

The level or concentration of a protein (e.g., an ocular protein (e.g., retinal protein)) expressed from a DNA vector described herein may be an expression level, presence, absence, truncation, or alteration of the administered vector. It can be advantageous to correlate the level with one or more clinical phenotypes or with an assay for a human disease biomarker. The assay may be performed using construct specific probes, cytometry, qRT-PCR, real-time PCR, PCR, flow cytometry, electrophoresis, mass spectrometry, or combinations thereof while the exosomes may be isolated using immunohistochemical methods such as enzyme linked immunosorbent assay (ELISA) methods. Therapeutic genes delivered by the nucleic acid vectors described herein may be quantified using methods such as, but not limited to, ultraviolet visible spectroscopy (UV/Vis). The quantified polynucleotide may be analyzed in order to determine if the polynucleotide may be of proper size, check that no degradation of the polynucleotide has occurred. Degradation of the polynucleotide may be checked by methods such as, but not limited to, agarose gel electrophoresis, HPLC based purification methods such as, but not limited to, strong anion exchange HPLC, weak anion exchange HPLC, reverse phase HPLC (RP-HPLC), hydrophobic interaction HPLC (HIC-HPLC), liquid chromatography-mass spectrometry (LCMS), capillary electrophoresis (CE), and capillary gel electrophoresis (CGE).

Efficacy of treatment can be monitored, assessed, and/or quantified using any suitable methods known in the art or provided herein. For example, an individual treated for a retinal disease or disorder may be monitored periodically to assess progression of retinal degeneration, e.g., by testing visual acuity and visual field using standard tests. Additionally, or alternatively, optical coherence tomography (OCT) (e.g., spectral domain OCT (SD-OCT)) can be conducted to assess changes in retinal structure. In some instances, an individual treated by the methods described herein exhibits improvement or no further degradation in retinal structure assessed by imaging endpoints, such as fundus autofluorescence (FAF) and/or SD-OCT.

Safety and tolerability of treatment can be monitored, assessed, and/or quantified using any suitable methods known in the art or provided herein. For instance, an individual treated for a retinal disease or disorder may be monitored periodically to assess cataract formation, intra-ocular inflammation, or retina damage such as RPE hypopigmentation. In some embodiments, an individual treated according to the methods described herein exhibits no cataract formation, no intraocular inflammation up to two months post-treatment (or less than grade 2 intraocular inflammation up to two months post-treatment), and/or minimal retina/RPE damage (e.g., RPE hypopigmentation).

Accordingly, methods of the present invention include, after administering any of the nucleic acid vectors, or pharmaceutical compositions thereof, described herein to a subject, subsequently detecting the expression of the transgene in the subject. Expression can be detected one week to four weeks after administration, one month to four months after administration, four months to one year after administration, one year to five years after administration, or five years to twenty years after administration (e.g., at least one week, at least two weeks, at least one month, at least four months, at least one year, at least two years, at least five years, at least ten years after administration). At any of these detection timepoints, persistence (e.g., episomal persistence) of the nucleic acid vector may be observed. In some embodiments, the persistence of a synthetic circular DNA vector is from 5% to 50% greater, 50% to 100% greater, one-fold to five-fold, or five-fold to ten-fold (e.g., at least 5%, 10%, 20%, 30%, 40%, 50%, 75%, one-fold, two-fold, three-fold, four-fold, five-fold, six-fold, seven-fold, eight-fold, nine-fold, ten-fold, or more) greater than a reference vector (e.g., a circular DNA vector produced in bacteria or having one or more bacterial signatures not present in the vector of the invention).

Expression constructs and nucleic acid vectors of the invention can be expressed in a particular target cell. Methods of the invention include expressing functional BEST1 in a target cell that would normally express BEST1. In certain instances, BEST1 is expressed preferentially in the target cell (e.g., as compared to a non-target cell). For instance, a target cell may be an RPE cell, and the methods provided herein include expressing the functional BEST1 in an RPE cells. In some instances, the number of BEST1 protein molecules expressed by the BEST1 transgene in RPE cells exceeds the number of BEST1 protein molecules expressed by the BEST1 transgene in photoreceptor cells by at least 20%, by at least 50%, by at least two-fold, by at least five-fold, by at least ten-fold, by at least 50-fold, by at least 100-fold, or more).

In some embodiments of methods involving preferential expression in a target ocular cell by administration of a nucleic acid vector, in vivo electrotransfer is performed as part of the method, or before or after the administration of the nucleic acid vector or pharmaceutical composition thereof. For example, methods involving preferential expression of BEST1 in RPE cells (e.g., compared to photoreceptor cells) include methods of administering any of the BEST1-encoding nucleic acid vectors described herein (e.g., a nonviral BEST1-encoding DNA vector (e.g., a naked BEST1-encoding DNA vector), a circular BEST1-encoding DNA vector (e.g., a supercoiled circular BEST1-encoding DNA vector), and/or a synthetic BEST1-encoding DNA vector (e.g., a synthetic circular BEST1-encoding DNA vector)) described herein, or pharmaceutical composition thereof, in combination with any of the ocular electrotransfer methods described herein (e.g., using an intra-ocular electrode).

In some instances, an individual is treated with nucleic acid vector, or pharmaceutical composition thereof, according to any of the embodiments described herein only once in their lifetime (e.g., treatment of the disease or disorder is sustained for several years (e.g., three to five years, five to ten years, ten to fifteen years, or at least 15 years)). Alternatively, an individual may be treated exactly twice in their lifetime. Additionally, or alternatively, an individual may be treated once every 2-3 years, every 3-5 years, or every 5-10 years.

### B. Ocular Diseases

Nucleic acid vectors and pharmaceutical compositions described herein can be used for treatment of various ocular diseases or disorders. In some instances, the ocular disease or disorder is a retinal disease or disorder, such as a retinal dystrophy (e.g., a retinal dystrophy characterized by reduced level of functional expression (e.g., a lack of functional expression) of BEST1 in the individual relative to a reference (e.g., a healthy level of functional expression)). BEST1-associated retinal disorders treatable by the compositions and methods described herein include recessive bestrophinopathies (e.g., autosomal recessive bestrophinopathy) and dominant bestrophinopathies (e.g., Best's vitelliform macular dystrophy, BEST1 adult-onset vitelliform macular dystrophy, and autosomal dominant vitreoretinochoroidopathy). In some instances, nucleic acid vectors that include shRNA-encoding sequences (e.g., a sequence comprising SEQ ID NO: 1, a sequence comprising SEQ ID NO: 1 and SEQ ID NO: 3, e.g., a sequence comprising SEQ ID NOs: 1-3; e.g., a sequence comprising or consisting of SEQ ID NO: 4) for knockdown of native, dysfunctional BEST1 expression, can be particularly useful in dominant bestrophinopathies.

In some embodiments, the ocular disease or disorder (e.g., retinal disease or disorder) is an autosomal recessive bestrophinopathy, a Best vitelliform macular dystrophy, an autosomal dominant vitreoretinochoroidopathy, an autosomal dominant microcornea, a rod-cone dystrophy, an early-onset cataract posterior staphyloma syndrome, or a retinitis pigmentosa.

In some embodiments, the individual to be treated is a human patient. In some embodiments, the individual is a pediatric human patient, e.g., a person aged 21 years or younger at the time of their diagnosis or treatment. In some embodiments, the pediatric human patient is aged 16 years or younger at the time of treatment. In other embodiments, the individual is aged 22 to 40 years at the time of treatment. In other embodiments, the individual is aged 41 to 60 years at the time of treatment. In other embodiments, the individual is aged 61 years or older at the time of treatment. In some instances, the individual is male. In other instances, the individual is female.

### VI. Kits and Articles of Manufacture

In another aspect of the invention, provided herein is an article of manufacture or a kit containing any of the nucleic acid vectors, or pharmaceutical compositions thereof, described herein. Thus, the present kits and articles of manufacture include a DNA vector having (a) a DNA sequence encoding a BEST1 RNA transcript and (b) a DNA sequence encoding a short hairpin RNA (shRNA) comprising SEQ ID NO: 1 (e.g., a sequence comprising SEQ ID NO: 1, a sequence comprising SEQ ID NO: 1 and SEQ ID NO: 3, e.g., a sequence comprising SEQ ID NOs: 1-3; e.g., a sequence comprising or consisting of SEQ ID NO: 4), wherein the shRNA is not capable of targeting the BEST1 RNA transcript, or a pharmaceutical composition thereof.

The article of manufacture or kit can include a container and a label or package insert on, or associated with, the container. Suitable containers include, for example, bottles, vials, syringes, IV solution bags, etc. The containers may be formed from a variety of materials, such as glass or plastic. The container holds a composition which is by itself or combined with another composition effective for treating, preventing and/or diagnosing a condition and may have a sterile access port (for example the container may be an intravenous solution bag or a vial having a stopper pierceable by a hypodermic injection needle). At least one active agent in the composition is a nucleic acid vector of the invention or a pharmaceutical composition comprising the nucleic acid vector.

The label or package insert indicates that the composition is used for treating the condition treatable by its contents (e.g., an ocular disease or disorder, e.g., an BEST1-associated retinal dystrophy).

Moreover, the kit or article of manufacture may comprise (a) a first container with a composition contained therein, wherein the composition comprises any of the nucleic acid vectors described herein; and (b) a second container with a composition contained therein, wherein the composition comprises an additional therapeutic agent. The article of manufacture may further comprise a package insert indicating that the compositions can be used to treat a particular condition (e.g., an ocular disease or disorder, e.g., an BEST-associated retinal dystrophy).

Alternatively, or additionally, the article of manufacture may further comprise a second (or third) container comprising a pharmaceutically acceptable carrier, such as bacteriostatic water for injection (BWFI), phosphate-buffered saline, Ringer's solution, dextrose solution, or any of the pharmaceutically acceptable carriers disclosed above. It may further include other materials desirable from a commercial and user standpoint, including other buffers, diluents, filters, needles, syringes, or other delivery devices.

### EXAMPLES

These examples are provided for illustrative purposes only and not to limit the scope of the claims provided herein.

### Example 1. Screening for Efficient BEST1 Knockdown

In this study, various shRNA sequences having complementarity to different portions of endogenous BEST1 DNA were tested in an in vitro culture of human induced pluripotent cell (iPSC)-derived retinal pigment epithelial (RPE) cells to select a lead shRNA sequence to be incorporated into a DNA vector along with a functional BEST1-encoding sequence.

iPSC-derived RPE cells (passage 4) were produced according to known methods and grown to confluence as a monolayer on laminin-coated, 6.5 mm transwell plates. Cells were transduced at 200000 MOI with AAV vectors containing an EGFP reporter gene and a U6 promoter driving expression of the following shRNA sequences: sh1 (scramble control); sh2 (GCCGGACATGTACTGGAATAACTCGAGTTATTCCAGTACATGTCCGGC), sh3 (TGGATTGTCGACAGGAATTTGCTCGAGCAAATTCCTGTCGACAATCCA), and sh4 (GCCTACGACTGGATTAGTATCCTCGAGGATACTAATCCAGTCGTAGGC). After 24 hours, media in the upper chambers was replaced with fresh media containing 5% fetal bovine serum. Media was refreshed again after 3 days, 7 days, and 13 days, and then twice per week. At 3 weeks post-treatment, media was removed and transwells washed. Cells were removed by conventional enzymatic detachment and resuspended in 1 mL PBS (Ca/Mg-free). Cells were filtered and sorted by fluorescence-activated cell sorting (FACS) at 700-1000 events per second. iPSC-derived RPE cells transfected with GFP-encoding synthetic circular DNA was used as a positive control (GFP control).

Percentages of GFP-positive cells for each group are shown below:

TABLE 1
Transduction efficiency (GFP expression at sorting)

| | | No. of GFP-positive |
|---|---|---|
| Treatment Group | % GFP-positive cells | RPE cells |
| GFP control | 90% | 270,000 |
| sh1 | 45% | 226,000 |
| sh2 | 46% | 270,000 |
| sh3 | 33% | 146,000 |
| sh4 | 46% | 200,000 |

Sorted cells from each group were centrifuged and processed for RNA extraction using RNeasy mini kit (QIAGEN®). 25 μL of RNase free water was eluted to measure RNA concentration using Nanodrop. 350 ng of each RNA sample was converted into cDNA using QUANTINOVA® Reverse Transcription kit (QIAGEN®). BEST1 and GFP expression by mRNA was quantified by TAQMAN® assay, and BEST1 expression was normalized to GFP expression.

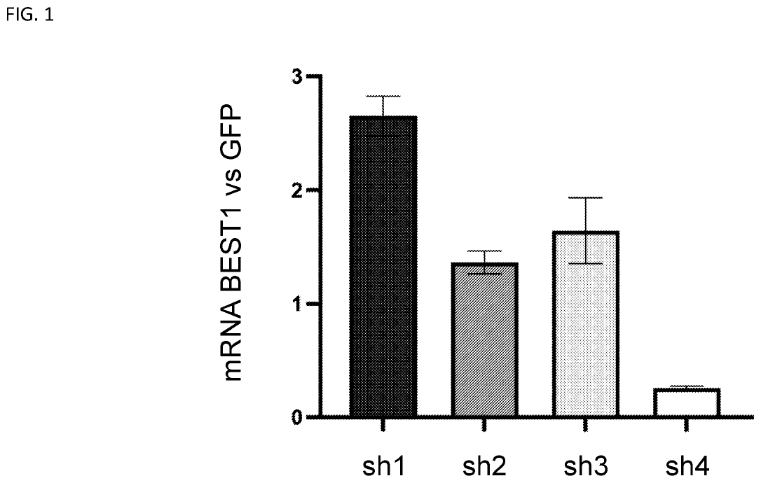

As shown in FIG. 1 and Table 2, sh2, sh3, and sh4 knocked down BEST1 to a greater extent than the sh1 control. Notably and unexpectedly, sh4 conferred a substantially greater knockdown of BEST1 than any other shRNA.

TABLE 2
Transduction efficiency (GFP expression at sorting)

| | Mean mRNA of | |
|---|---|---|
| Treatment Group | BEST1/GFP | Standard Deviation |
| sh1 | 2.652 | 0.174 |
| sh2 | 1.364 | 0.100 |
| sh3 | 1.643 | 0.292 |
| sh4 | 0.257 | 0.014 |

### Example 2. Pairing an shRNA with a BEST1 Coding Sequence

To produce BEST1-encoding nucleic acid vectors with endogenous BEST1 knockdown functionality, sh4 (SEQ ID NO: 4) was selected as an shRNA sequence to be paired with a BEST1 coding sequence, based on the results shown in Example 1. In order to mitigate knocking down expression of the BEST1 coding sequence, the BEST1 coding sequence was altered to reduce complementarity to the shRNA sequence. The full BEST1 coding sequence, which is further codon-optimized, is given by SEQ ID NO: 6.

In the DNA vector containing the shRNA-encoding sequence of SEQ ID NO: 4 and the BEST1-encoding sequence of SEQ ID NO: 6, a U6 promoter is positioned upstream of the shRNA sequence, a native BEST1 promoter sequence of SEQ ID NO: 40 is positioned upstream of the BEST1-encoding sequence, and a polyA sequence is positioned downstream of the BEST1-encoding sequence, such that the DNA vector contains, operably linked in a 5′ to 3′ direction, a U6 promoter, sh4 (SEQ ID NO: 4), a native BEST1 promoter, a modified BEST1-encoding sequence (SEQ ID NO: 6), and a polyA sequence.

### Example 3. Regulatory Element Screening and Selection

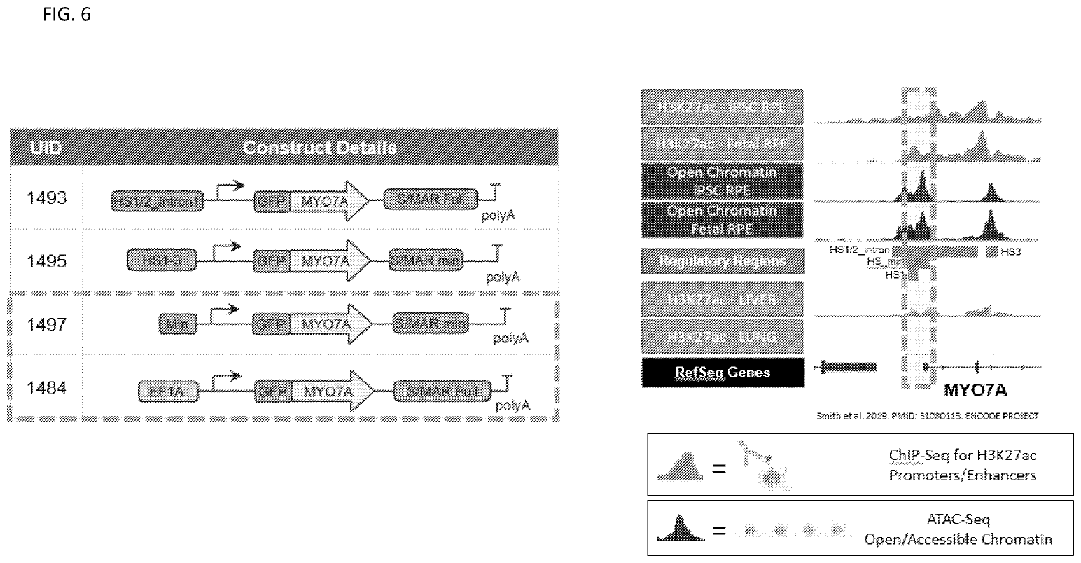

Potential native promoter constructs were selected by analyzing genomic datasets with chromatin structure data deposited in publicly available repositories. These datasets were searched for cell types of interest, e.g., RPE cells, where BEST1 expression can be targeted. Relevant datasets were chosen based on chromatin accessibility data, including histone post-translational modifications. Areas of enrichment for chromatin modification and/or open chromatin in the region of MYO7A were identified based on these data and potential regulatory sequences were selected in sizes ranging from ˜2 kb down to a few hundred bases. The following candidate native or constitutive promoter sequences were chosen: MYO7A Promoter HS1/2_Intron1 (SEQ ID NO: 21), MYO7A Promoter HS1-3 (SEQ ID NO: 22), and MYO7A Promoter Min (SEQ ID NO: 23). Examples of ChIP-Seq for H3K27ac and ATAC-Seq (the reference for those experiments are below the images) for open/accessible chromatin are shown to illustrate how candidate regulatory elements were mapped for MYO7A expression construct generation as shown in FIG. 6.

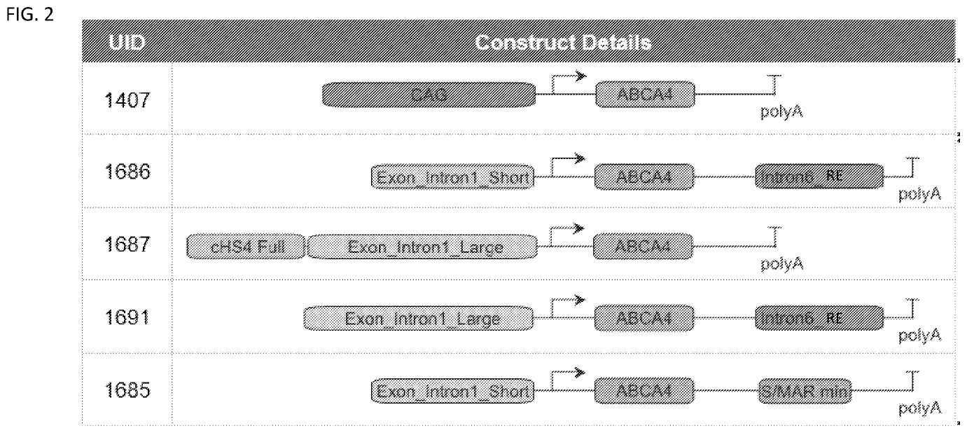

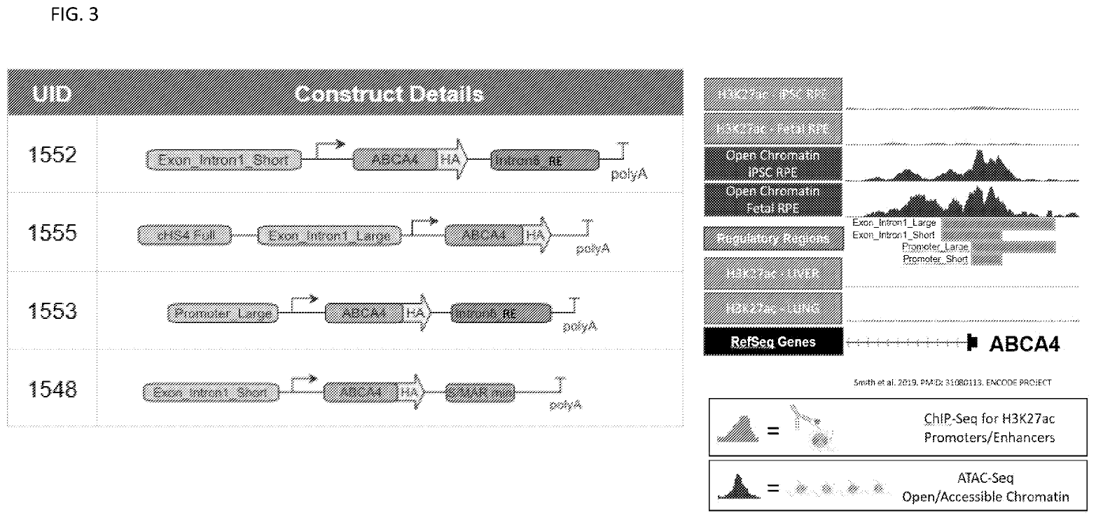

Various expression constructs were constructed including various promoters and regulatory elements, as shown in FIGS. 2-8. The following elements were included in constructs as indicated in the figures: S/MAR_Full, which is the full-length human interferon-β S/MAR (SEQ ID NO: 9); S/MAR min, which includes three repeats of a portion of the human interferon-β S/MAR (SEQ ID NO: 10); MYO7A Promoter HS1/2_Intron1 (SEQ ID NO: 21); Promoter HS1-3 (SEQ ID NO: 22); MYO7A Promoter Min (SEQ ID NO: 5)); ABCA4 Intron 6 RE (regulatory element derived from a nuclease-sensitive region of ABCA4 intron 6-see Examples 6 and 7 below) (SEQ ID NO: 8), ABCA4 Promoter Exon_intron1_Short (SEQ ID NO: 24), ABCA4 Promoter Exon_intron1_large (SEQ ID NO: 25), ABCA4 Promoter_Large (SEQ ID NO: 26), ABCA4 Promoter_Short (SEQ ID NO: 27), chicken β-globin insulator (cHS4; SEQ ID NO: 11); a CAG promoter (SEQ ID NO: 28), or an EF1A promoter (SEQ ID NO: 29).

### Example 4. Production of Synthetic DNA Vectors

The sequences of expression construct elements described above were synthesized into plasmids using standard manufacturing processes to produce plasmids each containing a single element. These single-element plasmids were then ligated together to produce template plasmids containing all elements of an expression construct using a standard cloning process. Briefly, each plasmid having an individual expression element contained restriction sites flanking the element and overhangs required to ligate the sequences in the right order and orientation. The restriction reaction cut each required element from its respective plasmid and ligated the fragments into a new plasmid at the same time. For example, to make expression construct 1686 shown in FIG. 1, three plasmids having the ABCA4 Exon_Intron1_Short sequence (SEQ ID NO: 24), the ABCA4 coding sequence, and the Intron 6 RE (SEQ ID NO: 8) sequences were mixed with a plasmid with restriction sites having appropriate overhangs and subjected to BsaI restriction and ligation. The restriction overhangs were designed such that the ligation of the expression elements together would generate a plasmid having an expression construct with the elements in the order shown in FIG. 1. The restriction and ligation reaction was prepared as follows: 2 μl of BSA buffer, 2 μl of T4 ligase buffer, 1.5 μl restriction enzyme, 0.5 μl of T4 ligase, and equimolar concentrations of each plasmid preparation and water to reach 20 μl total volume. This master mix was then briefly vortexed/mixed and briefly centrifuged. The master mix was then placed in a thermocycler with the following steps: (1) 37° C. for 15 minutes; (2) 37° C. for two minutes; (3) 16° C. for five minutes; and (4) repeat steps (2) and (3) 50 times.

One μl of the resulting product was then used to transform E. coli μsing protocols well known in the art. Resulting plasmids were then purified and the DNA digested with the relevant restriction enzyme to verify the accuracy of the final plasmid. Positive DNA sequence clones of interest were then verified using DNA sequencing and subsequently amplified following the verification. These sequences were then cloned into a type IIs restriction site-containing backbone to form template plasmids for generation of synthetic circular DNA vectors.

Synthetic circular DNA vectors were then produced from the template plasmids using methods generally taught in International Patent Publication Number WO 2019/178500 to remove plasmid backbone components, such as bacterial origins of replication and resistance genes. Briefly, template plasmids were amplified by rolling circle amplification using Phi29 polymerase, restriction enzymes were added to cut the amplified product at sites flanking the therapeutic sequence, and the therapeutic sequence was recircularized by ligation using a ligase. In the present examples, the process was carried out using a single restriction enzyme, BsaI, which cut recognition sites flanking the therapeutic sequence and within the plasmid backbone. Upon ligation, the linear therapeutic fragment circularizes into a therapeutic circular DNA vector, and the linear backbone fragment circularizes. Without being bound by theory, the circularized backbone fragment contains a BsaI cut site and ligation occurs in the presence of the BsaI enzyme, so BsaI can cut the backbone and does not cut the therapeutic circular DNA vector, thereby driving the reaction forward toward a purer therapeutic circular DNA product. Exonuclease was added to digest the remaining linear backbone, and gyrase was added to supercoil the therapeutic circular DNA vector.

### Example 5. Functional Testing of Regulatory Elements for Expression of Ocular Genes in RPE Cells

The expression constructs of interest identified in FIGS. 2-8, were selected and the expression and persistence of these expression constructs were then screened by transfecting relevant cell types, e.g., iRPE cells, using standard techniques, and testing the copy number of the vectors and expression of the target ocular gene in vitro.

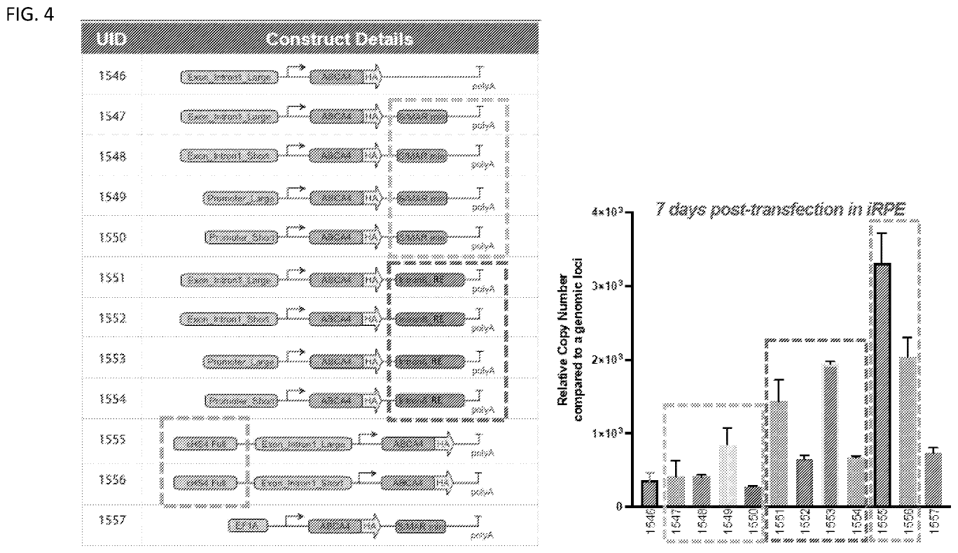

Transfection was performed with Lipofectamine 3000 following a standard protocol. 300K iRPE cells were seeded in Laminin coated 6-well plates in 800 μl of media. All DNA vectors were normalized to 0.2 pmol of DNA ranging from 4-9 μg. After 24 hours, cells were washed with PBS and fresh media was added. Transfected cells were grown for seven days post-transfection. DNA and RNA were extracted following standard protocols (MONARCH® Genomic DNA Purification Kit from NEB and RNeasy Mini Kit from Qiagen). Detection by qPCR was performed with LUNAR Universal One-Step RT-qPCR Kit in a Q7 thermocycler. Transfection efficiency was assessed using DNA copy number detected by qPCR for DNA, using primers for a genomic control region and specific target ocular gene primers. The DNA copy number was reported relative to the genomic control region loci (FIG. 4). Primers for the genomic control amplified a region in exon 27 of the RB1 human gene were (Fwd: CCTAGCCTTTAAGGGGCTCTA (SEQ ID NO: 30); Rev: TGACCTCTGTGAAAAGATCAGGG (SEQ ID NO: 31), and primers specific for the codon optimized ABCA4 sequence were (Fwd: TGGGAGTTAGACCCGGCGAGTG (SEQ ID NO: 32); Rev: TGTAGCATCTCCGCTTGTCACT (SEQ ID NO: 33)). Expression constructs containing a cHS4 Full insulator sequence (UID 1555 and 1556) resulted in the highest observed DNA copy number (see FIG. 4).

Gene expression was assessed by harvesting transfected cells and performing assays to detect protein and/or RNA relative expression. Protein detection was performed by standard western blot or immunofluorescence.

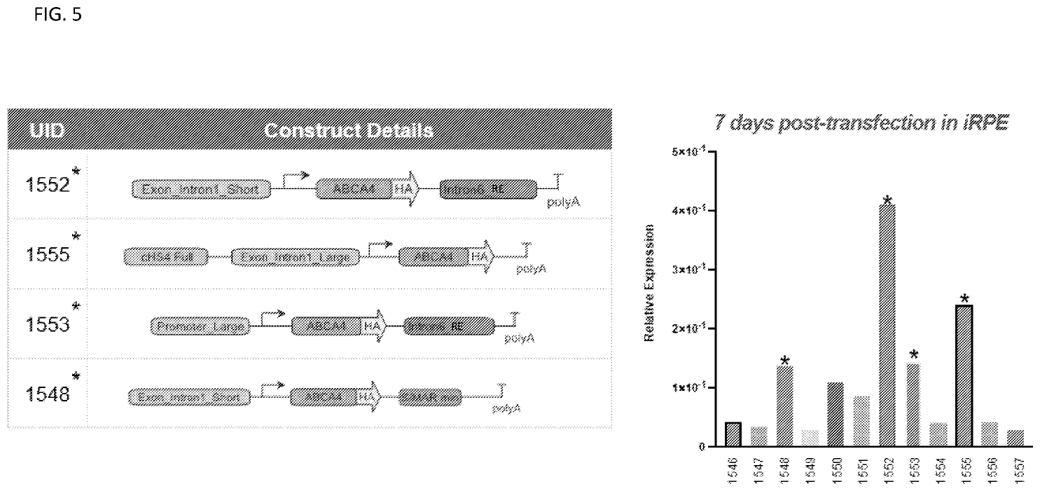

RNA expression of transfected cells was assessed by a relative quantification by RT-qPCR against a housekeeping gene, GAPDH, for selected constructs. RNA expression was then normalized to copy number by DNA content qPCR using a genomic control region and plasmid specific primers. For RT-qPCR: for the codon optimized ABCA4 (same primers as above); for Human GAPDH (Fwd: CAGTCTTCTGGGTGGCAGTG (SEQ ID NO: 34); Rev: AACCATGAGAAGTATGACAACAGC (SEQ ID NO: 35)). Constructs of interest were selected for further analysis based upon the efficacy of the constructs for increasing DNA copy number and RNA expression. The ABCA4 expression constructs UID 1552, 1555, 1553, and 1548, denoted by * in FIG. 5, generated the highest relative expression of mRNA and episomal persistence seven days following transfection into iRPE cells. Results for copy number and expression for ABCA4 expression constructs are shown in FIG. 4 and FIG. 5.

### Example 6. Functional Testing of Native Promoters in RPE Cells

Natural endogenous promoters for MYO7A were screened and were selected based on transcription factor binding and localization using ChIP-SEQ for H3K27ac, and ATAC-Seq for open/accessible chromatin (FIG. 6). EF1A was also selected as a strong constitutive promoter. Representative MYO7A expression constructs are shown in FIG. 5.

Localization of the expressed proteins was assessed using neon transfection in iRPE cells. iRPE cells were seeded at 1:3 to Laminin coated 6-well plate and cultured for 48 hours to 100% confluency. Cells were lifted with TrypLE and counted for cell numbers. Greater than 2.5×10⁵ cells were selected for one 24-well plate and resuspended in Buffer R (Thermo Scientific). Plasmid DNA was diluted to 1 μg/well in Buffer R and mixed with iRPE cells. Cells were electroporated (Neon Transfection System; 1100 V, 20 ms, 2 pulses) and seeded to a 24-well plate containing 0.5 mL conditional media and grown for at least 48 hours.

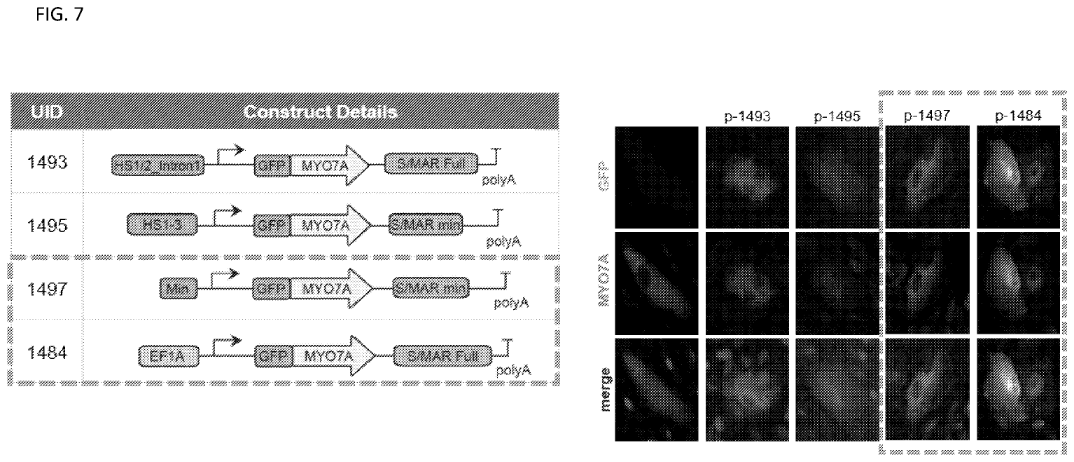

Immunocytochemistry was then performed on the transfected cells using the following protocol: Cells were fixed with 4% PFA at room temperature for 15 minutes. Cells were washed three times with PBS for five minutes and subsequently blocked with 5% BSA in 0.3 Triton-X100 in PBS at room temperature for at least 30 minutes. Cells were then incubated with primary antibody (Anti-MYO7A (Abcam, ab150386): 1:500) in blocking solution at 4° C. overnight. Following antibody incubation, cells were washed three times with 1×PBS for five minutes and then incubated with secondary antibody (Goat anti-rabbit 594 at 1:500) in blocking solution at room temperature for two hours in the dark. Cells were then washed three times with PBS for five minutes and stained with DAPI for 15 minutes. Following DAPI staining, cells were washed with PBS and imaged. Expression was observed for all constructs, and the pattern of expression between GFP and MYO7A was consistent across all constructs. The expression construct UID 1484 induced the highest expression of MYO7A as assessed by GFP expression, as shown in FIG. 7.

### Example 7. Expression of DNA Vectors Containing S/MAR and Truncated S/MAR

An interferon-β (IFN-β) Scaffold Matrix Attachment Region (S/MAR; SEQ ID NO: 9) and truncated variant thereof (S/MAR min; SEQ ID NO: 10) were included in select plasmid expression constructs: UID 1685, 1547, 1548, 1549, 1550, 1557, 1493, 1495, 1497, and 1484. The expression efficiency of S/MAR-containing constructs is demonstrated in FIGS. 4-9.

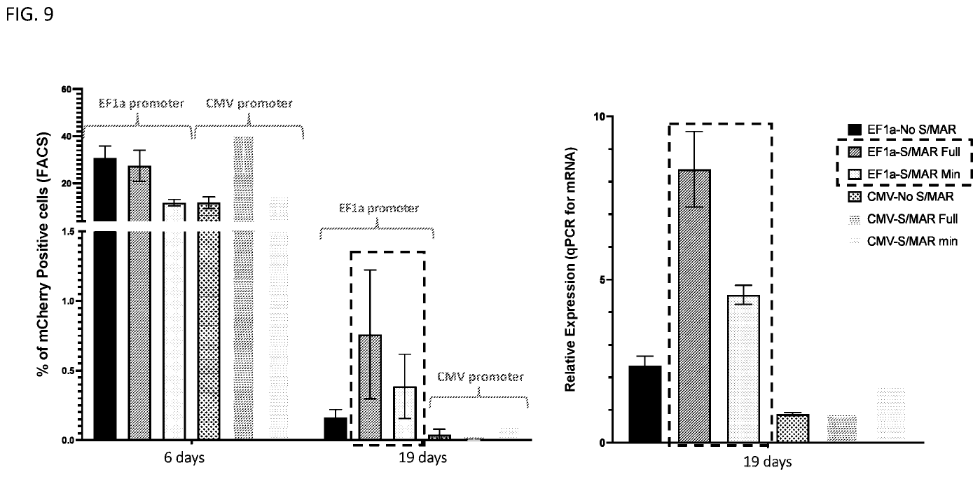

In vitro assays assessing the impact of the S/MAR were performed using K562 cells, which are rapidly dividing cells. Inclusion of S/MAR sequences in the expression construct with an EF1A promoter (SEQ ID NO: 29) resulted in expression of both mCherry (FACS) and relative mRNA (qPCR) after 19 days, while the CMV promoter constructs lost gene expression FIG. 9.

### Example 8. In Vivo Expression of a Synthetic Circular DNA Vector Containing S/MAR and Truncated S/MAR

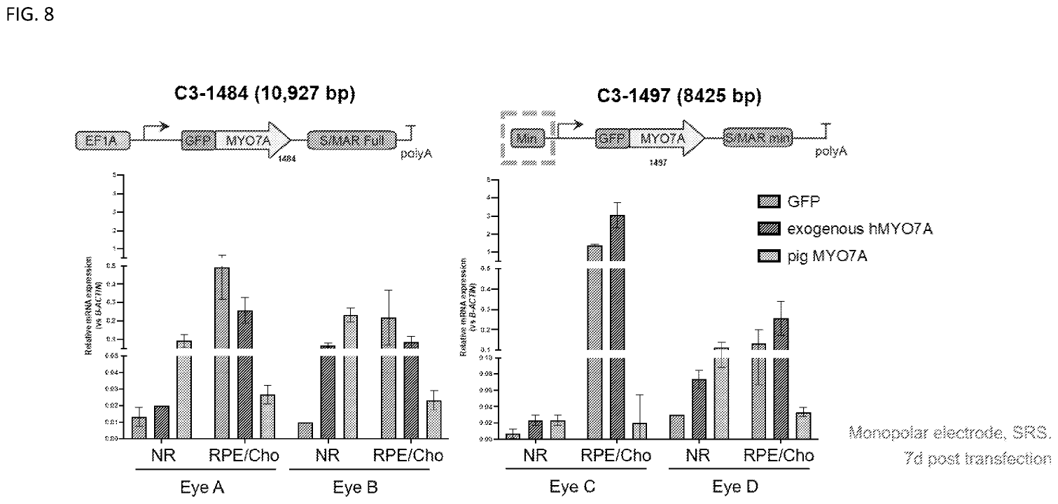

C³DNA vectors 1484 (containing full-length S/MAR; C³-1484; 10,927 bp) and 1497 (containing truncated S/MAR; C³-1497; 8,325 bp)) were selected for in vivo expression experiments. C³-1484 and C³-1497 were formulated in a solution at a concentration of 1.5 mg/mL. Naked vector was administered to Gottingen Minipigs by injecting two blebs (75 μL each) into the subretinal space (225 μg DNA; 2.53×10¹³ vector copies per eye). After injection, a monopolar needle electrode was placed within each subretinal bleb, and eight 20-ms electrical pulses were transmitted at 20V. Relative mRNA expression of GFP, exogenous human MY07A (hMYO7A) and pig MYO7A was measured in the RPE/choroid and NR layers of the eye seven days after the initial transfection. Robust detection of exogenous sequences (both GFP and MYO7A) was observed in RPE/choroid tissues in both C³-1484 and C³-1497-treated animals, indicating robust expression in RPE/choroid by vectors containing both forms of full-length and truncated S/MAR, using endogenous pig MYO7A as a control (FIG. 8).

### Example 9. Identification of Regulatory Sequences for Long-Term Episomal Persistence

Screening was conducted to identify novel, native regulatory sequences that could improve expression and/or persistence in RPE cells. DNA was purified from the nuclear matrix of ARPE-19 or iRPE cells and a library was generated by end-repair and ligation of DNA into barcoded plasmids. To obtain a manageable range of sizes (in bp) for library construction, the nuclear matrix isolated from iRPE cells was sheared prior to library construction. The library was then delivered to cells, e.g., iRPE cells, and fluorescent reporters were used for the long-term assessment of gene expression. Positive cells were selected and the DNA was sequenced to identify relevant regulatory sequences that confer long-term durability of gene expression and increased episomal persistence. This method revealed a sequence within intron 6 of ABCA4 (nucleotides 3158-4822 of ABCA4 intron 6; intron 6 RE (16RE)) as a potential regulatory element useful to improve expression of ocular genes in RPE cells.

### Example 10. RPE Cell Expression of DNA Vectors Containing ABCA4 Intron 6 RE

Plasmid DNA vectors encoding human protein (ABCA4) driven by various promoters and including an ABCA4 intron 6 RE (16RE) of SEQ ID NO: 8 were produced as described in Example 4. The ABCA4 i6RE was modified from the region of ABCA4 intron 6 from which it was derived to allow for BsaI restriction digest by deleting a G at position 3530 of native human ABCA4 intron 6 to remove a BsaI recognition site, as described above.

Four promoters were tested: Exon_Intron1_Large (SEQ ID NO: 25, within p-1551 (SEQ ID NO: 36)), Exon_Intron1_Short (SEQ ID NO: 24, within p-1552 (SEQ ID NO: 37)), Promoter_Large (SEQ ID NO: 26, within p-1553 (SEQ ID NO: 38)), and Promoter_Short (SEQ ID NO: 27, within p-1554 (SEQ ID NO: 39)).

In vitro expression was tested in the experiments described in Example 3, and results are shown in FIGS. 4 and 5. Observed expression by copy number of each plasmid vector containing 16RE (p-1551, p-1552, p-1553, and p-1554) were greater than their respective controls containing truncated S/MAR (p-1547, p-1548, p-1549, and p-1550) (FIG. 4).

The following table lists sequences included in this disclosure:

| SEQ | |
|---|---|
| ID | |
| NO | |
| 21 | CCTGTCCCCCATAACCTGCCCACCTCTCTGCCCCATTCCCACAGCTCACTGCATT |
| | TTGTCCCTGCTGCCAGTCGTGTGCCATGGGCTCTCCTGTGCTCCCCCGGGCTCTC |
| | ATTTCCCCATCCGTGTAGGGCTGACGCCACTGGCCCCGCCCAGCCTGGGAGGATG |
| | GAGAGGGGGGACTTGAGGAGTGGCCAGTACTGGGTTTCGGCAGCAGGTGCAAATC |
| | CCAGGTGGGGACTGTGTTCCAGGGACTCCTGGACTCCTGTTAGGCCAGCAGGCTA |
| | AGGGGCAGACGGCATCCTGGGGTCTTCCTGGGACAGGGTCGTCGGGTCTGAGGGG |
| | ACCCAGGCCCTAGCAGAAGCTGACTCCTGTCACGGTCACTTCCCAGCGGTGCTCA |
| | GCCCGCCTGACGGAAGCAGCTGGGCAGTGGGGCCTGTGACCAGCAGGCGGCGCTG |
| | GCGAGCTATAGCGCCCCTTTGGGCCCCCACCCCACCTTCTGGGCAGCTTCCCTGC |
| | AGACACCCCAGTTATGGGGGCTAGGGACCCAAAAGAGACATCCTTCTGCCACCCA |
| | GAGCTGCCCTGGCGAGGTGCACTATGGGGCCGCCGACAGCTGCGTGGCTGCCGAG |
| | GGCGGAAAGGAGAAACTGTCATGTCCCGATAGGGCCGCGCGAGCTCTCCATCCTC |
| | GACAACGCTAATAACAAAGACGTGTGCTCCTCTTTGCTTGGTTCCCCCCACTCCT |
| | TTAAATCACAGATTTCACTTCAGTTTATCTGTGTCGCTGTCACACGTGGGGTGGC |
| | TCCCAGTCAGCTGGTTTGGCAAAGTTTCTGGATGATTACGGAATAACATGTGTCC |
| | CCAACCCGCAGAGCAGGTTGTGGGGGCAATGTTGCATTGACCAGCGTCAGAGAAC |
| | ACACATCAGAGGCAAGGGTGGGTGTGCAGGAGGGAGAAGGCGCAGAAGGCAGGGC |
| | TTTAGCTCAGCACTCTCCCTCCTGCCATGCTCTGCCTGACCGTTCCCTCTCTGAG |
| | TCCCAAACAGCCAGGTAGAGGAGGAAGAAATGGGGCTGAGAgCCCAGCACATCAG |
| | TGATTAAGTCAGGATCAGGTGCGGTTTCCTGCTCAGGTGCTGAGACAGCAGGCGG |
| | TGTCCTGCAAACAACAGGAGGCACCTGAAGCTAGCCTGGGGGGCCCACGCCCAGG |
| | TGCGGTGCATTCAGCAGCACAGCCAGAGACAGACCCCAATGACCCCGCCTCCCTG |
| | TCGGCAGCCAGTGCTCTGCACAGAGCCCTGAGCAGCCTCTGGACATTAGTCCCAG |
| | CCCCAGCACGGCCCGTCCCCCACGCTGATGTCACCGCACCCAGACCTTGGAGGCC |
| | CCCTCCGGCTCCGCCTCCTGGGAGAAGGCTCTGGAGTGAGGAGGGGAGGGCAGCA |
| | GTGCTGGCTGGACAGCTGCTCTGGGCAGGAGAGAGAGGGAGAGACAAGAGACACA |
| | CACAGAGAGACGGCGAGGAAGGGAAAGACCCAGAGGGACGCCTAGAACGAGACTT |
| | GGAGCCAGACAGAGGAAGAGGGGACGTGTGTTTGCAGACTGGCTGGGCCCGTGAC |
| | CCAGCTTCCTGAGTCCTCCGTGCAGGTGGCAGCTGTACCAGGCTGGCAGGTCACT |
| | GAGAGTGGGCAGCTGGGCCCCAGGTAAGGATGGGCTGCCCACTGTCCTGGGCATT |
| | GGGAGGGGTTTGGATGTGGAGGAGTCATGGACTTGAGCTACCTCTAGAGCCTCTG |
| | CCCCACAGCCACTTGCTCCTGGGACTGGGCTTCCTGCCACCCTTGAGGGCTCAGC |
| | CACCACAGCCACTGAATGAAACTGTCCCGAGCCTGGGAAGATGGATGTGTGTCCC |
| | CTGGAGGAGGGAAGAGaCAAGGAGCATGTTGTCCATCGAATCTTCTCTGAGCTGG |
| | GGCTGGGGTTAGTGGCATCCTGGGGCCAGGGGAATAGACATGCTGTGGTGGCAGA |
| | GAGAAGAGTCCGTTCTCTCTGTCTCCTTTGCTTTCTCTCTGACACTCTTTATCTC |
| | CGTTTTTGGATAAGTCACTTCCTTCCTCTATGCCCCAAATATCCCATCTGTGAAA |
| | TGGGAGTATGAAGCCCCAACAGCCAGGGTTGTAGTGGGGAAGAGGTAAAATCAGG |
| | TATAGACATAGAAATACAAATACAGTCTATGCCCCCTGTTGTCAGTTGGAAAAGA |
| | AATTAACTTGAAGGTGGTCTAGTTCTCATTTTTAGAAATGAAATGTCTGTCTGGT |
| | CATTTTAAAATGTGGCCCTTAAATTTCACGCCCTCACCACTCTCCCCCATCCCTT |
| | GGAGCCCCATGTCTCTAGTGAAAGCACTGGCTCTGCCCCCAGCCCTCATGGCTCA |
| | TGCTGGCATAGGGCGCCTGCTCCACAGCCTGGGCACCATCTTCAGACAAGTGCCC |
| | GGTGGCAACTGCCTGCTGGCCCTGTTGAATCCACATCTCCACCAGGCATCCAGAC |
| | TAGTTCAGcTCTCTGGAAGGACTGTGGGTTTGCTGTGTCCCAGAGCTCCAGGGCA |
| | GGGGTCAGGGCTCGGATGTCGGGCAGTGTCATGGGCAGAGGATCGAATGCCCCGG |
| | CGGCTCTGAATGGGCCCTTGTGAAAAATTGATGCGCATTCTAGGAGACAGGTTGG |
| | GAGCCAGAGGGGCCTCATACCAGGGTCTGTAGGCTGGGGCTGCCTTTTAAGCTCC |
| | TTCCTGAGGCCGTCTCTGGGTCTGGCCCTGTGCTGGACAAGGCTGGAGACAAGGC |
| | AATGTCTCAGACCCTCTCCCATTGGCCACATCCTGCCCTGGATCAACTCGCCAAC |
| | TTTGGGGGCAGAGGTGGGACTGACCCTTACCCTGACAACATAATGCATATAGTCA |
| | AAATGGGATAAAGGGGAATATAGAGGCTCTTGGCAGCTTGGGAGTGGTCAGGGAA |
| | GGCTTCCTGGAGGAGGTATCATCTGAACTGAGCCATGAACCATAAGTGGAAATTC |
| | ACTAGTCAAAATTTCAGGTAGAAGGGCCAGTGTGTGAAGGCCAGGAGATGGCAAG |
| | AGCTGGCGTATTTCAGGAACAGTGAGTCACTGAGGATGTCCAAGTATAAGGGTAG |
| | GAAAGGGAGTGAGCAGTGAGAGAAAAGACCGAGGCATCAGCAGGGGCCAGATTGT |
| | GCTGGGCCTAGCGGGGGGGGCCCGGGCCCGGGCCCAGGCCCAGGTGCGGTGCATT |
| | CAGCAGCACAGCCAGAGACAGACCCCAATGACCCTGCCTCCCCGTCAGCAGCCAG |
| | TGCTCTGCACAGAGCCATCCTGAGGGCAGTGGGTGCTCTTGAGAGGTTTCAGGCA |
| | GGGTGTGCTGTGAGCAGGTCATGCCCAGCCCTTGACCTTCTGCTCAGTCAGGCTT |
| | GTCCTTGTCACCCACATTCCTGGGGCAGTCCCTAAGCTGAGTGCCGGAGATTAAG |
| | TCCTAGTCCTAAATTTGCTCTGGCTAGCTGTGTGACCCTGGGCAAGTCTTGGTCC |
| | CTCTCTGGGCCCCTTTGCCGTAGGTCCCTGGTGGGGCCAGACTTGCTACTTTCTA |
| | GGAGCCCTTTGGGAATCTCTGAATGACAGTGGCTGAGAGAAGAATTCAGCTGCTC |
| | TGGGCAGTGGTGCTGGTGACAGTGGCTGAGGCTCAGGTCACACAGGCTGGGCAGT |
| | GGTCAGAGGGAGAGAAGCCAAGGAGGGTTCCCTTGAGGGAGGAGGAGCTGGGGCT |
| | TTGGGAGGAGCCCAGGTGACCCCAGCCAGGCTCAAGGCTTCCAGGGCTGGCCTGC |
| | CCAGAAGCATGACATGcTCTCTCTCCCTGCAGAACTGTGCCTGGCCCAGTGGGCA |
| | GCAGGAGCTCCTGACTTGGGACC |
| 22 | GGGCGGAAAGGAGAAACTGTCATGTCCCGATAGGGCCGCGCGAGCTCTCCATCCT |
| | CGACAACGCTAATAACAAAGACGTGTGCTCCTCTTTGCTTGGTTCCCCCCACTCC |
| | TTTAAATCACAGATTTCACTTCAGTTTATCTGTGTCGCTGTCACACGTGGGGTGG |
| | CTCCCAGTCAGCTGGTTTGGCAAAGTTTCTGGATGATTACGGAATAACATGTGTC |
| | CCCAACCCGCAGAGCAGGTTGTGGGGGCAATGTTGCATTGACCAGCGTCAGAGAA |
| | CACACATCAGAGGCAAGGGTGGGTGTGCAGGAGGGAGAAGGCGCAGAAGGCAGGG |
| | CTTTAGCTCAGCACTCTCCCTCCTGCCATGCTCTGCCTGACCGTTCCCTCTCTGA |
| | GTCCCAAACAGCCAGGTAGAGGAGGAAGAAATGGGGCTGAGAgCCCAGCACATCA |
| | GTGATTAAGTCAGGATCAGGTGCGGTTTCCTGCTCAGGTGCTGAGACAGCAGGCG |
| | GTGTCCTGCAAACAACAGGAGGCACCTGAAGCTAGCCTGGGGGGCCCACGCCCAG |
| | GTGCGGTGCATTCAGCAGCACAGCCAGAGACAGACCCCAATGACCCCGCCTCCCT |
| | GTCGGCAGCCAGTGCTCTGCACAGAGCCCTGAGCAGCCTCTGGACATTAGTCCCA |
| | GCCCCAGCACGGCCCGTCCCCCACGCTGATGTCACCGCACCCAGACCTTGGAGGC |
| | CCCCTCCGGCTCCGCCTCCTGGGAGAAGGCTCTGGAGTGAGGAGGGGAGGGCAGC |
| | AGTGCTGGCTGGACAGCTGCTCTGGGCAGGAGAGAGAGGGAGAGACAAGAGACAC |
| | ACACAGAGAGACGGCGAGGAAGGGAAAGACCCAGAGGGACGCCTAGAACGAGACT |
| | TGGAGCCAGACAGAGGAAGAGGGGACGTGTGTTTGCAGACTGGCTGGGCCCGCCG |
| | AGCTGAGTCCTCCAGCCCCCCTTCCTACTGAGGGCCACAGTGGCCTCTGACCCTC |
| | AGCCCTTCCCCGTGATCAGTCTGAGGCTCTAATGAGCTCCTGGCCTCCAGAGCAG |
| | GAGCCAGGAATGTGGGCCCTGAGCCAGGTGGGCCTGGCAGCAGGCCGTAGGGCTT |
| | GTGGGGCTGTGCATTATGGGGGCTTGGCCAGCAGCTTCTCGcTCTCCAGGGTCAC |
| | CAGAGGCCAATGGCTCAGCCTTGCCTGGCCACCCTGAGGCTGCACGGACCAGCAT |
| | GTTTGCATATGAGTGCGTGCGCGTGTGTGTGTCTGTGTGTGTGAGTGAGAGGCAG |
| | G |
| 23 | CCCCAATGACCCCGCCTCCCTGTCGGCAGCCAGTGCTCTGCACAGAGCCCTGAGC |
| | AGCCTCTGGACATTAGTCCCAGCCCCAGCACGGCCCGTCCCCCACGCTGATGTCA |
| | CCGCACCCAGACCTTGGAGGCCCCCTCCGGCTCCGCCTCCTGGGAGAAGGCTCTG |
| | GAGTGAGGAGGGGAGGGCAGCAGTGCTGGCTGGACAGCTGCTCTGGGCAGGAGAG |
| | AGAGGGAGAGACAAGAGACACACACAGAGAGACGGCGAGGAAGGGAAAGACCCAG |
| | AGGGACGCCTAGAACGAGACTTGGAGCCAGACAGAGGAAGAGGGGACGTGTGTTT |
| | GCAGACTGGCTGGGCCCG |
| 24 | AGGGATCACTTCAGATCTGCCGAGTGAGTCGATTGGACTTAAAGGGCCAGTCAAA |
| | CCCTGACTGCCGGCTCATGGCAGGCTCTTGCCGAGGACAAATGCCCAGCCTATAT |
| | TTATGCAAAGAGATTTTGTTCCAAACTTAAGGTCAAAGATACCTAAAGACATCCC |
| | CCTCAGGAACCCCTCTCATGGAGGAGAGTGCCTGAGGGTCTTGGTTTCCCATTGC |
| | ATCCCCCACCTCAATTTCCCTGGTGCCCAGCCACTTGTGTCTTTAGGGTTCTCTT |
| | TCTCTCCATAAAAGGGAGCCAACACAGTGTCGGCCTCCTCTCCCCAACTAAGGGC |
| | TTATGTGTAATTAAAAGGGATTATGCTTTGAAGGGGAAAAGTAGCCTTTAATCAC |
| | CAGGAGAAGGACACAGCGTCCGGAGCCAGAGGCGCTCTTAACGGCGTTTATGTCC |
| | TTTGCTGTCTGAGGGGCCTCAGCTCTGACCAATCTGGTCTTCGTGTGGTCATTAG |
| | CGTAACAGTTACTGTCTGTGGTTTAAAAATGAGGTGTGGAGCAAATAAACAGGTT |
| | GGAAGTGTGGGGTGGGGTGGTGGGGTAGGGTGGTGGGGCAGGGTGGGGGGTTGTG |
| | AGCAGTCAGTGGGCTTGTCGCCGATTAGCACTGAAGCAGTGTTTAGCTGGACGGC |
| | CTTTCTGTGGGCCCCTCTGACAGTGCCCTTCCCAGGAAGATGTGTTTCTCTGTCC |
| | TCAGCCACATGAAAATCTTTTGCCTACCGTGCCTGTCAATCCATTGCCTGCCCGC |
| | CCCTCCCCCACCCCCCGTTTTACACCTGCCTGTCCAGTCTACCGCTCTCTAGGGC |
| | ATCCACGCTGAGCAGTGGGAAGAACTTTAAGCCCTGAtGAGCAGGCCAAAGGCAA |
| | GCAAGAACCCCCTCGAACAGCTTCCCAGCTTAGTGAGGCCTTA |
| 25 | CAAGATAAGCTATTCCTCCCTGTCTTTGCAGAGTTTAAATGACTCACTTGTTCAA |
| | GTACCCACCGTTGCCATGTGGGACCGTGAGCAAAGTACTTAATCTCACTAAGCTT |
| | CACGTTCCTCATCTGTAAAACAGCAAATATGGACCTCACAAAATTGTAGTGAGGC |
| | TAAAATGAAATAACATATGCAAAAGCAGTTTATAAATAATAAACTTACTATAAAA |
| | TATTATTTTGTAATTCTGCAAGCTTGTCTTAAATGCCATCACCTCCAAGGAGCCT |
| | TTTTGCCATCATAAGCAGAAACTATCTCTCTCTTCTTGGAAGCTCCACCATGCAC |
| | AGCCTATGGGCCCTCATCACACTCCTTGAGTTATTCGAGTTCAAGTCCCGTGTTT |
| | ACAACCAGACCGCAAACTCTATGAAGTCAGCATCCATTCCTCTCTGTGGTTCTCC |
| | CTCCGCCCCATCCAGGCTCAAGGGTCTAGAGTCTTTCAAAGAGAACACATTCTGA |
| | GATTTGAGGAGGCAGAGACAAAAAGTTCCACTGCGAAGTGCCAGGGAGGCTTCTG |
| | TTTGGGGTGTCCCTTGGGATCACAGATCCCCCACCTGGTGATGAGTCAACCCAGC |
| | ACCACCCCATTGCAGGGCTGGAATGACAGTAATGGGCCCACCTGCTGCCTCTCCT |
| | CATACCCGCACCCCAGTCAGACATTGCAAGTCAGTCACGGCTCTGTCCTGCTGGG |
| | CCTGGAGTGTTCCAGTGCCTTTTCCATCACAGCACCAAGCAGCCACTACTAGTCG |
| | ATCAATTTCAGCACAAGAGATAAACATCATTACCCTCTGCTAAGCTCAGAGATAA |
| | CCCAACTAGCTGACCATAATGACTTCAGTCATTACGGAGCAAGATAAAAGACTAA |
| | AAGAGGGAGGGATCACTTCAGATCTGCCGAGTGAGTCGATTGGACTTAAAGGGCC |
| | AGTCAAACCCTGACTGCCGGCTCATGGCAGGCTCTTGCCGAGGACAAATGCCCAG |
| | CCTATATTTATGCAAAGAGATTTTGTTCCAAACTTAAGGTCAAAGATACCTAAAG |
| | ACATCCCCCTCAGGAACCCCTCTCATGGAGGAGAGTGCCTGAGGGTCTTGGTTTC |
| | CCATTGCATCCCCCACCTCAATTTCCCTGGTGCCCAGCCACTTGTGTCTTTAGGG |
| | TTCTCTTTCTCTCCATAAAAGGGAGCCAACACAGTGTCGGCCTCCTCTCCCCAAC |
| | TAAGGGCTTATGTGTAATTAAAAGGGATTATGCTTTGAAGGGGAAAAGTAGCCTT |
| | TAATCACCAGGAGAAGGACACAGCGTCCGGAGCCAGAGGCGCTCTTAACGGCGTT |
| | TATGTCCTTTGCTGTCTGAGGGGCCTCAGCTCTGACCAATCTGGTCTTCGTGTGG |
| | TCATTAGCGTAACAGTTACTGTCTGTGGTTTAAAAATGAGGTGTGGAGCAAATAA |
| | ACAGGTTGGAAGTGTGGGGTGGGGTGGTGGGGTAGGGTGGTGGGGCAGGGTGGGG |
| | GGTTGTGAGCAGTCAGTGGGCTTGTCGCCGATTAGCACTGAAGCAGTGTTTAGCT |
| | GGACGGCCTTTCTGTGGGCCCCTCTGACAGTGCCCTTCCCAGGAAGATGTGTTTC |
| | TCTGTCCTCAGCCACATGAAAATCTTTTGCCTACCGTGCCTGTCAATCCATTGCC |
| | TGCCCGCCCCTCCCCCACCCCCCGTTTTACACCTGCCTGTCCAGTCTACCGCTCT |
| | CTAGGGCATCCACGCTGAGCAGTGGGAAGAACTTTAAGCCCTGAtGAGCAGGCCA |
| | AAGGCAAGCAAGAACCCCCTCGAACAGCTTCCCAGCTTAGTGAGGCCTTA |
| 26 | CAAGATAAGCTATTCCTCCCTGTCTTTGCAGAGTTTAAATGACTCACTTGTTCAA |
| | GTACCCACCGTTGCCATGTGGGACCGTGAGCAAAGTACTTAATCTCACTAAGCTT |
| | CACGTTCCTCATCTGTAAAACAGCAAATATGGACCTCACAAAATTGTAGTGAGGC |
| | TAAAATGAAATAACATATGCAAAAGCAGTTTATAAATAATAAACTTACTATAAAA |
| | TATTATTTTGTAATTCTGCAAGCTTGTCTTAAATGCCATCACCTCCAAGGAGCCT |
| | TTTTGCCATCATAAGCAGAAACTATCTCTCTCTTCTTGGAAGCTCCACCATGCAC |
| | AGCCTATGGGCCCTCATCACACTCCTTGAGTTATTCGAGTTCAAGTCCCGTGTTT |
| | ACAACCAGACCGCAAACTCTATGAAGTCAGCATCCATTCCTCTCTGTGGTTCTCC |
| | CTCCGCCCCATCCAGGCTCAAGGGTCTAGAGTCTTTCAAAGAGAACACATTCTGA |
| | GATTTGAGGAGGCAGAGACAAAAAGTTCCACTGCGAAGTGCCAGGGAGGCTTCTG |
| | TTTGGGGTGTCCCTTGGGATCACAGATCCCCCACCTGGTGATGAGTCAACCCAGC |
| | ACCACCCCATTGCAGGGCTGGAATGACAGTAATGGGCCCACCTGCTGCCTCTCCT |
| | CATACCCGCACCCCAGTCAGACATTGCAAGTCAGTCACGGCTCTGTCCTGCTGGG |
| | CCTGGAGTGTTCCAGTGCCTTTTCCATCACAGCACCAAGCAGCCACTACTAGTCG |
| | ATCAATTTCAGCACAAGAGATAAACATCATTACCCTCTGCTAAGCTCAGAGATAA |
| | CCCAACTAGCTGACCATAATGACTTCAGTCATTACGGAGCAAGATAAAAGACTAA |
| | AAGAGGGAGGGATCACTTCAGATCTGCCGAGTGAGTCGATTGGACTTAAAGGGCC |
| | AGTCAAACCCTGACTGCCGGCTCATGGCAGGCTCTTGCCGAGGACAAATGCCCAG |
| | CCTATATTTATGCAAAGAGATTTTGTTCCAAACTTAAGGTCAAAGATACCTAAAG |
| | ACATCCCCCTCAGGAACCCCTCTCATGGAGGAGAGTGCCTGAGGGTCTTGGTTTC |
| | CCATTGCATCCCCCACCTCAATTTCCCTGGTGCCCAGCCACTTGTGTCTTTAGGG |
| | TTCTCTTTCTCTCCATAAAAGGGAGCCAACACAGTGTCGGCCTCCTCTCCCCAAC |
| | TAAGGGCTTATGTGTAATTAAAAGGGATTATGCTTTGAAGGGGAAAAGTAGCCTT |
| | TAATCACCAGGAGAAGGACACAGCGTCCGGAGCCAGAGGCGCTCTTAACGGCGTT |
| | TATGTCCTTTGCTGTCTGAGGGGCCTCAGCTCTGACCAATCTGGTCTTCGTGTGG |
| | TCATTAG |
| 27 | AGGGATCACTTCAGATCTGCCGAGTGAGTCGATTGGACTTAAAGGGCCAGTCAAA |
| | CCCTGACTGCCGGCTCATGGCAGGCTCTTGCCGAGGACAAATGCCCAGCCTATAT |
| | TTATGCAAAGAGATTTTGTTCCAAACTTAAGGTCAAAGATACCTAAAGACATCCC |
| | CCTCAGGAACCCCTCTCATGGAGGAGAGTGCCTGAGGGTCTTGGTTTCCCATTGC |
| | ATCCCCCACCTCAATTTCCCTGGTGCCCAGCCACTTGTGTCTTTAGGGTTCTCTT |
| | TCTCTCCATAAAAGGGAGCCAACACAGTGTCGGCCTCCTCTCCCCAACTAAGGGC |
| | TTATGTGTAATTAAAAGGGATTATGCTTTGAAGGGGAAAAGTAGCCTTTAATCAC |
| | CAGGAGAAGGACACAGCGTCCGGAGCCAGAGGCGCTCTTAACGGCGTTTATGTCC |
| | TTTGCTGTCTGAGGGGCCTCAGCTCTGACCAATCTGGTCTTCGTGTGGTCATTAG |
| 9 | GAATTCAGCAAGGTCGCCACGCACAAGATCAATATTAACAATCAGTCATCTCTCT |
| | TTAGCAATAAAAAGGTGAAAAATTACATTTTAAAAATGACACCATAGACGATGTA |
| | TGAAAATAATCTACTTGGAAATAAATCTAGGCAAAGAAGTGCAAGACTGTTACCC |
| | AGAAAACTTACAAATTGTAAATGAGAGGTTAGTGAAGATTTAAATGAATGAAGAT |
| | CTAAATAAACTTATAAATTGTGAGAGAAATTAATGAATGTCTAAGTTAATGCAGA |
| | AACGGAGAGACATACTATATTCATGAACTAAAAGACTTAATATTGTGAAGGTATA |
| | CTTTCTTTTCACATAAATTTGTAGTCAATATGTTCACCCCAAAAAAGCTGTTTGT |
| | TAACTTGTCAACCTCATTTCAAAATGTATATAGAAAGCCCAAAGACAATAACAAA |
| | AATATTCTTGTAGAACAAAATGGGAAAGAATGTTCCACTAAATATCAAGATTTAG |
| | AGCAAAGCATGAGATGTGTGGGGATAGACAGTGAGGCTGATAAAATAGAGTAGAG |
| | CTCAGAAACAGACCCATTGATATATGTAAGTGACCTATGAAAAAAATATGGCATT |
| | TTACAATGGGAAAATGATGATCTTTTTCTTTTTTAGAAAAACAGGGAAATATATT |
| | TATATGTAAAAAATAAAAGGGAACCCATATGTCATACCATACACACAAAAAAATT |
| | CCAGTGAATTATAAGTCTAAATGGAGAAGGCAAAACTTTAAATCTTTTAGAAAAT |
| | AATATAGAAGCATGCCATCATGACTTCAGTGTAGAGAAAAATTTCTTATGACTCA |
| | AAGTCCTAACCACAAAGAAAAGATTGTTAATTAGATTGCATGAATATTAAGACTT |
| | ATTTTTAAAATTAAAAAACCATTAAGAAAAGTCAGGCCATAGAATGACAGAAAAT |
| | ATTTGCAACACCCCAGTAAAGAGAATTGTAATATGCAGATTATAAAAAGAAGTCT |
| | TACAAATCAGTAAAAAATAAAACTAGACAAAAATTTGAACAGATGAAAGAGAAAC |
| | TCTAAATAATCATTACACATGAGAAACTCAATCTCAGAAATCAGAGAACTATCAT |
| | TGCATATACACTAAATTAGAGAAATATTAAAAGGCTAAGTAACATCTGTGGCAAT |
| | ATTGATGGTATATAACCTTGATATGATGTGATGAGAACAGTACTTTACCCCATGG |
| | GCTTCCTCCCCAAACCCTTACCCCAGTATAAATCATGACAAATATACTTTAAAAA |
| | CCATTACCCTATATCTAACCAGTACTCCTCAAAACTGTCAAGGTCATCAAAAATA |
| | AGAAAAGTCTGAGGAACTGTCAAAACTAAGAGGAACCCAAGGAGACATGAGAATT |
| | ATATGTAATGTGGCATTCTGAATGAGATCCCAGAACAGAAAAAGAACAGTAGCTA |
| | AAAAACTAATGAAATATAAATAAAGTTTGAACTTTAGTTTTTTTTAAAAAAGAGT |
| | AGCATTAACACGGCAAAGTCATTTTCATATTTTTCTTGAACATTAAGTACAAGTC |
| | TATAATTAAAAATTTTTTAAATGTAGTCTGGAACATTGCCAGAAACAGAAGTACA |
| | GCAGCTATCTGTGCTGTCGCCTAACTATCCATAGCTGATTGGTCTAAAATGAGAT |
| | ACATCAACGCTCCTCCATGTTTTTTGTTTTCTTTTTAAATGAAAAACTTTATTTT |
| | TTAAGAGGAGTTTCAGGTTCATAGCAAAATTGAGAGGAAGGTACATTCAAGCTGA |
| | GGAAGTTTTCCTCTATTCCTAGTTTACTGAGAGATTGCATCATGATTGGGTGTTA |
| | AATTTTGTCAAATGCTTTTTCTGTGTCTATCAATATGACCATGTGATTTTCTTCT |
| | TTAACCTGTTGATGGGACAAATTACGTTAATTGATTTTCAAACGTTGAACCACCC |
| | TTACATATCTGGAATAAATTCTACTTGGTTGTGGTGTATATTTTTTGATACATTC |
| | TTGGATTCTTTTTGCTAATATTTTGTTGAAAATGTTTGTATCTTTGTTCATGAGA |
| | GATATTGGTCTGTTGTTTTCTTTTCTTGTAATGTCATTTTCTAGTTCCGGTATTA |
| | AGGTAATGCTGGCCTAGTGGAATGATTTAGGAAGTATTCCCTCTGCTTCTGTCTT |
| | CTGAAAGAGATTGTAGAAAGTTGATACAATTTTTTTTTCTTTAAATATCTTGATA |
| | GAATTC |
| 10 | GCAGGCATGCTTCTATATTATTTTCTAAAAGATTTAAAGTTTTGCCTTCTCCATT |
| | TAGACTTATAATTCACTGGAATTTTTTTGTGTGTATGGTATGACATATGGGTTCC |
| | CTTTTATTTTTTACATATAAATATATTTCCCTGTTTTTCTAAAAAAGAGGGGATC |
| | CTCTAGAGTCGACCTGCAGGCATGCTTCTATATTATTTTCTAAAAGATTTAAAGT |
| | TTTGCCTTCTCCATTTAGACTTATAATTCACTGGAATTTTTTTGTGTGTATGGTA |
| | TGACATATGGGTTCCCTTTTATTTTTTACATATAAATATATTTCCCTGTTTTTCT |
| | AAAAAAGAGGGGATCCTCTAGAGTCGACCTGCAGGCATGCTTCTATATTATTTTC |
| | TAAAAGATTTAAAGTTTTGCCTTCTCCATTTAGACTTATAATTCACTGGAATTTT |
| | TTTGTGTGTATGGTATGACATATGGGTTCCCTTTTATTTTTTACATATAAATATA |
| | TTTCCCTGTTTTTCTAAAAAAGAGGGGATCCTCTAGAGTCGACCT |
| 11 | ACACAGCCTGAAGCCCAGCGGCTGTGCTATGGGCTGGGGACGGCTCTGTGCTCAG |
| | CATCCTTCAATCCACGGCGGCTCCGCTGCACCTCCTCTGCAAAACGCCTCCAGGA |
| | AACCACTCGGCCGAGTGCCCTCTCCCGGAGCCTTACTTAGAGCCTCAGGAAAAGG |
| | AAGAAAGTGCCGAAAGGTTGAAGAAAAGAAGCAGGCTTTCCTGGAAGGTCCTGGA |
| | AGGGGGCGTCCGCGGGAGCTCACGGGGACAGCCCCCCCCCAAAGCCCCCAGGGCT |
| | GTAATTACGTCCCTCCCCCGCTAGGGGGCAGCAGCGAGCCGCCCGGGGCTCCGCT |
| | CCGGTCCGGCGCTCCCCCCGCATCCCCGAGCCGGCAGCGTGCGGGGACAGCCCGG |
| | GCACGGGGAAGGTGGCACGGGATCGCTTTCCTCTGAACGCTTCTCGCTGCTCTTT |
| | GAGCCTGCAGACACCTGGGGGATACGGGGAAAAAGCTTTAGGCTGAAAGAGAGAT |
| | TTAGAATGACAGAATCATAGAACGGCCTGGGTTGCAAGGAGCACAGTGCTCATCC |
| | AGATCCAACCCCCTGCTATGTGCAGGGTCATCAACCAGCAGCCCAGCGCGTCAGA |
| | GCCACATCCAGCCTGGCCTTGAATGCCTGCCTGCAGGGATGGGGCATCCACAGCC |
| | TCCTTGGGCAACCTGTTCAGTGCGTCACCACCCTCTGGGGAAAAACTGCCTCCTC |
| | ATATCCAACCCAAACCTCCCCTGTCTCAGTGTAAAGCCATTCCCCCTTGTCCTAT |
| | CAAGGGGGAGTTTGCTGTGACATTGTTGGTCTGGGGTGACACATGTTTGCCAATT |
| | CAGTGCTCACGGAGAGGCAGATCTTGGGATAAGGAAGTGCAGGACAGCATGGACG |
| | TGGACATGCAGGTGTTGAGGCTCTGGACACTCCAAGTCACAGCGTTCAGAACAGC |
| | CTTAAGGTCAAGAAGATAGGATAGAAGGACAAAGAGCAAGTTAAAACCCAGCATG |
| | GAGAGGAGCACAAAAAGGCCACAGACACTGCTGGTCCCTGTGTCTGAGCCTGCAT |
| | GTTTGATGGTGTCTGGATGCAAGCAGAAGGGGTGGAtGAGCTTGCCTGGAGAGAT |
| | ACAGGCTGGGTCGTAGGACTGGGACAGGCAGCTGGAGAATTGCCATGTAGATGTT |
| | CATACAATCGTCAAATCATGAAGGCTGGAAAAGCCCTCCAAGATCCCCAAGACCA |
| | ACCCCAACCCACCCACCGTGCCACTGGCCATGTCCCTCAGTGCCACATCCCCACA |
| | GTTCTTCATCACCTCCAGGGACGGTGACCCCCCACCTCCGTGGGCAGCTGTGCCA |
| | CTGCAGCACCGCTCTTTGGAGAAGGTAAATCTTGCTAAATCCAGCCCGACCCTCC |
| | CCTGGGCACAACGTTAAGGCCATTATCTCTCATCCTACTCCAGGACGGAGTCAGT |
| | TGAGGATGGGGGAATATTGCTGTGGGTGGCAGATGGCCACATCCCATGTCCCTCA |
| | GTGCCACATCCCCACAGTTCTTCATCACCTCCAGGGACGGTGACCCCCCACCTCC |
| | GTGGGCAGCTGTGCCACTGCAGCACCGCTCTTTGGAGAAGGTAAATCTTGCTAAA |
| | TCCAGCCCGACCCTCCCCTGGGCACAACGTTAAGGCCATTATCTCTCATCCTACT |
| | CCAGGACGGAGTCAGTTGAGGATGGGGGAATATTGCTGTGGGTGGCAGATGGCCA |
| | CATCCTGGTCAAAGGCTATAGGCAGAACGAAGACACATTCAATGTCTGTGAGGCT |
| | GTGAGTGAAAAGCATTTGGAAGAGGCAGGTGCTAAGCTGATCTCTCTGGCTCCGG |
| | CCACAGCATCCTCAGTGTGGTTGGGATTGCAGATGTGGACAAGGCCAGGACTGCC |
| | ACAGCTGGCATAGCGAGGAGGAGGTACATGGCACGTGCAGGCTCTGCTCTGCCTT |
| | AATGACAACAGGGAACATCCAGTTCCCCAGCAGCACCAGGAGGTACATGGAGGGG |
| | GCAGCTGAGTGGTTTTCCATGATGGAAAAGCCACCAAGAAGAATATCAGCATCCT |
| | TGGTCTGGCTGCTGTTGGTCCAAGGTG |
| 8 | AGACCCTCCGATCCTCTGAAAGGAGCATCAGGCAGGCAGGGGTTGCTCCGGGGCC |
| | AGGGAAGGGGCCCCGCTGGGACGCGGCTGTTATTGCAGCTGGTTGGCGCGCAGCC |
| | ATGCTTAGCTGCAGTGCGGGAATGCTGGGCCTTCTGTTCTGGGCTGTTTCTCATA |
| | CGCACGTAGGCCAGTGTATAAATAAGGTTTTATTAAATGCCAAATGAGTTCTCAT |
| | TAACAAAGAAAGAGGGAAAATCTCAGTAAACCACCGTGACGGCATCTACCCACTT |
| | TGAGTCAGGAGCTGGGGGTGTGAGTGCAACCTCCGAGACAAGGGAACCTGTGGAG |
| | CCCAGAGAATCGGAGGGGGGCGCTGGGGTTAGCACCGACTGAACCAGCTGTGTTT |
| | TCTCTCGGTTCCTTGGAGATCAGAAGTGAGTGTTGTCATCTTCAAACAATCCAAA |
| | GGCAGTACCCATGGCCTTACTACATCCCTCCCACACCATCCCACCCATCCCCGCG |
| | CGTACACTCACACGCTCATTTGCACACTATCGCACACGCTCACTTGCGTGCGCAC |
| | ACACAGATTGGTGACCTAGGTGGACTGGGAGAGAAATAAGAGCCAAATGACTGGA |
| | TTTTCTCCAAGGAAATTTATTAATAGCCCCTCTTGGTTTCACCTGAAGGAGCTTG |
| | TCTTCACCTGCGGCCTTTGCAGGCTTAACGCCCCCAGCTTGAAACCCAGAAGCTC |
| | AGACTTGGGCCCAAGGTATTATTAGTGCCAACACTACCTGAAATGTTTCGCACCT |
| | CATAAAAATGGTGTGTCAGTTTCGGGTGAGAGGTTGGGACGCTTCCCATCTGATT |
| | TGGCCCAAGGCATGCATGCCCCTCCTTCTCCTTCCCCTCCTCCTCCCCCTCTTCC |
| | CCCTACCATCCTTCCTGTTTTCTCTCCAACTCTGGTGCACAGCTTTGAAATCTTG |
| | CTGAGAAGCAAATCTGTCCCTTCTGCTTTGAATGTTTATTTGTGGAAGTTCGGCA |
| | GGGGAACCGAGGCGGGTGCCAAGACCTGCCATGCTGCTGGGAAGTCTGAGTCTCC |
| | CTCCCTTCCCCCTCCTAAATGCTTGTTGATAGAGAAAAGTCAGCCTCCTCGGCAT |
| | TTGGGCTCACGGTTTTCCTTTGAAAATGCTTCCAGTGTGGCATGATTCAGCTTTC |
| | TTTTCTGTCCCCCAACCACTGCTCTGTTGTCATTTTTACTTTTCTGATTGCATTT |
| | TATCCGTGTCTCTTTGACTACGGGGTGGCTGGACGTTGAGTTCCAGGAAGAAAAG |
| | GGCCCAATCTTGGGGTTCTGACTACATGCGCCCATCAATGTCCTGTTTCATTCTT |
| | GGCTCTGGCTCCCTGAATTCCTGAGTCACTGGGGAGAAGCGTGGGTGGACCGCCC |
| | CCTACCCAGTGAGAGTTGCCACAGTTGCTGCTCTCCTGGGTCATTGGTTGCAGAT |
| | TGTTAAACTTCACCTATGCATTTCAACTTTCGGGTGGATATTGCTACGTCAAGTG |
| | TCTGGGAAAGCCCCCACAGCTACAGGATTTTACAGTGAGGTCCCACTAATGACTT |
| | GATGTCATGACTTCCTCATTCTTTCCAATTTCTCCCACTTCTCCATAAGGGTTTT |
| | GGGAAGGGGAGAAGAGAAAGGAGTGATTCCTGAGTGCCAGTACCAGGGAACAGCA |
| | GGGCTGTTGGGAGG |
| 28 | ACATTGATTATTGACTAGTTATTAATAGTAATCAATTACGGGGTCATTAGTTCAT |
| | AGCCCATATATGGAGTTCCGCGTTACATAACTTACGGTAAATGGCCCGCCTGGCT |
| | GACCGCCCAACGACCCCCGCCCATTGACGTCAATAATGACGTATGTTCCCATAGT |
| | AACGCCAATAGGGACTTTCCATTGACGTCAATGGGTGGAGTATTTACGGTAAACT |
| | GCCCACTTGGCAGTACATCAAGTGTATCATATGCCAAGTACGCCCCCTATTGACG |
| | TCAATGACGGTAAATGGCCCGCCTGGCATTATGCCCAGTACATGACCTTATGGGA |
| | CTTTCCTACTTGGCAGTACATCTACGTATTAGTCATCGCTATTACCATGTCGAGG |
| | TGAGCCCCACGTTCTGCTTCACTCTCCCCATCTCCCCCCCCTCCCCACCCCCAAT |
| | TTTGTATTTATTTATTTTTTAATTATTTTGTGCAGCGATGGGGGCGGGGGGGGGG |
| | GGGGGGCGCGCGCCAGGCGGGGCGGGGCGGGGCGAGGGGCGGGGCGGGGCGAGGC |
| | GGAGAGGTGCGGCGGCAGCCAATCAGAGCGGCGCGCTCCGAAAGTTTCCTTTTAT |
| | GGCGAGGCGGCGGCGGCGGCGGCCCTATAAAAAGCGAAGCGCGCGGCGGGCGGGA |
| | GTCGCTGCGACGCTGCCTTCGCCCCGTGCCCCGCTCCGCCGCCGCCTCGCGCCGC |
| | CCGCCCCGGCTCTGACTGACCGCGTTACTCCCACAGGTGAGCGGGCGGGACGGCC |
| | CTTCTCCTCCGGGCTGTAATTAGCGCTTGGTTTAATGACGGCTTGTTTCTTTTCT |
| | GTGGCTGCGTGAAAGCCTTGAGGGGCTCCGGGAGGGCCCTTTGTGCGGGGGGAGC |
| | GGCTCGGGGGGTGCGTGCGTGTGTGTGTGCGTGGGGAGCGCCGCGTGCGGCTCCG |
| | CGCTGCCCGGCGGCTGTGAGCGCTGCGGGCGCGGCGCGGGGCTTTGTGCGCTCCG |
| | CAGTGTGCGCGAGGGGAGCGCGGCCGGGGGCGGTGCCCCGCGGTGCGGGGGGGGC |
| | TGCGAGGGGAACAAAGGCTGCGTGCGGGGTGTGTGCGTGGGGGGGTGAGCAGGGG |
| | GTGTGGGCGCGTCGGTCGGGCTGCAACCCCCCCTGCACCCCCCTCCCCGAGTTGC |
| | TGAGCACGGCCCGGCTTCGGGTGCGGGGCTCCGTACGGGGCGTGGCGCGGGGCTC |
| | GCCGTGCCGGGCGGGGGGTGGCGGCAGGTGGGGGTGCCGGGGGGGGCGGGGCCGC |
| | CTCGGGCCGGGGAGGGCTCGGGGGAGGGGCGCGGCGGCCCCCGGAGCGCCGGCGG |
| | CTGTCGAGGCGCGGCGAGCCGCAGCCATTGCCTTTTATGGTAATCGTGCGAGAGG |
| | GCGCAGGGACTTCCTTTGTCCCAAATCTGTGCGGAGCCGAAATCTGGGAGGCGCC |
| | GCCGCACCCCCTCTAGCGGGCGCGGGGCGAAGCGGTGCGGCGCCGGCAGGAAGGA |
| | AATGGGGGGGGAGGGCCTTCGTGCGTCGCCGCGCCGCCGTCCCCTTCTCCCTCTC |
| | CAGCCTCGGGGCTGTCCGCGGGGGGACGGCTGCCTTCGGGGGGGACGGGGCAGGG |
| | CGGGGTTCGGCTTCTGGCGTGTGACCGGCGGCTCTAGACAATTGTACTAACCTTC |
| | TTCTCTTTCCTCTCCTGACAGGGAGTTTAAA |
| 29 | GGCTCCGGTGCCCGTCAGTGGGCAGAGCGCACATCGCCCACAGTCCCCGAGAAGT |
| | TGGGGGGAGGGGTCGGCAATTGAACCGGTGCCTAGAGAAGGTGGCGCGGGGTAAA |
| | CTGGGAAAGTGATGTCGTGTACTGGCTCCGCCTTTTTCCCGAGGGTGGGGGAGAA |
| | CCGTATATAAGTGCAGTAGTCGCCGTGAACGTTCTTTTTCGCAACGGGTTTGCCG |
| | CCAGAACACAGGTAAGTGCCGTGTGTGGTTCCCGCGGGCCTGGCCTCTTTACGGG |
| | TTATGGCCCTTGCGTGCCTTGAATTACTTCCACCTGGCTGCAGTACGTGATTCTT |
| | GATCCCGAGCTTCGGGTTGGAAGTGGGTGGGAGAGTTCGAGGCCTTGCGCTTAAG |
| | GAGCCCCTTCGCCTCGTGCTTGAGTTGAGGCCTGGCCTGGGCGCTGGGGCCGCCG |
| | CGTGCGAATCTGGTGGCACCTTCGCGCCTGTCTCGCTGCTTTCGATAAGTCTCTA |
| | GCCATTTAAAATTTTTGATGACCTGCTGCGACGCTTTTTTTCTGGCAAGATAGTC |
| | TTGTAAATGCGGGCCAAGATCTGCACACTGGTATTTCGGTTTTTGGGGCCGCGGG |
| | CGGCGACGGGGCCCGTGCGTCCCAGCGCACATGTTCGGCGAGGCGGGGCCTGCGA |
| | GCGCGGCCACCGAGAATCGGACGGGGGTAGTCTCAAGCTGGCCGGCCTGCTCTGG |
| | TGCCTGGCCTCGCGCCGCCGTGTATCGCCCCGCCCTGGGCGGCAAGGCTGGCCCG |
| | GTCGGCACCAGTTGCGTGAGCGGAAAGATGGCCGCTTCCCGGCCCTGCTGCAGGG |
| | AGCTCAAAATGGAGGACGCGGCGCTCGGGAGAGCGGGCGGGTGAGTCACCCACAC |
| | AAAGGAAAAGGGCCTTTCCGTCCTCAGCCGTCGCTTCATGTGACTCCACGGAGTA |
| | CCGGGCGCCGTCCAGGCACCTCGATTAGTTCTCGAGCTTTTGGAGTACGTCGTCT |
| | TTAGGTTGGGGGGAGGGGTTTTATGCGATGGAGTTTCCCCACACTGAGTGGGTGG |
| | AGACTGAAGTTAGGCCAGCTTGGCACTTGATGTAATTCTCCTTGGAATTTGCCCT |
| | TTTTGAGTTTGGATCTTGGTTCATTCTCAAGCCTCAGACAGTGGTTCAAAGTTTT |
| | TTTCTTCCATTTCAGGTGTCGTGA |
| 30 | CCTAGCCTTTAAGGGGCTCTA |
| 31 | TGACCTCTGTGAAAAGATCAGGG |
| 32 | TGGGAGTTAGACCCGGCGAGTG |
| 33 | TGTAGCATCTCCGCTTGTCACT |
| 34 | CAGTCTTCTGGGTGGCAGTG |
| 35 | AACCATGAGAAGTATGACAACAGC |
| 36 | TTCGgtatcacaggagaatttcagGGAGCAAGATAAGCTATTCCTCCCTGTCTTT |
| | GCAGAGTTTAAATGACTCACTTGTTCAAGTACCCACCGTTGCCATGTGGGACCGT |
| | GAGCAAAGTACTTAATCTCACTAAGCTTCACGTTCCTCATCTGTAAAACAGCAAA |
| | TATGGACCTCACAAAATTGTAGTGAGGCTAAAATGAAATAACATATGCAAAAGCA |
| | GTTTATAAATAATAAACTTACTATAAAATATTATTTTGTAATTCTGCAAGCTTGT |
| | CTTAAATGCCATCACCTCCAAGGAGCCTTTTTGCCATCATAAGCAGAAACTATCT |
| | CTCTCTTCTTGGAAGCTCCACCATGCACAGCCTATGGGCCCTCATCACACTCCTT |
| | GAGTTATTCGAGTTCAAGTCCCGTGTTTACAACCAGACCGCAAACTCTATGAAGT |
| | CAGCATCCATTCCTCTCTGTGGTTCTCCCTCCGCCCCATCCAGGCTCAAGGGTCT |
| | AGAGTCTTTCAAAGAGAACACATTCTGAGATTTGAGGAGGCAGAGACAAAAAGTT |
| | CCACTGCGAAGTGCCAGGGAGGCTTCTGTTTGGGGTGTCCCTTGGGATCACAGAT |
| | CCCCCACCTGGTGATGAGTCAACCCAGCACCACCCCATTGCAGGGCTGGAATGAC |
| | AGTAATGGGCCCACCTGCTGCCTCTCCTCATACCCGCACCCCAGTCAGACATTGC |
| | AAGTCAGTCACGGCTCTGTCCTGCTGGGCCTGGAGTGTTCCAGTGCCTTTTCCAT |
| | CACAGCACCAAGCAGCCACTACTAGTCGATCAATTTCAGCACAAGAGATAAACAT |
| | CATTACCCTCTGCTAAGCTCAGAGATAACCCAACTAGCTGACCATAATGACTTCA |
| | GTCATTACGGAGCAAGATAAAAGACTAAAAGAGGGAGGGATCACTTCAGATCTGC |
| | CGAGTGAGTCGATTGGACTTAAAGGGCCAGTCAAACCCTGACTGCCGGCTCATGG |
| | CAGGCTCTTGCCGAGGACAAATGCCCAGCCTATATTTATGCAAAGAGATTTTGTT |
| | CCAAACTTAAGGTCAAAGATACCTAAAGACATCCCCCTCAGGAACCCCTCTCATG |
| | GAGGAGAGTGCCTGAGGGTCTTGGTTTCCCATTGCATCCCCCACCTCAATTTCCC |
| | TGGTGCCCAGCCACTTGTGTCTTTAGGGTTCTCTTTCTCTCCATAAAAGGGAGCC |
| | AACACAGTGTCGGCCTCCTCTCCCCAACTAAGGGCTTATGTGTAATTAAAAGGGA |
| | TTATGCTTTGAAGGGGAAAAGTAGCCTTTAATCACCAGGAGAAGGACACAGCGTC |
| | CGGAGCCAGAGGCGCTCTTAACGGCGTTTATGTCCTTTGCTGTCTGAGGGGCCTC |
| | AGCTCTGACCAATCTGGTCTTCGTGTGGTCATTAGCGTAACAGTTACTGTCTGTG |
| | GTTTAAAAATGAGGTGTGGAGCAAATAAACAGGTTGGAAGTGTGGGGTGGGGTGG |
| | TGGGGTAGGGTGGTGGGGCAGGGTGGGGGGTTGTGAGCAGTCAGTGGGCTTGTCG |
| | CCGATTAGCACTGAAGCAGTGTTTAGCTGGACGGCCTTTCTGTGGGCCCCTCTGA |
| | CAGTGCCCTTCCCAGGAAGATGTGTTTCTCTGTCCTCAGCCACATGAAAATCTTT |
| | TGCCTACCGTGCCTGTCAATCCATTGCCTGCCCGCCCCTCCCCCACCCCCCGTTT |
| | TACACCTGCCTGTCCAGTCTACCGCTCTCTAGGGCATCCACGCTGAGCAGTGGGA |
| | AGAACTTTAAGCCCTGATGAGCAGGCCAAAGGCAAGCAAGAACCCCCTCGAACAG |
| | CTTCCCAGCTTAGTGAGGCCTTACAGATAAGTTTGTACAAAAAAGAGAGGTGCCA |
| | CCATGGGCTTTGTGCGACAGATTCAGCTGCTGCTGTGGAAGAACTGGACCCTGCG |
| | GAAGCGGCAGAAAATCAGATTCGTGGTGGAACTCGTGTGGCCCCTGAGCCTGTTT |
| | CTGGTGCTGATCTGGCTGCGGAACGCCAATCCTCTGTACAGCCACCACGAGTGTC |
| | ACTTCCCCAACAAGGCCATGCCTTCTGCCGGAATGCTGCCTTGGCTGCAGGGCAT |
| | CTTCTGCAACGTGAACAACCCCTGCTTTCAGAGCCCCACACCTGGCGAAAGCCCT |
| | GGCATCGTGTCCAACTACAACAACAGCATCCTGGCCAGAGTGTACCGGGACTTCC |
| | AAGAGCTGCTGATGAACGCCCCTGAGTCTCAGCACCTGGGCAGAATCTGGACCGA |
| | GCTGCACATCCTGAGCCAGTTCATGGACACCCTGAGAACACACCCCGAGAGAATC |
| | GCCGGCAGGGGCATCAGAATCCGGGACATCCTGAAGGACGAGGAAACCCTGACAC |
| | TGTTCCTCATCAAGAACATCGGCCTGAGCGACAGCGTGGTGTACCTGCTGATCAA |
| | CAGCCAAGTGCGGCCCGAGCAGTTTGCTCATGGCGTGCCgGATCTCGCCCTGAAG |
| | GATATCGCCTGTTCTGAGGCCCTGCTGGAACGGTTCATCATCTTCAGCCAGCGGA |
| | GAGGCGCCAAGACCGTCAGATATGCCCTGTGCAGTCTGAGCCAGGGAACCCTGCA |
| | GTGGATCGAGGATACCCTGTACGCCAACGTGGACTTCTTCAAGCTGTTCCGGGTG |
| | CTGCCCACACTGCTGGATTCTAGATCCCAGGGCATCAACCTGAGAAGCTGGGGCG |
| | GCATCCTGTCCGACATGAGCCCAAGAATCCAAGAGTTCATCCACCGGCCTAGCAT |
| | GCAGGACCTGCTGTGGGTTACCAGACCTCTGATGCAGAACGGCGGACCCGAGACA |
| | TTCACCAAGCTGATGGGCATTCTGAGCGATCTGCTGTGCGGCTACCCTGAAGGCG |
| | GAGGATCTAGAGTGCTGAGCTTCAATTGGTACGAGGACAACAACTACAAGGCCTT |
| | CCTGGGCATCGACTCCACCAGAAAGGACCCCATCTACAGCTACGACCGGCGGACA |
| | ACCAGCTTCTGCAATGCCCTGATCCAGAGCCTGGAAAGCAACCCTCTGACCAAGA |
| | TCGCTTGGAGGGCCGCCAAACCTCTGCTGATGGGAAAGATCCTGTACACCCCTGA |
| | CAGCCCTGCCGCCAGAAGAATCCTGAAGAACGCCAACAGCACCTTCGAGGAACTG |
| | GAACACGTGCGCAAGCTGGTCAAGGCCTGGGAAGAAGTGGGACCTCAGATtTGGT |
| | ACTTCTTCGACAATAGCACCCAGATGAACATGATCAGAGACACCCTGGGCAACCC |
| | TACCGTGAAGGACTTCCTGAACAGACAGCTGGGCGAAGAGGGCATTACCGCCGAG |
| | GCCATCCTGAACTTTCTGTACAAGGGCCCCAGAGAGTCCCAGGCCGACGACATGG |
| | CCAACTTCGATTGGCGGGACATCTTCAACATCACCGACAGAACCCTGCGGCTGGT |
| | CAACCAGTACCTGGAATGCCTGGTGCTGGACAAGTTCGAGAGCTACAACGACGAG |
| | ACACAGCTGACCCAGAGAGCCCTGTCTCTGCTGGAAGAGAATATGTTCTGGGCTG |
| | GCGTGGTGTTCCCCGACATGTACCCTTGGACAAGCAGCCTGCCTCCTCACGTGAA |
| | GTACAAGATCCGGATGGACATCGACGTGGTCGAAAAGACCAACAAGATCAAGGAC |
| | CGGTACTGGGACAGCGGCCCTAGAGCTGATCCCGTGGAAGATTTTCGGTACATCT |
| | GGGGCGGATTCGCATACCTGCAGGACATGGTGGAACAGGGAATCACACGGTCCCA |
| | GGTGCAGGCTGAAGCTCCTGTGGGAATCTACCTGCAGCAGATGCCTTATCCTTGC |
| | TTCGTGGACGACAGCTTCATGATCATCCTGAATCGGTGCTTCCCCATCTTCATGG |
| | TGCTGGCCTGGATCTACTCCGTGTCTATGACCGTGAAGTCCATCGTGCTGGAAAA |
| | AGAGCTGCGGCTGAAAGAGACACTGAAGAACCAGGGCGTGTCCAATGCCGTGATC |
| | TGGTGCACCTGGTTTCTGGACAGCTTCTCCATTATGAGCATGAGCATCTTTCTGC |
| | TGACGATCTTCATCATGCACGGCCGaATCCTGCACTACAGCGACCCCTTTATCCT |
| | CTTCCTGTTCCTGCTGGCCTTCAGCACCGCTACAATCATGCTGTGTTTTCTGCTG |
| | TCCACCTTCTTCAGCAAGGCCTCTCTGGCCGCTGCTTGTAGCGGCGTGATCTACT |
| | TCACCCTGTACCTGCCTCACATCCTGTGCTTCGCATGGCAGGACAGAATGACCGC |
| | CGAGCTGAAGAAAGCTGTGTCCCTGCTGAGCCCTGTGGCCTTTGGCTTTGGCACC |
| | GAGTACCTCGTCAGATTTGAGGAACAAGGACTGGGACTGCAGTGGTCCAACATCG |
| | GCAATAGCCCTACAGAGGGCGACGAGTTCAGCTTCCTGCTGTCTATGCAGATGAT |
| | GCTGCTGGACGCCGCCGTGTATGGACTGCTGGCTTGGTATCTGGACCAGGTGTTC |
| | CCAGGCGATTACGGCACTCCTCTGCCTTGGTATTTCCTGCTGCAAGAGAGCTACT |
| | GGCTCGGCGGCGAGGGATGTAGCACCAGAGAAGAAAGAGCCCTGGAAAAGACCGA |
| | GCCTCTGACCGAGGAAACAGAGGACCCTGAACACCCAGAGGGCATCCACGATAGC |
| | TTTTTCGAGAGAGAACACCCCGGCTGGGTGCCAGGCGTGTGTGTGAAGAATCTGG |
| | TCAAGATtTTCGAGCCCTGCGGCAGACCTGCCGTGGACAGACTGAACATCACCTT |
| | CTACGAGAACCAGATTACCGCCTTTCTGGGCCACAACGGCGCTGGCAAGACAACC |
| | ACAtTGAGCATCCTCACAGGCCTGCTGCCTCCAACAAGCGGCACAGTTCTCGTTG |
| | GCGGCAGAGACATCGAGACAAGCCTGGATGCCGTCAGACAGTCCCTGGGCATGTG |
| | CCCTCAGCACAACATCCTGTTTCACCACCTGACCGTGGCCGAGCACATGCTGTTT |
| | TATGCCCAGCTGAAGGGCAAGAGCCAAGAAGAGGCTCAGCTGGAAATGGAAGCCA |
| | TGtTqGAGGACACCGGCCTGCACCACAAGAGAAATGAGGAAGCCCAGGATCTGAG |
| | CGGCGGCATGCAGAGAAAACTGAGCGTGGCCATTGCCTTCGTGGGCGACGCCAAG |
| | GTTGTGATCCTGGATGAGCCTACAAGCGGCGTGGACCCTTACAGCAGAAGATCCA |
| | TCTGGGATCTGCTGCTGAAGTACAGAtcaGGCCGGACCATCATCATGAGCACCCA |
| | CCACATGGACGAGGCCGATCTGCTCGGAGACAGAATCGCCATCATTGCTCAGGGC |
| | AGACTGTACTGCAGCGGCACCCCACTGTTTCTGAAGAACTGTTTCGGCACCGGAC |
| | TGTATCTGACCCTCGTGCGGAAGATGAAGAACATCCAGTCTCAGCGGAAGGGCAG |
| | CGAGGGCACCTGTAGCTGTTCTAGCAAGGGCTTTAGCACCACCTGTCCAGCTCAC |
| | GTGGACGATCTGACCCCTGAACAGGTGCTGGATGGCGACGTGAACGAGCTGATGG |
| | ACGTGGTGCTGCACCATGTGCCTGAGGCCAAGCTGGTGGAATGCATCGGCCAAGA |
| | ACTGATTTTTCTGCTCCCGAACAAGAACTTCAAGCACCGGGCCTACGCCAGCCTG |
| | TTCAGAGAGCTGGAAGAAACCCTGGCCGACCTGGGCCTGTCTAGCTTTGGCATCA |
| | GCGACACCCCTCTCGAAGAGATtTTCCTGAAAGTGACAGAGGACAGCGATAGCGG |
| | CCCTCTGTTTGCTGGCGGAGCACAGCAAAAGCGCGAGAACGTGAACCCTAGACAC |
| | CCCTGTCTGGGCCCAAGAGAGAAAGCCGGACAGACCCCTCAGGACAGCAATGTGT |
| | GCTCTCCTGGTGCTCCTGCCGCTCATCCTGAGGGACAACCTCCACCTGAACCTGA |
| | GTGTCCTGGACCTCAGCTGAACACCGGAACACAGCTGGTTCTGCAGCACGTGCAG |
| | GCTCTGCTCGTGAAGAGATTCCAGCACACCATCAGAAGCCACAAGGACTTTCTGG |
| | CCCAGATCGTGCTGCCCGCCACCTTTGTTTTTCTGGCTCTGATGCTGAGCATCGT |
| | GATCCCTCCATTCGGCGAGTACCCCGCTCTGACACTGCACCCTTGGATCTACGGC |
| | CAGCAGTACACCTTTTTCTCCATGGACGAACCCGGCAGCGAGCAGTTCACAGTGC |
| | TGGCTGATGTCCTGCTGAACAAGCCCGGCTTCGGCAACCGGTGTCTGAAAGAAGG |
| | ATGGCTGCCTGAGTACCCTTGCGGCAACAGCACACCTTGGAAAACCCCTAGCGTG |
| | TCCCCTAACATCACCCAGCTGTTCCAAAAGCAGAAATGGACCCAAGTGAACCCCT |
| | CTCCATCCTGCCGGTGCTCCACAAGGGAAAAGCTGACCATGCTGCCCGAGTGTCC |
| | AGAAGGCGCTGGCGGACTTCCTCCACCTCAGAGAACACAGAGATCCACCGAGATT |
| | CTCCAGGACCTGACCGACCGGAATATCAGCGACTTCCTGGTTAAGACATACCCCG |
| | CACTGATCCGGTCCAGCCTGAAGTCCAAGTTCTGGGTCAACGAACAGAGATACGG |
| | CGGCATCAGCATCGGCGGAAAACTGCCTGTGGTGCCTATCACAGGCGAGGCCCTT |
| | GTGGGCTTTCTGTCCGATCTGGGGAGAATCATGAACGTGTCCGGCGGACCTATCA |
| | CCAGGGAAGCCAGCAAAGAGATCCCCGATTTCCTGAAGCACCTGGAAACCGAGGA |
| | CAATATCAAAGTGTGGTTCAACAACAAAGGATGGCACGCCCTCGTGTCTTTTCTG |
| | AACGTGGCCCACAATGCCATCCTGCGGGCTAGCCTGCCTAAGGACAGAAGCCCTG |
| | AGGAATACGGCATCACCGTGATCTCCCAGCCTCTGAATCTGACCAAAGAGCAGCT |
| | GAGCGAGATCACCGTGCTGACCACCTCTGTGGATGCTGTGGTGGCCATCTGCGTG |
| | ATCTTCAGCATGAGCTTCGTGCCCGCCTCCTTCGTGCTGTACCTGATTCAAGAGA |
| | GAGTGAACAAGAGCAAGCACCTCCAGTTCATCTCCGGGGTGTCCCCAACCACCTA |
| | CTGGGTCACCAATTTTCTGTGGGACATCATGAACTACAGCGTGTCAGCCGGCCTG |
| | GTCGTGGGCATCTTTATCGGCTTTCAGAAGAAGGCCTACACGAGCCCCGAGAACC |
| | TGCCTGCTTTGGTTGCTCTGCTGCTCCTGTATGGCTGGGCCGTGATTCCCATGAT |
| | GTACCCCGCCAGCTTTCTGTTTGACGTGCCCAGCACAGCCTACGTGGCCCTGTCT |
| | TGCGCCAATCTGTTCATCGGCATCAACAGCAGCGCCATCACATTCATCCTGGAAC |
| | TGTTCGAGAACAACAGGACCCTGCTGCGGTTCAACGCCGTGCTGCGGAAACTGCT |
| | GATCGTGTTCCCTCACTTCTGTCTCGGCAGAGGCCTGATCGACCTGGCTCTGTCT |
| | CAGGCCGTGACCGATGTGTACGCCAGATTTGGCGAGGAACACTCCGCCAATCCAT |
| | TCCACTGGGACCTGATCGGCAAGAACCTGTTCGCCATGGTGGTGGAAGGCGTCGT |
| | GTACTTCCTGCTCACTCTGCTGGTGCAGAGACACTTTTTTCTGTCCCAATGGATC |
| | GCCGAGCCTACCAAAGAACCCATTGTGGACGAGGACGACGATGTGGCCGAGGAAA |
| | GACAGAGAATCATCACCGGCGGCAACAAGACCGATATCCTGAGACTGCACGAGCT |
| | GACAAAGATtTACCCCGGCACAAGCTCCCCAGCCGTGGATAGGCTTTGTGTGGGA |
| | GTTAGACCCGGCGAGTGCTTTGGCCTGCTGGGAGTTAATGGCGCCGGAAAGACCA |
| | CCACCTTCAAGATGCTGACCGGCGACACCACAGTGACAAGCGGAGATGCTACAGT |
| | GGCCGGCAAGAGCATCCTGACCAACATCAGCGAAGTGCATCAGAACATGGGCTAC |
| | TGCCCTCAGTTCGACGCCATCGACGAACTGCTGACAGGCCGCGAACACCTGTATC |
| | TGTATGCCAGACTGAGAGGCGTGCCCGCTGAAGAGATCGAGAAGGTGGCCAACTG |
| | GTCCATCAAGTCTCTGGGCCTGACAGTGTACGCCGACTGTCTGGCCGGAACATAC |
| | AGCGGAGGAAACAAGCGGAAGCTGAGCACCGCCATTGCTCTGATCGGATGCCCAC |
| | CTCTGGTCCTGCTGGATGAACCCACCACCGGAATGGAcCCCCAGGCTAGAAGAAT |
| | GCTCTGGAACGTGATCGTGTCTATCATCCGCGAGGGCAGAGCTGTGGTGCTGACC |
| | TCTCACAGCATGGAAGAGTGCGAGGCTCTGTGTACCCGGCTGGCCATTATGGTCA |
| | AGGGCGCCTTCAGATGCATGGGCACCATTCAGCATCTGAAAAGCAAGTTCGGCGA |
| | CGGCTACATCGTGACAATGAAGATCAAGAGCCCCAAGGACGACCTCCTGCCTGAT |
| | CTGAACCCCGTGGAACAGTTTTTTCAGGGCAACTTCCCCGGCTCCGTGCAGCGGG |
| | AAAGACACTATAACATGCTGCAGTTTCAGGTGTCCTCCTCCAGCCTGGCTCGGAT |
| | CTTTCAACTGCTGCTCTCTCACAAGGACAGCCTGCTGATTGAAGAGTACAGCGTG |
| | ACACAGACCACACTCGACCAGGTTTTCGTGAACTTCGCCAAGCAGCAGACCGAGA |
| | GCCACGACCTGCCTCTGCATCCTAGAGCCGCTGGTGCCTCTAGACAAGCTCAGGA |
| | CGGCGGAGGCAGCTACCCTTATGATGTGCCTGATTACGCCTAAGCTTAGACCCTC |
| | CGATCCTCTGAAAGGAGCATCAGGCAGGCAGGGGTTGCTCCGGGGCCAGGGAAGG |
| | GGCCCCGCTGGGACGCGGCTGTTATTGCAGCTGGTTGGCGCGCAGCCATGCTTAG |
| | CTGCAGTGCGGGAATGCTGGGCCTTCTGTTCTGGGCTGTTTCTCATACGCACGTA |
| | GGCCAGTGTATAAATAAGGTTTTATTAAATGCCAAATGAGTTCTCATTAACAAAG |
| | AAAGAGGGAAAATCTCAGTAAACCACCGTGACGGCATCTACCCACTTTGAGTCAG |
| | GAGCTGGGGGTGTGAGTGCAACCTCCGAGACAAGGGAACCTGTGGAGCCCAGAGA |
| | ATCGGAGGGGGGCGCTGGGGTTAGCACCGACTGAACCAGCTGTGTTTTCTCTCGG |
| | TTCCTTGGAGATCAGAAGTGAGTGTTGTCATCTTCAAACAATCCAAAGGCAGTAC |
| | CCATGGCCTTACTACATCCCTCCCACACCATCCCACCCATCCCCGCGCGTACACT |
| | CACACGCTCATTTGCACACTATCGCACACGCTCACTTGCGTGCGCACACACAGAT |
| | TGGTGACCTAGGTGGACTGGGAGAGAAATAAGAGCCAAATGACTGGATTTTCTCC |
| | AAGGAAATTTATTAATAGCCCCTCTTGGTTTCACCTGAAGGAGCTTGTCTTCACC |
| | TGCGGCCTTTGCAGGCTTAACGCCCCCAGCTTGAAACCCAGAAGCTCAGACTTGG |
| | GCCCAAGGTATTATTAGTGCCAACACTACCTGAAATGTTTCGCACCTCATAAAAA |
| | TGGTGTGTCAGTTTCGGGTGAGAGGTTGGGACGCTTCCCATCTGATTTGGCCCAA |
| | GGCATGCATGCCCCTCCTTCTCCTTCCCCTCCTCCTCCCCCTCTTCCCCCTACCA |
| | TCCTTCCTGTTTTCTCTCCAACTCTGGTGCACAGCTTTGAAATCTTGCTGAGAAG |
| | CAAATCTGTCCCTTCTGCTTTGAATGTTTATTTGTGGAAGTTCGGCAGGGGAACC |
| | GAGGCGGGTGCCAAGACCTGCCATGCTGCTGGGAAGTCTGAGTCTCCCTCCCTTC |
| | CCCCTCCTAAATGCTTGTTGATAGAGAAAAGTCAGCCTCCTCGGCATTTGGGCTC |
| | ACGGTTTTCCTTTGAAAATGCTTCCAGTGTGGCATGATTCAGCTTTCTTTTCTGT |
| | CCCCCAACCACTGCTCTGTTGTCATTTTTACTTTTCTGATTGCATTTTATCCGTG |
| | TCTCTTTGACTACGGGGTGGCTGGACGTTGAGTTCCAGGAAGAAAAGGGCCCAAT |
| | CTTGGGGTTCTGACTACATGCGCCCATCAATGTCCTGTTTCATTCTTGGCTCTGG |
| | CTCCCTGAATTCCTGAGTCACTGGGGAGAAGCGTGGGTGGACCGCCCCCTACCCA |
| | GTGAGAGTTGCCACAGTTGCTGCTCTCCTGGGTCATTGGTTGCAGATTGTTAAAC |
| | TTCACCTATGCATTTCAACTTTCGGGTGGATATTGCTACGTCAAGTGTCTGGGAA |
| | AGCCCCCACAGCTACAGGATTTTACAGTGAGGTCCCACTAATGACTTGATGTCAT |
| | GACTTCCTCATTCTTTCCAATTTCTCCCACTTCTCCATAAGGGTTTTGGGAAGGG |
| | GAGAAGAGAAAGGAGTGATTCCTGAGTGCCAGTACCAGGGAACAGCAGGGCTGTT |
| | GGGAGGATAAAATAAAATATCTTTATTTTCATTACATCTGTGTGTTGGTTTTTTG |
| | TGTGCGCTACTtgaagagcgaattctctaggatctgccaggaaccgtaaaaaGCG |
| | CTAGCGGAGTGTATACTGGCTTACTATGTTGGCACTGATGAGGGTGTCAGTGAAG |
| | TGCTTCATGTGGCAGGAGAAAAAAGGCTGCACCGGTGCGTCAGCAGAATATGTGA |
| | TACAGGATATATTCCGCTTCCTCGCTCACTGACTCGCTACGCTCGGTCGTTCGAC |
| | TGCGGCGAGCGGAAATGGCTTACGAACGGGGCGGAGATTTCCTGGAAGATGCCAG |
| | GAAGATACTTAACAGGGAAGTGAGAGGGCCGCGGCAAAGCCGTTTTTCCATAGGC |
| | TCCGCCCCCCTGACAAGCATCACGAAATCTGACGCTCAAATCAGTGGTGGCGAAA |
| | CCCGACAGGACTATAAAGATACCAGGCGTTTCCCCCTGGCGGCTCCCTCGTGCGC |
| | TCTCCTGTTCCTGCCTTTCGGTTTACCGGTGTCATTCCGCTGTTATGGCCGCGTT |
| | TGTCTCATTCCACGCCTGACACTCAGTTCCGGGTAGGCAGTTCGCTCCAAGCTGG |
| | ACTGTATGCACGAACCCCCCGTTCAGTCCGACCGCTGCGCCTTATCCGGTAACTA |
| | TCGTCTTGAGTCCAACCCGGAAAGACATGCAAAAGCACCACTGGCAGCAGCCACT |
| | GGTAATTGATTTAGAGGAGTTAGTCTTGAAGTCATGCGCCGGTTAAGGCTAAACT |
| | GAAAGGACAAGTTTTGGTGACTGCGCTCCTCCAAGCCAGTTACCTCGGTTCAAAG |
| | AGTTGGTAGCTCAGAGAACCTTCGAAAAACCGCCCTGCAAGGCGGTTTTTTCGTT |
| | TTCAGAGCAAGAGATTACGCGCAGACCAAAACGATCTCAAGAAGATCATCTTATT |
| | AATCAGATAAAATATTTCTAGATTTCAGTGCAATTTATCTCTTCAAATGTAGCAC |
| | CTGAAGTCAGCCCCATACGATATAAGTTGTgattatcaaaaaggatcttcaccta |
| | gatccttttaaattaaaaatgaagttttaaatcaatctaaagtatatatgagtaa |
| | acttggtctgacagttaccaatgcttaatcagtgaggcacctatctcagcgatct |
| | gtctatttcgttcatccatagttgcctgactccccgtcgtgtagataactacgat |
| | acgggagggcttaccatctggccccagtgctgcaatgataccgcgagaaccacgc |
| | tcaccggctccagatttatcagcaataaaccagccagccggaagggccgagcgca |
| | gaagtggtcctgcaactttatccgcctccatccagtctattaattgttgccggga |
| | agctagagtaagtagttcgccagttaatagtttgcgcaacgttgttgccattgct |
| | acaggcatcgtggtgtcacgctcgtcgtttggtatggcttcattcagctccggtt |
| | cccaacgatcaaggcgagttacatgatcccccatgttgtgcaaaaaagcggttag |
| | ctccttcggtcctccgatcgttgtcagaagtaagttggccgcagtgttatcactc |
| | atggttatggcagcactgcataattctcttactgtcatgccatccgtaagatgct |
| | tttctgtgactggtgagtactcaaccaagtcattctgagaatagtgtatgcggcg |
| | accgagttgctcttgcccggcgtcaatacgggataataccgcgccacatagcaga |
| | actttaaaagtgctcatcattggaaaacgttcttcggggcgaaaactctcaagga |
| | tcttaccgctgttgagatccagttcgatgtaacccactcgtgcacccaactgatc |
| | ttcagcatcttttactttcaccagcgtttctgggtgagcaaaaacaggaaggcaa |
| | aatgccgcaaaaaagggaataagggcgacacggaaatgttgaatactcatactct |
| | tcctttttcaatattattgaagcatttatcagggttattgtctcatgagcggata |
| | catatttgaatgtatttagaaaaataaacaaataggggttccgcgcacgaattcg |
| | ctcttcaCTC |
| 37 | TTCGgtatcacaggagaatttcagGGAGAGGGATCACTTCAGATCTGCCGAGTGA |
| | GTCGATTGGACTTAAAGGGCCAGTCAAACCCTGACTGCCGGCTCATGGCAGGCTC |
| | TTGCCGAGGACAAATGCCCAGCCTATATTTATGCAAAGAGATTTTGTTCCAAACT |
| | TAAGGTCAAAGATACCTAAAGACATCCCCCTCAGGAACCCCTCTCATGGAGGAGA |
| | GTGCCTGAGGGTCTTGGTTTCCCATTGCATCCCCCACCTCAATTTCCCTGGTGCC |
| | CAGCCACTTGTGTCTTTAGGGTTCTCTTTCTCTCCATAAAAGGGAGCCAACACAG |
| | TGTCGGCCTCCTCTCCCCAACTAAGGGCTTATGTGTAATTAAAAGGGATTATGCT |
| | TTGAAGGGGAAAAGTAGCCTTTAATCACCAGGAGAAGGACACAGCGTCCGGAGCC |
| | AGAGGCGCTCTTAACGGCGTTTATGTCCTTTGCTGTCTGAGGGGCCTCAGCTCTG |
| | ACCAATCTGGTCTTCGTGTGGTCATTAGCGTAACAGTTACTGTCTGTGGTTTAAA |
| | AATGAGGTGTGGAGCAAATAAACAGGTTGGAAGTGTGGGGTGGGGTGGTGGGGTA |
| | GGGTGGTGGGGCAGGGTGGGGGGTTGTGAGCAGTCAGTGGGCTTGTCGCCGATTA |
| | GCACTGAAGCAGTGTTTAGCTGGACGGCCTTTCTGTGGGCCCCTCTGACAGTGCC |
| | CTTCCCAGGAAGATGTGTTTCTCTGTCCTCAGCCACATGAAAATCTTTTGCCTAC |
| | CGTGCCTGTCAATCCATTGCCTGCCCGCCCCTCCCCCACCCCCCGTTTTACACCT |
| | GCCTGTCCAGTCTACCGCTCTCTAGGGCATCCACGCTGAGCAGTGGGAAGAACTT |
| | TAAGCCCTGATGAGCAGGCCAAAGGCAAGCAAGAACCCCCTCGAACAGCTTCCCA |
| | GCTTAGTGAGGCCTTACAGATAAGTTTGTACAAAAAAGAGAGGTGCCACCATGGG |
| | CTTTGTGCGACAGATTCAGCTGCTGCTGTGGAAGAACTGGACCCTGCGGAAGCGG |
| | CAGAAAATCAGATTCGTGGTGGAACTCGTGTGGCCCCTGAGCCTGTTTCTGGTGC |
| | TGATCTGGCTGCGGAACGCCAATCCTCTGTACAGCCACCACGAGTGTCACTTCCC |
| | CAACAAGGCCATGCCTTCTGCCGGAATGCTGCCTTGGCTGCAGGGCATCTTCTGC |
| | AACGTGAACAACCCCTGCTTTCAGAGCCCCACACCTGGCGAAAGCCCTGGCATCG |
| | TGTCCAACTACAACAACAGCATCCTGGCCAGAGTGTACCGGGACTTCCAAGAGCT |
| | GCTGATGAACGCCCCTGAGTCTCAGCACCTGGGCAGAATCTGGACCGAGCTGCAC |
| | ATCCTGAGCCAGTTCATGGACACCCTGAGAACACACCCCGAGAGAATCGCCGGCA |
| | GGGGCATCAGAATCCGGGACATCCTGAAGGACGAGGAAACCCTGACACTGTTCCT |
| | CATCAAGAACATCGGCCTGAGCGACAGCGTGGTGTACCTGCTGATCAACAGCCAA |
| | GTGCGGCCCGAGCAGTTTGCTCATGGCGTGCCgGATCTCGCCCTGAAGGATATCG |
| | CCTGTTCTGAGGCCCTGCTGGAACGGTTCATCATCTTCAGCCAGCGGAGAGGCGC |
| | CAAGACCGTCAGATATGCCCTGTGCAGTCTGAGCCAGGGAACCCTGCAGTGGATC |
| | GAGGATACCCTGTACGCCAACGTGGACTTCTTCAAGCTGTTCCGGGTGCTGCCCA |
| | CACTGCTGGATTCTAGATCCCAGGGCATCAACCTGAGAAGCTGGGGCGGCATCCT |
| | GTCCGACATGAGCCCAAGAATCCAAGAGTTCATCCACCGGCCTAGCATGCAGGAC |
| | CTGCTGTGGGTTACCAGACCTCTGATGCAGAACGGCGGACCCGAGACATTCACCA |
| | AGCTGATGGGCATTCTGAGCGATCTGCTGTGCGGCTACCCTGAAGGCGGAGGATC |
| | TAGAGTGCTGAGCTTCAATTGGTACGAGGACAACAACTACAAGGCCTTCCTGGGC |
| | ATCGACTCCACCAGAAAGGACCCCATCTACAGCTACGACCGGCGGACAACCAGCT |
| | TCTGCAATGCCCTGATCCAGAGCCTGGAAAGCAACCCTCTGACCAAGATCGCTTG |
| | GAGGGCCGCCAAACCTCTGCTGATGGGAAAGATCCTGTACACCCCTGACAGCCCT |
| | GCCGCCAGAAGAATCCTGAAGAACGCCAACAGCACCTTCGAGGAACTGGAACACG |
| | TGCGCAAGCTGGTCAAGGCCTGGGAAGAAGTGGGACCTCAGATETGGTACTTCTT |
| | CGACAATAGCACCCAGATGAACATGATCAGAGACACCCTGGGCAACCCTACCGTG |
| | AAGGACTTCCTGAACAGACAGCTGGGCGAAGAGGGCATTACCGCCGAGGCCATCC |
| | TGAACTTTCTGTACAAGGGCCCCAGAGAGTCCCAGGCCGACGACATGGCCAACTT |
| | CGATTGGCGGGACATCTTCAACATCACCGACAGAACCCTGCGGCTGGTCAACCAG |
| | TACCTGGAATGCCTGGTGCTGGACAAGTTCGAGAGCTACAACGACGAGACACAGC |
| | TGACCCAGAGAGCCCTGTCTCTGCTGGAAGAGAATATGTTCTGGGCTGGCGTGGT |
| | GTTCCCCGACATGTACCCTTGGACAAGCAGCCTGCCTCCTCACGTGAAGTACAAG |
| | ATCCGGATGGACATCGACGTGGTCGAAAAGACCAACAAGATCAAGGACCGGTACT |
| | GGGACAGCGGCCCTAGAGCTGATCCCGTGGAAGATTTTCGGTACATCTGGGGCGG |
| | ATTCGCATACCTGCAGGACATGGTGGAACAGGGAATCACACGGTCCCAGGTGCAG |
| | GCTGAAGCTCCTGTGGGAATCTACCTGCAGCAGATGCCTTATCCTTGCTTCGTGG |
| | ACGACAGCTTCATGATCATCCTGAATCGGTGCTTCCCCATCTTCATGGTGCTGGC |
| | CTGGATCTACTCCGTGTCTATGACCGTGAAGTCCATCGTGCTGGAAAAAGAGCTG |
| | CGGCTGAAAGAGACACTGAAGAACCAGGGCGTGTCCAATGCCGTGATCTGGTGCA |
| | CCTGGTTTCTGGACAGCTTCTCCATTATGAGCATGAGCATCTTTCTGCTGACGAT |
| | CTTCATCATGCACGGCCGaATCCTGCACTACAGCGACCCCTTTATCCTCTTCCTG |
| | TTCCTGCTGGCCTTCAGCACCGCTACAATCATGCTGTGTTTTCTGCTGTCCACCT |
| | TCTTCAGCAAGGCCTCTCTGGCCGCTGCTTGTAGCGGCGTGATCTACTTCACCCT |
| | GTACCTGCCTCACATCCTGTGCTTCGCATGGCAGGACAGAATGACCGCCGAGCTG |
| | AAGAAAGCTGTGTCCCTGCTGAGCCCTGTGGCCTTTGGCTTTGGCACCGAGTACC |
| | TCGTCAGATTTGAGGAACAAGGACTGGGACTGCAGTGGTCCAACATCGGCAATAG |
| | CCCTACAGAGGGCGACGAGTTCAGCTTCCTGCTGTCTATGCAGATGATGCTGCTG |
| | GACGCCGCCGTGTATGGACTGCTGGCTTGGTATCTGGACCAGGTGTTCCCAGGCG |
| | ATTACGGCACTCCTCTGCCTTGGTATTTCCTGCTGCAAGAGAGCTACTGGCTCGG |
| | CGGCGAGGGATGTAGCACCAGAGAAGAAAGAGCCCTGGAAAAGACCGAGCCTCTG |
| | ACCGAGGAAACAGAGGACCCTGAACACCCAGAGGGCATCCACGATAGCTTTTTCG |
| | AGAGAGAACACCCCGGCTGGGTGCCAGGCGTGTGTGTGAAGAATCTGGTCAAGAT |
| | tTTCGAGCCCTGCGGCAGACCTGCCGTGGACAGACTGAACATCACCTTCTACGAG |
| | AACCAGATTACCGCCTTTCTGGGCCACAACGGCGCTGGCAAGACAACCACAtTGA |
| | GCATCCTCACAGGCCTGCTGCCTCCAACAAGCGGCACAGTTCTCGTTGGCGGCAG |
| | AGACATCGAGACAAGCCTGGATGCCGTCAGACAGTCCCTGGGCATGTGCCCTCAG |
| | CACAACATCCTGTTTCACCACCTGACCGTGGCCGAGCACATGCTGTTTTATGCCC |
| | AGCTGAAGGGCAAGAGCCAAGAAGAGGCTCAGCTGGAAATGGAAGCCATGtTqGA |
| | GGACACCGGCCTGCACCACAAGAGAAATGAGGAAGCCCAGGATCTGAGCGGCGGC |
| | ATGCAGAGAAAACTGAGCGTGGCCATTGCCTTCGTGGGCGACGCCAAGGTTGTGA |
| | TCCTGGATGAGCCTACAAGCGGCGTGGACCCTTACAGCAGAAGATCCATCTGGGA |
| | TCTGCTGCTGAAGTACAGAtcaGGCCGGACCATCATCATGAGCACCCACCACATG |
| | GACGAGGCCGATCTGCTCGGAGACAGAATCGCCATCATTGCTCAGGGCAGACTGT |
| | ACTGCAGCGGCACCCCACTGTTTCTGAAGAACTGTTTCGGCACCGGACTGTATCT |
| | GACCCTCGTGCGGAAGATGAAGAACATCCAGTCTCAGCGGAAGGGCAGCGAGGGC |
| | ACCTGTAGCTGTTCTAGCAAGGGCTTTAGCACCACCTGTCCAGCTCACGTGGACG |
| | ATCTGACCCCTGAACAGGTGCTGGATGGCGACGTGAACGAGCTGATGGACGTGGT |
| | GCTGCACCATGTGCCTGAGGCCAAGCTGGTGGAATGCATCGGCCAAGAACTGATT |
| | TTTCTGCTCCCGAACAAGAACTTCAAGCACCGGGCCTACGCCAGCCTGTTCAGAG |
| | AGCTGGAAGAAACCCTGGCCGACCTGGGCCTGTCTAGCTTTGGCATCAGCGACAC |
| | CCCTCTCGAAGAGATtTTCCTGAAAGTGACAGAGGACAGCGATAGCGGCCCTCTG |
| | TTTGCTGGCGGAGCACAGCAAAAGCGCGAGAACGTGAACCCTAGACACCCCTGTC |
| | TGGGCCCAAGAGAGAAAGCCGGACAGACCCCTCAGGACAGCAATGTGTGCTCTCC |
| | TGGTGCTCCTGCCGCTCATCCTGAGGGACAACCTCCACCTGAACCTGAGTGTCCT |
| | GGACCTCAGCTGAACACCGGAACACAGCTGGTTCTGCAGCACGTGCAGGCTCTGC |
| | TCGTGAAGAGATTCCAGCACACCATCAGAAGCCACAAGGACTTTCTGGCCCAGAT |
| | CGTGCTGCCCGCCACCTTTGTTTTTCTGGCTCTGATGCTGAGCATCGTGATCCCT |
| | CCATTCGGCGAGTACCCCGCTCTGACACTGCACCCTTGGATCTACGGCCAGCAGT |
| | ACACCTTTTTCTCCATGGACGAACCCGGCAGCGAGCAGTTCACAGTGCTGGCTGA |
| | TGTCCTGCTGAACAAGCCCGGCTTCGGCAACCGGTGTCTGAAAGAAGGATGGCTG |
| | CCTGAGTACCCTTGCGGCAACAGCACACCTTGGAAAACCCCTAGCGTGTCCCCTA |
| | ACATCACCCAGCTGTTCCAAAAGCAGAAATGGACCCAAGTGAACCCCTCTCCATC |
| | CTGCCGGTGCTCCACAAGGGAAAAGCTGACCATGCTGCCCGAGTGTCCAGAAGGC |
| | GCTGGCGGACTTCCTCCACCTCAGAGAACACAGAGATCCACCGAGATTCTCCAGG |
| | ACCTGACCGACCGGAATATCAGCGACTTCCTGGTTAAGACATACCCCGCACTGAT |
| | CCGGTCCAGCCTGAAGTCCAAGTTCTGGGTCAACGAACAGAGATACGGCGGCATC |
| | AGCATCGGCGGAAAACTGCCTGTGGTGCCTATCACAGGCGAGGCCCTTGTGGGCT |
| | TTCTGTCCGATCTGGGGAGAATCATGAACGTGTCCGGCGGACCTATCACCAGGGA |
| | AGCCAGCAAAGAGATCCCCGATTTCCTGAAGCACCTGGAAACCGAGGACAATATC |
| | AAAGTGTGGTTCAACAACAAAGGATGGCACGCCCTCGTGTCTTTTCTGAACGTGG |
| | CCCACAATGCCATCCTGCGGGCTAGCCTGCCTAAGGACAGAAGCCCTGAGGAATA |
| | CGGCATCACCGTGATCTCCCAGCCTCTGAATCTGACCAAAGAGCAGCTGAGCGAG |
| | ATCACCGTGCTGACCACCTCTGTGGATGCTGTGGTGGCCATCTGCGTGATCTTCA |
| | GCATGAGCTTCGTGCCCGCCTCCTTCGTGCTGTACCTGATTCAAGAGAGAGTGAA |
| | CAAGAGCAAGCACCTCCAGTTCATCTCCGGGGTGTCCCCAACCACCTACTGGGTC |
| | ACCAATTTTCTGTGGGACATCATGAACTACAGCGTGTCAGCCGGCCTGGTCGTGG |
| | GCATCTTTATCGGCTTTCAGAAGAAGGCCTACACGAGCCCCGAGAACCTGCCTGC |
| | TTTGGTTGCTCTGCTGCTCCTGTATGGCTGGGCCGTGATTCCCATGATGTACCCC |
| | GCCAGCTTTCTGTTTGACGTGCCCAGCACAGCCTACGTGGCCCTGTCTTGCGCCA |
| | ATCTGTTCATCGGCATCAACAGCAGCGCCATCACATTCATCCTGGAACTGTTCGA |
| | GAACAACAGGACCCTGCTGCGGTTCAACGCCGTGCTGCGGAAACTGCTGATCGTG |
| | TTCCCTCACTTCTGTCTCGGCAGAGGCCTGATCGACCTGGCTCTGTCTCAGGCCG |
| | TGACCGATGTGTACGCCAGATTTGGCGAGGAACACTCCGCCAATCCATTCCACTG |
| | GGACCTGATCGGCAAGAACCTGTTCGCCATGGTGGTGGAAGGCGTCGTGTACTTC |
| | CTGCTCACTCTGCTGGTGCAGAGACACTTTTTTCTGTCCCAATGGATCGCCGAGC |
| | CTACCAAAGAACCCATTGTGGACGAGGACGACGATGTGGCCGAGGAAAGACAGAG |
| | AATCATCACCGGCGGCAACAAGACCGATATCCTGAGACTGCACGAGCTGACAAAG |
| | ATtTACCCCGGCACAAGCTCCCCAGCCGTGGATAGGCTTTGTGTGGGAGTTAGAC |
| | CCGGCGAGTGCTTTGGCCTGCTGGGAGTTAATGGCGCCGGAAAGACCACCACCTT |
| | CAAGATGCTGACCGGCGACACCACAGTGACAAGCGGAGATGCTACAGTGGCCGGC |
| | AAGAGCATCCTGACCAACATCAGCGAAGTGCATCAGAACATGGGCTACTGCCCTC |
| | AGTTCGACGCCATCGACGAACTGCTGACAGGCCGCGAACACCTGTATCTGTATGC |
| | CAGACTGAGAGGCGTGCCCGCTGAAGAGATCGAGAAGGTGGCCAACTGGTCCATC |
| | AAGTCTCTGGGCCTGACAGTGTACGCCGACTGTCTGGCCGGAACATACAGCGGAG |
| | GAAACAAGCGGAAGCTGAGCACCGCCATTGCTCTGATCGGATGCCCACCTCTGGT |
| | CCTGCTGGATGAACCCACCACCGGAATGGAcCCCCAGGCTAGAAGAATGCTCTGG |
| | AACGTGATCGTGTCTATCATCCGCGAGGGCAGAGCTGTGGTGCTGACCTCTCACA |
| | GCATGGAAGAGTGCGAGGCTCTGTGTACCCGGCTGGCCATTATGGTCAAGGGCGC |
| | CTTCAGATGCATGGGCACCATTCAGCATCTGAAAAGCAAGTTCGGCGACGGCTAC |
| | ATCGTGACAATGAAGATCAAGAGCCCCAAGGACGACCTCCTGCCTGATCTGAACC |
| | CCGTGGAACAGTTTTTTCAGGGCAACTTCCCCGGCTCCGTGCAGCGGGAAAGACA |
| | CTATAACATGCTGCAGTTTCAGGTGTCCTCCTCCAGCCTGGCTCGGATCTTTCAA |
| | CTGCTGCTCTCTCACAAGGACAGCCTGCTGATTGAAGAGTACAGCGTGACACAGA |
| | CCACACTCGACCAGGTTTTCGTGAACTTCGCCAAGCAGCAGACCGAGAGCCACGA |
| | CCTGCCTCTGCATCCTAGAGCCGCTGGTGCCTCTAGACAAGCTCAGGACGGCGGA |
| | GGCAGCTACCCTTATGATGTGCCTGATTACGCCTAAGCTTAGACCCTCCGATCCT |
| | CTGAAAGGAGCATCAGGCAGGCAGGGGTTGCTCCGGGGCCAGGGAAGGGGCCCCG |
| | CTGGGACGCGGCTGTTATTGCAGCTGGTTGGCGCGCAGCCATGCTTAGCTGCAGT |
| | GCGGGAATGCTGGGCCTTCTGTTCTGGGCTGTTTCTCATACGCACGTAGGCCAGT |
| | GTATAAATAAGGTTTTATTAAATGCCAAATGAGTTCTCATTAACAAAGAAAGAGG |
| | GAAAATCTCAGTAAACCACCGTGACGGCATCTACCCACTTTGAGTCAGGAGCTGG |
| | GGGTGTGAGTGCAACCTCCGAGACAAGGGAACCTGTGGAGCCCAGAGAATCGGAG |
| | GGGGGCGCTGGGGTTAGCACCGACTGAACCAGCTGTGTTTTCTCTCGGTTCCTTG |
| | GAGATCAGAAGTGAGTGTTGTCATCTTCAAACAATCCAAAGGCAGTACCCATGGC |
| | CTTACTACATCCCTCCCACACCATCCCACCCATCCCCGCGCGTACACTCACACGC |
| | TCATTTGCACACTATCGCACACGCTCACTTGCGTGCGCACACACAGATTGGTGAC |
| | CTAGGTGGACTGGGAGAGAAATAAGAGCCAAATGACTGGATTTTCTCCAAGGAAA |
| | TTTATTAATAGCCCCTCTTGGTTTCACCTGAAGGAGCTTGTCTTCACCTGCGGCC |
| | TTTGCAGGCTTAACGCCCCCAGCTTGAAACCCAGAAGCTCAGACTTGGGCCCAAG |
| | GTATTATTAGTGCCAACACTACCTGAAATGTTTCGCACCTCATAAAAATGGTGTG |
| | TCAGTTTCGGGTGAGAGGTTGGGACGCTTCCCATCTGATTTGGCCCAAGGCATGC |
| | ATGCCCCTCCTTCTCCTTCCCCTCCTCCTCCCCCTCTTCCCCCTACCATCCTTCC |
| | TGTTTTCTCTCCAACTCTGGTGCACAGCTTTGAAATCTTGCTGAGAAGCAAATCT |
| | GTCCCTTCTGCTTTGAATGTTTATTTGTGGAAGTTCGGCAGGGGAACCGAGGCGG |
| | GTGCCAAGACCTGCCATGCTGCTGGGAAGTCTGAGTCTCCCTCCCTTCCCCCTCC |
| | TAAATGCTTGTTGATAGAGAAAAGTCAGCCTCCTCGGCATTTGGGCTCACGGTTT |
| | TCCTTTGAAAATGCTTCCAGTGTGGCATGATTCAGCTTTCTTTTCTGTCCCCCAA |
| | CCACTGCTCTGTTGTCATTTTTACTTTTCTGATTGCATTTTATCCGTGTCTCTTT |
| | GACTACGGGGTGGCTGGACGTTGAGTTCCAGGAAGAAAAGGGCCCAATCTTGGGG |
| | TTCTGACTACATGCGCCCATCAATGTCCTGTTTCATTCTTGGCTCTGGCTCCCTG |
| | AATTCCTGAGTCACTGGGGAGAAGCGTGGGTGGACCGCCCCCTACCCAGTGAGAG |
| | TTGCCACAGTTGCTGCTCTCCTGGGTCATTGGTTGCAGATTGTTAAACTTCACCT |
| | ATGCATTTCAACTTTCGGGTGGATATTGCTACGTCAAGTGTCTGGGAAAGCCCCC |
| | ACAGCTACAGGATTTTACAGTGAGGTCCCACTAATGACTTGATGTCATGACTTCC |
| | TCATTCTTTCCAATTTCTCCCACTTCTCCATAAGGGTTTTGGGAAGGGGAGAAGA |
| | GAAAGGAGTGATTCCTGAGTGCCAGTACCAGGGAACAGCAGGGCTGTTGGGAGGA |
| | TAAAATAAAATATCTTTATTTTCATTACATCTGTGTGTTGGTTTTTTGTGTGCGC |
| | TACTtgaagagcgaattctctaggatctgccaggaaccgtaaaaaGCGCTAGCGG |
| | AGTGTATACTGGCTTACTATGTTGGCACTGATGAGGGTGTCAGTGAAGTGCTTCA |
| | TGTGGCAGGAGAAAAAAGGCTGCACCGGTGCGTCAGCAGAATATGTGATACAGGA |
| | TATATTCCGCTTCCTCGCTCACTGACTCGCTACGCTCGGTCGTTCGACTGCGGCG |
| | AGCGGAAATGGCTTACGAACGGGGCGGAGATTTCCTGGAAGATGCCAGGAAGATA |
| | CTTAACAGGGAAGTGAGAGGGCCGCGGCAAAGCCGTTTTTCCATAGGCTCCGCCC |
| | CCCTGACAAGCATCACGAAATCTGACGCTCAAATCAGTGGTGGCGAAACCCGACA |
| | GGACTATAAAGATACCAGGCGTTTCCCCCTGGCGGCTCCCTCGTGCGCTCTCCTG |
| | TTCCTGCCTTTCGGTTTACCGGTGTCATTCCGCTGTTATGGCCGCGTTTGTCTCA |
| | TTCCACGCCTGACACTCAGTTCCGGGTAGGCAGTTCGCTCCAAGCTGGACTGTAT |
| | GCACGAACCCCCCGTTCAGTCCGACCGCTGCGCCTTATCCGGTAACTATCGTCTT |
| | GAGTCCAACCCGGAAAGACATGCAAAAGCACCACTGGCAGCAGCCACTGGTAATT |
| | GATTTAGAGGAGTTAGTCTTGAAGTCATGCGCCGGTTAAGGCTAAACTGAAAGGA |
| | CAAGTTTTGGTGACTGCGCTCCTCCAAGCCAGTTACCTCGGTTCAAAGAGTTGGT |
| | AGCTCAGAGAACCTTCGAAAAACCGCCCTGCAAGGCGGTTTTTTCGTTTTCAGAG |
| | CAAGAGATTACGCGCAGACCAAAACGATCTCAAGAAGATCATCTTATTAATCAGA |
| | TAAAATATTTCTAGATTTCAGTGCAATTTATCTCTTCAAATGTAGCACCTGAAGT |
| | CAGCCCCATACGATATAAGTTGTgattatcaaaaaggatottcacctagatcctt |
| | ttaaattaaaaatgaagttttaaatcaatctaaagtatatatgagtaaacttggt |
| | ctgacagttaccaatgcttaatcagtgaggcacctatctcagcgatctgtctatt |
| | tcgttcatccatagttgcctgactccccgtcgtgtagataactacgatacgggag |
| | ggcttaccatctggccccagtgctgcaatgataccgcgagaaccacgctcaccgg |
| | ctccagatttatcagcaataaaccagccagccggaagggccgagcgcagaagtgg |
| | tcctgcaactttatccgcctccatccagtctattaattgttgccgggaagctaga |
| | gtaagtagttcgccagttaatagtttgcgcaacgttgttgccattgctacaggca |
| | tcgtggtgtcacgctcgtcgtttggtatggcttcattcagctccggttcccaacg |
| | atcaaggcgagttacatgatcccccatgttgtgcaaaaaagcggttagctccttc |
| | ggtcctccgatcgttgtcagaagtaagttggccgcagtgttatcactcatggtta |
| | tggcagcactgcataattctcttactgtcatgccatccgtaagatgcttttctgt |
| | gactggtgagtactcaaccaagtcattctgagaatagtgtatgcggcgaccgagt |
| | tgctcttgcccggcgtcaatacgggataataccgcgccacatagcagaactttaa |
| | aagtgctcatcattggaaaacgttcttcggggcgaaaactctcaaggatcttacc |
| | gctgttgagatccagttcgatgtaacccactcgtgcacccaactgatcttcagca |
| | tcttttactttcaccagcgtttctgggtgagcaaaaacaggaaggcaaaatgccg |
| | caaaaaagggaataagggcgacacggaaatgttgaatactcatactcttcctttt |
| | tcaatattattgaagcatttatcagggttattgtctcatgagcggatacatattt |
| | gaatgtatttagaaaaataaacaaataggggttccgcgcacgaattcgctcttca |
| | CTC |
| 38 | TTCGgtatcacaggagaatttcagGGAGCAAGATAAGCTATTCCTCCCTGTCTTT |
| | GCAGAGTTTAAATGACTCACTTGTTCAAGTACCCACCGTTGCCATGTGGGACCGT |
| | GAGCAAAGTACTTAATCTCACTAAGCTTCACGTTCCTCATCTGTAAAACAGCAAA |
| | TATGGACCTCACAAAATTGTAGTGAGGCTAAAATGAAATAACATATGCAAAAGCA |
| | GTTTATAAATAATAAACTTACTATAAAATATTATTTTGTAATTCTGCAAGCTTGT |
| | CTTAAATGCCATCACCTCCAAGGAGCCTTTTTGCCATCATAAGCAGAAACTATCT |
| | CTCTCTTCTTGGAAGCTCCACCATGCACAGCCTATGGGCCCTCATCACACTCCTT |
| | GAGTTATTCGAGTTCAAGTCCCGTGTTTACAACCAGACCGCAAACTCTATGAAGT |
| | CAGCATCCATTCCTCTCTGTGGTTCTCCCTCCGCCCCATCCAGGCTCAAGGGTCT |
| | AGAGTCTTTCAAAGAGAACACATTCTGAGATTTGAGGAGGCAGAGACAAAAAGTT |
| | CCACTGCGAAGTGCCAGGGAGGCTTCTGTTTGGGGTGTCCCTTGGGATCACAGAT |
| | CCCCCACCTGGTGATGAGTCAACCCAGCACCACCCCATTGCAGGGCTGGAATGAC |
| | AGTAATGGGCCCACCTGCTGCCTCTCCTCATACCCGCACCCCAGTCAGACATTGC |
| | AAGTCAGTCACGGCTCTGTCCTGCTGGGCCTGGAGTGTTCCAGTGCCTTTTCCAT |
| | CACAGCACCAAGCAGCCACTACTAGTCGATCAATTTCAGCACAAGAGATAAACAT |
| | CATTACCCTCTGCTAAGCTCAGAGATAACCCAACTAGCTGACCATAATGACTTCA |
| | GTCATTACGGAGCAAGATAAAAGACTAAAAGAGGGAGGGATCACTTCAGATCTGC |
| | CGAGTGAGTCGATTGGACTTAAAGGGCCAGTCAAACCCTGACTGCCGGCTCATGG |
| | CAGGCTCTTGCCGAGGACAAATGCCCAGCCTATATTTATGCAAAGAGATTTTGTT |
| | CCAAACTTAAGGTCAAAGATACCTAAAGACATCCCCCTCAGGAACCCCTCTCATG |
| | GAGGAGAGTGCCTGAGGGTCTTGGTTTCCCATTGCATCCCCCACCTCAATTTCCC |
| | TGGTGCCCAGCCACTTGTGTCTTTAGGGTTCTCTTTCTCTCCATAAAAGGGAGCC |
| | AACACAGTGTCGGCCTCCTCTCCCCAACTAAGGGCTTATGTGTAATTAAAAGGGA |
| | TTATGCTTTGAAGGGGAAAAGTAGCCTTTAATCACCAGGAGAAGGACACAGCGTC |
| | CGGAGCCAGAGGCGCTCTTAACGGCGTTTATGTCCTTTGCTGTCTGAGGGGCCTC |
| | AGCTCTGACCAATCTGGTCTTCGTGTGGTCATTAGCAGATAAGTTTGTACAAAAA |
| | AGAGAGGTGCCACCATGGGCTTTGTGCGACAGATTCAGCTGCTGCTGTGGAAGAA |
| | CTGGACCCTGCGGAAGCGGCAGAAAATCAGATTCGTGGTGGAACTCGTGTGGCCC |
| | CTGAGCCTGTTTCTGGTGCTGATCTGGCTGCGGAACGCCAATCCTCTGTACAGCC |
| | ACCACGAGTGTCACTTCCCCAACAAGGCCATGCCTTCTGCCGGAATGCTGCCTTG |
| | GCTGCAGGGCATCTTCTGCAACGTGAACAACCCCTGCTTTCAGAGCCCCACACCT |
| | GGCGAAAGCCCTGGCATCGTGTCCAACTACAACAACAGCATCCTGGCCAGAGTGT |
| | ACCGGGACTTCCAAGAGCTGCTGATGAACGCCCCTGAGTCTCAGCACCTGGGCAG |
| | AATCTGGACCGAGCTGCACATCCTGAGCCAGTTCATGGACACCCTGAGAACACAC |
| | CCCGAGAGAATCGCCGGCAGGGGCATCAGAATCCGGGACATCCTGAAGGACGAGG |
| | AAACCCTGACACTGTTCCTCATCAAGAACATCGGCCTGAGCGACAGCGTGGTGTA |
| | CCTGCTGATCAACAGCCAAGTGCGGCCCGAGCAGTTTGCTCATGGCGTGCCqGAT |
| | CTCGCCCTGAAGGATATCGCCTGTTCTGAGGCCCTGCTGGAACGGTTCATCATCT |
| | TCAGCCAGCGGAGAGGCGCCAAGACCGTCAGATATGCCCTGTGCAGTCTGAGCCA |
| | GGGAACCCTGCAGTGGATCGAGGATACCCTGTACGCCAACGTGGACTTCTTCAAG |
| | CTGTTCCGGGTGCTGCCCACACTGCTGGATTCTAGATCCCAGGGCATCAACCTGA |
| | GAAGCTGGGGCGGCATCCTGTCCGACATGAGCCCAAGAATCCAAGAGTTCATCCA |
| | CCGGCCTAGCATGCAGGACCTGCTGTGGGTTACCAGACCTCTGATGCAGAACGGC |
| | GGACCCGAGACATTCACCAAGCTGATGGGCATTCTGAGCGATCTGCTGTGCGGCT |
| | ACCCTGAAGGCGGAGGATCTAGAGTGCTGAGCTTCAATTGGTACGAGGACAACAA |
| | CTACAAGGCCTTCCTGGGCATCGACTCCACCAGAAAGGACCCCATCTACAGCTAC |
| | GACCGGCGGACAACCAGCTTCTGCAATGCCCTGATCCAGAGCCTGGAAAGCAACC |
| | CTCTGACCAAGATCGCTTGGAGGGCCGCCAAACCTCTGCTGATGGGAAAGATCCT |
| | GTACACCCCTGACAGCCCTGCCGCCAGAAGAATCCTGAAGAACGCCAACAGCACC |
| | TTCGAGGAACTGGAACACGTGCGCAAGCTGGTCAAGGCCTGGGAAGAAGTGGGAC |
| | CTCAGATtTGGTACTTCTTCGACAATAGCACCCAGATGAACATGATCAGAGACAC |
| | CCTGGGCAACCCTACCGTGAAGGACTTCCTGAACAGACAGCTGGGCGAAGAGGGC |
| | ATTACCGCCGAGGCCATCCTGAACTTTCTGTACAAGGGCCCCAGAGAGTCCCAGG |
| | CCGACGACATGGCCAACTTCGATTGGCGGGACATCTTCAACATCACCGACAGAAC |
| | CCTGCGGCTGGTCAACCAGTACCTGGAATGCCTGGTGCTGGACAAGTTCGAGAGC |
| | TACAACGACGAGACACAGCTGACCCAGAGAGCCCTGTCTCTGCTGGAAGAGAATA |
| | TGTTCTGGGCTGGCGTGGTGTTCCCCGACATGTACCCTTGGACAAGCAGCCTGCC |
| | TCCTCACGTGAAGTACAAGATCCGGATGGACATCGACGTGGTCGAAAAGACCAAC |
| | AAGATCAAGGACCGGTACTGGGACAGCGGCCCTAGAGCTGATCCCGTGGAAGATT |
| | TTCGGTACATCTGGGGCGGATTCGCATACCTGCAGGACATGGTGGAACAGGGAAT |
| | CACACGGTCCCAGGTGCAGGCTGAAGCTCCTGTGGGAATCTACCTGCAGCAGATG |
| | CCTTATCCTTGCTTCGTGGACGACAGCTTCATGATCATCCTGAATCGGTGCTTCC |
| | CCATCTTCATGGTGCTGGCCTGGATCTACTCCGTGTCTATGACCGTGAAGTCCAT |
| | CGTGCTGGAAAAAGAGCTGCGGCTGAAAGAGACACTGAAGAACCAGGGCGTGTCC |
| | AATGCCGTGATCTGGTGCACCTGGTTTCTGGACAGCTTCTCCATTATGAGCATGA |
| | GCATCTTTCTGCTGACGATCTTCATCATGCACGGCCGaATCCTGCACTACAGCGA |
| | CCCCTTTATCCTCTTCCTGTTCCTGCTGGCCTTCAGCACCGCTACAATCATGCTG |
| | TGTTTTCTGCTGTCCACCTTCTTCAGCAAGGCCTCTCTGGCCGCTGCTTGTAGCG |
| | GCGTGATCTACTTCACCCTGTACCTGCCTCACATCCTGTGCTTCGCATGGCAGGA |
| | CAGAATGACCGCCGAGCTGAAGAAAGCTGTGTCCCTGCTGAGCCCTGTGGCCTTT |
| | GGCTTTGGCACCGAGTACCTCGTCAGATTTGAGGAACAAGGACTGGGACTGCAGT |
| | GGTCCAACATCGGCAATAGCCCTACAGAGGGCGACGAGTTCAGCTTCCTGCTGTC |
| | TATGCAGATGATGCTGCTGGACGCCGCCGTGTATGGACTGCTGGCTTGGTATCTG |
| | GACCAGGTGTTCCCAGGCGATTACGGCACTCCTCTGCCTTGGTATTTCCTGCTGC |
| | AAGAGAGCTACTGGCTCGGCGGCGAGGGATGTAGCACCAGAGAAGAAAGAGCCCT |
| | GGAAAAGACCGAGCCTCTGACCGAGGAAACAGAGGACCCTGAACACCCAGAGGGC |
| | ATCCACGATAGCTTTTTCGAGAGAGAACACCCCGGCTGGGTGCCAGGCGTGTGTG |
| | TGAAGAATCTGGTCAAGATtTTCGAGCCCTGCGGCAGACCTGCCGTGGACAGACT |
| | GAACATCACCTTCTACGAGAACCAGATTACCGCCTTTCTGGGCCACAACGGCGCT |
| | GGCAAGACAACCACAtTGAGCATCCTCACAGGCCTGCTGCCTCCAACAAGCGGCA |
| | CAGTTCTCGTTGGCGGCAGAGACATCGAGACAAGCCTGGATGCCGTCAGACAGTC |
| | CCTGGGCATGTGCCCTCAGCACAACATCCTGTTTCACCACCTGACCGTGGCCGAG |
| | CACATGCTGTTTTATGCCCAGCTGAAGGGCAAGAGCCAAGAAGAGGCTCAGCTGG |
| | AAATGGAAGCCATGtTgGAGGACACCGGCCTGCACCACAAGAGAAATGAGGAAGC |
| | CCAGGATCTGAGCGGCGGCATGCAGAGAAAACTGAGCGTGGCCATTGCCTTCGTG |
| | GGCGACGCCAAGGTTGTGATCCTGGATGAGCCTACAAGCGGCGTGGACCCTTACA |
| | GCAGAAGATCCATCTGGGATCTGCTGCTGAAGTACAGAtcaGGCCGGACCATCAT |
| | CATGAGCACCCACCACATGGACGAGGCCGATCTGCTCGGAGACAGAATCGCCATC |
| | ATTGCTCAGGGCAGACTGTACTGCAGCGGCACCCCACTGTTTCTGAAGAACTGTT |
| | TCGGCACCGGACTGTATCTGACCCTCGTGCGGAAGATGAAGAACATCCAGTCTCA |
| | GCGGAAGGGCAGCGAGGGCACCTGTAGCTGTTCTAGCAAGGGCTTTAGCACCACC |
| | TGTCCAGCTCACGTGGACGATCTGACCCCTGAACAGGTGCTGGATGGCGACGTGA |
| | ACGAGCTGATGGACGTGGTGCTGCACCATGTGCCTGAGGCCAAGCTGGTGGAATG |
| | CATCGGCCAAGAACTGATTTTTCTGCTCCCGAACAAGAACTTCAAGCACCGGGCC |
| | TACGCCAGCCTGTTCAGAGAGCTGGAAGAAACCCTGGCCGACCTGGGCCTGTCTA |
| | GCTTTGGCATCAGCGACACCCCTCTCGAAGAGATtTTCCTGAAAGTGACAGAGGA |
| | CAGCGATAGCGGCCCTCTGTTTGCTGGCGGAGCACAGCAAAAGCGCGAGAACGTG |
| | AACCCTAGACACCCCTGTCTGGGCCCAAGAGAGAAAGCCGGACAGACCCCTCAGG |
| | ACAGCAATGTGTGCTCTCCTGGTGCTCCTGCCGCTCATCCTGAGGGACAACCTCC |
| | ACCTGAACCTGAGTGTCCTGGACCTCAGCTGAACACCGGAACACAGCTGGTTCTG |
| | CAGCACGTGCAGGCTCTGCTCGTGAAGAGATTCCAGCACACCATCAGAAGCCACA |
| | AGGACTTTCTGGCCCAGATCGTGCTGCCCGCCACCTTTGTTTTTCTGGCTCTGAT |
| | GCTGAGCATCGTGATCCCTCCATTCGGCGAGTACCCCGCTCTGACACTGCACCCT |
| | TGGATCTACGGCCAGCAGTACACCTTTTTCTCCATGGACGAACCCGGCAGCGAGC |
| | AGTTCACAGTGCTGGCTGATGTCCTGCTGAACAAGCCCGGCTTCGGCAACCGGTG |
| | TCTGAAAGAAGGATGGCTGCCTGAGTACCCTTGCGGCAACAGCACACCTTGGAAA |
| | ACCCCTAGCGTGTCCCCTAACATCACCCAGCTGTTCCAAAAGCAGAAATGGACCC |
| | AAGTGAACCCCTCTCCATCCTGCCGGTGCTCCACAAGGGAAAAGCTGACCATGCT |
| | GCCCGAGTGTCCAGAAGGCGCTGGCGGACTTCCTCCACCTCAGAGAACACAGAGA |
| | TCCACCGAGATTCTCCAGGACCTGACCGACCGGAATATCAGCGACTTCCTGGTTA |
| | AGACATACCCCGCACTGATCCGGTCCAGCCTGAAGTCCAAGTTCTGGGTCAACGA |
| | ACAGAGATACGGCGGCATCAGCATCGGCGGAAAACTGCCTGTGGTGCCTATCACA |
| | GGCGAGGCCCTTGTGGGCTTTCTGTCCGATCTGGGGAGAATCATGAACGTGTCCG |
| | GCGGACCTATCACCAGGGAAGCCAGCAAAGAGATCCCCGATTTCCTGAAGCACCT |
| | GGAAACCGAGGACAATATCAAAGTGTGGTTCAACAACAAAGGATGGCACGCCCTC |
| | GTGTCTTTTCTGAACGTGGCCCACAATGCCATCCTGCGGGCTAGCCTGCCTAAGG |
| | ACAGAAGCCCTGAGGAATACGGCATCACCGTGATCTCCCAGCCTCTGAATCTGAC |
| | CAAAGAGCAGCTGAGCGAGATCACCGTGCTGACCACCTCTGTGGATGCTGTGGTG |
| | GCCATCTGCGTGATCTTCAGCATGAGCTTCGTGCCCGCCTCCTTCGTGCTGTACC |
| | TGATTCAAGAGAGAGTGAACAAGAGCAAGCACCTCCAGTTCATCTCCGGGGTGTC |
| | CCCAACCACCTACTGGGTCACCAATTTTCTGTGGGACATCATGAACTACAGCGTG |
| | TCAGCCGGCCTGGTCGTGGGCATCTTTATCGGCTTTCAGAAGAAGGCCTACACGA |
| | GCCCCGAGAACCTGCCTGCTTTGGTTGCTCTGCTGCTCCTGTATGGCTGGGCCGT |
| | GATTCCCATGATGTACCCCGCCAGCTTTCTGTTTGACGTGCCCAGCACAGCCTAC |
| | GTGGCCCTGTCTTGCGCCAATCTGTTCATCGGCATCAACAGCAGCGCCATCACAT |
| | TCATCCTGGAACTGTTCGAGAACAACAGGACCCTGCTGCGGTTCAACGCCGTGCT |
| | GCGGAAACTGCTGATCGTGTTCCCTCACTTCTGTCTCGGCAGAGGCCTGATCGAC |
| | CTGGCTCTGTCTCAGGCCGTGACCGATGTGTACGCCAGATTTGGCGAGGAACACT |
| | CCGCCAATCCATTCCACTGGGACCTGATCGGCAAGAACCTGTTCGCCATGGTGGT |
| | GGAAGGCGTCGTGTACTTCCTGCTCACTCTGCTGGTGCAGAGACACTTTTTTCTG |
| | TCCCAATGGATCGCCGAGCCTACCAAAGAACCCATTGTGGACGAGGACGACGATG |
| | TGGCCGAGGAAAGACAGAGAATCATCACCGGCGGCAACAAGACCGATATCCTGAG |
| | ACTGCACGAGCTGACAAAGATTACCCCGGCACAAGCTCCCCAGCCGTGGATAGGC |
| | TTTGTGTGGGAGTTAGACCCGGCGAGTGCTTTGGCCTGCTGGGAGTTAATGGCGC |
| | CGGAAAGACCACCACCTTCAAGATGCTGACCGGCGACACCACAGTGACAAGCGGA |
| | GATGCTACAGTGGCCGGCAAGAGCATCCTGACCAACATCAGCGAAGTGCATCAGA |
| | ACATGGGCTACTGCCCTCAGTTCGACGCCATCGACGAACTGCTGACAGGCCGCGA |
| | ACACCTGTATCTGTATGCCAGACTGAGAGGCGTGCCCGCTGAAGAGATCGAGAAG |
| | GTGGCCAACTGGTCCATCAAGTCTCTGGGCCTGACAGTGTACGCCGACTGTCTGG |
| | CCGGAACATACAGCGGAGGAAACAAGCGGAAGCTGAGCACCGCCATTGCTCTGAT |
| | CGGATGCCCACCTCTGGTCCTGCTGGATGAACCCACCACCGGAATGGAcCCCCAG |
| | GCTAGAAGAATGCTCTGGAACGTGATCGTGTCTATCATCCGCGAGGGCAGAGCTG |
| | TGGTGCTGACCTCTCACAGCATGGAAGAGTGCGAGGCTCTGTGTACCCGGCTGGC |
| | CATTATGGTCAAGGGCGCCTTCAGATGCATGGGCACCATTCAGCATCTGAAAAGC |
| | AAGTTCGGCGACGGCTACATCGTGACAATGAAGATCAAGAGCCCCAAGGACGACC |
| | TCCTGCCTGATCTGAACCCCGTGGAACAGTTTTTTCAGGGCAACTTCCCCGGCTC |
| | CGTGCAGCGGGAAAGACACTATAACATGCTGCAGTTTCAGGTGTCCTCCTCCAGC |
| | CTGGCTCGGATCTTTCAACTGCTGCTCTCTCACAAGGACAGCCTGCTGATTGAAG |
| | AGTACAGCGTGACACAGACCACACTCGACCAGGTTTTCGTGAACTTCGCCAAGCA |
| | GCAGACCGAGAGCCACGACCTGCCTCTGCATCCTAGAGCCGCTGGTGCCTCTAGA |
| | CAAGCTCAGGACGGCGGAGGCAGCTACCCTTATGATGTGCCTGATTACGCCTAAG |
| | CTTAGACCCTCCGATCCTCTGAAAGGAGCATCAGGCAGGCAGGGGTTGCTCCGGG |
| | GCCAGGGAAGGGGCCCCGCTGGGACGCGGCTGTTATTGCAGCTGGTTGGCGCGCA |
| | GCCATGCTTAGCTGCAGTGCGGGAATGCTGGGCCTTCTGTTCTGGGCTGTTTCTC |
| | ATACGCACGTAGGCCAGTGTATAAATAAGGTTTTATTAAATGCCAAATGAGTTCT |
| | CATTAACAAAGAAAGAGGGAAAATCTCAGTAAACCACCGTGACGGCATCTACCCA |
| | CTTTGAGTCAGGAGCTGGGGGTGTGAGTGCAACCTCCGAGACAAGGGAACCTGTG |
| | GAGCCCAGAGAATCGGAGGGGGGCGCTGGGGTTAGCACCGACTGAACCAGCTGTG |
| | TTTTCTCTCGGTTCCTTGGAGATCAGAAGTGAGTGTTGTCATCTTCAAACAATCC |
| | AAAGGCAGTACCCATGGCCTTACTACATCCCTCCCACACCATCCCACCCATCCCC |
| | GCGCGTACACTCACACGCTCATTTGCACACTATCGCACACGCTCACTTGCGTGCG |
| | CACACACAGATTGGTGACCTAGGTGGACTGGGAGAGAAATAAGAGCCAAATGACT |
| | GGATTTTCTCCAAGGAAATTTATTAATAGCCCCTCTTGGTTTCACCTGAAGGAGC |
| | TTGTCTTCACCTGCGGCCTTTGCAGGCTTAACGCCCCCAGCTTGAAACCCAGAAG |
| | CTCAGACTTGGGCCCAAGGTATTATTAGTGCCAACACTACCTGAAATGTTTCGCA |
| | CCTCATAAAAATGGTGTGTCAGTTTCGGGTGAGAGGTTGGGACGCTTCCCATCTG |
| | ATTTGGCCCAAGGCATGCATGCCCCTCCTTCTCCTTCCCCTCCTCCTCCCCCTCT |
| | TCCCCCTACCATCCTTCCTGTTTTCTCTCCAACTCTGGTGCACAGCTTTGAAATC |
| | TTGCTGAGAAGCAAATCTGTCCCTTCTGCTTTGAATGTTTATTTGTGGAAGTTCG |
| | GCAGGGGAACCGAGGCGGGTGCCAAGACCTGCCATGCTGCTGGGAAGTCTGAGTC |
| | TCCCTCCCTTCCCCCTCCTAAATGCTTGTTGATAGAGAAAAGTCAGCCTCCTCGG |
| | CATTTGGGCTCACGGTTTTCCTTTGAAAATGCTTCCAGTGTGGCATGATTCAGCT |
| | TTCTTTTCTGTCCCCCAACCACTGCTCTGTTGTCATTTTTACTTTTCTGATTGCA |
| | TTTTATCCGTGTCTCTTTGACTACGGGGTGGCTGGACGTTGAGTTCCAGGAAGAA |
| | AAGGGCCCAATCTTGGGGTTCTGACTACATGCGCCCATCAATGTCCTGTTTCATT |
| | CTTGGCTCTGGCTCCCTGAATTCCTGAGTCACTGGGGAGAAGCGTGGGTGGACCG |
| | CCCCCTACCCAGTGAGAGTTGCCACAGTTGCTGCTCTCCTGGGTCATTGGTTGCA |
| | GATTGTTAAACTTCACCTATGCATTTCAACTTTCGGGTGGATATTGCTACGTCAA |
| | GTGTCTGGGAAAGCCCCCACAGCTACAGGATTTTACAGTGAGGTCCCACTAATGA |
| | CTTGATGTCATGACTTCCTCATTCTTTCCAATTTCTCCCACTTCTCCATAAGGGT |
| | TTTGGGAAGGGGAGAAGAGAAAGGAGTGATTCCTGAGTGCCAGTACCAGGGAACA |
| | GCAGGGCTGTTGGGAGGATAAAATAAAATATCTTTATTTTCATTACATCTGTGTG |
| | TTGGTTTTTTGTGTGCGCTACTtgaagagcgaattctctaggatctgccaggaac |
| | cgtaaaaaGCGCTAGCGGAGTGTATACTGGCTTACTATGTTGGCACTGATGAGGG |
| | TGTCAGTGAAGTGCTTCATGTGGCAGGAGAAAAAAGGCTGCACCGGTGCGTCAGC |
| | AGAATATGTGATACAGGATATATTCCGCTTCCTCGCTCACTGACTCGCTACGCTC |
| | GGTCGTTCGACTGCGGCGAGCGGAAATGGCTTACGAACGGGGCGGAGATTTCCTG |
| | GAAGATGCCAGGAAGATACTTAACAGGGAAGTGAGAGGGCCGCGGCAAAGCCGTT |
| | TTTCCATAGGCTCCGCCCCCCTGACAAGCATCACGAAATCTGACGCTCAAATCAG |
| | TGGTGGCGAAACCCGACAGGACTATAAAGATACCAGGCGTTTCCCCCTGGCGGCT |
| | CCCTCGTGCGCTCTCCTGTTCCTGCCTTTCGGTTTACCGGTGTCATTCCGCTGTT |
| | ATGGCCGCGTTTGTCTCATTCCACGCCTGACACTCAGTTCCGGGTAGGCAGTTCG |
| | CTCCAAGCTGGACTGTATGCACGAACCCCCCGTTCAGTCCGACCGCTGCGCCTTA |
| | TCCGGTAACTATCGTCTTGAGTCCAACCCGGAAAGACATGCAAAAGCACCACTGG |
| | CAGCAGCCACTGGTAATTGATTTAGAGGAGTTAGTCTTGAAGTCATGCGCCGGTT |
| | AAGGCTAAACTGAAAGGACAAGTTTTGGTGACTGCGCTCCTCCAAGCCAGTTACC |
| | TCGGTTCAAAGAGTTGGTAGCTCAGAGAACCTTCGAAAAACCGCCCTGCAAGGCG |
| | GTTTTTTCGTTTTCAGAGCAAGAGATTACGCGCAGACCAAAACGATCTCAAGAAG |
| | ATCATCTTATTAATCAGATAAAATATTTCTAGATTTCAGTGCAATTTATCTCTTC |
| | AAATGTAGCACCTGAAGTCAGCCCCATACGATATAAGTTGTgattatcaaaaagg |
| | atottcacctagatccttttaaattaaaaatgaagttttaaatcaatctaaagta |
| | tatatgagtaaacttggtctgacagttaccaatgcttaatcagtgaggcacctat |
| | ctcagcgatctgtctatttcgttcatccatagttgcctgactccccgtcgtgtag |
| | ataactacgatacgggagggcttaccatctggccccagtgctgcaatgataccgc |
| | gagaaccacgctcaccggctccagatttatcagcaataaaccagccagccggaag |
| | ggccgagcgcagaagtggtcctgcaactttatccgcctccatccagtctattaat |
| | tgttgccgggaagctagagtaagtagttcgccagttaatagtttgcgcaacgttg |
| | ttgccattgctacaggcatcgtggtgtcacgctcgtcgtttggtatggcttcatt |
| | cagctccggttcccaacgatcaaggcgagttacatgatcccccatgttgtgcaaa |
| | aaagcggttagctccttcggtcctccgatcgttgtcagaagtaagttggccgcag |
| | tgttatcactcatggttatggcagcactgcataattctcttactgtcatgccatc |
| | cgtaagatgcttttctgtgactggtgagtactcaaccaagtcattctgagaatag |
| | tgtatgcggcgaccgagttgctcttgcccggcgtcaatacgggataataccgcgc |
| | cacatagcagaactttaaaagtgctcatcattggaaaacgttcttcggggcgaaa |
| | actctcaaggatcttaccgctgttgagatccagttcgatgtaacccactcgtgca |
| | cccaactgatcttcagcatcttttactttcaccagcgtttctgggtgagcaaaaa |
| | caggaaggcaaaatgccgcaaaaaagggaataagggcgacacggaaatgttgaat |
| | actcatactcttcctttttcaatattattgaagcatttatcagggttattgtctc |
| | atgagcggatacatatttgaatgtatttagaaaaataaacaaataggggttccgc |
| | gcacgaattcgctcttcaCTC |
| 39 | TTCGgtatcacaggagaatttcagGGAGAGGGATCACTTCAGATCTGCCGAGTGA |
| | GTCGATTGGACTTAAAGGGCCAGTCAAACCCTGACTGCCGGCTCATGGCAGGCTC |
| | TTGCCGAGGACAAATGCCCAGCCTATATTTATGCAAAGAGATTTTGTTCCAAACT |
| | TAAGGTCAAAGATACCTAAAGACATCCCCCTCAGGAACCCCTCTCATGGAGGAGA |
| | GTGCCTGAGGGTCTTGGTTTCCCATTGCATCCCCCACCTCAATTTCCCTGGTGCC |
| | CAGCCACTTGTGTCTTTAGGGTTCTCTTTCTCTCCATAAAAGGGAGCCAACACAG |
| | TGTCGGCCTCCTCTCCCCAACTAAGGGCTTATGTGTAATTAAAAGGGATTATGCT |
| | TTGAAGGGGAAAAGTAGCCTTTAATCACCAGGAGAAGGACACAGCGTCCGGAGCC |
| | AGAGGCGCTCTTAACGGCGTTTATGTCCTTTGCTGTCTGAGGGGCCTCAGCTCTG |
| | ACCAATCTGGTCTTCGTGTGGTCATTAGCAGATAAGTTTGTACAAAAAAGAGAGG |
| | TGCCACCATGGGCTTTGTGCGACAGATTCAGCTGCTGCTGTGGAAGAACTGGACC |
| | CTGCGGAAGCGGCAGAAAATCAGATTCGTGGTGGAACTCGTGTGGCCCCTGAGCC |
| | TGTTTCTGGTGCTGATCTGGCTGCGGAACGCCAATCCTCTGTACAGCCACCACGA |
| | GTGTCACTTCCCCAACAAGGCCATGCCTTCTGCCGGAATGCTGCCTTGGCTGCAG |
| | GGCATCTTCTGCAACGTGAACAACCCCTGCTTTCAGAGCCCCACACCTGGCGAAA |
| | GCCCTGGCATCGTGTCCAACTACAACAACAGCATCCTGGCCAGAGTGTACCGGGA |
| | CTTCCAAGAGCTGCTGATGAACGCCCCTGAGTCTCAGCACCTGGGCAGAATCTGG |
| | ACCGAGCTGCACATCCTGAGCCAGTTCATGGACACCCTGAGAACACACCCCGAGA |
| | GAATCGCCGGCAGGGGCATCAGAATCCGGGACATCCTGAAGGACGAGGAAACCCT |
| | GACACTGTTCCTCATCAAGAACATCGGCCTGAGCGACAGCGTGGTGTACCTGCTG |
| | ATCAACAGCCAAGTGCGGCCCGAGCAGTTTGCTCATGGCGTGCCgGATCTCGCCC |
| | TGAAGGATATCGCCTGTTCTGAGGCCCTGCTGGAACGGTTCATCATCTTCAGCCA |
| | GCGGAGAGGCGCCAAGACCGTCAGATATGCCCTGTGCAGTCTGAGCCAGGGAACC |
| | CTGCAGTGGATCGAGGATACCCTGTACGCCAACGTGGACTTCTTCAAGCTGTTCC |
| | GGGTGCTGCCCACACTGCTGGATTCTAGATCCCAGGGCATCAACCTGAGAAGCTG |
| | GGGCGGCATCCTGTCCGACATGAGCCCAAGAATCCAAGAGTTCATCCACCGGCCT |
| | AGCATGCAGGACCTGCTGTGGGTTACCAGACCTCTGATGCAGAACGGCGGACCCG |
| | AGACATTCACCAAGCTGATGGGCATTCTGAGCGATCTGCTGTGCGGCTACCCTGA |
| | AGGCGGAGGATCTAGAGTGCTGAGCTTCAATTGGTACGAGGACAACAACTACAAG |
| | GCCTTCCTGGGCATCGACTCCACCAGAAAGGACCCCATCTACAGCTACGACCGGC |
| | GGACAACCAGCTTCTGCAATGCCCTGATCCAGAGCCTGGAAAGCAACCCTCTGAC |
| | CAAGATCGCTTGGAGGGCCGCCAAACCTCTGCTGATGGGAAAGATCCTGTACACC |
| | CCTGACAGCCCTGCCGCCAGAAGAATCCTGAAGAACGCCAACAGCACCTTCGAGG |
| | AACTGGAACACGTGCGCAAGCTGGTCAAGGCCTGGGAAGAAGTGGGACCTCAGAT |
| | tTGGTACTTCTTCGACAATAGCACCCAGATGAACATGATCAGAGACACCCTGGGC |
| | AACCCTACCGTGAAGGACTTCCTGAACAGACAGCTGGGCGAAGAGGGCATTACCG |
| | CCGAGGCCATCCTGAACTTTCTGTACAAGGGCCCCAGAGAGTCCCAGGCCGACGA |
| | CATGGCCAACTTCGATTGGCGGGACATCTTCAACATCACCGACAGAACCCTGCGG |
| | CTGGTCAACCAGTACCTGGAATGCCTGGTGCTGGACAAGTTCGAGAGCTACAACG |
| | ACGAGACACAGCTGACCCAGAGAGCCCTGTCTCTGCTGGAAGAGAATATGTTCTG |
| | GGCTGGCGTGGTGTTCCCCGACATGTACCCTTGGACAAGCAGCCTGCCTCCTCAC |
| | GTGAAGTACAAGATCCGGATGGACATCGACGTGGTCGAAAAGACCAACAAGATCA |
| | AGGACCGGTACTGGGACAGCGGCCCTAGAGCTGATCCCGTGGAAGATTTTCGGTA |
| | CATCTGGGGCGGATTCGCATACCTGCAGGACATGGTGGAACAGGGAATCACACGG |
| | TCCCAGGTGCAGGCTGAAGCTCCTGTGGGAATCTACCTGCAGCAGATGCCTTATC |
| | CTTGCTTCGTGGACGACAGCTTCATGATCATCCTGAATCGGTGCTTCCCCATCTT |
| | CATGGTGCTGGCCTGGATCTACTCCGTGTCTATGACCGTGAAGTCCATCGTGCTG |
| | GAAAAAGAGCTGCGGCTGAAAGAGACACTGAAGAACCAGGGCGTGTCCAATGCCG |
| | TGATCTGGTGCACCTGGTTTCTGGACAGCTTCTCCATTATGAGCATGAGCATCTT |
| | TCTGCTGACGATCTTCATCATGCACGGCCGaATCCTGCACTACAGCGACCCCTTT |
| | ATCCTCTTCCTGTTCCTGCTGGCCTTCAGCACCGCTACAATCATGCTGTGTTTTC |
| | TGCTGTCCACCTTCTTCAGCAAGGCCTCTCTGGCCGCTGCTTGTAGCGGCGTGAT |
| | CTACTTCACCCTGTACCTGCCTCACATCCTGTGCTTCGCATGGCAGGACAGAATG |
| | ACCGCCGAGCTGAAGAAAGCTGTGTCCCTGCTGAGCCCTGTGGCCTTTGGCTTTG |
| | GCACCGAGTACCTCGTCAGATTTGAGGAACAAGGACTGGGACTGCAGTGGTCCAA |
| | CATCGGCAATAGCCCTACAGAGGGCGACGAGTTCAGCTTCCTGCTGTCTATGCAG |
| | ATGATGCTGCTGGACGCCGCCGTGTATGGACTGCTGGCTTGGTATCTGGACCAGG |
| | TGTTCCCAGGCGATTACGGCACTCCTCTGCCTTGGTATTTCCTGCTGCAAGAGAG |
| | CTACTGGCTCGGCGGCGAGGGATGTAGCACCAGAGAAGAAAGAGCCCTGGAAAAG |
| | ACCGAGCCTCTGACCGAGGAAACAGAGGACCCTGAACACCCAGAGGGCATCCACG |
| | ATAGCTTTTTCGAGAGAGAACACCCCGGCTGGGTGCCAGGCGTGTGTGTGAAGAA |
| | TCTGGTCAAGATtTTCGAGCCCTGCGGCAGACCTGCCGTGGACAGACTGAACATC |
| | ACCTTCTACGAGAACCAGATTACCGCCTTTCTGGGCCACAACGGCGCTGGCAAGA |
| | CAACCACAtTGAGCATCCTCACAGGCCTGCTGCCTCCAACAAGCGGCACAGTTCT |
| | CGTTGGCGGCAGAGACATCGAGACAAGCCTGGATGCCGTCAGACAGTCCCTGGGC |
| | ATGTGCCCTCAGCACAACATCCTGTTTCACCACCTGACCGTGGCCGAGCACATGC |
| | TGTTTTATGCCCAGCTGAAGGGCAAGAGCCAAGAAGAGGCTCAGCTGGAAATGGA |
| | AGCCATGtTqGAGGACACCGGCCTGCACCACAAGAGAAATGAGGAAGCCCAGGAT |
| | CTGAGCGGCGGCATGCAGAGAAAACTGAGCGTGGCCATTGCCTTCGTGGGCGACG |
| | CCAAGGTTGTGATCCTGGATGAGCCTACAAGCGGCGTGGACCCTTACAGCAGAAG |
| | ATCCATCTGGGATCTGCTGCTGAAGTACAGAtcaGGCCGGACCATCATCATGAGC |
| | ACCCACCACATGGACGAGGCCGATCTGCTCGGAGACAGAATCGCCATCATTGCTC |
| | AGGGCAGACTGTACTGCAGCGGCACCCCACTGTTTCTGAAGAACTGTTTCGGCAC |
| | CGGACTGTATCTGACCCTCGTGCGGAAGATGAAGAACATCCAGTCTCAGCGGAAG |
| | GGCAGCGAGGGCACCTGTAGCTGTTCTAGCAAGGGCTTTAGCACCACCTGTCCAG |
| | CTCACGTGGACGATCTGACCCCTGAACAGGTGCTGGATGGCGACGTGAACGAGCT |
| | GATGGACGTGGTGCTGCACCATGTGCCTGAGGCCAAGCTGGTGGAATGCATCGGC |
| | CAAGAACTGATTTTTCTGCTCCCGAACAAGAACTTCAAGCACCGGGCCTACGCCA |
| | GCCTGTTCAGAGAGCTGGAAGAAACCCTGGCCGACCTGGGCCTGTCTAGCTTTGG |
| | CATCAGCGACACCCCTCTCGAAGAGATtTTCCTGAAAGTGACAGAGGACAGCGAT |
| | AGCGGCCCTCTGTTTGCTGGCGGAGCACAGCAAAAGCGCGAGAACGTGAACCCTA |
| | GACACCCCTGTCTGGGCCCAAGAGAGAAAGCCGGACAGACCCCTCAGGACAGCAA |
| | TGTGTGCTCTCCTGGTGCTCCTGCCGCTCATCCTGAGGGACAACCTCCACCTGAA |
| | CCTGAGTGTCCTGGACCTCAGCTGAACACCGGAACACAGCTGGTTCTGCAGCACG |
| | TGCAGGCTCTGCTCGTGAAGAGATTCCAGCACACCATCAGAAGCCACAAGGACTT |
| | TCTGGCCCAGATCGTGCTGCCCGCCACCTTTGTTTTTCTGGCTCTGATGCTGAGC |
| | ATCGTGATCCCTCCATTCGGCGAGTACCCCGCTCTGACACTGCACCCTTGGATCT |
| | ACGGCCAGCAGTACACCTTTTTCTCCATGGACGAACCCGGCAGCGAGCAGTTCAC |
| | AGTGCTGGCTGATGTCCTGCTGAACAAGCCCGGCTTCGGCAACCGGTGTCTGAAA |
| | GAAGGATGGCTGCCTGAGTACCCTTGCGGCAACAGCACACCTTGGAAAACCCCTA |
| | GCGTGTCCCCTAACATCACCCAGCTGTTCCAAAAGCAGAAATGGACCCAAGTGAA |
| | CCCCTCTCCATCCTGCCGGTGCTCCACAAGGGAAAAGCTGACCATGCTGCCCGAG |
| | TGTCCAGAAGGCGCTGGCGGACTTCCTCCACCTCAGAGAACACAGAGATCCACCG |
| | AGATTCTCCAGGACCTGACCGACCGGAATATCAGCGACTTCCTGGTTAAGACATA |
| | CCCCGCACTGATCCGGTCCAGCCTGAAGTCCAAGTTCTGGGTCAACGAACAGAGA |
| | TACGGCGGCATCAGCATCGGCGGAAAACTGCCTGTGGTGCCTATCACAGGCGAGG |
| | CCCTTGTGGGCTTTCTGTCCGATCTGGGGAGAATCATGAACGTGTCCGGCGGACC |
| | TATCACCAGGGAAGCCAGCAAAGAGATCCCCGATTTCCTGAAGCACCTGGAAACC |
| | GAGGACAATATCAAAGTGTGGTTCAACAACAAAGGATGGCACGCCCTCGTGTCTT |
| | TTCTGAACGTGGCCCACAATGCCATCCTGCGGGCTAGCCTGCCTAAGGACAGAAG |
| | CCCTGAGGAATACGGCATCACCGTGATCTCCCAGCCTCTGAATCTGACCAAAGAG |
| | CAGCTGAGCGAGATCACCGTGCTGACCACCTCTGTGGATGCTGTGGTGGCCATCT |
| | GCGTGATCTTCAGCATGAGCTTCGTGCCCGCCTCCTTCGTGCTGTACCTGATTCA |
| | AGAGAGAGTGAACAAGAGCAAGCACCTCCAGTTCATCTCCGGGGTGTCCCCAACC |
| | ACCTACTGGGTCACCAATTTTCTGTGGGACATCATGAACTACAGCGTGTCAGCCG |
| | GCCTGGTCGTGGGCATCTTTATCGGCTTTCAGAAGAAGGCCTACACGAGCCCCGA |
| | GAACCTGCCTGCTTTGGTTGCTCTGCTGCTCCTGTATGGCTGGGCCGTGATTCCC |
| | ATGATGTACCCCGCCAGCTTTCTGTTTGACGTGCCCAGCACAGCCTACGTGGCCC |
| | TGTCTTGCGCCAATCTGTTCATCGGCATCAACAGCAGCGCCATCACATTCATCCT |
| | GGAACTGTTCGAGAACAACAGGACCCTGCTGCGGTTCAACGCCGTGCTGCGGAAA |
| | CTGCTGATCGTGTTCCCTCACTTCTGTCTCGGCAGAGGCCTGATCGACCTGGCTC |
| | TGTCTCAGGCCGTGACCGATGTGTACGCCAGATTTGGCGAGGAACACTCCGCCAA |
| | TCCATTCCACTGGGACCTGATCGGCAAGAACCTGTTCGCCATGGTGGTGGAAGGC |
| | GTCGTGTACTTCCTGCTCACTCTGCTGGTGCAGAGACACTTTTTTCTGTCCCAAT |
| | GGATCGCCGAGCCTACCAAAGAACCCATTGTGGACGAGGACGACGATGTGGCCGA |
| | GGAAAGACAGAGAATCATCACCGGCGGCAACAAGACCGATATCCTGAGACTGCAC |
| | GAGCTGACAAAGATtTACCCCGGCACAAGCTCCCCAGCCGTGGATAGGCTTTGTG |
| | TGGGAGTTAGACCCGGCGAGTGCTTTGGCCTGCTGGGAGTTAATGGCGCCGGAAA |
| | GACCACCACCTTCAAGATGCTGACCGGCGACACCACAGTGACAAGCGGAGATGCT |
| | ACAGTGGCCGGCAAGAGCATCCTGACCAACATCAGCGAAGTGCATCAGAACATGG |
| | GCTACTGCCCTCAGTTCGACGCCATCGACGAACTGCTGACAGGCCGCGAACACCT |
| | GTATCTGTATGCCAGACTGAGAGGCGTGCCCGCTGAAGAGATCGAGAAGGTGGCC |
| | AACTGGTCCATCAAGTCTCTGGGCCTGACAGTGTACGCCGACTGTCTGGCCGGAA |
| | CATACAGCGGAGGAAACAAGCGGAAGCTGAGCACCGCCATTGCTCTGATCGGATG |
| | CCCACCTCTGGTCCTGCTGGATGAACCCACCACCGGAATGGAcCCCCAGGCTAGA |
| | AGAATGCTCTGGAACGTGATCGTGTCTATCATCCGCGAGGGCAGAGCTGTGGTGC |
| | TGACCTCTCACAGCATGGAAGAGTGCGAGGCTCTGTGTACCCGGCTGGCCATTAT |
| | GGTCAAGGGCGCCTTCAGATGCATGGGCACCATTCAGCATCTGAAAAGCAAGTTC |
| | GGCGACGGCTACATCGTGACAATGAAGATCAAGAGCCCCAAGGACGACCTCCTGC |
| | CTGATCTGAACCCCGTGGAACAGTTTTTTCAGGGCAACTTCCCCGGCTCCGTGCA |
| | GCGGGAAAGACACTATAACATGCTGCAGTTTCAGGTGTCCTCCTCCAGCCTGGCT |
| | CGGATCTTTCAACTGCTGCTCTCTCACAAGGACAGCCTGCTGATTGAAGAGTACA |
| | GCGTGACACAGACCACACTCGACCAGGTTTTCGTGAACTTCGCCAAGCAGCAGAC |
| | CGAGAGCCACGACCTGCCTCTGCATCCTAGAGCCGCTGGTGCCTCTAGACAAGCT |
| | CAGGACGGCGGAGGCAGCTACCCTTATGATGTGCCTGATTACGCCTAAGCTTAGA |
| | CCCTCCGATCCTCTGAAAGGAGCATCAGGCAGGCAGGGGTTGCTCCGGGGCCAGG |
| | GAAGGGGCCCCGCTGGGACGCGGCTGTTATTGCAGCTGGTTGGCGCGCAGCCATG |
| | CTTAGCTGCAGTGCGGGAATGCTGGGCCTTCTGTTCTGGGCTGTTTCTCATACGC |
| | ACGTAGGCCAGTGTATAAATAAGGTTTTATTAAATGCCAAATGAGTTCTCATTAA |
| | CAAAGAAAGAGGGAAAATCTCAGTAAACCACCGTGACGGCATCTACCCACTTTGA |
| | GTCAGGAGCTGGGGGTGTGAGTGCAACCTCCGAGACAAGGGAACCTGTGGAGCCC |
| | AGAGAATCGGAGGGGGGCGCTGGGGTTAGCACCGACTGAACCAGCTGTGTTTTCT |
| | CTCGGTTCCTTGGAGATCAGAAGTGAGTGTTGTCATCTTCAAACAATCCAAAGGC |
| | AGTACCCATGGCCTTACTACATCCCTCCCACACCATCCCACCCATCCCCGCGCGT |
| | ACACTCACACGCTCATTTGCACACTATCGCACACGCTCACTTGCGTGCGCACACA |
| | CAGATTGGTGACCTAGGTGGACTGGGAGAGAAATAAGAGCCAAATGACTGGATTT |
| | TCTCCAAGGAAATTTATTAATAGCCCCTCTTGGTTTCACCTGAAGGAGCTTGTCT |
| | TCACCTGCGGCCTTTGCAGGCTTAACGCCCCCAGCTTGAAACCCAGAAGCTCAGA |
| | CTTGGGCCCAAGGTATTATTAGTGCCAACACTACCTGAAATGTTTCGCACCTCAT |
| | AAAAATGGTGTGTCAGTTTCGGGTGAGAGGTTGGGACGCTTCCCATCTGATTTGG |
| | CCCAAGGCATGCATGCCCCTCCTTCTCCTTCCCCTCCTCCTCCCCCTCTTCCCCC |
| | TACCATCCTTCCTGTTTTCTCTCCAACTCTGGTGCACAGCTTTGAAATCTTGCTG |
| | AGAAGCAAATCTGTCCCTTCTGCTTTGAATGTTTATTTGTGGAAGTTCGGCAGGG |
| | GAACCGAGGCGGGTGCCAAGACCTGCCATGCTGCTGGGAAGTCTGAGTCTCCCTC |
| | CCTTCCCCCTCCTAAATGCTTGTTGATAGAGAAAAGTCAGCCTCCTCGGCATTTG |
| | GGCTCACGGTTTTCCTTTGAAAATGCTTCCAGTGTGGCATGATTCAGCTTTCTTT |
| | TCTGTCCCCCAACCACTGCTCTGTTGTCATTTTTACTTTTCTGATTGCATTTTAT |
| | CCGTGTCTCTTTGACTACGGGGTGGCTGGACGTTGAGTTCCAGGAAGAAAAGGGC |
| | CCAATCTTGGGGTTCTGACTACATGCGCCCATCAATGTCCTGTTTCATTCTTGGC |
| | TCTGGCTCCCTGAATTCCTGAGTCACTGGGGAGAAGCGTGGGTGGACCGCCCCCT |
| | ACCCAGTGAGAGTTGCCACAGTTGCTGCTCTCCTGGGTCATTGGTTGCAGATTGT |
| | TAAACTTCACCTATGCATTTCAACTTTCGGGTGGATATTGCTACGTCAAGTGTCT |
| | GGGAAAGCCCCCACAGCTACAGGATTTTACAGTGAGGTCCCACTAATGACTTGAT |
| | GTCATGACTTCCTCATTCTTTCCAATTTCTCCCACTTCTCCATAAGGGTTTTGGG |
| | AAGGGGAGAAGAGAAAGGAGTGATTCCTGAGTGCCAGTACCAGGGAACAGCAGGG |
| | CTGTTGGGAGGATAAAATAAAATATCTTTATTTTCATTACATCTGTGTGTTGGTT |
| | TTTTGTGTGCGCTACTtgaagagcgaattctctaggatctgccaggaaccgtaaa |
| | aaGCGCTAGCGGAGTGTATACTGGCTTACTATGTTGGCACTGATGAGGGTGTCAG |
| | TGAAGTGCTTCATGTGGCAGGAGAAAAAAGGCTGCACCGGTGCGTCAGCAGAATA |
| | TGTGATACAGGATATATTCCGCTTCCTCGCTCACTGACTCGCTACGCTCGGTCGT |
| | TCGACTGCGGCGAGCGGAAATGGCTTACGAACGGGGCGGAGATTTCCTGGAAGAT |
| | GCCAGGAAGATACTTAACAGGGAAGTGAGAGGGCCGCGGCAAAGCCGTTTTTCCA |
| | TAGGCTCCGCCCCCCTGACAAGCATCACGAAATCTGACGCTCAAATCAGTGGTGG |
| | CGAAACCCGACAGGACTATAAAGATACCAGGCGTTTCCCCCTGGCGGCTCCCTCG |
| | TGCGCTCTCCTGTTCCTGCCTTTCGGTTTACCGGTGTCATTCCGCTGTTATGGCC |
| | GCGTTTGTCTCATTCCACGCCTGACACTCAGTTCCGGGTAGGCAGTTCGCTCCAA |
| | GCTGGACTGTATGCACGAACCCCCCGTTCAGTCCGACCGCTGCGCCTTATCCGGT |
| | AACTATCGTCTTGAGTCCAACCCGGAAAGACATGCAAAAGCACCACTGGCAGCAG |
| | CCACTGGTAATTGATTTAGAGGAGTTAGTCTTGAAGTCATGCGCCGGTTAAGGCT |
| | AAACTGAAAGGACAAGTTTTGGTGACTGCGCTCCTCCAAGCCAGTTACCTCGGTT |
| | CAAAGAGTTGGTAGCTCAGAGAACCTTCGAAAAACCGCCCTGCAAGGCGGTTTTT |
| | TCGTTTTCAGAGCAAGAGATTACGCGCAGACCAAAACGATCTCAAGAAGATCATC |
| | TTATTAATCAGATAAAATATTTCTAGATTTCAGTGCAATTTATCTCTTCAAATGT |
| | AGCACCTGAAGTCAGCCCCATACGATATAAGTTGTgattatcaaaaaggatcttc |
| | acctagatccttttaaattaaaaatgaagttttaaatcaatctaaagtatatatg |
| | agtaaacttggtctgacagttaccaatgcttaatcagtgaggcacctatctcagc |
| | gatctgtctatttcgttcatccatagttgcctgactccccgtcgtgtagataact |
| | acgatacgggagggcttaccatctggccccagtgctgcaatgataccgcgagaac |
| | cacgctcaccggctccagatttatcagcaataaaccagccagccggaagggccga |
| | gcgcagaagtggtcctgcaactttatccgcctccatccagtctattaattgttgc |
| | cgggaagctagagtaagtagttcgccagttaatagtttgcgcaacgttgttgcca |
| | ttgctacaggcatcgtggtgtcacgctcgtcgtttggtatggcttcattcagctc |
| | cggttcccaacgatcaaggcgagttacatgatcccccatgttgtgcaaaaaagcg |
| | gttagctccttcggtcctccgatcgttgtcagaagtaagttggccgcagtgttat |
| | cactcatggttatggcagcactgcataattctcttactgtcatgccatccgtaag |
| | atgcttttctgtgactggtgagtactcaaccaagtcattctgagaatagtgtatg |
| | cggcgaccgagttgctcttgcccggcgtcaatacgggataataccgcgccacata |
| | gcagaactttaaaagtgctcatcattggaaaacgttcttcggggcgaaaactctc |
| | aaggatcttaccgctgttgagatccagttcgatgtaacccactcgtgcacccaac |
| | tgatcttcagcatcttttactttcaccagcgtttctgggtgagcaaaaacaggaa |
| | ggcaaaatgccgcaaaaaagggaataagggcgacacggaaatgttgaatactcat |
| | actcttcctttttcaatattattgaagcatttatcagggttattgtctcatgagc |
| | ggatacatatttgaatgtatttagaaaaataaacaaataggggttccgcgcacga |
| | attcgctcttcaCTC |
| 12 | GTAAGGGAGGGGTTTGGCTGCTCGCCAATTGCAAGGTGATTCCTGGGGTAGCAGA |
| | GCCTCACGAATTGACCTTGGGGAGGGCGTGAGCCTGGTGTTCTGGACAATCCTTG |
| | CAAAAGCTCCAGGCTCCCAGGGCTCAAAAAATCACAACTGATAGTATTTCTAGAA |
| | CAGTGGCCCAGGGACCCAGAAGTCACTATGAGGTTCACCATTAGGTATGTGGCTG |
| | TGGCATGTTTGTGTCCACTCTAAATGTGGGGATAATCCCCTTTACCTCCTCTAAC |
| | AGAGTGGTAAAGGAAGGAGGAGGCCTGGTTTGACTCCCTGACCTGCTATTTCCTA |
| | GCCAGGTGATCATGGTAAGATATTGAACCTTTTCTGGTCCCAGTACTCATCTATA |
| | AAACAAATATAATACTTTACAGAGTGGTAGGAATTATACAAGAAAAGTATACGCA |
| | AAACATTTCATAAATTTTAATAAATGATGGCCCCATGCTTCTTCCTCTGGAAATG |
| | GTCTCAACCTCAATGGTTGGTGTTTCTAGAGAGAAAAAACGACAGAGAAAGTTTC |
| | ATAGTCTCAAAAATTTGGAAAGCCCTGATCTAGCTCAACCCTTTGTTCTAGAACT |
| | GCATCCCAGACAGACTGCTTGGGACCTGAAAATATCTCCTCCTTTGCTAGAAGGA |
| | TAAGATGAGAAGGAATTAGATAAAGGAGGTGTAGAGCAGAGGTTTTCACACTGCA |
| | AAGTGCATAAAAACCATCAGAGGGCCGGGCGCAGTGGCTCACGCCTGTAATCCCA |
| | GCACTTTGGGAGGCCGAGGCGGGCGGATCATGAGGTCAGGAGATAGAGACCATCC |
| | TGGCTAACACGGTGAAACCCCGTCTCTACTAAAAAACACACACACACAAAAATTA |
| | GCCAGGTGTGGTGGCGGGCGCCTGTAATCCCAGCTACTGAGGAGGCTGAGGCCGG |
| | AGAATGGCGTGAACCCGGGAGGCGGAGCTTGCAGTGAGCCGAGATTGCGCCACTG |
| | CACTCCAGCCTGGGTGACAGAGCAAGACTCCGTCTCAATAAAAACAAACAAACAA |
| | ACAAACCAAAAAAACCCATCAGAGAAGTTGGTAAAAGATGCAAGTGCTAAATCCC |
| | CACCCCCAATCACTGTGATTCAGAAGAACCAGGCCAGGCCCAGAATCTATCCTGT |
| | TACCTTAGGCGATTCTGATGAAGACCATTGTAGGCCACACTTTCAGAAACACTCA |
| | AAATTAGAATCCTTCAGAGAAGGTGGCATATATAATATTTCTAGCATGGAATTAT |
| | GTTTTTTTTCTTTTGCCTACATTTTAATTTCTAGAACTGTGTTGTAGGGAATGTC |
| | AGTCACTAAGAACTTGATTGAGGAACTGTGTTTTGTCTGTTTCATGACTGCTCTC |
| | TCAAGTCCCAGGAAACTCACTTTCAGCTTGTCTTAAAAAGCAAGCTGAAGGCTTT |
| | TAAAAATGAAGCAACATGAAATAAGACACCGCAGTTTCTGGCACGGTCCACGCTT |
| | AATCCCCTTCAATGTGTGACTTTCCGTGGAAAGTTACTCTACGATTTTCCCAGCT |
| | CGTCAGGGTGGGGCCCCAGAGTGAGTATGAAGGGTCAGAGCCTAGGGATGCCACC |
| | ATCAGTGAGAGCCCAGGACCCCAGAAAAGGTCTCTTGGCTCACCACACTGTAGGA |
| | AAAATAAAAAGCAATGTAGTCCAAATGTCTCTATCCAAAGTTTCAAAAAGAACTT |
| | GATTTTAGACACGCTCCTTGACTTGTTTTCAGAATCAGACAGAAGAGTGAGGCAA |
| | CAAAGGTCCCTTATTCCAGGCAGCTGAATACCAGCACAGCCAGGAGTCCAGTGCT |
| | GGTGTTTGCAGAGCCACCAGAGGCTCCCTCTCAGGTGTCCAGGGCCCGCATGCTT |
| | TGTAGAATGGGCAGAATGAGCAATGTCTGTGCACCTGGGCTTTGCAGGCAGGGCC |
| | TGGGTACCCAGGTTCGTGCAATCCTCTCGTCACCATGAAGGGAGCAGCATCATTC |
| | TTCCCTTCTTGAAGCACCTTGGCCACCAGTATAGGTAAATTTACCTCCCAGGACA |
| | TGACCATTGATTCTGGGATGTCAATGCCAGAGATAGTAGGGTAAATCGGCACCTG |
| | GGTAAAACTTTCCATTGGAGACTAGAACCAAAACTCAGGACACTGGCTTCCAAAT |
| | GTTTCTTTATCAGACAAGAAAGACCAAGTCTTTCCTTACGTCTTCACATGCTGCC |
| | TTGGCAAATGCTAGCATTCACAAACCCTGGGCTACCTTGACCTGTCACCCTTGCA |
| | GACCTCAGACGGGTCCTGGGGGCTTGCTTTCTCGGTTTCTGTATGCAGGCACTCA |
| | AACCTGCATCAGGCACCTGTGAAGGGCCGGGCACTGTGCTGAGGCCAAGGCTCCA |
| | AATGTGAACCTTCCACCCTCACTGAACTCACAGCCAGACCAGAGACAAGCAAACA |
| | GGACATTTCACAGCAGTGCAGCCTAGAAAGGGCCAACACCAGCAGCATTTGTCCC |
| | CCCGAGCGGTAGCTTTTAGAAGCTTCCCCAGTGATTCAATGTGTCCTACAAATGC |
| | CTGGCCCCCACTCCCAGAGATTCTGAGTCAGCTGGCCTAGGGTGCAGCCTTGACT |
| | TCACTGTGTTAAAAAGCTTCCCAGATAAGTCCAATGTCCGGCCAAGATTGAGAAT |
| | CACTGACCTAGAGTTTAATTTACCACCTCAGTCTCTATAGACCACGCATAATAAT |
| | AGTACCCCACACACCTCTGAGGGTCCAAAGAACTTTCATTTGATCACCCATGAGA |
| | CCACCGTGGTGTGGAGATGCTTTCTCTCTCCTGTTCTCTTAACAAAGCTGGTGAG |
| | CGACAGAGCCTGCAGTGGACCGGGAGATGGCCCAGAGGAGAAAGCTCTGCCGTAG |
| | TCGGCCTCAGTTAACCACGGAGCACCACCCCTACCTGCTCTCCTCTCACTCCTGC |
| | TTCCGTCTCGGTGGAGAAAGATCCAACCGAAGCAGGACACATCTAGTCTTCTGGT |
| | GCCTTTAAAATGTACTTTTCCATTTGACAAATGGATTACACTAAAAACAAAAATT |
| | TACAAAAAAAAAAAAAAAACCTGAAAGAAATTGCAGGCATTAAAATGGGACTTTG |
| | CCTTTATTGCTCCTGGGCCCATCCTATTTGGGTTTTTAGAAAAACAAGCCTGAGG |
| | CAGGCCCAGAAAGGCTCAGGGCAGACCCTCCGATCCTCTGAAAGGAGCATCAGGC |
| | AGGCAGGGGTTGCTCCGGGGCCAGGGAAGGGGCCCCGCTGGGACGCGGCTGTTAT |
| | TGCAGCTGGTTGGCGCGCAGCCATGCTTAGCTGCAGTGCGGGAATGCTGGGCCTT |
| | CTGTTCTGGGCTGTTTCTCATACGCACGTAGGCCAGTGTATAAATAAGGTTTTAT |
| | TAAATGCCAAATGAGTTCTCATTAACAAAGAAAGAGGGAAAATCTCAGTAAACCA |
| | CCGTGACGGCATCTACCCACTTTGAGTCAGGAGCTGGGGGTGTGAGTGCAACCTC |
| | CGAGACAAGGGAACCTGTGGAGCCCAGAGAATCGGAGGGGGGCGCTGGGGTTAGC |
| | ACCGACTGAGACCAGCTGTGTTTTCTCTCGGTTCCTTGGAGATCAGAAGTGAGTG |
| | TTGTCATCTTCAAACAATCCAAAGGCAGTACCCATGGCCTTACTACATCCCTCCC |
| | ACACCATCCCACCCATCCCCGCGCGTACACTCACACGCTCATTTGCACACTATCG |
| | CACACGCTCACTTGCGTGCGCACACACAGATTGGTGACCTAGGTGGACTGGGAGA |
| | GAAATAAGAGCCAAATGACTGGATTTTCTCCAAGGAAATTTATTAATAGCCCCTC |
| | TTGGTTTCACCTGAAGGAGCTTGTCTTCACCTGCGGCCTTTGCAGGCTTAACGCC |
| | CCCAGCTTGAAACCCAGAAGCTCAGACTTGGGCCCAAGGTATTATTAGTGCCAAC |
| | ACTACCTGAAATGTTTCGCACCTCATAAAAATGGTGTGTCAGTTTCGGGTGAGAG |
| | GTTGGGACGCTTCCCATCTGATTTGGCCCAAGGCATGCATGCCCCTCCTTCTCCT |
| | TCCCCTCCTCCTCCCCCTCTTCCCCCTACCATCCTTCCTGTTTTCTCTCCAACTC |
| | TGGTGCACAGCTTTGAAATCTTGCTGAGAAGCAAATCTGTCCCTTCTGCTTTGAA |
| | TGTTTATTTGTGGAAGTTCGGCAGGGGAACCGAGGCGGGTGCCAAGACCTGCCAT |
| | GCTGCTGGGAAGTCTGAGTCTCCCTCCCTTCCCCCTCCTAAATGCTTGTTGATAG |
| | AGAAAAGTCAGCCTCCTCGGCATTTGGGCTCACGGTTTTCCTTTGAAAATGCTTC |
| | CAGTGTGGCATGATTCAGCTTTCTTTTCTGTCCCCCAACCACTGCTCTGTTGTCA |
| | TTTTTACTTTTCTGATTGCATTTTATCCGTGTCTCTTTGACTACGGGGTGGCTGG |
| | ACGTTGAGTTCCAGGAAGAAAAGGGCCCAATCTTGGGGTTCTGACTACATGCGCC |
| | CATCAATGTCCTGTTTCATTCTTGGCTCTGGCTCCCTGAATTCCTGAGTCACTGG |
| | GGAGAAGCGTGGGTGGACCGCCCCCTACCCAGTGAGAGTTGCCACAGTTGCTGCT |
| | CTCCTGGGTCATTGGTTGCAGATTGTTAAACTTCACCTATGCATTTCAACTTTCG |
| | GGTGGATATTGCTACGTCAAGTGTCTGGGAAAGCCCCCACAGCTACAGGATTTTA |
| | CAGTGAGGTCCCACTAATGACTTGATGTCATGACTTCCTCATTCTTTCCAATTTC |
| | TCCCACTTCTCCATAAGGGTTTTGGGAAGGGGAGAAGAGAAAGGAGTGATTCCTG |
| | AGTGCCAGTACCAGGGAACAGCAGGGCTGTTGGGAGGAAACAAAACTAAATCAGG |
| | AAGGTTTTTGTTGTTGTTTTTGGGGGGTTTTATGAAAATATTCAAGCCACAGCAA |
| | ATATATTTGATTTATAGCATTAGTATTTTTTCTGCCTGCATCTACAAAAATCTTT |
| | ACCTATTACCATCAAAATATCCTCTGGGTGAATGGATTTCAACAAAGAAGAAATA |
| | AAAATGAAATAGAAGAGAGGCCCCTTCGTGCACATTGAGCCTACTGGCTGGATTG |
| | TCACTTGCCTGCCTTGATGTCTTTTCAGCTCCAGGCAGGCAGTAGGCCAGGGCTT |
| | ATTTTCATGACAGATCAGATGTTCTTTTATGGATTTACAAAGAAAGAAATACTGA |
| | GAAGTCAAAACTGAAGTCACTTAAGACAAGAGCAGGCCCCTGGGAAGGCTGCCAT |
| | TGAGGATAATGAGTCCTGGGGTCCTGGCCTTTGTTCAGTAAATACGCACTAGGCG |
| | CCTACAATGTGTGCACCAATGTGTGAGGCGTCAGGTTCTCTCCAGGGTCAGTTGG |
| | TTTTAAGAAAGGTTTTGGCTTCTGATATGTTTTATCTCTACAGAACAGTAGCTCT |
| | TAACCTTTCTTATGGGTTAGGATTACCTTCGAGAATCTGACTACAGCTCTAGACC |
| | TGTTCCCTAAAGAAAACTAAGTTCACAGGGACACACAGGATGGGGCTCATGGAGC |
| | AGCTGAAGCCAGACCCCAGGTTAATAGCCTTTACATTAAAATGTTTTTCTACCTA |
| | CCACTAATATGCATTCTTTAGTAAGCGGTCTCAATATACACCGATTCTTCCTTAA |
| | CTCTGTTTATGAAGTATTCAGCATCCTCCCTGCCCCCTTCAGCATCCTCCCTGCC |
| | CCTGAGCACAGGATCCAATGGCGTGAGGACCACAGGCCTGGGCAGCTGCTGGGGC |
| | ATACAGGCATCTCTTAGTGGCTGAGAGACTGGGCCCTGGCTCTATGTTGGCTCCT |
| | AACTTGCTGCCATTTAAAGGAAATCTTAGCCTCCCATCCGTAAAATCGAGAAAAT |
| | AAGACTTGTCCTACACAGCTCATGAAATAGTAATGAAATTCACATTAGAGAAGAG |
| | ATGGAAAAACACTTTGAACAAAAAGCATTTTGCTCTTATAAAAGCACAGCCTCTT |
| | TTGAGAGGCCCTTTGCTCCCCATTTCTCCTTCTTCAGACCCCCCCAGACTAGGAG |
| | AAGGTCTGTCTCATGGAGTGACCTTTTGGCTGCCTCTAGATTCCAAGCTCAGTTT |
| | TGCTTTCATTAACCACAGATACTGGGACGGACAGAAAAAGACCTAGTTTCTGTTG |
| | AGCCAAAGAGTCTCATAACTTGTCTGTTCACATACCCAAGAGCCCACCCTCTAGT |
| | TGAGACACTCAGTTCCCTCTCATTCTGGGAGACTGCATGTCTCTGTGACCTCCTG |
| | GTAGAGACCGTTTGACATGTCCCCCAACCCCCCAGTGATTGAGTCTGAATTCTCC |
| | ACTGATGACGCATTTCCTAGCACTCAGGGTGTCCCCTCCTGGTTGCCCCCTCACC |
| | ACTGAAGCCCGCTTCCTCCCTTTTCATTTGATGCTTAACAACTGTCAGTTTGCAA |
| | GAAACATGCTTCAAATCCACATTCTCCCAGTTGCCTAGCAACAACTTCCCTCCCG |
| | GATAAATGTGGGTTTCCTGTAGCTCAGCCCAGGACTGAACACAGCAGCACACACT |
| | TCTGTCCACTGCTTCAACTGCTTTTCACCTCTGGTCTGCATGCCTTCAAGACTGC |
| | AGCTCATCCCTCCCTTCAGAACCTTCCATAGCCTGCAGAGGCCATGTCTGCCCCA |
| | AAAAGACACATTGAACCTGAGGCTACTTATTTACCCTTGTGTTAGGTATATCCTC |
| | AACTTAGAAATTAATACTGTTTCCAGATTGTCTTCTTTGAATCACAGAAAGTAAA |
| | ACAACAAAACATTCAATGCTTAAGACATTTCATGTGCGGTTGGGTGACATCTGTT |
| | TGATGAACACATTTGATCCAAAGCATCAGAAATACTATGCCAACAAGACTTTTTA |
| | GGAGGTGATAAACATGTCTGTTCTACCTTAAGAAAAAAATATTACACAGTCCCAA |
| | GGGAGAGACATGGTTTTGATCCCAGACAACCCAAGCAGAGACCTCTTAGGGCCGG |
| | AATCATCTTGGCTGCTGCCTAGGACCTTATATCAATTTCTTAAGCACAGGATCAA |
| | GGCCTAAAGGCCCCTTAGACTGACCTCAGTTAGTAGAGGCAGATCCCTTCACAGC |
| | CTTATCTTCCTTAGAGGTCTAGTCTGACCTTGAACTTCGGCTGGCAGTGCTGTCA |
| | GTTGTGATGTGTGACATGGAAGAGTTATTTGTTACTTGGAAAATTAAGAGAACTT |
| | ATTTGGCATAGGAAATTGTGTGTGTGTGTGTGTGTGTGTGTGTGTGTGTGTGTGT |
| | GTGTGTGTGAGATGATGTTTGCCATTTTGATCTGTGACTTTTTTTTCCAGAAATA |
| | GTTTCTCAGTTCCATTCCAACTAAACTTACAGTCTCTTCCGGTTCTTTGACAGAA |
| | ACAATTCATGTGAATTTGAACAGATAATAGGGAAGGGGGAACCAAAAGAAGAGGA |
| | GAGCCCTGGGAAAGTTATTTTATAATTTATGGCAACCTCAGTCAGGCAACTGTGA |
| | ACAGGTACATATGGAGGGCTCCCTCGGGACTAGGCAGTATTCAGAGATGTAAGGT |
| | GTGAGGACCGGACCCTCATCATTTACCATTCCCACTAAAAAGAGCTGGGAAGGAA |
| | ATTGTAGCTGTAGCACCAGGCACGTAACTGGAGCTTAGTAACTATTTGGTGAAGG |
| | AATATTATTAAATTATTAACAAGATGGAAAAAAGGGTATTAACCACACAAAAATA |
| | CATCTCAAGCTATTGTTTCTCTGTTCCCTTTCCCCCAAATTCCTAGTCTTGCTCT |
| | TATCTGGCTGTCTCTCTAGTCACTCTTTCTTGCTGACTCTCTTCACGTTCCTTTC |
| | TCCACCTGGAATTCCTGGGCCCTCCCCTTTTACTGACAGACACTGTCCTCACTCT |
| | CACAGTCATCAGTTTGTCTCTTTACAAACCTCAGCTCAAGTGTCACTTCCCCGTC |
| | CCCAGGTGAAACTGACTGCTCCCTCCCTGTAAGTCACCATGATGACTGCTATATA |
| | TAGCCCTCATGGAACCTAAAACCTCAACAGACACAGTCTCTTTCCTACTCTGTTA |
| | TAGTTTATTTACTCATTAATTACCACAACACGTATTATTGAGCACCTACTGTGTA |
| | CCATGCCCAGAAGATAAAAGACAAACAAAATAAAACCTATTCCTATGCTTAATGA |
| | GTTTACAGTCTAGTGGAGAGATAGATACATTAAAAAATAACAGCAAACCAAAATA |
| | AAAGTGGTAAATAAATGCACTGAGAAAGACAGGAATAGCTAGGAGGGGCACCTAA |
| | TCCCTAGGGAAGGAAAGCTGGAAGAGCATGGTGATGGGGGAAGAAGGCTTTCTGG |
| | AGAAGGTGAGGTAGTTTGAAATGAGTTGACTCTGGCCAGTAGGGGTAGAGTGAGA |
| | ATGGGGTGAGACAGGGTGGGTTGGTCATTTTGATCCATTAGTCCTCAAAGTGATA |
| | GGACTAGTGGCTAAGGACTGCAGGCTTTACAGAAGCCTACAAAACTATTTGAGAT |
| | TTGAAGTTTTTTTTTTTTTTTAATTGGCTCCAAAAGAAAATGAAAAAACTTTAGA |
| | ATTATAATGAATGAATATTAAATGAATATTTAAGGAAGGTAATTTTATTCAACTT |
| | CATTGTTAAATTTAGTTAAAACAAGCCCTTGAGTTTCATTCAACACTGTTTTATC |
| | ATACCGTTGATGAGAGAAAACAAAACTGATTCCTGGCCAGGGCCACTGTCAGCGT |
| | GGGGTTTGCACATCTTTCCCATGTCTGCTTGGGTTAGCTCCAGGTACTCCTGTTT |
| | CCCCCACATCCCCAAGATGTGCCCATTAGTGGAAACGGTGTGTCTGCATGATTCC |
| | AACGTGAGTGAGTGTGGGTGTGGGAGTGAGTGCCCCTGCCATGGGAGGGCATCCT |
| | GTCCAGGTTAGATTCCTACCTTGTGCCCTGAGCTGCTGGGATGGAATCCAGCCAC |
| | CCATGACTCTGAACTGAAATAATTGGGTGAATAATTATCTTACTTTTTAATTAAT |
| | CTTTGAAAATGTATGTATAGTTCACATGTATTTCAATATTTAATATTAGAAGTAT |
| | TTTAGTCTTTATTTTGAAGTTTGGTGATTTATTGTAACCAGAAACAAGCTATAGA |
| | AACTTAATTTTGGGCCAAGTGCAGTGGCTCACACCTATAATCCCAGCATTTTGGG |
| | AGGCCGAGGCAGACGCATCACTTGAGGTCCGGAGTTCAAGATCAGCCTGGCCAAC |
| | ATGGTAAAACCCTGTCTCTACTAAAAAATACAAAAATTAGCCAGATGTGGTGGGC |
| | ACCTGTAGTCCCAGCTACTTGGGTGGCTGAAGCAGGAGAATCACTTGAACCCGGG |
| | AGGCGGAGCAGTGAGCAGAGATCGTGCCACTGCACTCCCACCTAGGCGACAGTGT |
| | GACACTCCATCTCAAAAAAAAAAAAAAATAGAAAAGAAAGAAACTTAATTCTGGT |
| | TTATATCAATTAGCCTGTGGTAAAATTGGTTTCATTATAGCCATTTCACTTAGTT |
| | GAAGTTTCCAATAACCTGTGGATGAATTAAGTGAGGATTTACTATATTCATAAAA |
| | TCTTAAATTCCAAAGCCTGTTTGCAGTTCAGGTTTTTCCACTTTACAAACACTTC |
| | TAAGTATTCACAATGATTGCTTAAAATTCATACCAGATAAATCATTAAATAAGTT |
| | GTTCAAAGTCAAATAATTTCATAAGTAAAAATTAGGAGCTTTTAGAAAACTATAC |
| | CTACATAGACCTAGACCTATAGATAGACAGAGATCTGAATAGATATGGACACAGA |
| | TGCTTTCCAAAGTGTTCATGTGATGTGTGGTGGAGTTTCAAGACCAGAGTGTGCC |
| | TGGGGCCTGCAGAAGTAAAGGAGAGGGGATGGAGAGAAGATTGTCCACATGGCCA |
| | TGGGCAATCTCCCACCCACACTCAAGTGAGGAAGACAGGAAACAAATTCAGAAAG |
| | AAGAGAAAATAATCAAAACTGATGGGAGCTTGTGACTGATTTACTTATGCGCAGC |
| | CTCCCTGGAGACATGAGTGTGGCTGTTCCTTAGGTTGTGCCTCTGGGCTCCTACC |
| | CCCTCTTAGATGCCTTCCTATTATCTAGGACCTGGTTGCTTTTTGTCTGCATAGC |
| | TTCTTTGGATTCCAGTCTTTGATGCCAGCTTCCTCCTAAAGTAGCCTTTCAGATG |
| | TCCCTTGGTTACCCTCTGCTATCTAAGGGCTCATCCTACCCCACACTCATTCCCA |
| | GCACCAATTTCTGGATCTCCAGGCTGGAGATTTAGACAATGGGATGGGAAGAACC |
| | CATGATGGGTCCCAGACAGAAAGTGGTGCCAGCCACAGAAAGGGCACACAGGCAC |
| | AGAAGTTGGTTTGGGGTAAGACGATGTGGTCAGTTCAGAACACGCTGGATCTAGG |
| | CAGATGCCCAGCAGACAGTTGGATATGTAAGTCTGAAGCTCTGGGGAGAGGTCTA |
| | GGTTGGAGGTACAGATTTAGAAGTCATCAACAAAAAGGTAGCAGATTAAATGATA |
| | AAGGAAATGAGACTATCCGGGGAGTGTGCAGAGTGAGAGGAGCAAGGGAGGCCCT |
| | TGGGAACCTCAGCACTTCAGGGGAAGGTAGAGGTACAGTTGCTGGTGGGAAAGGC |
| | AGAGAAGTAGCAAAGCAAACCAGGCAAAAGCAGTGTCACAGACGACCAGGGAGGA |
| | AAAGGACATGATCAAAATGTTGAGAAAAGCAGAGAGGTTTGAAAATACAAGAAGC |
| | AAAAATGTCCACTAGACTTAAAAACCAGGAGAAAACTGGGGGGTTCTTGATAAAG |
| | CATCTTAGTAGGATGGTGAGGGTAGAAGCCAGGGAAGTGTTGGTGAGGAAGTGAA |
| | GTCACTGATTACGGACTATGCTTAAAAGAATGTGGGAATGAAGGGTGGAAGAGAG |
| | AAATTAGACTGTAGCTAGGGAGACATAAGCGATCAGAGGTAGATTCTTTCTCTCC |
| | TGTGGGAGAATCTTGCACGTATACACAGCATGACGACAGTGATGGAAGGGCTGGC |
| | GAAGCCTCAGGGAGACTCTTGGAGGTAAACCCCATGAAGGGAGGACTTTGTTTCA |
| | TTCACTGCCGTGTCCCCAGCACCTGGCACAATAGCAGACACTCAATACATATTTG |
| | TCAAATGTGGGATTTTATCATTTAGAAACTGCACCTGGCTGTGAGTAACAAAAGT |
| | CAGAGAAACCGTGGGTTTCATTTTTCTCCCCAGGCAGAGTCTGGAGCTGGGTCCT |
| | CCAAGAGGGGTTTGGAGCACCACAGGTTTCCTCAAGACCCCCAGGCTGCCCTGTG |
| | TTTCCCTCCTTCATCCCCAGCATATGCCTGTCATCTGGTGACCTCCAAACACCTG |
| | TGCTGCCTCCTCCAGCACATCCATGTTGCAGGCAGGGACCAGGCAAAGGGCAGAG |
| | GGGCCTACTTCAAAAGACCATTTCCAGAAACCCCATCCTATGACTTCTCCTGGTG |
| | TCTTGGTTACCATTGTGCCATAGGCTCACCCTGTATGCATGGGAGGCTGGGCCAG |
| | GCATTATGACTTTTAGCAATATTGCATAGATAAGCATCAATCTTTGTCACTGTGA |
| | CGAAGCCTAGTCACTCAGTGCTAGGCAAGGTTAATGGAATGGGTTGGTGTGTGCA |
| | TTATTCTTGAGGTCTTTCTTATGCTTCATGTTATACATTTATTAGGACGTTTAGG |
| | CAACAGGGGGATAAAAATGAAGAGGAGATGCATGCTATGATCTGAATGTTTGCAT |
| | CCTCCCCAAAATTCATATGTTGAAATCTTCATCCCCAAGATGATGGCATTAGGAG |
| | GTGGGGCCTTTCGGAGGCAATTAGGTCATGACTGGGATTAGTGCCCTTGTAAAAC |
| | CCCAGAAAGCCAGCTTGCCGCTTCCACCATATGAAGACACAGAGAGAAGATGCCA |
| | TCTACGAATCAGGAAATGAGCCCGCACCATGCAATAAACCTGCTGGAGCCTTGAT |
| | CTTAGACTTCCCAGCTGCCAGATCTGTGGGAAATAGATTTCTGTTGTTTACCCAG |
| | CTTATGGTATTTTGTTGTAGCAGCCAGAGTGAACTAAGACAGTGCTGATCTCGTA |
| | TTCTTGGAGGGAACCCTTAGTCTTTAGGGAAAGCAAAGCCACCATTTGGGGCAGG |
| | GTGTTCTCCAAGTGCTGCCACATATGCTGATGTGGTTAAACTGCAAACTATGGTA |
| | AAAATGTGGAGGTCTGTGGAATTGTCAATCAGGAAAAAGATATAAAAAGAAGTTA |
| | AAGTCTTCGTGCTTCTGGAAGGATATGTGCCAAATTGTTAACATTGATTATCCTT |
| | GGGTAGAGATGTGGGGAAGTTTGCAGAGACAGTTTTGCCTTGTACTTTATATAAG |
| | TAAACAGCTACTACTTCGTTGTCTTAAAAAAAAAAAACAGCCTATGTGCTCTTCA |
| | TGTGACTCAGAACTACCTAGGCAATACGATTAATTGAATTAGTAAAATTGAGTGA |
| | TTATGAATTTTCAGGAAGTCATTAATTTACCACTTCTTTATTACATCCACTTCTA |
| | ACAGGACTTCAATATAGGGGAATTTGACTTCAAGATAAAAAGACCAAATTTATTT |
| | ACCCTTTTAAAAAAAGACAACTTAAAAGCAGACTTGTCTTACAGAACCTTCCTTA |
| | GTTGGACATCGATGAGTGTACAGAAAATGCAATGGATAAAAAGCTTGGTGATACA |
| | AAGATAAAAAGTGGGGTCCTGTCCTTAATGAACATACCATTTCATGGAGTATCAG |
| | GTGTATAAACAATTATAATCAATCTGCTTGTTATTCTGATAAGATCATTTACTCA |
| | CACATCAAATACTGAGTGCCCACCACATGCCCAGCATACCTAGAAGTCATCCAGT |
| | ATGATTTCTGTCTACATGGAGCATAGAGTCTTACAGGGGAGATAGATGACAAGTA |
| | AACACCAGAATAATTACCAATGGTGAAGAGCACAAGGAAGGAAACAGAACTCCTA |
| | AAGAGAGCGTGGCTGGGCAGGGGTGAGCAAGAGGCATAGAAAAAGGGGCATCTAA |
| | ATCTACTTGGGAGGAAGCTGTTTCTCACATAGGTCATCATGTTAGGAATGAGACT |
| | TGAGGGATGAGTAGAAGTTTGCCAGGCAAAGAAGGAATGGGGGGGAATAGAGAGC |
| | AGAGCTAGGGGCAGGAGACAGCTGACGTGTGAGCAGACATAAAAAGAAGTCCACT |
| | GTGGCAGCAGAGAAGCAGGAGAGAAGGCAAGTGAGGGAGCCAGGCACCAGCTCAC |
| | AGAGGTCATGTGTGTCAAAACGTAGTAATGGCCTTCTCTTCTGGAGACAGTAGGG |
| | AGCCATGGAAGATGTTTGAGCAGGGAAAGCGACATGACTGGATTGGCCTGTTGGG |
| | TAACTCAGACCACAATGCATTGGAAGGGAGGGGGCTAGAGGCAAGGGGACTGGCA |
| | AGAAGGCCAGTCCTTTTTCTATGCCTATTTTGATGAAATATTCTAGAAGGGAAGT |
| | GAACAAAGGTAGTCCTAGAGAGGAAGAACAAAACAGATAGGATACTTCCTTAGTA |
| | TTTGCTCATTCGACAATTTATTTTTGCATATACACTAAAACCTTTTTTATTATTA |
| | AAACGTTTTATTGTAGGAAAAAAGTATGAAAGTAGAGTGAATAATAAAATGAGCT |
| | CCCATGGATCTATCACCCAGCTTCAACTATTATCAATATTTGGCTGTTCTTGTTT |
| | TAACTGTTCTCCACCTTTTTTTCCTGAAGTTTTTTTGAAGCAAATCACAGACAAC |
| | ATATCATTTCACCATATGTACTTCCCTCTGTATCTCTAACATGTAAGAACTTGTT |
| | TTAACAAAATCACCATGCTATGATCATACCCAACAAAATTTATCATAATGTCTTA |
| | ATAATACCTAATACCCATTTCATGTCCACTTTCCCCCAATTGCTACAGCTGGTTT |
| | GTTCAGATCAGAATCAAAATCCACCTGTGGCCATTTTACTGCTATGTCTCTCAGG |
| | TCTCTTTTCATCTCTAATAATCTCAGGGGAGACAGGAGGGAGGACGGGCAGGACT |
| | TGGGGCTAACTTGCTTATCGACACACAGTTTTGCCTACTTGCTTCCTCCCTTCAC |
| | ACCCACTCTTCTTCTCAGCCCCACCCTTGTATGGAAAAAACAGAAATTAAAGTGC |
| | TTTGCCCAGCACCCACTGAAGCTATTTCGAAGGAGTTTGAAGAGTACTCCCGGCA |
| | AGACAAATGCCTCGGTCCAGTGCTCAGGTCAAAGAGGGGAGACGCTTCTCAGTGA |
| | TGTGGTGTCAATAGCAGCTTAGTTGTTCTTTCCTCTGGAAAATTCTACCCATCTG |
| | CTTTGTAACTCCCATACCTAACAAGGCCTTTTATTTCACAATTAGAAAATAAGCC |
| | TGAAATATGAATGCTGCCTGAGTGTACCTACATTTATTCTAGAGTTTCAGGGTCA |
| | AAAAGAATACAAGGACCTCTGCATCTACAGCCAAGAGGAGAGGGGCAAAGACACA |
| | CAGCTACAAATGAGAACCTGGCTGGTCAAAGCCTAACTCCACCTGTTTGTCAGCA |
| | CTGATGCAAGTTAGGTCAGCCCAATGATCATTTAGGAGAACTGTGCTGGCAAATA |
| | AAAAGCAGAGGCTTTTGGTCCCCAGATACTTGGATGAGAATTACAAGTCCAGCTG |
| | GTTAAAAGGCACATGCCCAGTGCTCACTTCACACCTACTCAGGAAGCACACTTGA |
| | GTTGGAAAACCACTGTCTTTACACTTAGAACTCAGTCCTACATGACTCCTCTAGG |
| | ATCAGTGATTCCATCAGTTTTGAAACATGAAGCATGAAGTCAAACAGGACATGAC |
| | CTTGGTTTCCAGAAAACCAGATGTTCACATCAGTCTCTGGAGCTTGGAGGCAGCA |
| | CACCTGGGGACTTCCACATCCCCTGCCGAGGTGGCAAAAGCAGGAGCAGTGGTGA |
| | GTTCACATGGGCTGGGGTTTCCTGAACACTGCTGGCAATTGGAGAATCTGCAAGG |
| | GAACTTCTCCGACTCCTACCAGCAGCTGCTTTAAAATAAAGGTGATGTAGCTGGT |
| | CAAATCCTCCATGAGAGAGCAGTGTTGAATGGAGGAAGAGACACAACCTGTCTGA |
| | AAATGGCACAAAGGAAGAAAGATGTAAACAATGACGAGAAGACTGCAGTGTCTAC |
| | AAAGCTCCGAGGTGAACAGATGGGCACCCCAGGCCCGCAGCACTTCCTTCAGTCT |
| | CTGCCAGCTGCACTCTGTTTTCCTTCCTCCAGGAATCTTGTTTGGTGTCACTAAA |
| | ACAGCAATTAGAATCACTTTGAAATAGTGATAGTATTTAATATAACTATGAAACT |
| | ATCTGTGATTGACAAGTGCAGCAAGGAGTCTTGGAATGAGAGCCTTTATTTTTTC |
| | AATTAAATAAAAGAGTTTTTTGTTTCTAAAAGTAATCTTGCAGAAAAGATCCTGC |
| | GATCAGAAAGAAGGAGGGGGGGAGTTTTCAAACATATAGGAGATCAGACTGTGCC |
| | TATGTGTGTATATACCTACAAACATATATATATTTAAAAAATTGTTTTACTGTCA |
| | ATTACAG |
| 1 | GCCTACGACTGGATTAGTATC |
| 2 | CTCGAG |
| 3 | GATACTAATCCAGTCGTAGGC |
| 4 | GCCTACGACTGGATTAGTATCCTCGAGGATACTAATCCAGTCGTAGGC |
| 5 | ATGACCATCACTTACACAAGCCAAGTGGCTAATGCCCGCTTAGGCTCCTTCTCCC |
| | GCCTGCTGCTGTGCTGGCGGGGCAGCATCTACAAGCTGCTATATGGCGAGTTCTT |
| | AATCTTCCTGCTCTGCTACTACATCATCCGCTTTATTTATAGGCTGGCCCTCACG |
| | GAAGAACAACAGCTGATGTTTGAGAAACTGACTCTGTATTGCGACAGCTACATCC |
| | AGCTCATCCCCATTTCCTTCGTGCTGGGCTTCTACGTGACGCTGGTCGTGACCCG |
| | CTGGTGGAACCAGTACGAGAACCTGCCGTGGCCCGACCGCCTCATGAGCCTGGTG |
| | TCGGGCTTCGTCGAAGGCAAGGACGAGCAAGGCCGGCTGCTGCGGCGCACGCTCA |
| | TCCGCTACGCCAACCTGGGCAACGTGCTCATCCTGCGCAGCGTCAGCACCGCAGT |
| | CTACAAGCGCTTCCCCAGCGCCCAGCACCTGGTGCAAGCAGGCTTTATGACTCCG |
| | GCAGAACACAAGCAGTTGGAGAAACTGAGCCTACCACACAACATGTTCTGGGTGC |
| | CCTGGGTGTGGTTTGCCAACCTGTCAATGAAGGCGTGGCTTGGAGGTCGAATCCG |
| | GGACCCTATCCTGCTCCAGAGCCTGCTGAACGAGATGAACACCTTGCGTACTCAG |
| | TGTGGACACCTGTATGCCTACGACTGGATTAGTATCCCACTGGTGTATACACAGG |
| | TGGTGACTGTGGCGGTGTACAGCTTCTTCCTGACTTGTCTAGTTGGGCGGCAGTT |
| | TCTGAACCCAGCCAAGGCCTACCCTGGCCATGAGCTGGACCTCGTTGTGCCCGTC |
| | TTCACGTTCCTGCAGTTCTTCTTCTATGTTGGCTGGCTGAAGGTGGCAGAGCAGC |
| | TCATCAACCCCTTTGGAGAGGATGATGATGATTTTGAGACCAACTGGATTGTCGA |
| | CAGGAATTTGCAGGTGTCCCTGTTGGCTGTGGATGAGATGCACCAGGACCTGCCT |
| | CGGATGGAGCCGGACATGTACTGGAATAAGCCCGAGCCACAGCCCCCCTACACAG |
| | CTGCTTCCGCCCAGTTCCGTCGAGCCTCCTTTATGGGCTCCACCTTCAACATCAG |
| | CCTGAACAAAGAGGAGATGGAGTTCCAGCCCAATCAGGAGGACGAGGAGGATGCT |
| | CACGCTGGCATCATTGGCCGCTTCCTAGGCCTGCAGTCCCATGATCACCATCCTC |
| | CCAGGGCAAACTCAAGGACCAAACTACTGTGGCCCAAGAGGGAATCCCTTCTCCA |
| | CGAGGGCCTGCCCAAAAACCACAAGGCAGCCAAACAGAACGTTAGGGGCCAGGAA |
| | GACAACAAGGCCTGGAAGCTTAAGGCTGTGGACGCCTTCAAGTCTGCCCCACTGT |
| | ATCAGAGGCCAGGCTACTACAGTGCCCCACAGACGCCCCTCAGCCCCACTCCCAT |
| | GTTCTTCCCCCTAGAACCATCAGCGCCGTCAAAGCTTCACAGTGTCACAGGCATA |
| | GACACCAAAGACAAAAGCTTAAAGACTGTGAGTTCTGGGGCCAAGAAAAGTTTTG |
| | AATTGCTCTCAGAGAGCGATGGGGCCTTGATGGAGCACCCAGAAGTATCTCAAGT |
| | GAGGAGGAAAACTGTGGAGTTTAACCTGACGGATATGCCAGAGATCCCCGAAAAT |
| | CACCTCAAAGAACCTTTGGAACAATCACCAACCAACATACACACTACACTCAAAG |
| | ATCACATGGATCCTTATTGGGCCTTGGAAAACAGGGATGAAGCACATTCCTAA |
| 6 | ATGACCATCACCTACACCAGCCAGGTGGCCAATGCCAGACTGGGCAGCTTTAGCA |
| | GACTGCTGCTGTGTTGGCGGGGCAGCATCTACAAGCTGCTGTACGGCGAGTTCCT |
| | GATCTTCCTGCTGTGCTACTACATCATCCGGTTCATCTACCGGCTGGCCCTGACC |
| | GAAGAACAGCAGCTGATGTTTGAGAAGCTGACCCTGTACTGCGACAGCTACATCC |
| | AGCTGATCCCCATCAGCTTCGTGCTGGGCTTCTACGTGACCCTGGTGGTCACCAG |
| | ATGGTGGAACCAGTACGAGAACCTGCCTTGGCCTGACCGGCTGATGTCTCTGGTG |
| | TCCGGATTCGTGGAAGGCAAGGACGAACAGGGGAGACTGCTGAGGCGGACCCTGA |
| | TCAGATATGCCAACCTGGGCAACGTGCTGATCCTGAGATCTGTGTCCACCGCCGT |
| | GTACAAGAGATTCCCCAGCGCTCAGCATCTGGTGCAGGCCGGCTTTATGACACCA |
| | GCCGAGCACAAGCAGCTGGAAAAGCTGAGCCTGCCTCACAACATGTTCTGGGTGC |
| | CCTGGGTTTGGTTCGCCAACCTGTCTATGAAGGCCTGGCTCGGCGGCAGAATCAG |
| | AGATCCTATCCTGCTGCAGAGCCTGCTGAACGAGATGAACACCCTGAGAACCCAG |
| | TGCGGCCACCTGTACGCCTACGACTGGATCTCTATCCCTCTGGTGTACACACAGG |
| | TCGTGACCGTGGCCGTGTACTCCTTCTTCCTGACATGCCTCGTGGGCAGACAGTT |
| | TCTGAACCCCGCCAAGGCCTATCCTGGCCACGAACTGGATCTGGTGGTGCCCGTG |
| | TTTACCTTCCTGCAGTTCTTCTTCTACGTCGGCTGGCTGAAGGTGGCCGAACAGC |
| | TGATCAATCCCTTCGGCGAGGACGACGACGACTTCGAGACAAACTGGATCGTGGA |
| | CCGGAACCTCCAGGTGTCACTGCTGGCTGTGGATGAGATGCACCAGGACCTGCCT |
| | AGAATGGAACCCGACATGTACTGGAACAAGCCCGAGCCTCAGCCTCCTTACACAG |
| | CTGCTAGCGCCCAGTTTCGGAGAGCCAGCTTTATGGGCAGCACCTTCAACATCAG |
| | CCTGAACAAAGAAGAGATGGAATTTCAGCCCAATCAAGAGGACGAGGAAGATGCC |
| | CACGCCGGAATCATCGGCAGATTTCTGGGCCTGCAGTCCCACGACCACCATCCTC |
| | CAAGAGCCAACAGCCGGACCAAACTGCTGTGGCCCAAGAGAGAGTCTCTGCTGCA |
| | CGAGGGCCTGCCAAAGAATCACAAGGCCGCCAAGCAGAACGTGCGGGGCCAAGAG |
| | GATAACAAGGCCTGGAAGCTGAAGGCCGTGGACGCCTTTAAAAGCGCCCCTCTGT |
| | ACCAGCGGCCTGGCTACTATTCTGCCCCTCAGACACCTCTGAGCCCCACACCTAT |
| | GTTCTTCCCACTGGAACCTAGCGCTCCCAGCAAGCTGCATAGCGTGACCGGCATC |
| | GACACCAAGGACAAGAGCCTGAAAACCGTGTCCAGCGGCGCCAAGAAGTCCTTCG |
| | AGCTGCTGTCTGAATCCGACGGCGCCCTGATGGAACACCCCGAAGTTTCTCAAGT |
| | GCGGAGAAAGACCGTGGAgTTCAACCTGACCGACATGCCTGAGATCCCCGAGAAC |
| | CACCTGAAAGAGCCCCTGGAACAGTCCCCTACCAACATCCACACCACACTGAAGG |
| | ACCACATGGACCCCTATTGGGCCCTCGAGAACAGAGATGAGGCCCACTCTTGA |
| 7 | GCCTACGACTGGATTAGTATC |
| 13 | ATGACCATCACTTACACAAGCCAAGTGGCTAATGCCCGCTTAGGCTCCTTCTCCC |
| | GCCTGCTGCTGTGCTGGCGGGGCAGCATCTACAAGCTGCTATATGGCGAGTTCTT |
| | AATCTTCCTGCTCTGCTACTACATCATCCGCTTTATTTATAGGCTGGCCCTCACG |
| | GAAGAACAACAGCTGATGTTTGAGAAACTGACTCTGTATTGCGACAGCTACATCC |
| | AGCTCATCCCCATTTCCTTCGTGCTGGGCTTCTACGTGACGCTGGTCGTGACCCG |
| | CTGGTGGAACCAGTACGAGAACCTGCCGTGGCCCGACCGCCTCATGAGCCTGGTG |
| | TCGGGCTTCGTCGAAGGCAAGGACGAGCAAGGCCGGCTGCTGCGGCGCACGCTCA |
| | TCCGCTACGCCAACCTGGGCAACGTGCTCATCCTGCGCAGCGTCAGCACCGCAGT |
| | CTACAAGCGCTTCCCCAGCGCCCAGCACCTGGTGCAAGCAGGCTTTATGACTCCG |
| | GCAGAACACAAGCAGTTGGAGAAACTGAGCCTACCACACAACATGTTCTGGGTGC |
| | CCTGGGTGTGGTTTGCCAACCTGTCAATGAAGGCGTGGCTTGGAGGTCGAATCCG |
| | GGACCCTATCCTGCTCCAGAGCCTGCTGAACGAGATGAACACCTTGCGTACTCAG |
| | TGTGGACACCTGTATGCCTACGACTGGATTAGTATCCCACTGGTGTATACACAGG |
| | TGGTGACTGTGGCGGTGTACAGCTTCTTCCTGACTTGTCTAGTTGGGCGGCAGTT |
| | TCTGAACCCAGCCAAGGCCTACCCTGGCCATGAGCTGGACCTCGTTGTGCCCGTC |
| | TTCACGTTCCTGCAGTTCTTCTTCTATGTTGGCTGGCTGAAGGTGGCAGAGCAGC |
| | TCATCAACCCCTTTGGAGAGGATGATGATGATTTTGAGACCAACTGGATTGTCGA |
| | CAGGAATTTGCAGGTGTCCCTGTTGGCTGTGGATGAGATGCACCAGGACCTGCCT |
| | CGGATGGAGCCGGACATGTACTGGAATAAGCCCGAGCCACAGCCCCCCTACACAG |
| | CTGCTTCCGCCCAGTTCCGTCGAGCCTCCTTTATGGGCTCCACCTTCAACATCAG |
| | CCTGAACAAAGAGGAGATGGAGTTCCAGCCCAATCAGGAGGACGAGGAGGATGCT |
| | CACGCTGGCATCATTGGCCGCTTCCTAGGCCTGCAGTCCCATGATCACCATCCTC |
| | CCAGGGCAAACTCAAGGACCAAACTACTGTGGCCCAAGAGGGAATCCCTTCTCCA |
| | CGAGGGCCTGCCCAAAAACCACAAGGCAGCCAAACAGAACGTTAGGGGCCAGGAA |
| | GACAACAAGGCCTGGAAGCTTAAGGCTGTGGACGCCTTCAAGTCTGCCCCACTGT |
| | ATCAGAGGCCAGGCTACTACAGTGCCCCACAGACGCCCCTCAGCCCCACTCCCAT |
| | GTTCTTCCCCCTAGAACCATCAGCGCCGTCAAAGCTTCACAGTGTCACAGGCATA |
| | GACACCAAAGACAAAAGCTTAAAGACTGTGAGTTCTGGGGCCAAGAAAAGTTTTG |
| | AATTGCTCTCAGAGAGCGATGGGGCCTTGATGGAGCACCCAGAAGTATCTCAAGT |
| | GAGGAGGAAAACTGTGGAGTTTAACCTGACGGATATGCCAGAGATCCCCGAAAAT |
| | CACCTCAAAGAACCTTTGGAACAATCACCAACCAACATACACACTACACTCAAAG |
| | ATCACATGGATCCTTATTGGGCCTTGGAAAACAGGGATGAAGCACATTCCTAA |
| 14 | MTITYTSQVANARLGSFSRLLLCWRGSIYKLLYGEFLIFLLCYYIIRFIYRLALT |
| | EEQQLMFEKLTLYCDSYIQLIPISFVLGFYVTLVVTRWWNQYENLPWPDRLMSLV |
| | SGFVEGKDEQGRLLRRTLIRYANLGNVLILRSVSTAVYKRFPSAQHLVQAGFMTP |
| | AEHKQLEKLSLPHNMFWVPWVWFANLSMKAWLGGRIRDPILLQSLLNEMNTLRTQ |
| | CGHLYAYDWISIPLVYTQVVTVAVYSFFLTCLVGRQFLNPAKAYPGHELDLVVPV |
| | FTFLQFFFYVGWLKVAEQLINPFGEDDDDFETNWIVDRNLQVSLLAVDEMHQDLP |
| | RMEPDMYWNKPEPQPPYTAASAQFRRASFMGSTFNISLNKEEMEFQPNQEDEEDA |
| | HAGIIGRFLGLQSHDHHPPRANSRTKLLWPKRESLLHEGLPKNHKAAKQNVRGQE |
| | DNKAWKLKAVDAFKSAPLYQRPGYYSAPQTPLSPTPMFFPLEPSAPSKLHSVTGI |
| | DTKDKSLKTVSSGAKKSFELLSESDGALMEHPEVSQVRRKTVEFNLTDMPEIPEN |
| | HLKEPLEQSPTNIHTTLKDHMDPYWALENRDEAHS |
| 15 | MFEKLTLYCDSYIQLIPISFVLGFYVTLVVTRWWNQYENLPWPDRLMSLVSGFVE |
| | GKDEQGRLLRRTLIRYANLGNVLILRSVSTAVYKRFPSAQHLVQAGFMTPAEHKQ |
| | LEKLSLPHNMFWVPWVWFANLSMKAWLGGRIRDPILLQSLLNEMNTLRTQCGHLY |
| | AYDWISIPLVYTQVVTVAVYSFELTCLVGRQFLNPAKAYPGHELDLVVPVFTFLQ |
| | FFFYVGWLKVAEQLINPFGEDDDDFETNWIVDRNLQVSLLAVDEMHQDLPRMEPD |
| | MYWNKPEPQPPYTAASAQFRRASFMGSTFNISLNKEEMEFQPNQEDEEDAHAGII |
| | GRFLGLQSHDHHPPRANSRTKLLWPKRESLLHEGLPKNHKAAKQNVRGQEDNKAW |
| | KLKAVDAFKSAPLYQRPGYYSAPQTPLSPTPMFFPLEPSAPSKLHSVTGIDTKDK |
| | SLKTVSSGAKKSFELLSESDGALMEHPEVSQVRRKTVEFNLTDMPEIPENHLKEP |
| | LEQSPTNIHTTLKDHMDPYWALENRSVLHLNQGHCIALCPTPASLALSLPFLHNF |
| | LGFHHCQSTLDLRPALAWGIYLATFTGILGKCSGPFLTSPWYHPEDFLGPGEGR |
| 16 | MFEKLTLYCDSYIQLIPISFVLGFYVTLVVTRWWNQYENLPWPDRLMSLVSGFVE |
| | GKDEQGRLLRRTLIRYANLGNVLILRSVSTAVYKRFPSAQHLVQAGFMTPAEHKQ |
| | LEKLSLPHNMFWVPWVWFANLSMKAWLGGRIRDPILLQSLLNEMNTLRTQCGHLY |
| | AYDWISIPLVYTQVVTVAVYSFFLTCLVGRQFLNPAKAYPGHELDLVVPVFTFLQ |
| | FFFYVGWLKVSLLAVDEMHQDLPRMEPDMYWNKPEPQPPYTAASAQFRRASFMGS |
| | TFNISLNKEEMEFQPNQEDEEDAHAGIIGRFLGLQSHDHHPPRANSRTKLLWPKR |
| | ESLLHEGLPKNHKAAKQNVRGQEDNKAWKLKAVDAFKSAPLYQRPGYYSAPQTPL |
| | SPTPMFFPLEPSAPSKLHSVTGIDTKDKSLKTVSSGAKKSFELLSESDGALMEHP |
| | EVSQVRRKTVEFNLTDMPEIPENHLKEPLEQSPTNIHTTLKDHMDPYWALENRDE |
| | AHS |
| 17 | GGTCTCAGGAGTCAACCCCCACCAGACTCACCCCCCTCCACCAGCCCCGGCAGGC |
| | TTCTCCTTCCATCTCTCTGAAGCAACTTACTGATGGGCCCTGCCAGCCAATCACA |
| | GCCAGAATAACGTATGATGTCACCAGCAGCCAATCAGAGCTCCTCGTCAGCATAT |
| | GCAGAATTCTGTCATTTTACTAGGGTGATGAAATTCCCAAGCAACACCATCCTTT |
| | TCAGATAAGGGCACTGAGGCTGAGAGAGGAGCTGAAACCTACCCGGGGTCACCAC |
| | ACACAGGTGGCAGATGAGACC |
| 18 | GGTCTCAGGAGGAACCACTGCTGCCTAGAGCTCTGCTCTCTTCATTGAACAATAC |
| | AAGAGGAGTGTGTAGGTAGACACCCACCACTTCCAACAGCTTAGGAGAGCCCTTG |
| | AGTATGGATTGATGTATTAAAATTTATTGAATCACATGCTGAGATTTTCACCAGC |
| | TGCCCGTGGGGATCTGGGCATTTATTCCCATATTGCACTGGCTGGCTGGAAGCCA |
| | GCAGCATAAACTCCAGGGCTGTTCTGTCAACCCCCACCAGACTCACCCCCCTCCA |
| | CCAGCCCCGGCAGGCTTCTCCTTCCATCTCTCTGAAGCAACTTACTGATGGGCCC |
| | TGCCAGCCAATCACAGCCAGAATAACGTATGATGTCACCAGCAGCCAATCAGAGC |
| | TCCTCGTCAGCATATGCAGAATTCTGTCATTTTACTAGGGTGATGAAATTCCCAA |
| | GCAACACCATCCTTTTCAGATAAGGGCACTGAGGCTGAGAGAGGAGCTGAAACCT |
| | ACCCGGGGTCACCACACACAGGTGGCAAGGCTGGGACCAGAAACCAGGACTGTTG |
| | ACTGCAGCCCGGTATTCATTCTTTCCATAGCCCACAGGGCTGTCAAAGACCCCAG |
| | GGCCTAGTCAGAGGCTCCTCCTTCCTGGAGAGTTCCTGGCACAGAAGTTGAAGCT |
| | CAGCACAGCCCCCTAACCCCCAACTCTCTCTGCAAGGCCTCAGGGGTCAGAACAC |
| | TGGTGGAGCAGATCCTTTAGCCTCTGGATTTTAGGGCCATGGTAGAGGGGGTGTT |
| | GCCCTAAATTCCAGCCCTGGCCTCAGCCCAACACCCTCCAAGAAGAAATTAGAGG |
| | GGCCATGGCCAGGCTGTGCTAGCCGTTGCTTCTGAGCAGATTACAAGAAGGGACT |
| | AAGACAAGGACTCCTTTGTGGAGGTCCTGGCTTAGGGAGTCAAGTGACGGCGGCT |
| | CAGCACTCACGTGGGCAGTGCCAGCCTCTAAGAGTGGGCAGGGGCACTGGCCACA |
| | GAGTCCCAGGGACAGATGAGACC |
| 19 | GGTCTCAGGAGAAAAGACCTAGATCCTAGTCTCAAGCCATTTATAGCTGTGTGAC |
| | CTTGGACAAGGCAGGCCTTGAGCTTCAGTGTCCTCCTTTGTAAAATGTGGATAAA |
| | CATGCGATACTGAATCATCCTTCAGGAATAGCTCTATGACTTTCCGTAGGTTGTT |
| | CCCCTGATGTGTAATGCTCaTCCATTCTTCTCAGCTCCATGAACTCCTACTCATG |
| | CTTCAAGACCCAACCGAAATGCCCCATTGTCTGTAAAAATTTCCCTGACCTCTGT |
| | GCACTTGGCACAGATCTTAGGCCTTGCCATTCTGTGTTAGAGTTGTTATTTAAGT |
| | GTTTCCAAATTTTTTGACTATGAATACGTACAGGCAGGAATCAGATTAGGCTCAG |
| | TTCTGGAAGGAGAAATGGTTTGGCGAATATTTTCTTTGCGGAACGGAAAGCAGAA |
| | GAGGGCGACCTTGACTGGGTTGGGTGCCATCTCTGCAGGGGTCAATCCTGGGACT |
| | CTTGTTCCAGGCATGGCTTGGGTCAATCAGCACTCATGACTTTGCTGTGTGCCTG |
| | AGCCGTGCCTGGCTAACTGGGGACATTTTCATAGCTGAGAgCCAGTAGGAGAGAg |
| | CATGACTTGTGCCAGGGTGTGCCTGTCTCAGCTCTGGGGCAGGGCCAGGAGCAGC |
| | TGCTCATGGGATGTCTCTGGGGACATGCTGGGGCCTCTGGGAGGGGAAGGGAGAC |
| | TGGCCATGCACTGTCTATGGTCCTGGTGGTACTCCTGGGATATTGCCAAGGAGCC |
| | TGCGGTTCAGGGAACAGGGAGGAGGGACTCTAGTGTCTCCAACTCCCTCTCATGG |
| | CTCTATGGCTGCTGGTCGGGGTCAGGGAGATGAAGCAGTGCCAGCTCCGTGGTCT |
| | GCACTGATCCTGACCAGTGAACCAGGCTTCCCCAGCCAGAGCCAGCTAGTCAAGC |
| | ACGCTCTCTAGTGGCCACCACAGGGAAGGCAAGAACTTGTCTCCACAAAGGCAGG |
| | ATTTTCATTTATTCCTTCAGAGAGTACAGAATGAGCATCAATGTTCCAGGCATGG |
| | TGCCAGGCATGTGTGTTAACTTTAGTTAAAACATACTTATTGGCCGAGTGTGGCG |
| | GCTCACTCCTGTAATCCCACCACTTTGGGAGGCCTAGGTGGGAGGACTGCTTGAA |
| | CTTAGAGCTCAATACCAGCCTGGGTAATGTAGTGAGAgCTATCTCTATAAAAAAA |
| | AAAAAAATAGCTGGGTGTGGTGGTGTGCGTCTGTAGTCCCAGCGACTTGGGAGGC |
| | TGAGATGGGAGGATTGCTTGAGCCTGGGAAGTGGAGGTTGCAATGAGCCAAAATC |
| | ACACCCATTGCACTCCAGCCTGGGTGACCTTGTCTCAAAAATATAGTTATATATT |
| | TACTTATATATTTATATGTATAAATATATGTGTACATACAAATATATTTGTAATA |
| | TTATAGACATATTAATACATATTTACATATGTTAATATATAAATATATATAATAT |
| | ATATTTATCATCTTCTAAATGGAAAGCTCTTGAGTTTCAGGATGTGTTGGTTGGG |
| | AAGGGAATACAGGATAGGGAAATAATCCATTAAGCAATGACTCAGCACAGGCCAT |
| | AAAATCAGAATCACCTGGAGAACTTGAACAATCCCAACACCTGGGGTCCATCCAG |
| | ACCAATCAAATCAGAATTCGTAGGGGTCAGGCACAGGCACCAGTGGTTTGGAGAG |
| | TTGACGGACACATGTCCCATCCATCATTCTCATTCGGCTGCCAAGGTAGGCATCG |
| | TTGTCCCATTTTGCATATGAGGAGACTGAGGCTGAAGTGAACCTGCTTCTTTCCC |
| | CATATCAGTTGAGGACAGCGATGGGGCAGCGGGGAGGCAGTAAATACTCCTACAA |
| | GGCTGCCTCTAAAGAGAGTCTTATAATAACAACAGCTTTATTGTTTGCCATGATT |
| | AGAAAAGTATTCAAGGTCATTGCGAAATAGTGTTTTTTTTTCTAGGCAGATAATT |
| | ATATAAAGGCAAAAATGGTGTGTGGGGGTGGGGGACAAGGGGAGGGAGAGCATTA |
| | GGACAAATACCTAATGCACGCAAGGCTTAAAACCTAGATGATGGGTTGACAGGTG |
| | CAGCAAACCACCATGGCACACGTATACCTATGTAACAAACCTGCACATCATGTAT |
| | ACCTATGTAACAAACCTGTACATTCTGCACACGTATCCCAGAACTTAAAGTGAAA |
| | AAAAAAGTGGTGTGTAGAAAAATCACCTGCAATCTCAGCATAGTTAACGCTTAGT |
| | ACATTTCAGAGAGAGAGGGTGACAGGAAAGGGAGGATGAGAGTGGGTTTAAGACA |
| | CAAGGTCATATTATAAAATCAGGGCTTCTGGAAGTTTAGTCCCAAAACCACACAT |
| | CTCATAATCCCCTGCAGTGCTTGATTAAAATGCAACATCCCTAAGGCCACAGACT |
| | CAGACTCTGGAGAAAGATCCAGAAAACTGCCCGTTTAATAAACATTTGGGCGATT |
| | CTTACGGCCTCTAAAGACCAAGAACCACTGCTGCCTAGAGCTCTGCTCTCTTCAT |
| | TGAACAATACAAGAGGAGTGTGTAGGTAGACACCCACCACTTCCAACAGCTTAGG |
| | AGAGCCCTTGAGTATGGATTGATGTATTAAAATTTATTGAATCACATGCTGAGAT |
| | TTTCACCAGCTGCCCGTGGGGATCTGGGCATTTATTCCCATATTGCACTGGCTGG |
| | CTGGAAGCCAGCAGCATAAACTCCAGGGCTGTTCTGTCAACCCCCACCAGACTCA |
| | CCCCCCTCCACCAGCCCCGGCAGGCTTCTCCTTCCATCTCTCTGAAGCAACTTAC |
| | TGATGGGCCCTGCCAGCCAATCACAGCCAGAATAACGTATGATGTCACCAGCAGC |
| | CAATCAGAGCTCCTCGTCAGCATATGCAGAATTCTGTCATTTTACTAGGGTGATG |
| | AAATTCCCAAGCAACACCATCCTTTTCAGATAAGGGCACTGAGGCTGAGAGAGGA |
| | GCTGAAACCTACCCGGGGTCACCACACACAGGTGGCAGATGAGACC |
| 20 | GGTCTCAGGAGGAACCACTGCTGCCTAGAGCTCTGCTCTCTTCATTGAACAATAC |
| | AAGAGGAGTGTGTAGGTAGACACCCACCACTTCCAACAGCTTAGGAGAGCCCTTG |
| | AGTATGGATTGATGTATTAAAATTTATTGAATCACATGCTGAGATTTTCACCAGC |
| | TGCCCGTGGGGATCTGGGCATTTATTCCCATATTGCACTGGCTGGCTGGAAGCCA |
| | GCAGCATAAACTCCAGGGCTGTTCTGTCAACCCCCACCAGACTCACCCCCCTCCA |
| | CCAGCCCCGGCAGGCTTCTCCTTCCATCTCTCTGAAGCAACTTACTGATGGGCCC |
| | TGCCAGCCAATCACAGCCAGAATAACGTATGATGTCACCAGCAGCCAATCAGAGC |
| | TCCTCGTCAGCATATGCAGAATTCTGTCATTTTACTAGGGTGATGAAATTCCCAA |
| | GCAACACCATCCTTTTCAGATAAGGGCACTGAGGCTGAGAGAGGAGCTGAAACCT |
| | ACCCGGGGTCACCACACACAGGTGGCAAGGCTGGGACCAGAAACCAGGACTGTTG |
| | ACTGCAGCCCGGTATTCATTCTTTCCATAGCCCACAGGGCTGTCAAAGACCCCAG |
| | GGCCTAGTCAGAGGCTCCTCCTTCCTGGAGAGTTCCTGGCACAGAAGTTGAAGCT |
| | CAGCACAGCCCCCTAACCCCCAACTCTCTCTGCAAGGCCTCAGGGGTCAGAACAC |
| | TGGTGGAGCAGATCCTTTAGCCTCTGGATTTTAGGGCCATGGTAGAGGGGGTGTT |
| | GCCCTAAATTCCAGCCCTGGCCTCAGCCCAACACCCTCCAAGAAGAAATTAGAGG |
| | GGCCATGGCCAGGCTGTGCTAGCCGTTGCTTCTGAGCAGATTACAAGAAGGGACT |
| | AAGACAAGGACTCCTTTGTGGAGGTCCTGGCTTAGGGAGTCAAGTGACGGCGGCT |
| | CAGCACTCACGTGGGCAGTGCCAGCCTCTAAGAGTGGGCAGGGGCACTGGCCACA |
| | GAGTCCCAGGGAGTCCCACCAGCCTAGTCGCCAGACCTTCTGTGGGATCATCGGA |
| | CCCACCTGGAACCCCACCTGTGAGTACAAGGTGCCCCAGGTGGACTGGGCTGGGG |
| | CTTTGAGGCCTTCAGGGTTGGATGGCCATCTTGCGTATTTGTGTGGGATATGCAC |
| | ACACAGGCAGCACATGCGCAGGTGTGTGGGCACCTGTGTGTCTGTGCAAATGCCC |
| | TGAGGTGGGAATGAGCTTGGTGTGCATCAGGCACAGCCAGCCAGTGTGGCTGCAG |
| | CAAAACACACAGGGAAAGAATGGAGGGGGCATCAATCACTGCTTCAGTAAATTTT |
| | TATTGAGCGCCTTCTACGAGAACACAAGAGGAGCTTCCATTCTGAGGAGGAAACA |
| | GGCAGGAAACAGGCAGATATCCTGTATAATTTCAAGTAGTGATAAGTGCTCTCTA |
| | GAAATATCAAGCAAGGTGAGGAGACACAGAGCACCGGTGGCAGTGGGaCTCAGGA |
| | GGGGCTCTATTTCCAGGTTGGATGGTTGGGAACATCCTTTCTAAAGGGAACCTGG |
| | AGTGGGAAGGAACCATGCAGGTATCTCAGGAgGAGCTTCCTCCAGGCAGGAAGAT |
| | CAGCAGGTGGAAAGGCCCTGGAGCCACCATTCAGTAAACATCATTTGAGCATCTC |
| | TACCAGCTAGGTTCCATTATGGGAATGGGAATATGGTGGTGGACAGGGCTGCCTG |
| | GTCCCTTCCATACTTCTCACACTAGGGTGGTTGAGAGAGCTTGGGAGCTAACGAA |
| | CAAGATGGGCTGAGAACACTGCCTAGCCCAGAGGACCTGAGCTTAGTGTGTAGAC |
| | ATTGCTGCTGTTACTGCCTTTGTCATTGTATTATTTATTTATTTATTTATTTATT |
| | TTTAGACAGAGTTTTGCTCcTCTTACCCAGGCTGGAGTGCAATGGCGTGATCTCA |
| | GCTCACTGCAACCTCCACCTCCTGGGTTCAAGCGATTCTCCTGCCTCAGCCTCCT |
| | GAGTAGCTGGGATTACAGGCACCCGCACCACGCCTGGATAATTTTTTTGTATTTT |
| | TAGTAGAGACAGGGTTTCACCATGTTGGCCAGGCTGGcCTCGAACTCCTGACCTT |
| | AGGTGATCCACCTGCCTCGACTTCCCAAAGTGCTGGGATTATAGGCATGAGCCAC |
| | TGCGCCCAGTGATTATAGAAAGTTAAAGGCACATGGCAATGCACACGCCTATCTA |
| | CGTCTTCCCTGCCAAAGCAAAGGGCAGCCTCTGGGCTCACTTTCTTGCGTTTCTA |
| | CTTCCAAAAGGCAGTCAGAACTGGCAGGGCTTGGAGgCCACTTCATCCACCTCCT |
| | AGGGTCCCTATGGGAGAGTTGAGGTCCAGAGCAGGGAAGGGTCCTGACAGGCTCT |
| | GACCAGGGCCTCTGATCCCTACAAACCCCCAATCGGTGTCCCTCTCTACCAGGAC |
| | CCAAGCCCACCTGCTGCAGCCCACTGCCTGGCCCAGATGAGACC |
| 40 | GAACCACTGCTGCCTAGAGCTCTGCTCTCTTCATTGAACAATACAAGAGGAGTGT |
| | GTAGGTAGACACCCACCACTTCCAACAGCTTAGGAGAGCCCTTGAGTATGGATTG |
| | ATGTATTAAAATTTATTGAATCACATGCTGAGATTTTCACCAGCTGCCCGTGGGG |
| | ATCTGGGCATTTATTCCCATATTGCACTGGCTGGCTGGAAGCCAGCAGCATAAAC |
| | TCCAGGGCTGTTCTGTCAACCCCCACCAGACTCACCCCCCTCCACCAGCCCCGGC |
| | AGGCTTCTCCTTCCATCTCTCTGAAGCAACTTACTGATGGGCCCTGCCAGCCAAT |
| | CACAGCCAGAATAACGTATGATGTCACCAGCAGCCAATCAGAGCTCCTCGTCAGC |
| | ATATGCAGAATTCTGTCATTTTACTAGGGTGATGAAATTCCCAAGCAACACCATC |
| | CTTTTCAGATAAGGGCACTGAGGCTGAGAGAGGAGCTGAAACCTACCCGGGGTCA |
| | CCACACACAGGTGGCAAGGCTGGGACCAGAAACCAGGACTGTTGACTGCAGCCCG |
| | GTATTCATTCTTTCCATAGCCCACAGGGCTGTCAAAGACCCCAGGGCCTAGTCAG |
| | AGGCTCCTCCTTCCTGGAGAGTTCCTGGCACAGAAGTTGAAGCTCAGCACAGCCC |
| | CCTAACCCCCAACTCTCTCTGCAAGGCCTCAGGGGTCAGAACACTGGTGGAGCAG |
| | ATCCTTTAGCCTCTGGATTTTAGGGCCATGGTAGAGGGGGTGTTGCCCTAAATTC |
| | CAGCCCTGGCCTCAGCCCAACACCCTCCAAGAAGAAATTAGAGGGGCCATGGCCA |
| | GGCTGTGCTAGCCGTTGCTTCTGAGCAGATTACAAGAAGGGACTAAGACAAGGAC |
| | TCCTTTGTGGAGGTCCTGGCTTAGGGAGTCAAGTGACGGCGGCTCAGCACTCACG |
| | TGGGCAGTGCCAGCCTCTAAGAGTGGGCAGGGGCACTGGCCACAGAGTCCCAGGG |
| | A |
| 41 | MSAVLQRFREKLPHKPYCTNDFAYGVRILPKNIAILARFIQQNQPHALYWLPFDV |
| | DRTGASIDWSDRNCPAPNITVKNPRNGHAHLLYALALPVRTAPDASASALRYAAA |
| | IERALCEKLGADVNYSGLICKNPCHPEWQEVEWREEPYTLDELADYLDLSASARR |
| | SVDKNYGLGRNYHLFEKVRKWAYRAIRQGWPVFSQWLDAVIQRVEMYNASLPVPL |
| | SPAECRAIGKSIAKYTHRKFSPEGFSAVQAARGRKGGTKSKRAAVPTSARSLKPW |
| | EALGISRATYYRKLKCDPDLAK |
| 42 | AGGGCGCTGTTATCTGATAAGGCTTATCTGGTCTCATTTT |
| 43 | AGGGCGCTGTTATCTGATAAGGCTTATCTGGTCTCA |
| 44 | GCGCTGTTATCTGATAAGGCTTATCTGGTCTCA |
| 45 | TGTTATCTGATAAGGCTTATCTGGTCT |
| 46 | TGTTATCTGATAAGGCTTATCTGGTCTC |
| 47 | TGTTATCTGATAAGGCTTATCTGGTCTCA |
| 48 | CTGTTATCTGATAAGGCTTATCTGGTCTCA |
| 49 | GCTGTTATCTGATAAGGCTTATCTGGTCTCA |
| 50 | CGCTGTTATCTGATAAGGCTTATCTGGTCTCA |
| 51 | X1X2X3X4X5TGTTATCTGATAAGGCTTATCTGGTCTX6X7 |

1. A DNA vector comprising:
(a) a BEST 1-encoding sequence, wherein the BEST 1-encoding sequence is a DNA sequence encoding a bestrophin 1 (BEST1) RNA transcript; and
(b) an shRNA-encoding sequence, wherein the shRNA-encoding sequence is a DNA sequence encoding a short hairpin RNA (shRNA), wherein the shRNA-encoding sequence comprises SEQ ID NO: 1 and/or SEQ ID NO: 3; wherein the shRNA is not capable of targeting the BEST1 RNA transcript.

2. The DNA vector of claim 1, wherein the shRNA-encoding sequence comprises SEQ ID NO: 1 and SEQ ID NO: 3 connected by a loop-encoding sequence.

3. The DNA vector of claim 2, wherein the loop-encoding sequence comprises SEQ ID NO: 2.

4. The DNA vector of claim 1, wherein the shRNA-encoding sequence comprises SEQ ID NO: 4.

5.-69. (canceled)

70. A DNA vector comprising:
(a) a BEST 1-encoding sequence; and
(b) a regulatory element operably linked to the BEST 1-encoding sequence, wherein the regulatory element comprises a sequence derived from intron 6 of ABCA4.

71. The DNA vector of claim 70, wherein the sequence derived from ABCA4 intron 6 comprises SEQ ID NO: 8 or a functional variant thereof.

72. The DNA vector of claim 70, further comprising a promoter operably linked to the BEST 1-encoding sequence.

73. The DNA vector of claim 72, wherein the promoter comprises a native BEST1 promoter, a native MY07A promoter, a retroviral Rous sarcoma virus (RSV) LTR promoter, an SV40 promoter, a dihydrofolate reductase promoter, a P-actin promoter, a phosphoglycerol kinase (PGK) promoter, a cytomegalovirus (CMV) enhancer/beta-actin (CAG) promoter, an elongation factor 1 alpha (EF1A) promoter, an interphotoreceptor retinoid-binding protein (IBRP) promoter, a rhodopsin kinase (RK) promoter, or a functional variant thereof.

74.-77. (canceled)

78. A DNA vector comprising:
(a) a BEST 1-encoding sequence; and
(b) a promoter operably linked to the BEST 1-encoding sequence, wherein the promoter comprises a native MY07A promoter or a functional variant thereof or a native ABCA4 promoter or functional variant thereof.

79. (canceled)

80. The DNA vector of claim 78, wherein the native ABC A4 promoter or functional variant thereof comprises SEQ ID NO: 24, 25, 26, or 27, or a functional variant thereof.

81. The DNA vector of claim 78, wherein the DNA vector further comprises a regulatory element operably linked to the BEST 1-encoding sequence.

82. The DNA vector of claim 81, wherein the regulatory element comprises a sequence derived from intron 6 of ABCA4.

83. The DNA vector of claim 82, wherein the sequence derived from ABCA4 intron 6 comprises SEQ ID NO: 8 or a functional variant thereof.

84. The DNA vector of claim 81, wherein the regulatory element comprises a scaffold/matrix attachment region (S/MAR) sequence.

85. The DNA vector of claim 84, wherein the S/MAR sequence comprises an interferon-beta S/MAR sequence or a functional variant thereof.

86.-103. (canceled)


SEQUENCE LISTING (51 sequences)

SEQ ID NO: 1 - DNA, 21 bp, Synthetic construct
  source 1..21: mol_type = other DNA
gcctacgactggattagtatc

SEQ ID NO: 2 - skipped

SEQ ID NO: 3 - DNA, 21 bp, Synthetic construct
  source 1..21: mol_type = other DNA
gatactaatccagtcgtaggc

SEQ ID NO: 4 - DNA, 48 bp, Synthetic construct
  source 1..48: mol_type = other DNA
gcctacgactggattagtatcctcgaggatactaatccagtcgtaggc

SEQ ID NO: 5 - DNA, 1758 bp, Synthetic construct
  source 1..1758: mol_type = other DNA
atgaccatcacttacacaagccaagtggctaatgcccgcttaggctccttctcccgcctgctgctgtgctggcggggcagcatctacaagctgctatatggcgagttcttaatcttcctgctctgctactacatcatccgctttatttataggctggccctcacggaagaacaacagctgatgtttgagaaactgactctgtattgcgacagctacatccagctcatccccatttccttcgtgctgggcttctacgtgacgctggtcgtgacccgctggtggaaccagtacgagaacctgccgtggcccgaccgcctcatgagcctggtgtcgggcttcgtcgaaggcaaggacgagcaaggccggctgctgcggcgcacgctcatccgctacgccaacctgggcaacgtgctcatcctgcgcagcgtcagcaccgcagtctacaagcgcttccccagcgcccagcacctggtgcaagcaggctttatgactccggcagaacacaagcagttggagaaactgagcctaccacacaacatgttctgggtgccctgggtgtggtttgccaacctgtcaatgaaggcgtggcttggaggtcgaatccgggaccctatcctgctccagagcctgctgaacgagatgaacaccttgcgtactcagtgtggacacctgtatgcctacgactggattagtatcccactggtgtatacacaggtggtgactgtggcggtgtacagcttcttcctgacttgtctagttgggcggcagtttctgaacccagccaaggcctaccctggccatgagctggacctcgttgtgcccgtcttcacgttcctgcagttcttcttctatgttggctggctgaaggtggcagagcagctcatcaacccctttggagaggatgatgatgattttgagaccaactggattgtcgacaggaatttgcaggtgtccctgttggctgtggatgagatgcaccaggacctgcctcggatggagccggacatgtactggaataagcccgagccacagcccccctacacagctgcttccgcccagttccgtcgagcctcctttatgggctccaccttcaacatcagcctgaacaaagaggagatggagttccagcccaatcaggaggacgaggaggatgctcacgctggcatcattggccgcttcctaggcctgcagtcccatgatcaccatcctcccagggcaaactcaaggaccaaactactgtggcccaagagggaatcccttctccacgagggcctgcccaaaaaccacaaggcagccaaacagaacgttaggggccaggaagacaacaaggcctggaagcttaaggctgtggacgccttcaagtctgccccactgtatcagaggccaggctactacagtgccccacagacgcccctcagccccactcccatgttcttccccctagaaccatcagcgccgtcaaagcttcacagtgtcacaggcatagacaccaaagacaaaagcttaaagactgtgagttctggggccaagaaaagttttgaattgctctcagagagcgatggggccttgatggagcacccagaagtatctcaagtgaggaggaaaactgtggagtttaacctgacggatatgccagagatccccgaaaatcacctcaaagaacctttggaacaatcaccaaccaacatacacactacactcaaagatcacatggatccttattgggccttggaaaacagggatgaagcacattcctaa

SEQ ID NO: 6 - DNA, 1758 bp, Synthetic construct
  source 1..1758: mol_type = other DNA
atgaccatcacctacaccagccaggtggccaatgccagactgggcagctttagcagactgctgctgtgttggcggggcagcatctacaagctgctgtacggcgagttcctgatcttcctgctgtgctactacatcatccggttcatctaccggctggccctgaccgaagaacagcagctgatgtttgagaagctgaccctgtactgcgacagctacatccagctgatccccatcagcttcgtgctgggcttctacgtgaccctggtggtcaccagatggtggaaccagtacgagaacctgccttggcctgaccggctgatgtctctggtgtccggattcgtggaaggcaaggacgaacaggggagactgctgaggcggaccctgatcagatatgccaacctgggcaacgtgctgatcctgagatctgtgtccaccgccgtgtacaagagattccccagcgctcagcatctggtgcaggccggctttatgacaccagccgagcacaagcagctggaaaagctgagcctgcctcacaacatgttctgggtgccctgggtttggttcgccaacctgtctatgaaggcctggctcggcggcagaatcagagatcctatcctgctgcagagcctgctgaacgagatgaacaccctgagaacccagtgcggccacctgtacgcctacgactggatctctatccctctggtgtacacacaggtcgtgaccgtggccgtgtactccttcttcctgacatgcctcgtgggcagacagtttctgaaccccgccaaggcctatcctggccacgaactggatctggtggtgcccgtgtttaccttcctgcagttcttcttctacgtcggctggctgaaggtggccgaacagctgatcaatcccttcggcgaggacgacgacgacttcgagacaaactggatcgtggaccggaacctccaggtgtcactgctggctgtggatgagatgcaccaggacctgcctagaatggaacccgacatgtactggaacaagcccgagcctcagcctccttacacagctgctagcgcccagtttcggagagccagctttatgggcagcaccttcaacatcagcctgaacaaagaagagatggaatttcagcccaatcaagaggacgaggaagatgcccacgccggaatcatcggcagatttctgggcctgcagtcccacgaccaccatcctccaagagccaacagccggaccaaactgctgtggcccaagagagagtctctgctgcacgagggcctgccaaagaatcacaaggccgccaagcagaacgtgcggggccaagaggataacaaggcctggaagctgaaggccgtggacgcctttaaaagcgcccctctgtaccagcggcctggctactattctgcccctcagacacctctgagccccacacctatgttcttcccactggaacctagcgctcccagcaagctgcatagcgtgaccggcatcgacaccaaggacaagagcctgaaaaccgtgtccagcggcgccaagaagtccttcgagctgctgtctgaatccgacggcgccctgatggaacaccccgaagtttctcaagtgcggagaaagaccgtggagttcaacctgaccgacatgcctgagatccccgagaaccacctgaaagagcccctggaacagtcccctaccaacatccacaccacactgaaggaccacatggacccctattgggccctcgagaacagagatgaggcccactcttga

SEQ ID NO: 7 - DNA, 21 bp, Synthetic construct
  source 1..21: mol_type = other DNA
gcctacgactggattagtatc

SEQ ID NO: 8 - DNA, 1664 bp, Synthetic construct
  source 1..1664: mol_type = other DNA
agaccctccgatcctctgaaaggagcatcaggcaggcaggggttgctccggggccagggaaggggccccgctgggacgcggctgttattgcagctggttggcgcgcagccatgcttagctgcagtgcgggaatgctgggccttctgttctgggctgtttctcatacgcacgtaggccagtgtataaataaggttttattaaatgccaaatgagttctcattaacaaagaaagagggaaaatctcagtaaaccaccgtgacggcatctacccactttgagtcaggagctgggggtgtgagtgcaacctccgagacaagggaacctgtggagcccagagaatcggaggggggcgctggggttagcaccgactgaaccagctgtgttttctctcggttccttggagatcagaagtgagtgttgtcatcttcaaacaatccaaaggcagtacccatggccttactacatccctcccacaccatcccacccatccccgcgcgtacactcacacgctcatttgcacactatcgcacacgctcacttgcgtgcgcacacacagattggtgacctaggtggactgggagagaaataagagccaaatgactggattttctccaaggaaatttattaatagcccctcttggtttcacctgaaggagcttgtcttcacctgcggcctttgcaggcttaacgcccccagcttgaaacccagaagctcagacttgggcccaaggtattattagtgccaacactacctgaaatgtttcgcacctcataaaaatggtgtgtcagtttcgggtgagaggttgggacgcttcccatctgatttggcccaaggcatgcatgcccctccttctccttcccctcctcctccccctcttccccctaccatccttcctgttttctctccaactctggtgcacagctttgaaatcttgctgagaagcaaatctgtcccttctgctttgaatgtttatttgtggaagttcggcaggggaaccgaggcgggtgccaagacctgccatgctgctgggaagtctgagtctccctcccttccccctcctaaatgcttgttgatagagaaaagtcagcctcctcggcatttgggctcacggttttcctttgaaaatgcttccagtgtggcatgattcagctttcttttctgtcccccaaccactgctctgttgtcatttttacttttctgattgcattttatccgtgtctctttgactacggggtggctggacgttgagttccaggaagaaaagggcccaatcttggggttctgactacatgcgcccatcaatgtcctgtttcattcttggctctggctccctgaattcctgagtcactggggagaagcgtgggtggaccgccccctacccagtgagagttgccacagttgctgctctcctgggtcattggttgcagattgttaaacttcacctatgcatttcaactttcgggtggatattgctacgtcaagtgtctgggaaagcccccacagctacaggattttacagtgaggtcccactaatgacttgatgtcatgacttcctcattctttccaatttctcccacttctccataagggttttgggaaggggagaagagaaaggagtgattcctgagtgccagtaccagggaacagcagggctgttgggagg

SEQ ID NO: 9 - DNA, 2206 bp, Synthetic construct
  source 1..2206: mol_type = other DNA
gaattcagcaaggtcgccacgcacaagatcaatattaacaatcagtcatctctctttagcaataaaaaggtgaaaaattacattttaaaaatgacaccatagacgatgtatgaaaataatctacttggaaataaatctaggcaaagaagtgcaagactgttacccagaaaacttacaaattgtaaatgagaggttagtgaagatttaaatgaatgaagatctaaataaacttataaattgtgagagaaattaatgaatgtctaagttaatgcagaaacggagagacatactatattcatgaactaaaagacttaatattgtgaaggtatactttcttttcacataaatttgtagtcaatatgttcaccccaaaaaagctgtttgttaacttgtcaacctcatttcaaaatgtatatagaaagcccaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatgatctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcatgacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcaatattgatggtatataaccttgatatgatgtgatgagaacagtactttaccccatgggcttcctccccaaacccttaccccagtataaatcatgacaaatatactttaaaaaccattaccctatatctaaccagtactcctcaaaactgtcaaggtcatcaaaaataagaaaagtctgaggaactgtcaaaactaagaggaacccaaggagacatgagaattatatgtaatgtggcattctgaatgagatcccagaacagaaaaagaacagtagctaaaaaactaatgaaatataaataaagtttgaactttagttttttttaaaaaagagtagcattaacacggcaaagtcattttcatatttttcttgaacattaagtacaagtctataattaaaaattttttaaatgtagtctggaacattgccagaaacagaagtacagcagctatctgtgctgtcgcctaactatccatagctgattggtctaaaatgagatacatcaacgctcctccatgttttttgttttctttttaaatgaaaaactttattttttaagaggagtttcaggttcatagcaaaattgagaggaaggtacattcaagctgaggaagttttcctctattcctagtttactgagagattgcatcatgattgggtgttaaattttgtcaaatgctttttctgtgtctatcaatatgaccatgtgattttcttctttaacctgttgatgggacaaattacgttaattgattttcaaacgttgaaccacccttacatatctggaataaattctacttggttgtggtgtatattttttgatacattcttggattctttttgctaatattttgttgaaaatgtttgtatctttgttcatgagagatattggtctgttgttttcttttcttgtaatgtcattttctagttccggtattaaggtaatgctggcctagtggaatgatttaggaagtattccctctgcttctgtcttctgaaagagattgtagaaagttgatacaatttttttttctttaaatatcttgatagaattc

SEQ ID NO: 10 - DNA, 540 bp, Synthetic construct
  source 1..540: mol_type = other DNA
gcaggcatgcttctatattattttctaaaagatttaaagttttgccttctccatttagacttataattcactggaatttttttgtgtgtatggtatgacatatgggttcccttttattttttacatataaatatatttccctgtttttctaaaaaagaggggatcctctagagtcgacctgcaggcatgcttctatattattttctaaaagatttaaagttttgccttctccatttagacttataattcactggaatttttttgtgtgtatggtatgacatatgggttcccttttattttttacatataaatatatttccctgtttttctaaaaaagaggggatcctctagagtcgacctgcaggcatgcttctatattattttctaaaagatttaaagttttgccttctccatttagacttataattcactggaatttttttgtgtgtatggtatgacatatgggttcccttttattttttacatataaatatatttccctgtttttctaaaaaagaggggatcctctagagtcgacct

SEQ ID NO: 11 - DNA, 2062 bp, Synthetic construct
  source 1..2062: mol_type = other DNA
acacagcctgaagcccagcggctgtgctatgggctggggacggctctgtgctcagcatccttcaatccacggcggctccgctgcacctcctctgcaaaacgcctccaggaaaccactcggccgagtgccctctcccggagccttacttagagcctcaggaaaaggaagaaagtgccgaaaggttgaagaaaagaagcaggctttcctggaaggtcctggaagggggcgtccgcgggagctcacggggacagcccccccccaaagcccccagggctgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctgggggatacggggaaaaagctttaggctgaaagagagatttagaatgacagaatcatagaacggcctgggttgcaaggagcacagtgctcatccagatccaaccccctgctatgtgcagggtcatcaaccagcagcccagcgcgtcagagccacatccagcctggccttgaatgcctgcctgcagggatggggcatccacagcctccttgggcaacctgttcagtgcgtcaccaccctctggggaaaaactgcctcctcatatccaacccaaacctcccctgtctcagtgtaaagccattcccccttgtcctatcaagggggagtttgctgtgacattgttggtctggggtgacacatgtttgccaattcagtgctcacggagaggcagatcttgggataaggaagtgcaggacagcatggacgtggacatgcaggtgttgaggctctggacactccaagtcacagcgttcagaacagccttaaggtcaagaagataggatagaaggacaaagagcaagttaaaacccagcatggagaggagcacaaaaaggccacagacactgctggtccctgtgtctgagcctgcatgtttgatggtgtctggatgcaagcagaaggggtggatgagcttgcctggagagatacaggctgggtcgtaggactgggacaggcagctggagaattgccatgtagatgttcatacaatcgtcaaatcatgaaggctggaaaagccctccaagatccccaagaccaaccccaacccacccaccgtgccactggccatgtccctcagtgccacatccccacagttcttcatcacctccagggacggtgaccccccacctccgtgggcagctgtgccactgcagcaccgctctttggagaaggtaaatcttgctaaatccagcccgaccctcccctgggcacaacgttaaggccattatctctcatcctactccaggacggagtcagttgaggatgggggaatattgctgtgggtggcagatggccacatcccatgtccctcagtgccacatccccacagttcttcatcacctccagggacggtgaccccccacctccgtgggcagctgtgccactgcagcaccgctctttggagaaggtaaatcttgctaaatccagcccgaccctcccctgggcacaacgttaaggccattatctctcatcctactccaggacggagtcagttgaggatgggggaatattgctgtgggtggcagatggccacatcctggtcaaaggctataggcagaacgaagacacattcaatgtctgtgaggctgtgagtgaaaagcatttggaagaggcaggtgctaagctgatctctctggctccggccacagcatcctcagtgtggttgggattgcagatgtggacaaggccaggactgccacagctggcatagcgaggaggaggtacatggcacgtgcaggctctgctctgccttaatgacaacagggaacatccagttccccagcagcaccaggaggtacatggagggggcagctgagtggttttccatgatggaaaagccaccaagaagaatatcagcatccttggtctggctgctgttggtccaaggtg

SEQ ID NO: 12 - DNA, 15352 bp, Synthetic construct
  source 1..15352: mol_type = other DNA
gtaagggaggggtttggctgctcgccaattgcaaggtgattcctggggtagcagagcctcacgaattgaccttggggagggcgtgagcctggtgttctggacaatccttgcaaaagctccaggctcccagggctcaaaaaatcacaactgatagtatttctagaacagtggcccagggacccagaagtcactatgaggttcaccattaggtatgtggctgtggcatgtttgtgtccactctaaatgtggggataatcccctttacctcctctaacagagtggtaaaggaaggaggaggcctggtttgactccctgacctgctatttcctagccaggtgatcatggtaagatattgaaccttttctggtcccagtactcatctataaaacaaatataatactttacagagtggtaggaattatacaagaaaagtatacgcaaaacatttcataaattttaataaatgatggccccatgcttcttcctctggaaatggtctcaacctcaatggttggtgtttctagagagaaaaaacgacagagaaagtttcatagtctcaaaaatttggaaagccctgatctagctcaaccctttgttctagaactgcatcccagacagactgcttgggacctgaaaatatctcctcctttgctagaaggataagatgagaaggaattagataaaggaggtgtagagcagaggttttcacactgcaaagtgcataaaaaccatcagagggccgggcgcagtggctcacgcctgtaatcccagcactttgggaggccgaggcgggcggatcatgaggtcaggagatagagaccatcctggctaacacggtgaaaccccgtctctactaaaaaacacacacacacaaaaattagccaggtgtggtggcgggcgcctgtaatcccagctactgaggaggctgaggccggagaatggcgtgaacccgggaggcggagcttgcagtgagccgagattgcgccactgcactccagcctgggtgacagagcaagactccgtctcaataaaaacaaacaaacaaacaaaccaaaaaaacccatcagagaagttggtaaaagatgcaagtgctaaatccccacccccaatcactgtgattcagaagaaccaggccaggcccagaatctatcctgttaccttaggcgattctgatgaagaccattgtaggccacactttcagaaacactcaaaattagaatccttcagagaaggtggcatatataatatttctagcatggaattatgttttttttcttttgcctacattttaatttctagaactgtgttgtagggaatgtcagtcactaagaacttgattgaggaactgtgttttgtctgtttcatgactgctctctcaagtcccaggaaactcactttcagcttgtcttaaaaagcaagctgaaggcttttaaaaatgaagcaacatgaaataagacaccgcagtttctggcacggtccacgcttaatccccttcaatgtgtgactttccgtggaaagttactctacgattttcccagctcgtcagggtggggccccagagtgagtatgaagggtcagagcctagggatgccaccatcagtgagagcccaggaccccagaaaaggtctcttggctcaccacactgtaggaaaaataaaaagcaatgtagtccaaatgtctctatccaaagtttcaaaaagaacttgattttagacacgctccttgacttgttttcagaatcagacagaagagtgaggcaacaaaggtcccttattccaggcagctgaataccagcacagccaggagtccagtgctggtgtttgcagagccaccagaggctccctctcaggtgtccagggcccgcatgctttgtagaatgggcagaatgagcaatgtctgtgcacctgggctttgcaggcagggcctgggtacccaggttcgtgcaatcctctcgtcaccatgaagggagcagcatcattcttcccttcttgaagcaccttggccaccagtataggtaaatttacctcccaggacatgaccattgattctgggatgtcaatgccagagatagtagggtaaatcggcacctgggtaaaactttccattggagactagaaccaaaactcaggacactggcttccaaatgtttctttatcagacaagaaagaccaagtctttccttacgtcttcacatgctgccttggcaaatgctagcattcacaaaccctgggctaccttgacctgtcacccttgcagacctcagacgggtcctgggggcttgctttctcggtttctgtatgcaggcactcaaacctgcatcaggcacctgtgaagggccgggcactgtgctgaggccaaggctccaaatgtgaaccttccaccctcactgaactcacagccagaccagagacaagcaaacaggacatttcacagcagtgcagcctagaaagggccaacaccagcagcatttgtccccccgagcggtagcttttagaagcttccccagtgattcaatgtgtcctacaaatgcctggcccccactcccagagattctgagtcagctggcctagggtgcagccttgacttcactgtgttaaaaagcttcccagataagtccaatgtccggccaagattgagaatcactgacctagagtttaatttaccacctcagtctctatagaccacgcataataatagtaccccacacacctctgagggtccaaagaactttcatttgatcacccatgagaccaccgtggtgtggagatgctttctctctcctgttctcttaacaaagctggtgagcgacagagcctgcagtggaccgggagatggcccagaggagaaagctctgccgtagtcggcctcagttaaccacggagcaccacccctacctgctctcctctcactcctgcttccgtctcggtggagaaagatccaaccgaagcaggacacatctagtcttctggtgcctttaaaatgtacttttccatttgacaaatggattacactaaaaacaaaaatttacaaaaaaaaaaaaaaaacctgaaagaaattgcaggcattaaaatgggactttgcctttattgctcctgggcccatcctatttgggtttttagaaaaacaagcctgaggcaggcccagaaaggctcagggcagaccctccgatcctctgaaaggagcatcaggcaggcaggggttgctccggggccagggaaggggccccgctgggacgcggctgttattgcagctggttggcgcgcagccatgcttagctgcagtgcgggaatgctgggccttctgttctgggctgtttctcatacgcacgtaggccagtgtataaataaggttttattaaatgccaaatgagttctcattaacaaagaaagagggaaaatctcagtaaaccaccgtgacggcatctacccactttgagtcaggagctgggggtgtgagtgcaacctccgagacaagggaacctgtggagcccagagaatcggaggggggcgctggggttagcaccgactgagaccagctgtgttttctctcggttccttggagatcagaagtgagtgttgtcatcttcaaacaatccaaaggcagtacccatggccttactacatccctcccacaccatcccacccatccccgcgcgtacactcacacgctcatttgcacactatcgcacacgctcacttgcgtgcgcacacacagattggtgacctaggtggactgggagagaaataagagccaaatgactggattttctccaaggaaatttattaatagcccctcttggtttcacctgaaggagcttgtcttcacctgcggcctttgcaggcttaacgcccccagcttgaaacccagaagctcagacttgggcccaaggtattattagtgccaacactacctgaaatgtttcgcacctcataaaaatggtgtgtcagtttcgggtgagaggttgggacgcttcccatctgatttggcccaaggcatgcatgcccctccttctccttcccctcctcctccccctcttccccctaccatccttcctgttttctctccaactctggtgcacagctttgaaatcttgctgagaagcaaatctgtcccttctgctttgaatgtttatttgtggaagttcggcaggggaaccgaggcgggtgccaagacctgccatgctgctgggaagtctgagtctccctcccttccccctcctaaatgcttgttgatagagaaaagtcagcctcctcggcatttgggctcacggttttcctttgaaaatgcttccagtgtggcatgattcagctttcttttctgtcccccaaccactgctctgttgtcatttttacttttctgattgcattttatccgtgtctctttgactacggggtggctggacgttgagttccaggaagaaaagggcccaatcttggggttctgactacatgcgcccatcaatgtcctgtttcattcttggctctggctccctgaattcctgagtcactggggagaagcgtgggtggaccgccccctacccagtgagagttgccacagttgctgctctcctgggtcattggttgcagattgttaaacttcacctatgcatttcaactttcgggtggatattgctacgtcaagtgtctgggaaagcccccacagctacaggattttacagtgaggtcccactaatgacttgatgtcatgacttcctcattctttccaatttctcccacttctccataagggttttgggaaggggagaagagaaaggagtgattcctgagtgccagtaccagggaacagcagggctgttgggaggaaacaaaactaaatcaggaaggtttttgttgttgtttttggggggttttatgaaaatattcaagccacagcaaatatatttgatttatagcattagtattttttctgcctgcatctacaaaaatctttacctattaccatcaaaatatcctctgggtgaatggatttcaacaaagaagaaataaaaatgaaatagaagagaggccccttcgtgcacattgagcctactggctggattgtcacttgcctgccttgatgtcttttcagctccaggcaggcagtaggccagggcttattttcatgacagatcagatgttcttttatggatttacaaagaaagaaatactgagaagtcaaaactgaagtcacttaagacaagagcaggcccctgggaaggctgccattgaggataatgagtcctggggtcctggcctttgttcagtaaatacgcactaggcgcctacaatgtgtgcaccaatgtgtgaggcgtcaggttctctccagggtcagttggttttaagaaaggttttggcttctgatatgttttatctctacagaacagtagctcttaacctttcttatgggttaggattaccttcgagaatctgactacagctctagacctgttccctaaagaaaactaagttcacagggacacacaggatggggctcatggagcagctgaagccagaccccaggttaatagcctttacattaaaatgtttttctacctaccactaatatgcattctttagtaagcggtctcaatatacaccgattcttccttaactctgtttatgaagtattcagcatcctccctgcccccttcagcatcctccctgcccctgagcacaggatccaatggcgtgaggaccacaggcctgggcagctgctggggcatacaggcatctcttagtggctgagagactgggccctggctctatgttggctcctaacttgctgccatttaaaggaaatcttagcctcccatccgtaaaatcgagaaaataagacttgtcctacacagctcatgaaatagtaatgaaattcacattagagaagagatggaaaaacactttgaacaaaaagcattttgctcttataaaagcacagcctcttttgagaggccctttgctccccatttctccttcttcagacccccccagactaggagaaggtctgtctcatggagtgaccttttggctgcctctagattccaagctcagttttgctttcattaaccacagatactgggacggacagaaaaagacctagtttctgttgagccaaagagtctcataacttgtctgttcacatacccaagagcccaccctctagttgagacactcagttccctctcattctgggagactgcatgtctctgtgacctcctggtagagaccgtttgacatgtcccccaaccccccagtgattgagtctgaattctccactgatgacgcatttcctagcactcagggtgtcccctcctggttgccccctcaccactgaagcccgcttcctcccttttcatttgatgcttaacaactgtcagtttgcaagaaacatgcttcaaatccacattctcccagttgcctagcaacaacttccctcccggataaatgtgggtttcctgtagctcagcccaggactgaacacagcagcacacacttctgtccactgcttcaactgcttttcacctctggtctgcatgccttcaagactgcagctcatccctcccttcagaaccttccatagcctgcagaggccatgtctgccccaaaaagacacattgaacctgaggctacttatttacccttgtgttaggtatatcctcaacttagaaattaatactgtttccagattgtcttctttgaatcacagaaagtaaaacaacaaaacattcaatgcttaagacatttcatgtgcggttgggtgacatctgtttgatgaacacatttgatccaaagcatcagaaatactatgccaacaagactttttaggaggtgataaacatgtctgttctaccttaagaaaaaaatattacacagtcccaagggagagacatggttttgatcccagacaacccaagcagagacctcttagggccggaatcatcttggctgctgcctaggaccttatatcaatttcttaagcacaggatcaaggcctaaaggccccttagactgacctcagttagtagaggcagatcccttcacagccttatcttccttagaggtctagtctgaccttgaacttcggctggcagtgctgtcagttgtgatgtgtgacatggaagagttatttgttacttggaaaattaagagaacttatttggcataggaaattgtgtgtgtgtgtgtgtgtgtgtgtgtgtgtgtgtgtgtgtgtgtgtgagatgatgtttgccattttgatctgtgacttttttttccagaaatagtttctcagttccattccaactaaacttacagtctcttccggttctttgacagaaacaattcatgtgaatttgaacagataatagggaagggggaaccaaaagaagaggagagccctgggaaagttattttataatttatggcaacctcagtcaggcaactgtgaacaggtacatatggagggctccctcgggactaggcagtattcagagatgtaaggtgtgaggaccggaccctcatcatttaccattcccactaaaaagagctgggaaggaaattgtagctgtagcaccaggcacgtaactggagcttagtaactatttggtgaaggaatattattaaattattaacaagatggaaaaaagggtattaaccacacaaaaatacatctcaagctattgtttctctgttccctttcccccaaattcctagtcttgctcttatctggctgtctctctagtcactctttcttgctgactctcttcacgttcctttctccacctggaattcctgggccctccccttttactgacagacactgtcctcactctcacagtcatcagtttgtctctttacaaacctcagctcaagtgtcacttccccgtccccaggtgaaactgactgctccctccctgtaagtcaccatgatgactgctatatatagccctcatggaacctaaaacctcaacagacacagtctctttcctactctgttatagtttatttactcattaattaccacaacacgtattattgagcacctactgtgtaccatgcccagaagataaaagacaaacaaaataaaacctattcctatgcttaatgagtttacagtctagtggagagatagatacattaaaaaataacagcaaaccaaaataaaagtggtaaataaatgcactgagaaagacaggaatagctaggaggggcacctaatccctagggaaggaaagctggaagagcatggtgatgggggaagaaggctttctggagaaggtgaggtagtttgaaatgagttgactctggccagtaggggtagagtgagaatggggtgagacagggtgggttggtcattttgatccattagtcctcaaagtgataggactagtggctaaggactgcaggctttacagaagcctacaaaactatttgagatttgaagtttttttttttttttaattggctccaaaagaaaatgaaaaaactttagaattataatgaatgaatattaaatgaatatttaaggaaggtaattttattcaacttcattgttaaatttagttaaaacaagcccttgagtttcattcaacactgttttatcataccgttgatgagagaaaacaaaactgattcctggccagggccactgtcagcgtggggtttgcacatctttcccatgtctgcttgggttagctccaggtactcctgtttcccccacatccccaagatgtgcccattagtggaaacggtgtgtctgcatgattccaacgtgagtgagtgtgggtgtgggagtgagtgcccctgccatgggagggcatcctgtccaggttagattcctaccttgtgccctgagctgctgggatggaatccagccacccatgactctgaactgaaataattgggtgaataattatcttactttttaattaatctttgaaaatgtatgtatagttcacatgtatttcaatatttaatattagaagtattttagtctttattttgaagtttggtgatttattgtaaccagaaacaagctatagaaacttaattttgggccaagtgcagtggctcacacctataatcccagcattttgggaggccgaggcagacgcatcacttgaggtccggagttcaagatcagcctggccaacatggtaaaaccctgtctctactaaaaaatacaaaaattagccagatgtggtgggcacctgtagtcccagctacttgggtggctgaagcaggagaatcacttgaacccgggaggcggagcagtgagcagagatcgtgccactgcactcccacctaggcgacagtgtgacactccatctcaaaaaaaaaaaaaaatagaaaagaaagaaacttaattctggtttatatcaattagcctgtggtaaaattggtttcattatagccatttcacttagttgaagtttccaataacctgtggatgaattaagtgaggatttactatattcataaaatcttaaattccaaagcctgtttgcagttcaggtttttccactttacaaacacttctaagtattcacaatgattgcttaaaattcataccagataaatcattaaataagttgttcaaagtcaaataatttcataagtaaaaattaggagcttttagaaaactatacctacatagacctagacctatagatagacagagatctgaatagatatggacacagatgctttccaaagtgttcatgtgatgtgtggtggagtttcaagaccagagtgtgcctggggcctgcagaagtaaaggagaggggatggagagaagattgtccacatggccatgggcaatctcccacccacactcaagtgaggaagacaggaaacaaattcagaaagaagagaaaataatcaaaactgatgggagcttgtgactgatttacttatgcgcagcctccctggagacatgagtgtggctgttccttaggttgtgcctctgggctcctaccccctcttagatgccttcctattatctaggacctggttgctttttgtctgcatagcttctttggattccagtctttgatgccagcttcctcctaaagtagcctttcagatgtcccttggttaccctctgctatctaagggctcatcctaccccacactcattcccagcaccaatttctggatctccaggctggagatttagacaatgggatgggaagaacccatgatgggtcccagacagaaagtggtgccagccacagaaagggcacacaggcacagaagttggtttggggtaagacgatgtggtcagttcagaacacgctggatctaggcagatgcccagcagacagttggatatgtaagtctgaagctctggggagaggtctaggttggaggtacagatttagaagtcatcaacaaaaaggtagcagattaaatgataaaggaaatgagactatccggggagtgtgcagagtgagaggagcaagggaggcccttgggaacctcagcacttcaggggaaggtagaggtacagttgctggtgggaaaggcagagaagtagcaaagcaaaccaggcaaaagcagtgtcacagacgaccagggaggaaaaggacatgatcaaaatgttgagaaaagcagagaggtttgaaaatacaagaagcaaaaatgtccactagacttaaaaaccaggagaaaactggggggttcttgataaagcatcttagtaggatggtgagggtagaagccagggaagtgttggtgaggaagtgaagtcactgattacggactatgcttaaaagaatgtgggaatgaagggtggaagagagaaattagactgtagctagggagacataagcgatcagaggtagattctttctctcctgtgggagaatcttgcacgtatacacagcatgacgacagtgatggaagggctggcgaagcctcagggagactcttggaggtaaaccccatgaagggaggactttgtttcattcactgccgtgtccccagcacctggcacaatagcagacactcaatacatatttgtcaaatgtgggattttatcatttagaaactgcacctggctgtgagtaacaaaagtcagagaaaccgtgggtttcatttttctccccaggcagagtctggagctgggtcctccaagaggggtttggagcaccacaggtttcctcaagacccccaggctgccctgtgtttccctccttcatccccagcatatgcctgtcatctggtgacctccaaacacctgtgctgcctcctccagcacatccatgttgcaggcagggaccaggcaaagggcagaggggcctacttcaaaagaccatttccagaaaccccatcctatgacttctcctggtgtcttggttaccattgtgccataggctcaccctgtatgcatgggaggctgggccaggcattatgacttttagcaatattgcatagataagcatcaatctttgtcactgtgacgaagcctagtcactcagtgctaggcaaggttaatggaatgggttggtgtgtgcattattcttgaggtctttcttatgcttcatgttatacatttattaggacgtttaggcaacagggggataaaaatgaagaggagatgcatgctatgatctgaatgtttgcatcctccccaaaattcatatgttgaaatcttcatccccaagatgatggcattaggaggtggggcctttcggaggcaattaggtcatgactgggattagtgcccttgtaaaaccccagaaagccagcttgccgcttccaccatatgaagacacagagagaagatgccatctacgaatcaggaaatgagcccgcaccatgcaataaacctgctggagccttgatcttagacttcccagctgccagatctgtgggaaatagatttctgttgtttacccagcttatggtattttgttgtagcagccagagtgaactaagacagtgctgatctcgtattcttggagggaacccttagtctttagggaaagcaaagccaccatttggggcagggtgttctccaagtgctgccacatatgctgatgtggttaaactgcaaactatggtaaaaatgtggaggtctgtggaattgtcaatcaggaaaaagatataaaaagaagttaaagtcttcgtgcttctggaaggatatgtgccaaattgttaacattgattatccttgggtagagatgtggggaagtttgcagagacagttttgccttgtactttatataagtaaacagctactacttcgttgtcttaaaaaaaaaaaacagcctatgtgctcttcatgtgactcagaactacctaggcaatacgattaattgaattagtaaaattgagtgattatgaattttcaggaagtcattaatttaccacttctttattacatccacttctaacaggacttcaatataggggaatttgacttcaagataaaaagaccaaatttatttacccttttaaaaaaagacaacttaaaagcagacttgtcttacagaaccttccttagttggacatcgatgagtgtacagaaaatgcaatggataaaaagcttggtgatacaaagataaaaagtggggtcctgtccttaatgaacataccatttcatggagtatcaggtgtataaacaattataatcaatctgcttgttattctgataagatcatttactcacacatcaaatactgagtgcccaccacatgcccagcatacctagaagtcatccagtatgatttctgtctacatggagcatagagtcttacaggggagatagatgacaagtaaacaccagaataattaccaatggtgaagagcacaaggaaggaaacagaactcctaaagagagcgtggctgggcaggggtgagcaagaggcatagaaaaaggggcatctaaatctacttgggaggaagctgtttctcacataggtcatcatgttaggaatgagacttgagggatgagtagaagtttgccaggcaaagaaggaatgggggggaatagagagcagagctaggggcaggagacagctgacgtgtgagcagacataaaaagaagtccactgtggcagcagagaagcaggagagaaggcaagtgagggagccaggcaccagctcacagaggtcatgtgtgtcaaaacgtagtaatggccttctcttctggagacagtagggagccatggaagatgtttgagcagggaaagcgacatgactggattggcctgttgggtaactcagaccacaatgcattggaagggagggggctagaggcaaggggactggcaagaaggccagtcctttttctatgcctattttgatgaaatattctagaagggaagtgaacaaaggtagtcctagagaggaagaacaaaacagataggatacttccttagtatttgctcattcgacaatttatttttgcatatacactaaaaccttttttattattaaaacgttttattgtaggaaaaaagtatgaaagtagagtgaataataaaatgagctcccatggatctatcacccagcttcaactattatcaatatttggctgttcttgttttaactgttctccacctttttttcctgaagtttttttgaagcaaatcacagacaacatatcatttcaccatatgtacttccctctgtatctctaacatgtaagaacttgttttaacaaaatcaccatgctatgatcatacccaacaaaatttatcataatgtcttaataatacctaatacccatttcatgtccactttcccccaattgctacagctggtttgttcagatcagaatcaaaatccacctgtggccattttactgctatgtctctcaggtctcttttcatctctaataatctcaggggagacaggagggaggacgggcaggacttggggctaacttgcttatcgacacacagttttgcctacttgcttcctcccttcacacccactcttcttctcagccccacccttgtatggaaaaaacagaaattaaagtgctttgcccagcacccactgaagctatttcgaaggagtttgaagagtactcccggcaagacaaatgcctcggtccagtgctcaggtcaaagaggggagacgcttctcagtgatgtggtgtcaatagcagcttagttgttctttcctctggaaaattctacccatctgctttgtaactcccatacctaacaaggccttttatttcacaattagaaaataagcctgaaatatgaatgctgcctgagtgtacctacatttattctagagtttcagggtcaaaaagaatacaaggacctctgcatctacagccaagaggagaggggcaaagacacacagctacaaatgagaacctggctggtcaaagcctaactccacctgtttgtcagcactgatgcaagttaggtcagcccaatgatcatttaggagaactgtgctggcaaataaaaagcagaggcttttggtccccagatacttggatgagaattacaagtccagctggttaaaaggcacatgcccagtgctcacttcacacctactcaggaagcacacttgagttggaaaaccactgtctttacacttagaactcagtcctacatgactcctctaggatcagtgattccatcagttttgaaacatgaagcatgaagtcaaacaggacatgaccttggtttccagaaaaccagatgttcacatcagtctctggagcttggaggcagcacacctggggacttccacatcccctgccgaggtggcaaaagcaggagcagtggtgagttcacatgggctggggtttcctgaacactgctggcaattggagaatctgcaagggaacttctccgactcctaccagcagctgctttaaaataaaggtgatgtagctggtcaaatcctccatgagagagcagtgttgaatggaggaagagacacaacctgtctgaaaatggcacaaaggaagaaagatgtaaacaatgacgagaagactgcagtgtctacaaagctccgaggtgaacagatgggcaccccaggcccgcagcacttccttcagtctctgccagctgcactctgttttccttcctccaggaatcttgtttggtgtcactaaaacagcaattagaatcactttgaaatagtgatagtatttaatataactatgaaactatctgtgattgacaagtgcagcaaggagtcttggaatgagagcctttattttttcaattaaataaaagagttttttgtttctaaaagtaatcttgcagaaaagatcctgcgatcagaaagaaggagggggggagttttcaaacatataggagatcagactgtgcctatgtgtgtatatacctacaaacatatatatatttaaaaaattgttttactgtcaattacag

SEQ ID NO: 13 - DNA, 1758 bp, Synthetic construct; identical to SEQ ID NO: 5
  source 1..1758: mol_type = other DNA

SEQ ID NO: 14 - protein, 585 aa, Synthetic construct
MTITYTSQVANARLGSFSRLLLCWRGSIYKLLYGEFLIFLLCYYIIRFIYRLALTEEQQLMFEKLTLYCDSYIQLIPISFVLGFYVTLVVTRWWNQYENLPWPDRLMSLVSGFVEGKDEQGRLLRRTLIRYANLGNVLILRSVSTAVYKRFPSAQHLVQAGFMTPAEHKQLEKLSLPHNMFWVPWVWFANLSMKAWLGGRIRDPILLQSLLNEMNTLRTQCGHLYAYDWISIPLVYTQVVTVAVYSFFLTCLVGRQFLNPAKAYPGHELDLVVPVFTFLQFFFYVGWLKVAEQLINPFGEDDDDFETNWIVDRNLQVSLLAVDEMHQDLPRMEPDMYWNKPEPQPPYTAASAQFRRASFMGSTFNISLNKEEMEFQPNQEDEEDAHAGIIGRFLGLQSHDHHPPRANSRTKLLWPKRESLLHEGLPKNHKAAKQNVRGQEDNKAWKLKAVDAFKSAPLYQRPGYYSAPQTPLSPTPMFFPLEPSAPSKLHSVTGIDTKDKSLKTVSSGAKKSFELLSESDGALMEHPEVSQVRRKTVEFNLTDMPEIPENHLKEPLEQSPTNIHTTLKDHMDPYWALENRDEAHS

SEQ ID NO: 15 - protein, 604 aa, Synthetic construct
MFEKLTLYCDSYIQLIPISFVLGFYVTLVVTRWWNQYENLPWPDRLMSLVSGFVEGKDEQGRLLRRTLIRYANLGNVLILRSVSTAVYKRFPSAQHLVQAGFMTPAEHKQLEKLSLPHNMFWVPWVWFANLSMKAWLGGRIRDPILLQSLLNEMNTLRTQCGHLYAYDWISIPLVYTQVVTVAVYSFFLTCLVGRQFLNPAKAYPGHELDLVVPVFTFLQFFFYVGWLKVAEQLINPFGEDDDDFETNWIVDRNLQVSLLAVDEMHQDLPRMEPDMYWNKPEPQPPYTAASAQFRRASFMGSTFNISLNKEEMEFQPNQEDEEDAHAGIIGRFLGLQSHDHHPPRANSRTKLLWPKRESLLHEGLPKNHKAAKQNVRGQEDNKAWKLKAVDAFKSAPLYQRPGYYSAPQTPLSPTPMFFPLEPSAPSKLHSVTGIDTKDKSLKTVSSGAKKSFELLSESDGALMEHPEVSQVRRKTVEFNLTDMPEIPENHLKEPLEQSPTNIHTTLKDHMDPYWALENRSVLHLNQGHCIALCPTPASLALSLPFLHNFLGFHHCQSTLDLRPALAWGIYLATFTGILGKCSGPFLTSPWYHPEDFLGPGEGR

SEQ ID NO: 16 - protein, 498 aa, Synthetic construct
MFEKLTLYCDSYIQLIPISFVLGFYVTLVVTRWWNQYENLPWPDRLMSLVSGFVEGKDEQGRLLRRTLIRYANLGNVLILRSVSTAVYKRFPSAQHLVQAGFMTPAEHKQLEKLSLPHNMFWVPWVWFANLSMKAWLGGRIRDPILLQSLLNEMNTLRTQCGHLYAYDWISIPLVYTQVVTVAVYSFFLTCLVGRQFLNPAKAYPGHELDLVVPVFTFLQFFFYVGWLKVSLLAVDEMHQDLPRMEPDMYWNKPEPQPPYTAASAQFRRASFMGSTFNISLNKEEMEFQPNQEDEEDAHAGIIGRFLGLQSHDHHPPRANSRTKLLWPKRESLLHEGLPKNHKAAKQNVRGQEDNKAWKLKAVDAFKSAPLYQRPGYYSAPQTPLSPTPMFFPLEPSAPSKLHSVTGIDTKDKSLKTVSSGAKKSFELLSESDGALMEHPEVSQVRRKTVEFNLTDMPEIPENHLKEPLEQSPTNIHTTLKDHMDPYWALENRDEAHS

SEQ ID NO: 17 - DNA, 296 bp, Synthetic construct
  source 1..296: mol_type = other DNA
ggtctcaggagtcaacccccaccagactcacccccctccaccagccccggcaggcttctccttccatctctctgaagcaacttactgatgggccctgccagccaatcacagccagaataacgtatgatgtcaccagcagccaatcagagctcctcgtcagcatatgcagaattctgtcattttactagggtgatgaaattcccaagcaacaccatccttttcagataagggcactgaggctgagagaggagctgaaacctacccggggtcaccacacacaggtggcagatgagacc

SEQ ID NO: 18 - DNA, 1013 bp, Synthetic construct
  source 1..1013: mol_type = other DNA
ggtctcaggaggaaccactgctgcctagagctctgctctcttcattgaacaatacaagaggagtgtgtaggtagacacccaccacttccaacagcttaggagagcccttgagtatggattgatgtattaaaatttattgaatcacatgctgagattttcaccagctgcccgtggggatctgggcatttattcccatattgcactggctggctggaagccagcagcataaactccagggctgttctgtcaacccccaccagactcacccccctccaccagccccggcaggcttctccttccatctctctgaagcaacttactgatgggccctgccagccaatcacagccagaataacgtatgatgtcaccagcagccaatcagagctcctcgtcagcatatgcagaattctgtcattttactagggtgatgaaattcccaagcaacaccatccttttcagataagggcactgaggctgagagaggagctgaaacctacccggggtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggcctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggacagatgagacc

SEQ ID NO: 19 - DNA, 3016 bp, Synthetic construct
  source 1..3016: mol_type = other DNA
ggtctcaggagaaaagacctagatcctagtctcaagccatttatagctgtgtgaccttggacaaggcaggccttgagcttcagtgtcctcctttgtaaaatgtggataaacatgcgatactgaatcatccttcaggaatagctctatgactttccgtaggttgttcccctgatgtgtaatgctcatccattcttctcagctccatgaactcctactcatgcttcaagacccaaccgaaatgccccattgtctgtaaaaatttccctgacctctgtgcacttggcacagatcttaggccttgccattctgtgttagagttgttatttaagtgtttccaaattttttgactatgaatacgtacaggcaggaatcagattaggctcagttctggaaggagaaatggtttggcgaatattttctttgcggaacggaaagcagaagagggcgaccttgactgggttgggtgccatctctgcaggggtcaatcctgggactcttgttccaggcatggcttgggtcaatcagcactcatgactttgctgtgtgcctgagccgtgcctggctaactggggacattttcatagctgagagccagtaggagagagcatgacttgtgccagggtgtgcctgtctcagctctggggcagggccaggagcagctgctcatgggatgtctctggggacatgctggggcctctgggaggggaagggagactggccatgcactgtctatggtcctggtggtactcctgggatattgccaaggagcctgcggttcagggaacagggaggagggactctagtgtctccaactccctctcatggctctatggctgctggtcggggtcagggagatgaagcagtgccagctccgtggtctgcactgatcctgaccagtgaaccaggcttccccagccagagccagctagtcaagcacgctctctagtggccaccacagggaaggcaagaacttgtctccacaaaggcaggattttcatttattccttcagagagtacagaatgagcatcaatgttccaggcatggtgccaggcatgtgtgttaactttagttaaaacatacttattggccgagtgtggcggctcactcctgtaatcccaccactttgggaggcctaggtgggaggactgcttgaacttagagctcaataccagcctgggtaatgtagtgagagctatctctataaaaaaaaaaaaaatagctgggtgtggtggtgtgcgtctgtagtcccagcgacttgggaggctgagatgggaggattgcttgagcctgggaagtggaggttgcaatgagccaaaatcacacccattgcactccagcctgggtgaccttgtctcaaaaatatagttatatatttacttatatatttatatgtataaatatatgtgtacatacaaatatatttgtaatattatagacatattaatacatatttacatatgttaatatataaatatatataatatatatttatcatcttctaaatggaaagctcttgagtttcaggatgtgttggttgggaagggaatacaggatagggaaataatccattaagcaatgactcagcacaggccataaaatcagaatcacctggagaacttgaacaatcccaacacctggggtccatccagaccaatcaaatcagaattcgtaggggtcaggcacaggcaccagtggtttggagagttgacggacacatgtcccatccatcattctcattcggctgccaaggtaggcatcgttgtcccattttgcatatgaggagactgaggctgaagtgaacctgcttctttccccatatcagttgaggacagcgatggggcagcggggaggcagtaaatactcctacaaggctgcctctaaagagagtcttataataacaacagctttattgtttgccatgattagaaaagtattcaaggtcattgcgaaatagtgtttttttttctaggcagataattatataaaggcaaaaatggtgtgtgggggtgggggacaaggggagggagagcattaggacaaatacctaatgcacgcaaggcttaaaacctagatgatgggttgacaggtgcagcaaaccaccatggcacacgtatacctatgtaacaaacctgcacatcatgtatacctatgtaacaaacctgtacattctgcacacgtatcccagaacttaaagtgaaaaaaaaagtggtgtgtagaaaaatcacctgcaatctcagcatagttaacgcttagtacatttcagagagagagggtgacaggaaagggaggatgagagtgggtttaagacacaaggtcatattataaaatcagggcttctggaagtttagtcccaaaaccacacatctcataatcccctgcagtgcttgattaaaatgcaacatccctaaggccacagactcagactctggagaaagatccagaaaactgcccgtttaataaacatttgggcgattcttacggcctctaaagaccaagaaccactgctgcctagagctctgctctcttcattgaacaatacaagaggagtgtgtaggtagacacccaccacttccaacagcttaggagagcccttgagtatggattgatgtattaaaatttattgaatcacatgctgagattttcaccagctgcccgtggggatctgggcatttattcccatattgcactggctggctggaagccagcagcataaactccagggctgttctgtcaacccccaccagactcacccccctccaccagccccggcaggcttctccttccatctctctgaagcaacttactgatgggccctgccagccaatcacagccagaataacgtatgatgtcaccagcagccaatcagagctcctcgtcagcatatgcagaattctgtcattttactagggtgatgaaattcccaagcaacaccatccttttcagataagggcactgaggctgagagaggagctgaaacctacccggggtcaccacacacaggtggcagatgagacc

SEQ ID NO: 20 - DNA, 2464 bp, Synthetic construct
  source 1..2464: mol_type = other DNA
ggtctcaggaggaaccactgctgcctagagctctgctctcttcattgaacaatacaagaggagtgtgtaggtagacacccaccacttccaacagcttaggagagcccttgagtatggattgatgtattaaaatttattgaatcacatgctgagattttcaccagctgcccgtggggatctgggcatttattcccatattgcactggctggctggaagccagcagcataaactccagggctgttctgtcaacccccaccagactcacccccctccaccagccccggcaggcttctccttccatctctctgaagcaacttactgatgggccctgccagccaatcacagccagaataacgtatgatgtcaccagcagccaatcagagctcctcgtcagcatatgcagaattctgtcattttactagggtgatgaaattcccaagcaacaccatccttttcagataagggcactgaggctgagagaggagctgaaacctacccggggtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggcctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagaccttctgtgggatcatcggacccacctggaaccccacctgtgagtacaaggtgccccaggtggactgggctggggctttgaggccttcagggttggatggccatcttgcgtatttgtgtgggatatgcacacacaggcagcacatgcgcaggtgtgtgggcacctgtgtgtctgtgcaaatgccctgaggtgggaatgagcttggtgtgcatcaggcacagccagccagtgtggctgcagcaaaacacacagggaaagaatggagggggcatcaatcactgcttcagtaaatttttattgagcgccttctacgagaacacaagaggagcttccattctgaggaggaaacaggcaggaaacaggcagatatcctgtataatttcaagtagtgataagtgctctctagaaatatcaagcaaggtgaggagacacagagcaccggtggcagtgggactcaggaggggctctatttccaggttggatggttgggaacatcctttctaaagggaacctggagtgggaaggaaccatgcaggtatctcaggaggagcttcctccaggcaggaagatcagcaggtggaaaggccctggagccaccattcagtaaacatcatttgagcatctctaccagctaggttccattatgggaatgggaatatggtggtggacagggctgcctggtcccttccatacttctcacactagggtggttgagagagcttgggagctaacgaacaagatgggctgagaacactgcctagcccagaggacctgagcttagtgtgtagacattgctgctgttactgcctttgtcattgtattatttatttatttatttatttatttttagacagagttttgctcctcttacccaggctggagtgcaatggcgtgatctcagctcactgcaacctccacctcctgggttcaagcgattctcctgcctcagcctcctgagtagctgggattacaggcacccgcaccacgcctggataatttttttgtatttttagtagagacagggtttcaccatgttggccaggctggcctcgaactcctgaccttaggtgatccacctgcctcgacttcccaaagtgctgggattataggcatgagccactgcgcccagtgattatagaaagttaaaggcacatggcaatgcacacgcctatctacgtcttccctgccaaagcaaagggcagcctctgggctcactttcttgcgtttctacttccaaaaggcagtcagaactggcagggcttggaggccacttcatccacctcctagggtccctatgggagagttgaggtccagagcagggaagggtcctgacaggctctgaccagggcctctgatccctacaaacccccaatcggtgtccctctctaccaggacccaagcccacctgctgcagcccactgcctggcccagatgagacc

SEQ ID NO: 21 - DNA, 3763 bp, Synthetic construct
  source 1..3763: mol_type = other DNA
cctgtcccccataacctgcccacctctctgccccattcccacagctcactgcattttgtccctgctgccagtcgtgtgccatgggctctcctgtgctcccccgggctctcatttccccatccgtgtagggctgacgccactggccccgcccagcctgggaggatggagaggggggacttgaggagtggccagtactgggtttcggcagcaggtgcaaatcccaggtggggactgtgttccagggactcctggactcctgttaggccagcaggctaaggggcagacggcatcctggggtcttcctgggacagggtcgtcgggtctgaggggacccaggccctagcagaagctgactcctgtcacggtcacttcccagcggtgctcagcccgcctgacggaagcagctgggcagtggggcctgtgaccagcaggcggcgctggcgagctatagcgcccctttgggcccccaccccaccttctgggcagcttccctgcagacaccccagttatgggggctagggacccaaaagagacatccttctgccacccagagctgccctggcgaggtgcactatggggccgccgacagctgcgtggctgccgagggcggaaaggagaaactgtcatgtcccgatagggccgcgcgagctctccatcctcgacaacgctaataacaaagacgtgtgctcctctttgcttggttccccccactcctttaaatcacagatttcacttcagtttatctgtgtcgctgtcacacgtggggtggctcccagtcagctggtttggcaaagtttctggatgattacggaataacatgtgtccccaacccgcagagcaggttgtgggggcaatgttgcattgaccagcgtcagagaacacacatcagaggcaagggtgggtgtgcaggagggagaaggcgcagaaggcagggctttagctcagcactctccctcctgccatgctctgcctgaccgttccctctctgagtcccaaacagccaggtagaggaggaagaaatggggctgagagcccagcacatcagtgattaagtcaggatcaggtgcggtttcctgctcaggtgctgagacagcaggcggtgtcctgcaaacaacaggaggcacctgaagctagcctggggggcccacgcccaggtgcggtgcattcagcagcacagccagagacagaccccaatgaccccgcctccctgtcggcagccagtgctctgcacagagccctgagcagcctctggacattagtcccagccccagcacggcccgtcccccacgctgatgtcaccgcacccagaccttggaggccccctccggctccgcctcctgggagaaggctctggagtgaggaggggagggcagcagtgctggctggacagctgctctgggcaggagagagagggagagacaagagacacacacagagagacggcgaggaagggaaagacccagagggacgcctagaacgagacttggagccagacagaggaagaggggacgtgtgtttgcagactggctgggcccgtgacccagcttcctgagtcctccgtgcaggtggcagctgtaccaggctggcaggtcactgagagtgggcagctgggccccaggtaaggatgggctgcccactgtcctgggcattgggaggggtttggatgtggaggagtcatggacttgagctacctctagagcctctgccccacagccacttgctcctgggactgggcttcctgccacccttgagggctcagccaccacagccactgaatgaaactgtcccgagcctgggaagatggatgtgtgtcccctggaggagggaagagacaaggagcatgttgtccatcgaatcttctctgagctggggctggggttagtggcatcctggggccaggggaatagacatgctgtggtggcagagagaagagtccgttctctctgtctcctttgctttctctctgacactctttatctccgtttttggataagtcacttccttcctctatgccccaaatatcccatctgtgaaatgggagtatgaagccccaacagccagggttgtagtggggaagaggtaaaatcaggtatagacatagaaatacaaatacagtctatgccccctgttgtcagttggaaaagaaattaacttgaaggtggtctagttctcatttttagaaatgaaatgtctgtctggtcattttaaaatgtggcccttaaatttcacgccctcaccactctcccccatcccttggagccccatgtctctagtgaaagcactggctctgcccccagccctcatggctcatgctggcatagggcgcctgctccacagcctgggcaccatcttcagacaagtgcccggtggcaactgcctgctggccctgttgaatccacatctccaccaggcatccagactagttcagctctctggaaggactgtgggtttgctgtgtcccagagctccagggcaggggtcagggctcggatgtcgggcagtgtcatgggcagaggatcgaatgccccggcggctctgaatgggcccttgtgaaaaattgatgcgcattctaggagacaggttgggagccagaggggcctcataccagggtctgtaggctggggctgccttttaagctccttcctgaggccgtctctgggtctggccctgtgctggacaaggctggagacaaggcaatgtctcagaccctctcccattggccacatcctgccctggatcaactcgccaactttgggggcagaggtgggactgacccttaccctgacaacataatgcatatagtcaaaatgggataaaggggaatatagaggctcttggcagcttgggagtggtcagggaaggcttcctggaggaggtatcatctgaactgagccatgaaccataagtggaaattcactagtcaaaatttcaggtagaagggccagtgtgtgaaggccaggagatggcaagagctggcgtatttcaggaacagtgagtcactgaggatgtccaagtataagggtaggaaagggagtgagcagtgagagaaaagaccgaggcatcagcaggggccagattgtgctgggcctagcggggcgggcccgggcccgggcccaggcccaggtgcggtgcattcagcagcacagccagagacagaccccaatgaccctgcctccccgtcagcagccagtgctctgcacagagccatcctgagggcagtgggtgctcttgagaggtttcaggcagggtgtgctgtgagcaggtcatgcccagcccttgaccttctgctcagtcaggcttgtccttgtcacccacattcctggggcagtccctaagctgagtgccggagattaagtcctagtcctaaatttgctctggctagctgtgtgaccctgggcaagtcttggtccctctctgggcccctttgccgtaggtccctggtggggccagacttgctactttctaggagccctttgggaatctctgaatgacagtggctgagagaagaattcagctgctctgggcagtggtgctggtgacagtggctgaggctcaggtcacacaggctgggcagtggtcagagggagagaagccaaggagggttcccttgagggaggaggagctggggctttgggaggagcccaggtgaccccagccaggctcaaggcttccagggctggcctgcccagaagcatgacatgctctctctccctgcagaactgtgcctggcccagtgggcagcaggagctcctgacttgggacc

SEQ ID NO: 22 - DNA, 1266 bp, Synthetic construct
  source 1..1266: mol_type = other DNA
gggcggaaaggagaaactgtcatgtcccgatagggccgcgcgagctctccatcctcgacaacgctaataacaaagacgtgtgctcctctttgcttggttccccccactcctttaaatcacagatttcacttcagtttatctgtgtcgctgtcacacgtggggtggctcccagtcagctggtttggcaaagtttctggatgattacggaataacatgtgtccccaacccgcagagcaggttgtgggggcaatgttgcattgaccagcgtcagagaacacacatcagaggcaagggtgggtgtgcaggagggagaaggcgcagaaggcagggctttagctcagcactctccctcctgccatgctctgcctgaccgttccctctctgagtcccaaacagccaggtagaggaggaagaaatggggctgagagcccagcacatcagtgattaagtcaggatcaggtgcggtttcctgctcaggtgctgagacagcaggcggtgtcctgcaaacaacaggaggcacctgaagctagcctggggggcccacgcccaggtgcggtgcattcagcagcacagccagagacagaccccaatgaccccgcctccctgtcggcagccagtgctctgcacagagccctgagcagcctctggacattagtcccagccccagcacggcccgtcccccacgctgatgtcaccgcacccagaccttggaggccccctccggctccgcctcctgggagaaggctctggagtgaggaggggagggcagcagtgctggctggacagctgctctgggcaggagagagagggagagacaagagacacacacagagagacggcgaggaagggaaagacccagagggacgcctagaacgagacttggagccagacagaggaagaggggacgtgtgtttgcagactggctgggcccgccgagctgagtcctccagccccccttcctactgagggccacagtggcctctgaccctcagcccttccccgtgatcagtctgaggctctaatgagctcctggcctccagagcaggagccaggaatgtgggccctgagccaggtgggcctggcagcaggccgtagggcttgtggggctgtgcattatgggggcttggccagcagcttctcgctctccagggtcaccagaggccaatggctcagccttgcctggccaccctgaggctgcacggaccagcatgtttgcatatgagtgcgtgcgcgtgtgtgtgtctgtgtgtgtgagtgagaggcagg

SEQ ID NO: 23 - DNA, 348 bp, Synthetic construct
  source 1..348: mol_type = other DNA
ccccaatgaccccgcctccctgtcggcagccagtgctctgcacagagccctgagcagcctctggacattagtcccagccccagcacggcccgtcccccacgctgatgtcaccgcacccagaccttggaggccccctccggctccgcctcctgggagaaggctctggagtgaggaggggagggcagcagtgctggctggacagctgctctgggcaggagagagagggagagacaagagacacacacagagagacggcgaggaagggaaagacccagagggacgcctagaacgagacttggagccagacagaggaagaggggacgtgtgtttgcagactggctgggcccg

SEQ ID NO: 24 - DNA, 923 bp, Synthetic construct
  source 1..923: mol_type = other DNA
agggatcacttcagatctgccgagtgagtcgattggacttaaagggccagtcaaaccctgactgccggctcatggcaggctcttgccgaggacaaatgcccagcctatatttatgcaaagagattttgttccaaacttaaggtcaaagatacctaaagacatccccctcaggaacccctctcatggaggagagtgcctgagggtcttggtttcccattgcatcccccacctcaatttccctggtgcccagccacttgtgtctttagggttctctttctctccataaaagggagccaacacagtgtcggcctcctctccccaactaagggcttatgtgtaattaaaagggattatgctttgaaggggaaaagtagcctttaatcaccaggagaaggacacagcgtccggagccagaggcgctcttaacggcgtttatgtcctttgctgtctgaggggcctcagctctgaccaatctggtcttcgtgtggtcattagcgtaacagttactgtctgtggtttaaaaatgaggtgtggagcaaataaacaggttggaagtgtggggtggggtggtggggtagggtggtggggcagggtggggggttgtgagcagtcagtgggcttgtcgccgattagcactgaagcagtgtttagctggacggcctttctgtgggcccctctgacagtgcccttcccaggaagatgtgtttctctgtcctcagccacatgaaaatcttttgcctaccgtgcctgtcaatccattgcctgcccgcccctcccccaccccccgttttacacctgcctgtccagtctaccgctctctagggcatccacgctgagcagtgggaagaactttaagccctgatgagcaggccaaaggcaagcaagaaccccctcgaacagcttcccagcttagtgaggcctta

SEQ ID NO: 25 - DNA, 1810 bp, Synthetic construct
  source 1..1810: mol_type = other DNA
caagataagctattcctccctgtctttgcagagtttaaatgactcacttgttcaagtacccaccgttgccatgtgggaccgtgagcaaagtacttaatctcactaagcttcacgttcctcatctgtaaaacagcaaatatggacctcacaaaattgtagtgaggctaaaatgaaataacatatgcaaaagcagtttataaataataaacttactataaaatattattttgtaattctgcaagcttgtcttaaatgccatcacctccaaggagcctttttgccatcataagcagaaactatctctctcttcttggaagctccaccatgcacagcctatgggccctcatcacactccttgagttattcgagttcaagtcccgtgtttacaaccagaccgcaaactctatgaagtcagcatccattcctctctgtggttctccctccgccccatccaggctcaagggtctagagtctttcaaagagaacacattctgagatttgaggaggcagagacaaaaagttccactgcgaagtgccagggaggcttctgtttggggtgtcccttgggatcacagatcccccacctggtgatgagtcaacccagcaccaccccattgcagggctggaatgacagtaatgggcccacctgctgcctctcctcatacccgcaccccagtcagacattgcaagtcagtcacggctctgtcctgctgggcctggagtgttccagtgccttttccatcacagcaccaagcagccactactagtcgatcaatttcagcacaagagataaacatcattaccctctgctaagctcagagataacccaactagctgaccataatgacttcagtcattacggagcaagataaaagactaaaagagggagggatcacttcagatctgccgagtgagtcgattggacttaaagggccagtcaaaccctgactgccggctcatggcaggctcttgccgaggacaaatgcccagcctatatttatgcaaagagattttgttccaaacttaaggtcaaagatacctaaagacatccccctcaggaacccctctcatggaggagagtgcctgagggtcttggtttcccattgcatcccccacctcaatttccctggtgcccagccacttgtgtctttagggttctctttctctccataaaagggagccaacacagtgtcggcctcctctccccaactaagggcttatgtgtaattaaaagggattatgctttgaaggggaaaagtagcctttaatcaccaggagaaggacacagcgtccggagccagaggcgctcttaacggcgtttatgtcctttgctgtctgaggggcctcagctctgaccaatctggtcttcgtgtggtcattagcgtaacagttactgtctgtggtttaaaaatgaggtgtggagcaaataaacaggttggaagtgtggggtggggtggtggggtagggtggtggggcagggtggggggttgtgagcagtcagtgggcttgtcgccgattagcactgaagcagtgtttagctggacggcctttctgtgggcccctctgacagtgcccttcccaggaagatgtgtttctctgtcctcagccacatgaaaatcttttgcctaccgtgcctgtcaatccattgcctgcccgcccctcccccaccccccgttttacacctgcctgtccagtctaccgctctctagggcatccacgctgagcagtgggaagaactttaagccctgatgagcaggccaaaggcaagcaagaaccccctcgaacagcttcccagcttagtgaggcctta

SEQ ID NO: 26 - DNA, 1382 bp, Synthetic construct
  source 1..1382: mol_type = other DNA
caagataagctattcctccctgtctttgcagagtttaaatgactcacttgttcaagtacccaccgttgccatgtgggaccgtgagcaaagtacttaatctcactaagcttcacgttcctcatctgtaaaacagcaaatatggacctcacaaaattgtagtgaggctaaaatgaaataacatatgcaaaagcagtttataaataataaacttactataaaatattattttgtaattctgcaagcttgtcttaaatgccatcacctccaaggagcctttttgccatcataagcagaaactatctctctcttcttggaagctccaccatgcacagcctatgggccctcatcacactccttgagttattcgagttcaagtcccgtgtttacaaccagaccgcaaactctatgaagtcagcatccattcctctctgtggttctccctccgccccatccaggctcaagggtctagagtctttcaaagagaacacattctgagatttgaggaggcagagacaaaaagttccactgcgaagtgccagggaggcttctgtttggggtgtcccttgggatcacagatcccccacctggtgatgagtcaacccagcaccaccccattgcagggctggaatgacagtaatgggcccacctgctgcctctcctcatacccgcaccccagtcagacattgcaagtcagtcacggctctgtcctgctgggcctggagtgttccagtgccttttccatcacagcaccaagcagccactactagtcgatcaatttcagcacaagagataaacatcattaccctctgctaagctcagagataacccaactagctgaccataatgacttcagtcattacggagcaagataaaagactaaaagagggagggatcacttcagatctgccgagtgagtcgattggacttaaagggccagtcaaaccctgactgccggctcatggcaggctcttgccgaggacaaatgcccagcctatatttatgcaaagagattttgttccaaacttaaggtcaaagatacctaaagacatccccctcaggaacccctctcatggaggagagtgcctgagggtcttggtttcccattgcatcccccacctcaatttccctggtgcccagccacttgtgtctttagggttctctttctctccataaaagggagccaacacagtgtcggcctcctctccccaactaagggcttatgtgtaattaaaagggattatgctttgaaggggaaaagtagcctttaatcaccaggagaaggacacagcgtccggagccagaggcgctcttaacggcgtttatgtcctttgctgtctgaggggcctcagctctgaccaatctggtcttcgtgtggtcattag

SEQ ID NO: 27 - DNA, 495 bp, Synthetic construct
  source 1..495: mol_type = other DNA
agggatcacttcagatctgccgagtgagtcgattggacttaaagggccagtcaaaccctgactgccggctcatggcaggctcttgccgaggacaaatgcccagcctatatttatgcaaagagattttgttccaaacttaaggtcaaagatacctaaagacatccccctcaggaacccctctcatggaggagagtgcctgagggtcttggtttcccattgcatcccccacctcaatttccctggtgcccagccacttgtgtctttagggttctctttctctccataaaagggagccaacacagtgtcggcctcctctccccaactaagggcttatgtgtaattaaaagggattatgctttgaaggggaaaagtagcctttaatcaccaggagaaggacacagcgtccggagccagaggcgctcttaacggcgtttatgtcctttgctgtctgaggggcctcagctctgaccaatctggtcttcgtgtggtcattag

SEQ ID NO: 28 - DNA, 1681 bp, Synthetic construct
  source 1..1681: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacagggagtttaaa

SEQ ID NO: 29 - DNA, 1179 bp, Synthetic construct
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 30 - DNA, 21 bp, Synthetic construct
  source 1..21: mol_type = other DNA
cctagcctttaaggggctcta

SEQ ID NO: 31 - DNA, 23 bp, Synthetic construct
  source 1..23: mol_type = other DNA
tgacctctgtgaaaagatcaggg

SEQ ID NO: 32 - DNA, 22 bp, Synthetic construct
  source 1..22: mol_type = other DNA
tgggagttagacccggcgagtg

SEQ ID NO: 33 - DNA, 22 bp, Synthetic construct
  source 1..22: mol_type = other DNA
tgtagcatctccgcttgtcact

SEQ ID NO: 34 - DNA, 20 bp, Synthetic construct
  source 1..20: mol_type = other DNA
cagtcttctgggtggcagtg

SEQ ID NO: 35 - DNA, 24 bp, Synthetic construct
  source 1..24: mol_type = other DNA
aaccatgagaagtatgacaacagc

SEQ ID NO: 36 - DNA, 12495 bp, Synthetic construct
  source 1..12495: mol_type = other DNA
ttcggtatcacaggagaatttcagggagcaagataagctattcctccctgtctttgcagagtttaaatgactcacttgttcaagtacccaccgttgccatgtgggaccgtgagcaaagtacttaatctcactaagcttcacgttcctcatctgtaaaacagcaaatatggacctcacaaaattgtagtgaggctaaaatgaaataacatatgcaaaagcagtttataaataataaacttactataaaatattattttgtaattctgcaagcttgtcttaaatgccatcacctccaaggagcctttttgccatcataagcagaaactatctctctcttcttggaagctccaccatgcacagcctatgggccctcatcacactccttgagttattcgagttcaagtcccgtgtttacaaccagaccgcaaactctatgaagtcagcatccattcctctctgtggttctccctccgccccatccaggctcaagggtctagagtctttcaaagagaacacattctgagatttgaggaggcagagacaaaaagttccactgcgaagtgccagggaggcttctgtttggggtgtcccttgggatcacagatcccccacctggtgatgagtcaacccagcaccaccccattgcagggctggaatgacagtaatgggcccacctgctgcctctcctcatacccgcaccccagtcagacattgcaagtcagtcacggctctgtcctgctgggcctggagtgttccagtgccttttccatcacagcaccaagcagccactactagtcgatcaatttcagcacaagagataaacatcattaccctctgctaagctcagagataacccaactagctgaccataatgacttcagtcattacggagcaagataaaagactaaaagagggagggatcacttcagatctgccgagtgagtcgattggacttaaagggccagtcaaaccctgactgccggctcatggcaggctcttgccgaggacaaatgcccagcctatatttatgcaaagagattttgttccaaacttaaggtcaaagatacctaaagacatccccctcaggaacccctctcatggaggagagtgcctgagggtcttggtttcccattgcatcccccacctcaatttccctggtgcccagccacttgtgtctttagggttctctttctctccataaaagggagccaacacagtgtcggcctcctctccccaactaagggcttatgtgtaattaaaagggattatgctttgaaggggaaaagtagcctttaatcaccaggagaaggacacagcgtccggagccagaggcgctcttaacggcgtttatgtcctttgctgtctgaggggcctcagctctgaccaatctggtcttcgtgtggtcattagcgtaacagttactgtctgtggtttaaaaatgaggtgtggagcaaataaacaggttggaagtgtggggtggggtggtggggtagggtggtggggcagggtggggggttgtgagcagtcagtgggcttgtcgccgattagcactgaagcagtgtttagctggacggcctttctgtgggcccctctgacagtgcccttcccaggaagatgtgtttctctgtcctcagccacatgaaaatcttttgcctaccgtgcctgtcaatccattgcctgcccgcccctcccccaccccccgttttacacctgcctgtccagtctaccgctctctagggcatccacgctgagcagtgggaagaactttaagccctgatgagcaggccaaaggcaagcaagaaccccctcgaacagcttcccagcttagtgaggccttacagataagtttgtacaaaaaagagaggtgccaccatgggctttgtgcgacagattcagctgctgctgtggaagaactggaccctgcggaagcggcagaaaatcagattcgtggtggaactcgtgtggcccctgagcctgtttctggtgctgatctggctgcggaacgccaatcctctgtacagccaccacgagtgtcacttccccaacaaggccatgccttctgccggaatgctgccttggctgcagggcatcttctgcaacgtgaacaacccctgctttcagagccccacacctggcgaaagccctggcatcgtgtccaactacaacaacagcatcctggccagagtgtaccgggacttccaagagctgctgatgaacgcccctgagtctcagcacctgggcagaatctggaccgagctgcacatcctgagccagttcatggacaccctgagaacacaccccgagagaatcgccggcaggggcatcagaatccgggacatcctgaaggacgaggaaaccctgacactgttcctcatcaagaacatcggcctgagcgacagcgtggtgtacctgctgatcaacagccaagtgcggcccgagcagtttgctcatggcgtgccggatctcgccctgaaggatatcgcctgttctgaggccctgctggaacggttcatcatcttcagccagcggagaggcgccaagaccgtcagatatgccctgtgcagtctgagccagggaaccctgcagtggatcgaggataccctgtacgccaacgtggacttcttcaagctgttccgggtgctgcccacactgctggattctagatcccagggcatcaacctgagaagctggggcggcatcctgtccgacatgagcccaagaatccaagagttcatccaccggcctagcatgcaggacctgctgtgggttaccagacctctgatgcagaacggcggacccgagacattcaccaagctgatgggcattctgagcgatctgctgtgcggctaccctgaaggcggaggatctagagtgctgagcttcaattggtacgaggacaacaactacaaggccttcctgggcatcgactccaccagaaaggaccccatctacagctacgaccggcggacaaccagcttctgcaatgccctgatccagagcctggaaagcaaccctctgaccaagatcgcttggagggccgccaaacctctgctgatgggaaagatcctgtacacccctgacagccctgccgccagaagaatcctgaagaacgccaacagcaccttcgaggaactggaacacgtgcgcaagctggtcaaggcctgggaagaagtgggacctcagatttggtacttcttcgacaatagcacccagatgaacatgatcagagacaccctgggcaaccctaccgtgaaggacttcctgaacagacagctgggcgaagagggcattaccgccgaggccatcctgaactttctgtacaagggccccagagagtcccaggccgacgacatggccaacttcgattggcgggacatcttcaacatcaccgacagaaccctgcggctggtcaaccagtacctggaatgcctggtgctggacaagttcgagagctacaacgacgagacacagctgacccagagagccctgtctctgctggaagagaatatgttctgggctggcgtggtgttccccgacatgtacccttggacaagcagcctgcctcctcacgtgaagtacaagatccggatggacatcgacgtggtcgaaaagaccaacaagatcaaggaccggtactgggacagcggccctagagctgatcccgtggaagattttcggtacatctggggcggattcgcatacctgcaggacatggtggaacagggaatcacacggtcccaggtgcaggctgaagctcctgtgggaatctacctgcagcagatgccttatccttgcttcgtggacgacagcttcatgatcatcctgaatcggtgcttccccatcttcatggtgctggcctggatctactccgtgtctatgaccgtgaagtccatcgtgctggaaaaagagctgcggctgaaagagacactgaagaaccagggcgtgtccaatgccgtgatctggtgcacctggtttctggacagcttctccattatgagcatgagcatctttctgctgacgatcttcatcatgcacggccgaatcctgcactacagcgacccctttatcctcttcctgttcctgctggccttcagcaccgctacaatcatgctgtgttttctgctgtccaccttcttcagcaaggcctctctggccgctgcttgtagcggcgtgatctacttcaccctgtacctgcctcacatcctgtgcttcgcatggcaggacagaatgaccgccgagctgaagaaagctgtgtccctgctgagccctgtggcctttggctttggcaccgagtacctcgtcagatttgaggaacaaggactgggactgcagtggtccaacatcggcaatagccctacagagggcgacgagttcagcttcctgctgtctatgcagatgatgctgctggacgccgccgtgtatggactgctggcttggtatctggaccaggtgttcccaggcgattacggcactcctctgccttggtatttcctgctgcaagagagctactggctcggcggcgagggatgtagcaccagagaagaaagagccctggaaaagaccgagcctctgaccgaggaaacagaggaccctgaacacccagagggcatccacgatagctttttcgagagagaacaccccggctgggtgccaggcgtgtgtgtgaagaatctggtcaagattttcgagccctgcggcagacctgccgtggacagactgaacatcaccttctacgagaaccagattaccgcctttctgggccacaacggcgctggcaagacaaccacattgagcatcctcacaggcctgctgcctccaacaagcggcacagttctcgttggcggcagagacatcgagacaagcctggatgccgtcagacagtccctgggcatgtgccctcagcacaacatcctgtttcaccacctgaccgtggccgagcacatgctgttttatgcccagctgaagggcaagagccaagaagaggctcagctggaaatggaagccatgttggaggacaccggcctgcaccacaagagaaatgaggaagcccaggatctgagcggcggcatgcagagaaaactgagcgtggccattgccttcgtgggcgacgccaaggttgtgatcctggatgagcctacaagcggcgtggacccttacagcagaagatccatctgggatctgctgctgaagtacagatcaggccggaccatcatcatgagcacccaccacatggacgaggccgatctgctcggagacagaatcgccatcattgctcagggcagactgtactgcagcggcaccccactgtttctgaagaactgtttcggcaccggactgtatctgaccctcgtgcggaagatgaagaacatccagtctcagcggaagggcagcgagggcacctgtagctgttctagcaagggctttagcaccacctgtccagctcacgtggacgatctgacccctgaacaggtgctggatggcgacgtgaacgagctgatggacgtggtgctgcaccatgtgcctgaggccaagctggtggaatgcatcggccaagaactgatttttctgctcccgaacaagaacttcaagcaccgggcctacgccagcctgttcagagagctggaagaaaccctggccgacctgggcctgtctagctttggcatcagcgacacccctctcgaagagattttcctgaaagtgacagaggacagcgatagcggccctctgtttgctggcggagcacagcaaaagcgcgagaacgtgaaccctagacacccctgtctgggcccaagagagaaagccggacagacccctcaggacagcaatgtgtgctctcctggtgctcctgccgctcatcctgagggacaacctccacctgaacctgagtgtcctggacctcagctgaacaccggaacacagctggttctgcagcacgtgcaggctctgctcgtgaagagattccagcacaccatcagaagccacaaggactttctggcccagatcgtgctgcccgccacctttgtttttctggctctgatgctgagcatcgtgatccctccattcggcgagtaccccgctctgacactgcacccttggatctacggccagcagtacacctttttctccatggacgaacccggcagcgagcagttcacagtgctggctgatgtcctgctgaacaagcccggcttcggcaaccggtgtctgaaagaaggatggctgcctgagtacccttgcggcaacagcacaccttggaaaacccctagcgtgtcccctaacatcacccagctgttccaaaagcagaaatggacccaagtgaacccctctccatcctgccggtgctccacaagggaaaagctgaccatgctgcccgagtgtccagaaggcgctggcggacttcctccacctcagagaacacagagatccaccgagattctccaggacctgaccgaccggaatatcagcgacttcctggttaagacataccccgcactgatccggtccagcctgaagtccaagttctgggtcaacgaacagagatacggcggcatcagcatcggcggaaaactgcctgtggtgcctatcacaggcgaggcccttgtgggctttctgtccgatctggggagaatcatgaacgtgtccggcggacctatcaccagggaagccagcaaagagatccccgatttcctgaagcacctggaaaccgaggacaatatcaaagtgtggttcaacaacaaaggatggcacgccctcgtgtcttttctgaacgtggcccacaatgccatcctgcgggctagcctgcctaaggacagaagccctgaggaatacggcatcaccgtgatctcccagcctctgaatctgaccaaagagcagctgagcgagatcaccgtgctgaccacctctgtggatgctgtggtggccatctgcgtgatcttcagcatgagcttcgtgcccgcctccttcgtgctgtacctgattcaagagagagtgaacaagagcaagcacctccagttcatctccggggtgtccccaaccacctactgggtcaccaattttctgtgggacatcatgaactacagcgtgtcagccggcctggtcgtgggcatctttatcggctttcagaagaaggcctacacgagccccgagaacctgcctgctttggttgctctgctgctcctgtatggctgggccgtgattcccatgatgtaccccgccagctttctgtttgacgtgcccagcacagcctacgtggccctgtcttgcgccaatctgttcatcggcatcaacagcagcgccatcacattcatcctggaactgttcgagaacaacaggaccctgctgcggttcaacgccgtgctgcggaaactgctgatcgtgttccctcacttctgtctcggcagaggcctgatcgacctggctctgtctcaggccgtgaccgatgtgtacgccagatttggcgaggaacactccgccaatccattccactgggacctgatcggcaagaacctgttcgccatggtggtggaaggcgtcgtgtacttcctgctcactctgctggtgcagagacacttttttctgtcccaatggatcgccgagcctaccaaagaacccattgtggacgaggacgacgatgtggccgaggaaagacagagaatcatcaccggcggcaacaagaccgatatcctgagactgcacgagctgacaaagatttaccccggcacaagctccccagccgtggataggctttgtgtgggagttagacccggcgagtgctttggcctgctgggagttaatggcgccggaaagaccaccaccttcaagatgctgaccggcgacaccacagtgacaagcggagatgctacagtggccggcaagagcatcctgaccaacatcagcgaagtgcatcagaacatgggctactgccctcagttcgacgccatcgacgaactgctgacaggccgcgaacacctgtatctgtatgccagactgagaggcgtgcccgctgaagagatcgagaaggtggccaactggtccatcaagtctctgggcctgacagtgtacgccgactgtctggccggaacatacagcggaggaaacaagcggaagctgagcaccgccattgctctgatcggatgcccacctctggtcctgctggatgaacccaccaccggaatggacccccaggctagaagaatgctctggaacgtgatcgtgtctatcatccgcgagggcagagctgtggtgctgacctctcacagcatggaagagtgcgaggctctgtgtacccggctggccattatggtcaagggcgccttcagatgcatgggcaccattcagcatctgaaaagcaagttcggcgacggctacatcgtgacaatgaagatcaagagccccaaggacgacctcctgcctgatctgaaccccgtggaacagttttttcagggcaacttccccggctccgtgcagcgggaaagacactataacatgctgcagtttcaggtgtcctcctccagcctggctcggatctttcaactgctgctctctcacaaggacagcctgctgattgaagagtacagcgtgacacagaccacactcgaccaggttttcgtgaacttcgccaagcagcagaccgagagccacgacctgcctctgcatcctagagccgctggtgcctctagacaagctcaggacggcggaggcagctacccttatgatgtgcctgattacgcctaagcttagaccctccgatcctctgaaaggagcatcaggcaggcaggggttgctccggggccagggaaggggccccgctgggacgcggctgttattgcagctggttggcgcgcagccatgcttagctgcagtgcgggaatgctgggccttctgttctgggctgtttctcatacgcacgtaggccagtgtataaataaggttttattaaatgccaaatgagttctcattaacaaagaaagagggaaaatctcagtaaaccaccgtgacggcatctacccactttgagtcaggagctgggggtgtgagtgcaacctccgagacaagggaacctgtggagcccagagaatcggaggggggcgctggggttagcaccgactgaaccagctgtgttttctctcggttccttggagatcagaagtgagtgttgtcatcttcaaacaatccaaaggcagtacccatggccttactacatccctcccacaccatcccacccatccccgcgcgtacactcacacgctcatttgcacactatcgcacacgctcacttgcgtgcgcacacacagattggtgacctaggtggactgggagagaaataagagccaaatgactggattttctccaaggaaatttattaatagcccctcttggtttcacctgaaggagcttgtcttcacctgcggcctttgcaggcttaacgcccccagcttgaaacccagaagctcagacttgggcccaaggtattattagtgccaacactacctgaaatgtttcgcacctcataaaaatggtgtgtcagtttcgggtgagaggttgggacgcttcccatctgatttggcccaaggcatgcatgcccctccttctccttcccctcctcctccccctcttccccctaccatccttcctgttttctctccaactctggtgcacagctttgaaatcttgctgagaagcaaatctgtcccttctgctttgaatgtttatttgtggaagttcggcaggggaaccgaggcgggtgccaagacctgccatgctgctgggaagtctgagtctccctcccttccccctcctaaatgcttgttgatagagaaaagtcagcctcctcggcatttgggctcacggttttcctttgaaaatgcttccagtgtggcatgattcagctttcttttctgtcccccaaccactgctctgttgtcatttttacttttctgattgcattttatccgtgtctctttgactacggggtggctggacgttgagttccaggaagaaaagggcccaatcttggggttctgactacatgcgcccatcaatgtcctgtttcattcttggctctggctccctgaattcctgagtcactggggagaagcgtgggtggaccgccccctacccagtgagagttgccacagttgctgctctcctgggtcattggttgcagattgttaaacttcacctatgcatttcaactttcgggtggatattgctacgtcaagtgtctgggaaagcccccacagctacaggattttacagtgaggtcccactaatgacttgatgtcatgacttcctcattctttccaatttctcccacttctccataagggttttgggaaggggagaagagaaaggagtgattcctgagtgccagtaccagggaacagcagggctgttgggaggataaaataaaatatctttattttcattacatctgtgtgttggttttttgtgtgcgctacttgaagagcgaattctctaggatctgccaggaaccgtaaaaagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtgattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacgaattcgctcttcactc

SEQ ID NO: 37 - DNA, 11608 bp, Synthetic construct
  source 1..11608: mol_type = other DNA
ttcggtatcacaggagaatttcagggagagggatcacttcagatctgccgagtgagtcgattggacttaaagggccagtcaaaccctgactgccggctcatggcaggctcttgccgaggacaaatgcccagcctatatttatgcaaagagattttgttccaaacttaaggtcaaagatacctaaagacatccccctcaggaacccctctcatggaggagagtgcctgagggtcttggtttcccattgcatcccccacctcaatttccctggtgcccagccacttgtgtctttagggttctctttctctccataaaagggagccaacacagtgtcggcctcctctccccaactaagggcttatgtgtaattaaaagggattatgctttgaaggggaaaagtagcctttaatcaccaggagaaggacacagcgtccggagccagaggcgctcttaacggcgtttatgtcctttgctgtctgaggggcctcagctctgaccaatctggtcttcgtgtggtcattagcgtaacagttactgtctgtggtttaaaaatgaggtgtggagcaaataaacaggttggaagtgtggggtggggtggtggggtagggtggtggggcagggtggggggttgtgagcagtcagtgggcttgtcgccgattagcactgaagcagtgtttagctggacggcctttctgtgggcccctctgacagtgcccttcccaggaagatgtgtttctctgtcctcagccacatgaaaatcttttgcctaccgtgcctgtcaatccattgcctgcccgcccctcccccaccccccgttttacacctgcctgtccagtctaccgctctctagggcatccacgctgagcagtgggaagaactttaagccctgatgagcaggccaaaggcaagcaagaaccccctcgaacagcttcccagcttagtgaggccttacagataagtttgtacaaaaaagagaggtgccaccatgggctttgtgcgacagattcagctgctgctgtggaagaactggaccctgcggaagcggcagaaaatcagattcgtggtggaactcgtgtggcccctgagcctgtttctggtgctgatctggctgcggaacgccaatcctctgtacagccaccacgagtgtcacttccccaacaaggccatgccttctgccggaatgctgccttggctgcagggcatcttctgcaacgtgaacaacccctgctttcagagccccacacctggcgaaagccctggcatcgtgtccaactacaacaacagcatcctggccagagtgtaccgggacttccaagagctgctgatgaacgcccctgagtctcagcacctgggcagaatctggaccgagctgcacatcctgagccagttcatggacaccctgagaacacaccccgagagaatcgccggcaggggcatcagaatccgggacatcctgaaggacgaggaaaccctgacactgttcctcatcaagaacatcggcctgagcgacagcgtggtgtacctgctgatcaacagccaagtgcggcccgagcagtttgctcatggcgtgccggatctcgccctgaaggatatcgcctgttctgaggccctgctggaacggttcatcatcttcagccagcggagaggcgccaagaccgtcagatatgccctgtgcagtctgagccagggaaccctgcagtggatcgaggataccctgtacgccaacgtggacttcttcaagctgttccgggtgctgcccacactgctggattctagatcccagggcatcaacctgagaagctggggcggcatcctgtccgacatgagcccaagaatccaagagttcatccaccggcctagcatgcaggacctgctgtgggttaccagacctctgatgcagaacggcggacccgagacattcaccaagctgatgggcattctgagcgatctgctgtgcggctaccctgaaggcggaggatctagagtgctgagcttcaattggtacgaggacaacaactacaaggccttcctgggcatcgactccaccagaaaggaccccatctacagctacgaccggcggacaaccagcttctgcaatgccctgatccagagcctggaaagcaaccctctgaccaagatcgcttggagggccgccaaacctctgctgatgggaaagatcctgtacacccctgacagccctgccgccagaagaatcctgaagaacgccaacagcaccttcgaggaactggaacacgtgcgcaagctggtcaaggcctgggaagaagtgggacctcagatttggtacttcttcgacaatagcacccagatgaacatgatcagagacaccctgggcaaccctaccgtgaaggacttcctgaacagacagctgggcgaagagggcattaccgccgaggccatcctgaactttctgtacaagggccccagagagtcccaggccgacgacatggccaacttcgattggcgggacatcttcaacatcaccgacagaaccctgcggctggtcaaccagtacctggaatgcctggtgctggacaagttcgagagctacaacgacgagacacagctgacccagagagccctgtctctgctggaagagaatatgttctgggctggcgtggtgttccccgacatgtacccttggacaagcagcctgcctcctcacgtgaagtacaagatccggatggacatcgacgtggtcgaaaagaccaacaagatcaaggaccggtactgggacagcggccctagagctgatcccgtggaagattttcggtacatctggggcggattcgcatacctgcaggacatggtggaacagggaatcacacggtcccaggtgcaggctgaagctcctgtgggaatctacctgcagcagatgccttatccttgcttcgtggacgacagcttcatgatcatcctgaatcggtgcttccccatcttcatggtgctggcctggatctactccgtgtctatgaccgtgaagtccatcgtgctggaaaaagagctgcggctgaaagagacactgaagaaccagggcgtgtccaatgccgtgatctggtgcacctggtttctggacagcttctccattatgagcatgagcatctttctgctgacgatcttcatcatgcacggccgaatcctgcactacagcgacccctttatcctcttcctgttcctgctggccttcagcaccgctacaatcatgctgtgttttctgctgtccaccttcttcagcaaggcctctctggccgctgcttgtagcggcgtgatctacttcaccctgtacctgcctcacatcctgtgcttcgcatggcaggacagaatgaccgccgagctgaagaaagctgtgtccctgctgagccctgtggcctttggctttggcaccgagtacctcgtcagatttgaggaacaaggactgggactgcagtggtccaacatcggcaatagccctacagagggcgacgagttcagcttcctgctgtctatgcagatgatgctgctggacgccgccgtgtatggactgctggcttggtatctggaccaggtgttcccaggcgattacggcactcctctgccttggtatttcctgctgcaagagagctactggctcggcggcgagggatgtagcaccagagaagaaagagccctggaaaagaccgagcctctgaccgaggaaacagaggaccctgaacacccagagggcatccacgatagctttttcgagagagaacaccccggctgggtgccaggcgtgtgtgtgaagaatctggtcaagattttcgagccctgcggcagacctgccgtggacagactgaacatcaccttctacgagaaccagattaccgcctttctgggccacaacggcgctggcaagacaaccacattgagcatcctcacaggcctgctgcctccaacaagcggcacagttctcgttggcggcagagacatcgagacaagcctggatgccgtcagacagtccctgggcatgtgccctcagcacaacatcctgtttcaccacctgaccgtggccgagcacatgctgttttatgcccagctgaagggcaagagccaagaagaggctcagctggaaatggaagccatgttggaggacaccggcctgcaccacaagagaaatgaggaagcccaggatctgagcggcggcatgcagagaaaactgagcgtggccattgccttcgtgggcgacgccaaggttgtgatcctggatgagcctacaagcggcgtggacccttacagcagaagatccatctgggatctgctgctgaagtacagatcaggccggaccatcatcatgagcacccaccacatggacgaggccgatctgctcggagacagaatcgccatcattgctcagggcagactgtactgcagcggcaccccactgtttctgaagaactgtttcggcaccggactgtatctgaccctcgtgcggaagatgaagaacatccagtctcagcggaagggcagcgagggcacctgtagctgttctagcaagggctttagcaccacctgtccagctcacgtggacgatctgacccctgaacaggtgctggatggcgacgtgaacgagctgatggacgtggtgctgcaccatgtgcctgaggccaagctggtggaatgcatcggccaagaactgatttttctgctcccgaacaagaacttcaagcaccgggcctacgccagcctgttcagagagctggaagaaaccctggccgacctgggcctgtctagctttggcatcagcgacacccctctcgaagagattttcctgaaagtgacagaggacagcgatagcggccctctgtttgctggcggagcacagcaaaagcgcgagaacgtgaaccctagacacccctgtctgggcccaagagagaaagccggacagacccctcaggacagcaatgtgtgctctcctggtgctcctgccgctcatcctgagggacaacctccacctgaacctgagtgtcctggacctcagctgaacaccggaacacagctggttctgcagcacgtgcaggctctgctcgtgaagagattccagcacaccatcagaagccacaaggactttctggcccagatcgtgctgcccgccacctttgtttttctggctctgatgctgagcatcgtgatccctccattcggcgagtaccccgctctgacactgcacccttggatctacggccagcagtacacctttttctccatggacgaacccggcagcgagcagttcacagtgctggctgatgtcctgctgaacaagcccggcttcggcaaccggtgtctgaaagaaggatggctgcctgagtacccttgcggcaacagcacaccttggaaaacccctagcgtgtcccctaacatcacccagctgttccaaaagcagaaatggacccaagtgaacccctctccatcctgccggtgctccacaagggaaaagctgaccatgctgcccgagtgtccagaaggcgctggcggacttcctccacctcagagaacacagagatccaccgagattctccaggacctgaccgaccggaatatcagcgacttcctggttaagacataccccgcactgatccggtccagcctgaagtccaagttctgggtcaacgaacagagatacggcggcatcagcatcggcggaaaactgcctgtggtgcctatcacaggcgaggcccttgtgggctttctgtccgatctggggagaatcatgaacgtgtccggcggacctatcaccagggaagccagcaaagagatccccgatttcctgaagcacctggaaaccgaggacaatatcaaagtgtggttcaacaacaaaggatggcacgccctcgtgtcttttctgaacgtggcccacaatgccatcctgcgggctagcctgcctaaggacagaagccctgaggaatacggcatcaccgtgatctcccagcctctgaatctgaccaaagagcagctgagcgagatcaccgtgctgaccacctctgtggatgctgtggtggccatctgcgtgatcttcagcatgagcttcgtgcccgcctccttcgtgctgtacctgattcaagagagagtgaacaagagcaagcacctccagttcatctccggggtgtccccaaccacctactgggtcaccaattttctgtgggacatcatgaactacagcgtgtcagccggcctggtcgtgggcatctttatcggctttcagaagaaggcctacacgagccccgagaacctgcctgctttggttgctctgctgctcctgtatggctgggccgtgattcccatgatgtaccccgccagctttctgtttgacgtgcccagcacagcctacgtggccctgtcttgcgccaatctgttcatcggcatcaacagcagcgccatcacattcatcctggaactgttcgagaacaacaggaccctgctgcggttcaacgccgtgctgcggaaactgctgatcgtgttccctcacttctgtctcggcagaggcctgatcgacctggctctgtctcaggccgtgaccgatgtgtacgccagatttggcgaggaacactccgccaatccattccactgggacctgatcggcaagaacctgttcgccatggtggtggaaggcgtcgtgtacttcctgctcactctgctggtgcagagacacttttttctgtcccaatggatcgccgagcctaccaaagaacccattgtggacgaggacgacgatgtggccgaggaaagacagagaatcatcaccggcggcaacaagaccgatatcctgagactgcacgagctgacaaagatttaccccggcacaagctccccagccgtggataggctttgtgtgggagttagacccggcgagtgctttggcctgctgggagttaatggcgccggaaagaccaccaccttcaagatgctgaccggcgacaccacagtgacaagcggagatgctacagtggccggcaagagcatcctgaccaacatcagcgaagtgcatcagaacatgggctactgccctcagttcgacgccatcgacgaactgctgacaggccgcgaacacctgtatctgtatgccagactgagaggcgtgcccgctgaagagatcgagaaggtggccaactggtccatcaagtctctgggcctgacagtgtacgccgactgtctggccggaacatacagcggaggaaacaagcggaagctgagcaccgccattgctctgatcggatgcccacctctggtcctgctggatgaacccaccaccggaatggacccccaggctagaagaatgctctggaacgtgatcgtgtctatcatccgcgagggcagagctgtggtgctgacctctcacagcatggaagagtgcgaggctctgtgtacccggctggccattatggtcaagggcgccttcagatgcatgggcaccattcagcatctgaaaagcaagttcggcgacggctacatcgtgacaatgaagatcaagagccccaaggacgacctcctgcctgatctgaaccccgtggaacagttttttcagggcaacttccccggctccgtgcagcgggaaagacactataacatgctgcagtttcaggtgtcctcctccagcctggctcggatctttcaactgctgctctctcacaaggacagcctgctgattgaagagtacagcgtgacacagaccacactcgaccaggttttcgtgaacttcgccaagcagcagaccgagagccacgacctgcctctgcatcctagagccgctggtgcctctagacaagctcaggacggcggaggcagctacccttatgatgtgcctgattacgcctaagcttagaccctccgatcctctgaaaggagcatcaggcaggcaggggttgctccggggccagggaaggggccccgctgggacgcggctgttattgcagctggttggcgcgcagccatgcttagctgcagtgcgggaatgctgggccttctgttctgggctgtttctcatacgcacgtaggccagtgtataaataaggttttattaaatgccaaatgagttctcattaacaaagaaagagggaaaatctcagtaaaccaccgtgacggcatctacccactttgagtcaggagctgggggtgtgagtgcaacctccgagacaagggaacctgtggagcccagagaatcggaggggggcgctggggttagcaccgactgaaccagctgtgttttctctcggttccttggagatcagaagtgagtgttgtcatcttcaaacaatccaaaggcagtacccatggccttactacatccctcccacaccatcccacccatccccgcgcgtacactcacacgctcatttgcacactatcgcacacgctcacttgcgtgcgcacacacagattggtgacctaggtggactgggagagaaataagagccaaatgactggattttctccaaggaaatttattaatagcccctcttggtttcacctgaaggagcttgtcttcacctgcggcctttgcaggcttaacgcccccagcttgaaacccagaagctcagacttgggcccaaggtattattagtgccaacactacctgaaatgtttcgcacctcataaaaatggtgtgtcagtttcgggtgagaggttgggacgcttcccatctgatttggcccaaggcatgcatgcccctccttctccttcccctcctcctccccctcttccccctaccatccttcctgttttctctccaactctggtgcacagctttgaaatcttgctgagaagcaaatctgtcccttctgctttgaatgtttatttgtggaagttcggcaggggaaccgaggcgggtgccaagacctgccatgctgctgggaagtctgagtctccctcccttccccctcctaaatgcttgttgatagagaaaagtcagcctcctcggcatttgggctcacggttttcctttgaaaatgcttccagtgtggcatgattcagctttcttttctgtcccccaaccactgctctgttgtcatttttacttttctgattgcattttatccgtgtctctttgactacggggtggctggacgttgagttccaggaagaaaagggcccaatcttggggttctgactacatgcgcccatcaatgtcctgtttcattcttggctctggctccctgaattcctgagtcactggggagaagcgtgggtggaccgccccctacccagtgagagttgccacagttgctgctctcctgggtcattggttgcagattgttaaacttcacctatgcatttcaactttcgggtggatattgctacgtcaagtgtctgggaaagcccccacagctacaggattttacagtgaggtcccactaatgacttgatgtcatgacttcctcattctttccaatttctcccacttctccataagggttttgggaaggggagaagagaaaggagtgattcctgagtgccagtaccagggaacagcagggctgttgggaggataaaataaaatatctttattttcattacatctgtgtgttggttttttgtgtgcgctacttgaagagcgaattctctaggatctgccaggaaccgtaaaaagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtgattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacgaattcgctcttcactc

SEQ ID NO: 38 - DNA, 12067 bp, Synthetic construct
  source 1..12067: mol_type = other DNA
ttcggtatcacaggagaatttcagggagcaagataagctattcctccctgtctttgcagagtttaaatgactcacttgttcaagtacccaccgttgccatgtgggaccgtgagcaaagtacttaatctcactaagcttcacgttcctcatctgtaaaacagcaaatatggacctcacaaaattgtagtgaggctaaaatgaaataacatatgcaaaagcagtttataaataataaacttactataaaatattattttgtaattctgcaagcttgtcttaaatgccatcacctccaaggagcctttttgccatcataagcagaaactatctctctcttcttggaagctccaccatgcacagcctatgggccctcatcacactccttgagttattcgagttcaagtcccgtgtttacaaccagaccgcaaactctatgaagtcagcatccattcctctctgtggttctccctccgccccatccaggctcaagggtctagagtctttcaaagagaacacattctgagatttgaggaggcagagacaaaaagttccactgcgaagtgccagggaggcttctgtttggggtgtcccttgggatcacagatcccccacctggtgatgagtcaacccagcaccaccccattgcagggctggaatgacagtaatgggcccacctgctgcctctcctcatacccgcaccccagtcagacattgcaagtcagtcacggctctgtcctgctgggcctggagtgttccagtgccttttccatcacagcaccaagcagccactactagtcgatcaatttcagcacaagagataaacatcattaccctctgctaagctcagagataacccaactagctgaccataatgacttcagtcattacggagcaagataaaagactaaaagagggagggatcacttcagatctgccgagtgagtcgattggacttaaagggccagtcaaaccctgactgccggctcatggcaggctcttgccgaggacaaatgcccagcctatatttatgcaaagagattttgttccaaacttaaggtcaaagatacctaaagacatccccctcaggaacccctctcatggaggagagtgcctgagggtcttggtttcccattgcatcccccacctcaatttccctggtgcccagccacttgtgtctttagggttctctttctctccataaaagggagccaacacagtgtcggcctcctctccccaactaagggcttatgtgtaattaaaagggattatgctttgaaggggaaaagtagcctttaatcaccaggagaaggacacagcgtccggagccagaggcgctcttaacggcgtttatgtcctttgctgtctgaggggcctcagctctgaccaatctggtcttcgtgtggtcattagcagataagtttgtacaaaaaagagaggtgccaccatgggctttgtgcgacagattcagctgctgctgtggaagaactggaccctgcggaagcggcagaaaatcagattcgtggtggaactcgtgtggcccctgagcctgtttctggtgctgatctggctgcggaacgccaatcctctgtacagccaccacgagtgtcacttccccaacaaggccatgccttctgccggaatgctgccttggctgcagggcatcttctgcaacgtgaacaacccctgctttcagagccccacacctggcgaaagccctggcatcgtgtccaactacaacaacagcatcctggccagagtgtaccgggacttccaagagctgctgatgaacgcccctgagtctcagcacctgggcagaatctggaccgagctgcacatcctgagccagttcatggacaccctgagaacacaccccgagagaatcgccggcaggggcatcagaatccgggacatcctgaaggacgaggaaaccctgacactgttcctcatcaagaacatcggcctgagcgacagcgtggtgtacctgctgatcaacagccaagtgcggcccgagcagtttgctcatggcgtgccggatctcgccctgaaggatatcgcctgttctgaggccctgctggaacggttcatcatcttcagccagcggagaggcgccaagaccgtcagatatgccctgtgcagtctgagccagggaaccctgcagtggatcgaggataccctgtacgccaacgtggacttcttcaagctgttccgggtgctgcccacactgctggattctagatcccagggcatcaacctgagaagctggggcggcatcctgtccgacatgagcccaagaatccaagagttcatccaccggcctagcatgcaggacctgctgtgggttaccagacctctgatgcagaacggcggacccgagacattcaccaagctgatgggcattctgagcgatctgctgtgcggctaccctgaaggcggaggatctagagtgctgagcttcaattggtacgaggacaacaactacaaggccttcctgggcatcgactccaccagaaaggaccccatctacagctacgaccggcggacaaccagcttctgcaatgccctgatccagagcctggaaagcaaccctctgaccaagatcgcttggagggccgccaaacctctgctgatgggaaagatcctgtacacccctgacagccctgccgccagaagaatcctgaagaacgccaacagcaccttcgaggaactggaacacgtgcgcaagctggtcaaggcctgggaagaagtgggacctcagatttggtacttcttcgacaatagcacccagatgaacatgatcagagacaccctgggcaaccctaccgtgaaggacttcctgaacagacagctgggcgaagagggcattaccgccgaggccatcctgaactttctgtacaagggccccagagagtcccaggccgacgacatggccaacttcgattggcgggacatcttcaacatcaccgacagaaccctgcggctggtcaaccagtacctggaatgcctggtgctggacaagttcgagagctacaacgacgagacacagctgacccagagagccctgtctctgctggaagagaatatgttctgggctggcgtggtgttccccgacatgtacccttggacaagcagcctgcctcctcacgtgaagtacaagatccggatggacatcgacgtggtcgaaaagaccaacaagatcaaggaccggtactgggacagcggccctagagctgatcccgtggaagattttcggtacatctggggcggattcgcatacctgcaggacatggtggaacagggaatcacacggtcccaggtgcaggctgaagctcctgtgggaatctacctgcagcagatgccttatccttgcttcgtggacgacagcttcatgatcatcctgaatcggtgcttccccatcttcatggtgctggcctggatctactccgtgtctatgaccgtgaagtccatcgtgctggaaaaagagctgcggctgaaagagacactgaagaaccagggcgtgtccaatgccgtgatctggtgcacctggtttctggacagcttctccattatgagcatgagcatctttctgctgacgatcttcatcatgcacggccgaatcctgcactacagcgacccctttatcctcttcctgttcctgctggccttcagcaccgctacaatcatgctgtgttttctgctgtccaccttcttcagcaaggcctctctggccgctgcttgtagcggcgtgatctacttcaccctgtacctgcctcacatcctgtgcttcgcatggcaggacagaatgaccgccgagctgaagaaagctgtgtccctgctgagccctgtggcctttggctttggcaccgagtacctcgtcagatttgaggaacaaggactgggactgcagtggtccaacatcggcaatagccctacagagggcgacgagttcagcttcctgctgtctatgcagatgatgctgctggacgccgccgtgtatggactgctggcttggtatctggaccaggtgttcccaggcgattacggcactcctctgccttggtatttcctgctgcaagagagctactggctcggcggcgagggatgtagcaccagagaagaaagagccctggaaaagaccgagcctctgaccgaggaaacagaggaccctgaacacccagagggcatccacgatagctttttcgagagagaacaccccggctgggtgccaggcgtgtgtgtgaagaatctggtcaagattttcgagccctgcggcagacctgccgtggacagactgaacatcaccttctacgagaaccagattaccgcctttctgggccacaacggcgctggcaagacaaccacattgagcatcctcacaggcctgctgcctccaacaagcggcacagttctcgttggcggcagagacatcgagacaagcctggatgccgtcagacagtccctgggcatgtgccctcagcacaacatcctgtttcaccacctgaccgtggccgagcacatgctgttttatgcccagctgaagggcaagagccaagaagaggctcagctggaaatggaagccatgttggaggacaccggcctgcaccacaagagaaatgaggaagcccaggatctgagcggcggcatgcagagaaaactgagcgtggccattgccttcgtgggcgacgccaaggttgtgatcctggatgagcctacaagcggcgtggacccttacagcagaagatccatctgggatctgctgctgaagtacagatcaggccggaccatcatcatgagcacccaccacatggacgaggccgatctgctcggagacagaatcgccatcattgctcagggcagactgtactgcagcggcaccccactgtttctgaagaactgtttcggcaccggactgtatctgaccctcgtgcggaagatgaagaacatccagtctcagcggaagggcagcgagggcacctgtagctgttctagcaagggctttagcaccacctgtccagctcacgtggacgatctgacccctgaacaggtgctggatggcgacgtgaacgagctgatggacgtggtgctgcaccatgtgcctgaggccaagctggtggaatgcatcggccaagaactgatttttctgctcccgaacaagaacttcaagcaccgggcctacgccagcctgttcagagagctggaagaaaccctggccgacctgggcctgtctagctttggcatcagcgacacccctctcgaagagattttcctgaaagtgacagaggacagcgatagcggccctctgtttgctggcggagcacagcaaaagcgcgagaacgtgaaccctagacacccctgtctgggcccaagagagaaagccggacagacccctcaggacagcaatgtgtgctctcctggtgctcctgccgctcatcctgagggacaacctccacctgaacctgagtgtcctggacctcagctgaacaccggaacacagctggttctgcagcacgtgcaggctctgctcgtgaagagattccagcacaccatcagaagccacaaggactttctggcccagatcgtgctgcccgccacctttgtttttctggctctgatgctgagcatcgtgatccctccattcggcgagtaccccgctctgacactgcacccttggatctacggccagcagtacacctttttctccatggacgaacccggcagcgagcagttcacagtgctggctgatgtcctgctgaacaagcccggcttcggcaaccggtgtctgaaagaaggatggctgcctgagtacccttgcggcaacagcacaccttggaaaacccctagcgtgtcccctaacatcacccagctgttccaaaagcagaaatggacccaagtgaacccctctccatcctgccggtgctccacaagggaaaagctgaccatgctgcccgagtgtccagaaggcgctggcggacttcctccacctcagagaacacagagatccaccgagattctccaggacctgaccgaccggaatatcagcgacttcctggttaagacataccccgcactgatccggtccagcctgaagtccaagttctgggtcaacgaacagagatacggcggcatcagcatcggcggaaaactgcctgtggtgcctatcacaggcgaggcccttgtgggctttctgtccgatctggggagaatcatgaacgtgtccggcggacctatcaccagggaagccagcaaagagatccccgatttcctgaagcacctggaaaccgaggacaatatcaaagtgtggttcaacaacaaaggatggcacgccctcgtgtcttttctgaacgtggcccacaatgccatcctgcgggctagcctgcctaaggacagaagccctgaggaatacggcatcaccgtgatctcccagcctctgaatctgaccaaagagcagctgagcgagatcaccgtgctgaccacctctgtggatgctgtggtggccatctgcgtgatcttcagcatgagcttcgtgcccgcctccttcgtgctgtacctgattcaagagagagtgaacaagagcaagcacctccagttcatctccggggtgtccccaaccacctactgggtcaccaattttctgtgggacatcatgaactacagcgtgtcagccggcctggtcgtgggcatctttatcggctttcagaagaaggcctacacgagccccgagaacctgcctgctttggttgctctgctgctcctgtatggctgggccgtgattcccatgatgtaccccgccagctttctgtttgacgtgcccagcacagcctacgtggccctgtcttgcgccaatctgttcatcggcatcaacagcagcgccatcacattcatcctggaactgttcgagaacaacaggaccctgctgcggttcaacgccgtgctgcggaaactgctgatcgtgttccctcacttctgtctcggcagaggcctgatcgacctggctctgtctcaggccgtgaccgatgtgtacgccagatttggcgaggaacactccgccaatccattccactgggacctgatcggcaagaacctgttcgccatggtggtggaaggcgtcgtgtacttcctgctcactctgctggtgcagagacacttttttctgtcccaatggatcgccgagcctaccaaagaacccattgtggacgaggacgacgatgtggccgaggaaagacagagaatcatcaccggcggcaacaagaccgatatcctgagactgcacgagctgacaaagatttaccccggcacaagctccccagccgtggataggctttgtgtgggagttagacccggcgagtgctttggcctgctgggagttaatggcgccggaaagaccaccaccttcaagatgctgaccggcgacaccacagtgacaagcggagatgctacagtggccggcaagagcatcctgaccaacatcagcgaagtgcatcagaacatgggctactgccctcagttcgacgccatcgacgaactgctgacaggccgcgaacacctgtatctgtatgccagactgagaggcgtgcccgctgaagagatcgagaaggtggccaactggtccatcaagtctctgggcctgacagtgtacgccgactgtctggccggaacatacagcggaggaaacaagcggaagctgagcaccgccattgctctgatcggatgcccacctctggtcctgctggatgaacccaccaccggaatggacccccaggctagaagaatgctctggaacgtgatcgtgtctatcatccgcgagggcagagctgtggtgctgacctctcacagcatggaagagtgcgaggctctgtgtacccggctggccattatggtcaagggcgccttcagatgcatgggcaccattcagcatctgaaaagcaagttcggcgacggctacatcgtgacaatgaagatcaagagccccaaggacgacctcctgcctgatctgaaccccgtggaacagttttttcagggcaacttccccggctccgtgcagcgggaaagacactataacatgctgcagtttcaggtgtcctcctccagcctggctcggatctttcaactgctgctctctcacaaggacagcctgctgattgaagagtacagcgtgacacagaccacactcgaccaggttttcgtgaacttcgccaagcagcagaccgagagccacgacctgcctctgcatcctagagccgctggtgcctctagacaagctcaggacggcggaggcagctacccttatgatgtgcctgattacgcctaagcttagaccctccgatcctctgaaaggagcatcaggcaggcaggggttgctccggggccagggaaggggccccgctgggacgcggctgttattgcagctggttggcgcgcagccatgcttagctgcagtgcgggaatgctgggccttctgttctgggctgtttctcatacgcacgtaggccagtgtataaataaggttttattaaatgccaaatgagttctcattaacaaagaaagagggaaaatctcagtaaaccaccgtgacggcatctacccactttgagtcaggagctgggggtgtgagtgcaacctccgagacaagggaacctgtggagcccagagaatcggaggggggcgctggggttagcaccgactgaaccagctgtgttttctctcggttccttggagatcagaagtgagtgttgtcatcttcaaacaatccaaaggcagtacccatggccttactacatccctcccacaccatcccacccatccccgcgcgtacactcacacgctcatttgcacactatcgcacacgctcacttgcgtgcgcacacacagattggtgacctaggtggactgggagagaaataagagccaaatgactggattttctccaaggaaatttattaatagcccctcttggtttcacctgaaggagcttgtcttcacctgcggcctttgcaggcttaacgcccccagcttgaaacccagaagctcagacttgggcccaaggtattattagtgccaacactacctgaaatgtttcgcacctcataaaaatggtgtgtcagtttcgggtgagaggttgggacgcttcccatctgatttggcccaaggcatgcatgcccctccttctccttcccctcctcctccccctcttccccctaccatccttcctgttttctctccaactctggtgcacagctttgaaatcttgctgagaagcaaatctgtcccttctgctttgaatgtttatttgtggaagttcggcaggggaaccgaggcgggtgccaagacctgccatgctgctgggaagtctgagtctccctcccttccccctcctaaatgcttgttgatagagaaaagtcagcctcctcggcatttgggctcacggttttcctttgaaaatgcttccagtgtggcatgattcagctttcttttctgtcccccaaccactgctctgttgtcatttttacttttctgattgcattttatccgtgtctctttgactacggggtggctggacgttgagttccaggaagaaaagggcccaatcttggggttctgactacatgcgcccatcaatgtcctgtttcattcttggctctggctccctgaattcctgagtcactggggagaagcgtgggtggaccgccccctacccagtgagagttgccacagttgctgctctcctgggtcattggttgcagattgttaaacttcacctatgcatttcaactttcgggtggatattgctacgtcaagtgtctgggaaagcccccacagctacaggattttacagtgaggtcccactaatgacttgatgtcatgacttcctcattctttccaatttctcccacttctccataagggttttgggaaggggagaagagaaaggagtgattcctgagtgccagtaccagggaacagcagggctgttgggaggataaaataaaatatctttattttcattacatctgtgtgttggttttttgtgtgcgctacttgaagagcgaattctctaggatctgccaggaaccgtaaaaagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtgattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacgaattcgctcttcactc

SEQ ID NO: 39 - DNA, 11180 bp, Synthetic construct
  source 1..11180: mol_type = other DNA
ttcggtatcacaggagaatttcagggagagggatcacttcagatctgccgagtgagtcgattggacttaaagggccagtcaaaccctgactgccggctcatggcaggctcttgccgaggacaaatgcccagcctatatttatgcaaagagattttgttccaaacttaaggtcaaagatacctaaagacatccccctcaggaacccctctcatggaggagagtgcctgagggtcttggtttcccattgcatcccccacctcaatttccctggtgcccagccacttgtgtctttagggttctctttctctccataaaagggagccaacacagtgtcggcctcctctccccaactaagggcttatgtgtaattaaaagggattatgctttgaaggggaaaagtagcctttaatcaccaggagaaggacacagcgtccggagccagaggcgctcttaacggcgtttatgtcctttgctgtctgaggggcctcagctctgaccaatctggtcttcgtgtggtcattagcagataagtttgtacaaaaaagagaggtgccaccatgggctttgtgcgacagattcagctgctgctgtggaagaactggaccctgcggaagcggcagaaaatcagattcgtggtggaactcgtgtggcccctgagcctgtttctggtgctgatctggctgcggaacgccaatcctctgtacagccaccacgagtgtcacttccccaacaaggccatgccttctgccggaatgctgccttggctgcagggcatcttctgcaacgtgaacaacccctgctttcagagccccacacctggcgaaagccctggcatcgtgtccaactacaacaacagcatcctggccagagtgtaccgggacttccaagagctgctgatgaacgcccctgagtctcagcacctgggcagaatctggaccgagctgcacatcctgagccagttcatggacaccctgagaacacaccccgagagaatcgccggcaggggcatcagaatccgggacatcctgaaggacgaggaaaccctgacactgttcctcatcaagaacatcggcctgagcgacagcgtggtgtacctgctgatcaacagccaagtgcggcccgagcagtttgctcatggcgtgccggatctcgccctgaaggatatcgcctgttctgaggccctgctggaacggttcatcatcttcagccagcggagaggcgccaagaccgtcagatatgccctgtgcagtctgagccagggaaccctgcagtggatcgaggataccctgtacgccaacgtggacttcttcaagctgttccgggtgctgcccacactgctggattctagatcccagggcatcaacctgagaagctggggcggcatcctgtccgacatgagcccaagaatccaagagttcatccaccggcctagcatgcaggacctgctgtgggttaccagacctctgatgcagaacggcggacccgagacattcaccaagctgatgggcattctgagcgatctgctgtgcggctaccctgaaggcggaggatctagagtgctgagcttcaattggtacgaggacaacaactacaaggccttcctgggcatcgactccaccagaaaggaccccatctacagctacgaccggcggacaaccagcttctgcaatgccctgatccagagcctggaaagcaaccctctgaccaagatcgcttggagggccgccaaacctctgctgatgggaaagatcctgtacacccctgacagccctgccgccagaagaatcctgaagaacgccaacagcaccttcgaggaactggaacacgtgcgcaagctggtcaaggcctgggaagaagtgggacctcagatttggtacttcttcgacaatagcacccagatgaacatgatcagagacaccctgggcaaccctaccgtgaaggacttcctgaacagacagctgggcgaagagggcattaccgccgaggccatcctgaactttctgtacaagggccccagagagtcccaggccgacgacatggccaacttcgattggcgggacatcttcaacatcaccgacagaaccctgcggctggtcaaccagtacctggaatgcctggtgctggacaagttcgagagctacaacgacgagacacagctgacccagagagccctgtctctgctggaagagaatatgttctgggctggcgtggtgttccccgacatgtacccttggacaagcagcctgcctcctcacgtgaagtacaagatccggatggacatcgacgtggtcgaaaagaccaacaagatcaaggaccggtactgggacagcggccctagagctgatcccgtggaagattttcggtacatctggggcggattcgcatacctgcaggacatggtggaacagggaatcacacggtcccaggtgcaggctgaagctcctgtgggaatctacctgcagcagatgccttatccttgcttcgtggacgacagcttcatgatcatcctgaatcggtgcttccccatcttcatggtgctggcctggatctactccgtgtctatgaccgtgaagtccatcgtgctggaaaaagagctgcggctgaaagagacactgaagaaccagggcgtgtccaatgccgtgatctggtgcacctggtttctggacagcttctccattatgagcatgagcatctttctgctgacgatcttcatcatgcacggccgaatcctgcactacagcgacccctttatcctcttcctgttcctgctggccttcagcaccgctacaatcatgctgtgttttctgctgtccaccttcttcagcaaggcctctctggccgctgcttgtagcggcgtgatctacttcaccctgtacctgcctcacatcctgtgcttcgcatggcaggacagaatgaccgccgagctgaagaaagctgtgtccctgctgagccctgtggcctttggctttggcaccgagtacctcgtcagatttgaggaacaaggactgggactgcagtggtccaacatcggcaatagccctacagagggcgacgagttcagcttcctgctgtctatgcagatgatgctgctggacgccgccgtgtatggactgctggcttggtatctggaccaggtgttcccaggcgattacggcactcctctgccttggtatttcctgctgcaagagagctactggctcggcggcgagggatgtagcaccagagaagaaagagccctggaaaagaccgagcctctgaccgaggaaacagaggaccctgaacacccagagggcatccacgatagctttttcgagagagaacaccccggctgggtgccaggcgtgtgtgtgaagaatctggtcaagattttcgagccctgcggcagacctgccgtggacagactgaacatcaccttctacgagaaccagattaccgcctttctgggccacaacggcgctggcaagacaaccacattgagcatcctcacaggcctgctgcctccaacaagcggcacagttctcgttggcggcagagacatcgagacaagcctggatgccgtcagacagtccctgggcatgtgccctcagcacaacatcctgtttcaccacctgaccgtggccgagcacatgctgttttatgcccagctgaagggcaagagccaagaagaggctcagctggaaatggaagccatgttggaggacaccggcctgcaccacaagagaaatgaggaagcccaggatctgagcggcggcatgcagagaaaactgagcgtggccattgccttcgtgggcgacgccaaggttgtgatcctggatgagcctacaagcggcgtggacccttacagcagaagatccatctgggatctgctgctgaagtacagatcaggccggaccatcatcatgagcacccaccacatggacgaggccgatctgctcggagacagaatcgccatcattgctcagggcagactgtactgcagcggcaccccactgtttctgaagaactgtttcggcaccggactgtatctgaccctcgtgcggaagatgaagaacatccagtctcagcggaagggcagcgagggcacctgtagctgttctagcaagggctttagcaccacctgtccagctcacgtggacgatctgacccctgaacaggtgctggatggcgacgtgaacgagctgatggacgtggtgctgcaccatgtgcctgaggccaagctggtggaatgcatcggccaagaactgatttttctgctcccgaacaagaacttcaagcaccgggcctacgccagcctgttcagagagctggaagaaaccctggccgacctgggcctgtctagctttggcatcagcgacacccctctcgaagagattttcctgaaagtgacagaggacagcgatagcggccctctgtttgctggcggagcacagcaaaagcgcgagaacgtgaaccctagacacccctgtctgggcccaagagagaaagccggacagacccctcaggacagcaatgtgtgctctcctggtgctcctgccgctcatcctgagggacaacctccacctgaacctgagtgtcctggacctcagctgaacaccggaacacagctggttctgcagcacgtgcaggctctgctcgtgaagagattccagcacaccatcagaagccacaaggactttctggcccagatcgtgctgcccgccacctttgtttttctggctctgatgctgagcatcgtgatccctccattcggcgagtaccccgctctgacactgcacccttggatctacggccagcagtacacctttttctccatggacgaacccggcagcgagcagttcacagtgctggctgatgtcctgctgaacaagcccggcttcggcaaccggtgtctgaaagaaggatggctgcctgagtacccttgcggcaacagcacaccttggaaaacccctagcgtgtcccctaacatcacccagctgttccaaaagcagaaatggacccaagtgaacccctctccatcctgccggtgctccacaagggaaaagctgaccatgctgcccgagtgtccagaaggcgctggcggacttcctccacctcagagaacacagagatccaccgagattctccaggacctgaccgaccggaatatcagcgacttcctggttaagacataccccgcactgatccggtccagcctgaagtccaagttctgggtcaacgaacagagatacggcggcatcagcatcggcggaaaactgcctgtggtgcctatcacaggcgaggcccttgtgggctttctgtccgatctggggagaatcatgaacgtgtccggcggacctatcaccagggaagccagcaaagagatccccgatttcctgaagcacctggaaaccgaggacaatatcaaagtgtggttcaacaacaaaggatggcacgccctcgtgtcttttctgaacgtggcccacaatgccatcctgcgggctagcctgcctaaggacagaagccctgaggaatacggcatcaccgtgatctcccagcctctgaatctgaccaaagagcagctgagcgagatcaccgtgctgaccacctctgtggatgctgtggtggccatctgcgtgatcttcagcatgagcttcgtgcccgcctccttcgtgctgtacctgattcaagagagagtgaacaagagcaagcacctccagttcatctccggggtgtccccaaccacctactgggtcaccaattttctgtgggacatcatgaactacagcgtgtcagccggcctggtcgtgggcatctttatcggctttcagaagaaggcctacacgagccccgagaacctgcctgctttggttgctctgctgctcctgtatggctgggccgtgattcccatgatgtaccccgccagctttctgtttgacgtgcccagcacagcctacgtggccctgtcttgcgccaatctgttcatcggcatcaacagcagcgccatcacattcatcctggaactgttcgagaacaacaggaccctgctgcggttcaacgccgtgctgcggaaactgctgatcgtgttccctcacttctgtctcggcagaggcctgatcgacctggctctgtctcaggccgtgaccgatgtgtacgccagatttggcgaggaacactccgccaatccattccactgggacctgatcggcaagaacctgttcgccatggtggtggaaggcgtcgtgtacttcctgctcactctgctggtgcagagacacttttttctgtcccaatggatcgccgagcctaccaaagaacccattgtggacgaggacgacgatgtggccgaggaaagacagagaatcatcaccggcggcaacaagaccgatatcctgagactgcacgagctgacaaagatttaccccggcacaagctccccagccgtggataggctttgtgtgggagttagacccggcgagtgctttggcctgctgggagttaatggcgccggaaagaccaccaccttcaagatgctgaccggcgacaccacagtgacaagcggagatgctacagtggccggcaagagcatcctgaccaacatcagcgaagtgcatcagaacatgggctactgccctcagttcgacgccatcgacgaactgctgacaggccgcgaacacctgtatctgtatgccagactgagaggcgtgcccgctgaagagatcgagaaggtggccaactggtccatcaagtctctgggcctgacagtgtacgccgactgtctggccggaacatacagcggaggaaacaagcggaagctgagcaccgccattgctctgatcggatgcccacctctggtcctgctggatgaacccaccaccggaatggacccccaggctagaagaatgctctggaacgtgatcgtgtctatcatccgcgagggcagagctgtggtgctgacctctcacagcatggaagagtgcgaggctctgtgtacccggctggccattatggtcaagggcgccttcagatgcatgggcaccattcagcatctgaaaagcaagttcggcgacggctacatcgtgacaatgaagatcaagagccccaaggacgacctcctgcctgatctgaaccccgtggaacagttttttcagggcaacttccccggctccgtgcagcgggaaagacactataacatgctgcagtttcaggtgtcctcctccagcctggctcggatctttcaactgctgctctctcacaaggacagcctgctgattgaagagtacagcgtgacacagaccacactcgaccaggttttcgtgaacttcgccaagcagcagaccgagagccacgacctgcctctgcatcctagagccgctggtgcctctagacaagctcaggacggcggaggcagctacccttatgatgtgcctgattacgcctaagcttagaccctccgatcctctgaaaggagcatcaggcaggcaggggttgctccggggccagggaaggggccccgctgggacgcggctgttattgcagctggttggcgcgcagccatgcttagctgcagtgcgggaatgctgggccttctgttctgggctgtttctcatacgcacgtaggccagtgtataaataaggttttattaaatgccaaatgagttctcattaacaaagaaagagggaaaatctcagtaaaccaccgtgacggcatctacccactttgagtcaggagctgggggtgtgagtgcaacctccgagacaagggaacctgtggagcccagagaatcggaggggggcgctggggttagcaccgactgaaccagctgtgttttctctcggttccttggagatcagaagtgagtgttgtcatcttcaaacaatccaaaggcagtacccatggccttactacatccctcccacaccatcccacccatccccgcgcgtacactcacacgctcatttgcacactatcgcacacgctcacttgcgtgcgcacacacagattggtgacctaggtggactgggagagaaataagagccaaatgactggattttctccaaggaaatttattaatagcccctcttggtttcacctgaaggagcttgtcttcacctgcggcctttgcaggcttaacgcccccagcttgaaacccagaagctcagacttgggcccaaggtattattagtgccaacactacctgaaatgtttcgcacctcataaaaatggtgtgtcagtttcgggtgagaggttgggacgcttcccatctgatttggcccaaggcatgcatgcccctccttctccttcccctcctcctccccctcttccccctaccatccttcctgttttctctccaactctggtgcacagctttgaaatcttgctgagaagcaaatctgtcccttctgctttgaatgtttatttgtggaagttcggcaggggaaccgaggcgggtgccaagacctgccatgctgctgggaagtctgagtctccctcccttccccctcctaaatgcttgttgatagagaaaagtcagcctcctcggcatttgggctcacggttttcctttgaaaatgcttccagtgtggcatgattcagctttcttttctgtcccccaaccactgctctgttgtcatttttacttttctgattgcattttatccgtgtctctttgactacggggtggctggacgttgagttccaggaagaaaagggcccaatcttggggttctgactacatgcgcccatcaatgtcctgtttcattcttggctctggctccctgaattcctgagtcactggggagaagcgtgggtggaccgccccctacccagtgagagttgccacagttgctgctctcctgggtcattggttgcagattgttaaacttcacctatgcatttcaactttcgggtggatattgctacgtcaagtgtctgggaaagcccccacagctacaggattttacagtgaggtcccactaatgacttgatgtcatgacttcctcattctttccaatttctcccacttctccataagggttttgggaaggggagaagagaaaggagtgattcctgagtgccagtaccagggaacagcagggctgttgggaggataaaataaaatatctttattttcattacatctgtgtgttggttttttgtgtgcgctacttgaagagcgaattctctaggatctgccaggaaccgtaaaaagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtgattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacgaattcgctcttcactc

SEQ ID NO: 40 - DNA, 991 bp, Synthetic construct
  source 1..991: mol_type = other DNA
gaaccactgctgcctagagctctgctctcttcattgaacaatacaagaggagtgtgtaggtagacacccaccacttccaacagcttaggagagcccttgagtatggattgatgtattaaaatttattgaatcacatgctgagattttcaccagctgcccgtggggatctgggcatttattcccatattgcactggctggctggaagccagcagcataaactccagggctgttctgtcaacccccaccagactcacccccctccaccagccccggcaggcttctccttccatctctctgaagcaacttactgatgggccctgccagccaatcacagccagaataacgtatgatgtcaccagcagccaatcagagctcctcgtcagcatatgcagaattctgtcattttactagggtgatgaaattcccaagcaacaccatccttttcagataagggcactgaggctgagagaggagctgaaacctacccggggtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggcctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccaggga

SEQ ID NO: 41 - protein, 297 aa, Synthetic construct
MSAVLQRFREKLPHKPYCTNDFAYGVRILPKNIAILARFIQQNQPHALYWLPFDVDRTGASIDWSDRNCPAPNITVKNPRNGHAHLLYALALPVRTAPDASASALRYAAAIERALCEKLGADVNYSGLICKNPCHPEWQEVEWREEPYTLDELADYLDLSASARRSVDKNYGLGRNYHLFEKVRKWAYRAIRQGWPVFSQWLDAVIQRVEMYNASLPVPLSPAECRAIGKSIAKYTHRKFSPEGFSAVQAARGRKGGTKSKRAAVPTSARSLKPWEALGISRATYYRKLKCDPDLAK

SEQ ID NO: 42 - DNA, 40 bp, Synthetic construct
  source 1..40: mol_type = other DNA
agggcgctgttatctgataaggcttatctggtctcatttt

SEQ ID NO: 43 - DNA, 36 bp, Synthetic construct
  source 1..36: mol_type = other DNA
agggcgctgttatctgataaggcttatctggtctca

SEQ ID NO: 44 - DNA, 33 bp, Synthetic construct
  source 1..33: mol_type = other DNA
gcgctgttatctgataaggcttatctggtctca

SEQ ID NO: 45 - DNA, 27 bp, Synthetic construct
  source 1..27: mol_type = other DNA
tgttatctgataaggcttatctggtct

SEQ ID NO: 46 - DNA, 28 bp, Synthetic construct
  source 1..28: mol_type = other DNA
tgttatctgataaggcttatctggtctc

SEQ ID NO: 47 - DNA, 29 bp, Synthetic construct
  source 1..29: mol_type = other DNA
tgttatctgataaggcttatctggtctca

SEQ ID NO: 48 - DNA, 30 bp, Synthetic construct
  source 1..30: mol_type = other DNA
ctgttatctgataaggcttatctggtctca

SEQ ID NO: 49 - DNA, 31 bp, Synthetic construct
  source 1..31: mol_type = other DNA
gctgttatctgataaggcttatctggtctca

SEQ ID NO: 50 - DNA, 32 bp, Synthetic construct
  source 1..32: mol_type = other DNA
cgctgttatctgataaggcttatctggtctca

SEQ ID NO: 51 - DNA, 27 bp, Synthetic construct
  source 1..27: mol_type = other DNA
tgttatctgataaggcttatctggtct